(19) United States
(12) Patent Application Publication
(10) Pub. No.: US 2025/0177443 A1
(43) Pub. Date: Jun. 5, 2025

(54) T CELL RECEPTORS DIRECTED AGAINST JCHAIN AND USES THEREOF

(71) Applicant: ACADEMISCH ZIEKENHUIS LEIDEN (H.O.D.N. LUMC), Leiden (NL)

(72) Inventors: Mirjam H.M. HEEMSKERK, Leiden (NL); J.H. Frederik FALKENBURG, Leiden (NL)

(21) Appl. No.: 18/839,333

(22) PCT Filed: Feb. 16, 2023
(86) PCT No.: PCT/NL2023/050073

§ 371 Date: Aug. 16, 2024

(30) Foreign Application Priority Data
Feb. 17, 2022 (NL) 2030990

(51) Int. Cl. A61K 35/17; A61K 40/11; A61K 40/32; A61P 35/02; C07K 14/725; C07K 16/28; C07K 16/42; C12N 5/0783

(52) U.S. Cl. CPC A61K 35/17; A61K 40/11; A61K 40/32; A61P 35/02; C07K 14/7051; C07K 16/2833; C07K 16/4283; C12N 5/0636; A61K 2239/48; C07K 2317/32; C07K 2317/34; C07K 2317/565; C07K 2319/03; C12N 2510/00

(57) ABSTRACT

Novel nucleic acid compositions, vector systems, modified cells, isolated peptides, isolated nucleic acid sequences and pharmaceutical compositions that encode or express T cell receptor components directed against Jchain are provided herein. These novel components may be used to enhance an immune response in a subject diagnosed with a B cell associated disease or condition. Associated methods for treating such subjects are also provided herein.

Novel nucleic acid compositions, vector systems, modified cells, isolated peptides, isolated nucleic acid sequences and pharmaceutical compositions that encode or express T cell receptor components directed against Jchain are provided herein. These novel components may be used to enhance an immune response in a subject diagnosed with a B cell associated disease or condition. Associated methods for treating such subjects are also provided herein.

### BACKGROUND

Haematological malignancies are cancers that affect the cellular components of blood or immune system. The cancer may begin in blood-forming tissue (e.g. bone marrow), or in the cells of the immune system. Examples of haematological malignancies include forms or leukemia, lymphoma and multiple myeloma (MM).

Multiple myeloma is a malignancy of the bone marrow which is characterized by uncontrolled expansion of malignant plasma cells. Advances in treatment options have extended survival of MM patients, however curative therapies are lacking and are urgently needed(1). Impressive initial response rates to CD19 chimeric antigen receptor (CAR) T cell therapy for acute lymphoblastic leukemia (ALL) and B cell lymphoma demonstrate great promise for engineered T cell therapy for B cell malignancies. Despite the high response rates, antigen escape variants of the tumors may occur. Because CD19 is not expressed by MM, B cell maturation antigen (BCMA) targeting CAR T cells were investigated. BCMA is expressed in a subset of memory B cells and in plasma cells as well as MM. BCMA CAR T cells were effective and safe but long-term complete responses were rare(2-4). Relapses often resulted from heterogenous BCMA expression within tumors, which frequently led to outgrowth of antigen low or negative MM cells (5,6).

To date, allogeneic stem cell transplantation (allo-SCT) has been the only curative therapy for MM but is decreasingly performed due to high toxicity risks. Allo-SCT effectiveness has been attributed to polyclonal T cell responses simultaneously targeting multiple antigens thereby preventing tumor antigen escape. The curative potential of allo-SCT demonstrated that multi-antigen targeting T cell responses can be a curative treatment option for MM. The recent observation that single-antigen-targeting BCMA CAR T cells were insufficient to eradicate all MM cells strengthens the belief that multi-antigen therapy should be developed to prevent antigen escape of heterogenous MM tumors(7).

To generate multi-antigen T cell therapy for MM, novel target antigens need to be identified and validated. To extend CAR T therapy options for MM, alternative CAR targets like SLAMF7 and GPRC5D are currently being explored. However, applicability of these antigens needs to be assessed as the SLAMF7 expression profile causes concerns regarding on-target off-tumor toxicity(8). GPRC5D expression profiles implicate less risk for on-target off-tumor toxicity but efficacy of GPRC5D targeting remains to be determined(9). The requirement for CAR targets to be present on the cell surface largely restricts discovery of new CAR target antigens and thus CAR therapy alone might not result in safe and curative T cell therapy.

In addition to CAR T cells, T cell receptor (TCR) engineered T cells could be of major value since TCRs can recognize peptides derived from any antigen presented in human leukocyte antigen (HLA) molecules on the surface of target cells and therefore an additional pool of MM antigens derived from intracellular proteins can be targeted. Previously, TCRs targeting peptides from the MM associated transcription factor BOB1 presented in HLA-B*07:02 and HLA-B*35:01 have been identified(10, 11). T cells engineered to express BOB1 specific TCRs induced potent lysis of MM cells in vitro. BOB1 targeting is a promising therapy for MM, as the BOB1 protein has been implicated to be a critical factor in MM cell survival and antigen escape is therefore unlikely.

Furthermore, efficacy of TCR engineered T cells for MM was investigated in trials using TCR engineered T cells targeting NY-ESO antigen presented in HLA-A2. T cells were administered after autologous-stem cell transplantation, which resulted in short-term anti-tumor response in 80% of individuals (12, 13). While efficacy should be further investigated in randomized control trials, these initial results demonstrated that further improvements need to be made since antigen escape variants frequently developed. Novel TCRs with different HLA restrictions as well as TCRs targeting multiple antigens will be needed in order to generate multi-antigen targeting therapies for all individuals.

As is clear from the above, there is a need for novel immunotherapies for treating haematological malignancies, including B cell associated haematological malignancies, such as multiple myeloma.

Although most of the disease burden is caused by infiltration of the malignant cells, the immunoglobulins produced by the B-cell malignancies and MM can give rise to severe organ damage which may even be lethal by affecting critical organs like heart, kidney, blood cells or cells from the neural system. This organ damage may be caused by antigen specific targeting of the immunoglobulins or precipitation of immune complexes. This organ damaging production of aberrant immunoglobulins may also be caused by pre-malignant or non-malignant counterparts of these disorders. Such disorders may be categorized as autoimmune disorders. In these cases specific targeting of the damaging immunoglobulin producing cells is necessary and sufficient. Several disorders are caused by specific immunoglobulin subsets including IgA or IgM antibodies.

### BRIEF SUMMARY OF THE DISCLOSURE

The inventors recently identified the joining chain (Jchain) as a potential target antigen for immunotherapy of B cell associated diseases or conditions, particularly MM, as it was highly expressed in patient MM samples but not in healthy tissues of non-B cell origins(11). Jchain is a 159 amino acid long protein that links monomers of multimeric IgA and IgM when secreted by plasma cells, but Jchain expression in plasma cells is not exclusive for IgA and IgM secreting plasma cells(14, 15). Additionally, Jchain facilitates transport of dimeric IgA and pentameric IgM across mucosal barriers, poly-Ig receptor (plgR) binds the Jchain on the basolateral side of the membrane, after which transcytosis occurs and the complexes are secreted on the luminal side(16).

The presence of Jchain in MM has previously been reported in multiple studies. Jchain expression was maintained with disease progression even when immunoglobulin production was lost(17). Additionally, Jchain was observed in MM independent of the immunoglobulin isotype produced(18). These observations suggest that the Jchain could be a promising antigen for T cell-based targeting of MM. However, as Jchain is located intracellularly, TCR mediated targeting of Jchain derived peptides presented in HLA on the surface of target cells is required for it to be a bonafide antigen for T cell based targeting of MM.

By performing epitope discovery experiments as described herein, the inventors have now identified several Jchain derived antigens that are presented on MM cells in HLA-A*01:01 (HLA-A1), HLA-A*02:01 (HLA-A2), HLA-A*03:01 (HLA-A3), HLA-A*11:01 (HLA-A11), HLA-A*24:02 (HLA-A24), and HLA-B*40:01 (HLA-B40). Specifically, the inventors identified the Jchain derived peptides YTAVVPLVY (SEQ ID NO: 99), TAVVPLVY (SEQ ID NO: 102), VLAVFIKAVHV (SEQ ID NO: 103), VLAVFIKAV (SEQ ID NO: 104), YTAVVPLV (SEQ ID NO: 105), ISDPTSPLRTR (SEQ ID NO: 101), RIIVPLNNR (SEQ ID NO: 106), CYTAVVPLV (SEQ ID NO: 100) and GETKMVETAL (SEQ ID NO: 107).

The inventors identified that Jchain derived peptides YTAVVPLVY (SEQ ID NO: 99) and TAVVPLVY (SEQ ID NO: 102) are capable of being presented by HLA-A*01:01. Additionally, the inventors identified that Jchain derived peptides VLAVFIKAVHV (SEQ ID NO:103), VLAVFIKAV (SEQ ID NO: 104) and YTAVVPLV (SEQ ID NO: 105) are capable of being presented by HLA-A*02:01. The inventors further identified that Jchain derived peptide ISDPTSPLRTR (SEQ ID NO: 101) is capable of being presented by HLA-A*03:01 and/or HLA-A*11:01, and that the Jchain derived peptide RIIVPLNNR (SEQ ID NO: 106) is capable of being presented by HLA-A*11:01. The inventors further identified that Jchain derived peptide CYTAVVPLV (SEQ ID NO: 100) is capable of being presented by HLA-A*24:02, and that Jchain derived peptide GETKMVETAL (SEQ ID NO: 107) is capable of being presented by HLA-B*40:01.

Advantageously, the above mentioned peptides (e.g. SEQ ID NO:s 99 to 107) can be used as therapeutic agents (e.g. vaccines) to treat or prevent B cell associated diseases or conditions (as described elsewhere herein). The peptides themselves therefore have utility e.g. in isolated form, or when formulated as a pharmaceutical composition. Alternatively, said peptides can be used as a target antigen for treatment of such patients with modified cells described herein (e.g. peripheral blood lymphocytes or tumour-infiltrating lymphocytes (TILs)) having T cell receptors or other binding proteins that specifically recognize one of the specified peptides, for example T cell receptors that specifically recognize one of the specified peptides in the context of a specific HLA as described herein). Further examples of the utility of the peptides described herein are provided in detail elsewhere herein.

Using the methods described herein, the inventors successfully isolated T cells with Jchain specific recognition of these Jchain peptides when presented in HLA-A1, HLA-A3, HLA-A11 or HLA-A24 (as appropriate). Advantageously, upon sequencing and transfer of TCRs from selected T cell clones, in vitro as well as in vivo killing of MM was demonstrated without off-target effects.

The inventors have identified several TCRs that bind to a specific Jchain peptide presented in HLA-A1, HLA-A3, HLA-A11 and/or HLA-A24. Specifically, the inventors identified the following Jchain-specific TCR clones:
(i) TCR clone 4G8.8 which interacts with YTAVVPLVY (SEQ ID NO: 99) in the context of HLA-A*01:01;
(ii) TCR clone 5D12.9 which interacts with YTAVVPLVY (SEQ ID NO: 99) in the context of HLA-A*01:01;
(iii) TCR clone 13F6.6 which interacts with YTAVVPLVY (SEQ ID NO: 99) in the context of HLA-A*01:01;
(iv) TCR clone 10H11.11 which interacts with CYTAVVPLV (SEQ ID NO: 100) in the context of HLA-A*24:02;
(v) TCR clone TCR 5C8.16 which interacts with ISDPTSPLRTR (SEQ ID NO: 101) in the context of HLA-A*03:01;
(vi) TCR clone 16C7.9 which interacts with ISDPTSPLRTR (SEQ ID NO: 101) in the context of HLA-A*11:01; and
(vii) TCR clone 13D4.9 which interacts with ISDPTSPLRTR (SEQ ID NO: 101) in the context of HLA-A*11:01.

Accordingly, provided herein are isolated nucleic acid compositions encoding Jchain antigen-specific binding proteins (as well as corresponding vector systems, modified cells, pharmaceutical compositions etc). The TCR components of clones (i) to (vii) form the basis of the Jchain antigen-specific binding proteins of the invention (e.g. the isolated nucleic acid compositions encoding Jchain antigen-specific binding proteins of the invention) and are described in more detail elsewhere herein.

Advantageously, cells expressing the Jchain antigen-specific binding proteins described herein can be used as an effective immunotherapy in the treatment of B cell associated diseases or conditions, such as MM. Furthermore, they may be used in the treatment of B cell associated autoimmune diseases, as described in more detail below.

The invention therefore provides an isolated nucleic acid composition that encodes a Jchain antigen-specific binding protein having a TCR a chain variable (Vα) domain and a TCR β chain variable (Vβ) domain, the composition comprising:
(a) a nucleic acid sequence that encodes a TCR Vα domain comprising a CDR3 amino acid sequence; and
(b) a nucleic acid sequence that encodes a TCR Vβ domain comprising a CDR3 amino acid sequence;
wherein the CDR3 amino acid sequences of (a) and (b) together specifically bind to Jchain.

Suitably, the Jchain antigen may comprise an amino acid sequence selected from the group consisting of: YTAVVPLVY (SEQ ID NO:99), CYTAVVPLV (SEQ ID NO:100), ISDPTSPLRTR (SEQ ID NO:101), TAVVPLVY (SEQ ID NO:102), VLAVFIKAVHV (SEQ ID NO:103), VLAVFIKAV (SEQ ID NO:104), YTAVVPLV (SEQ ID NO:105), RIIVPLNNR (SEQ ID NO:106), and GETKMVETAL (SEQ ID NO:107).

Suitably, the Jchain antigen may comprise an amino acid sequence selected from the group consisting of: YTAVVPLVY (SEQ ID NO:99), CYTAVVPLV (SEQ ID NO:100), and ISDPTSPLRTR (SEQ ID NO:101).

Suitably, the encoded binding protein may be capable of specifically binding to a peptide:HLA complex selected from the group consisting of: a YTAVVPLVY:HLA-A*01:01 complex; a CYTAVVPLV:HLA-A*24:02 complex; a ISDPTSPLRTR:HLA-A*03:01 complex; a ISDPTSPLRTR:HLA-A*11:01 complex; a TAVVPLVY:HLA-A*01:01 complex; a VLAVFIKAVHV:HLA-A*02:01 complex; a VLAVFIKAV:HLA-A*02:01 complex; a YTAVVPLV:HLA-A*02:01 complex; a RIIVPLNNR:HLA-A*11:01 complex; and a GETKMVETAL:HLA-B*40:01 complex.

Suitably, the peptide:HLA complex may be selected from the group consisting of: a YTAVVPLVY:HLA-A*01:01 complex; a CYTAVVPLV:HLA-A*24:02 complex; a ISDPTSPLRTR:HLA-A*03:01 complex; and a ISDPTSPLRTR:HLA-A*11:01 complex.

Suitably, the composition may comprise:
(i) a nucleic acid sequence that encodes a TCR Vα domain comprising a CDR3 amino acid sequence having at least 80% sequence identity to SEQ ID NO:17, or a functional fragment thereof; and a nucleic acid sequence that encodes a TCR Vβ domain comprising a CDR3 amino acid sequence having at least 80% sequence identity to SEQ ID NO: 20, or a functional fragment thereof; or
(ii) a nucleic acid sequence that encodes a TCR Vα domain comprising a CDR3 amino acid sequence having at least 80% sequence identity to SEQ ID NO:87, or a functional fragment thereof; and a nucleic acid sequence that encodes a TCR Vβ domain comprising a CDR3 amino acid sequence having at least 80% sequence identity to SEQ ID NO: 90, or a functional fragment thereof; or
(iii) a nucleic acid sequence that encodes a TCR Vα domain comprising a CDR3 amino acid sequence having at least 80% sequence identity to SEQ ID NO:3, or a functional fragment thereof; and a nucleic acid sequence that encodes a TCR Vβ domain comprising a CDR3 amino acid sequence having at least 80% sequence identity to SEQ ID NO:6, or a functional fragment thereof.

Suitably:
(i) the CDR3 of the Vα domain may comprise or consist of the amino acid sequence of SEQ ID NO: 17, and the CDR3 of the Vβ domain may comprise or consist of the amino acid sequence of SEQ ID NO:20: or
(ii) the CDR3 of the Vα domain may comprise or consist of the amino acid sequence of SEQ ID NO: 87, and the CDR3 of the Vβ domain may comprise or consist of the amino acid sequence of SEQ ID NO:90; or
(iii) the CDR3 of the Vα domain may comprise or consist of the amino acid sequence of SEQ ID NO: 3, and the CDR3 of the Vβ domain may comprise or consist of the amino acid sequence of SEQ ID NO:6.

Suitably:
(i) the Vα domain may comprise an amino acid sequence having at least 80% sequence identity to, comprising, or consisting of, SEQ ID NO: 21; and (ii) the Vβ domain may comprise an amino acid sequence having at least 80% sequence identity to, comprising, or consisting of, SEQ ID NO: 23; or
(ii) the Vα domain may comprise an amino acid sequence having at least 80% sequence identity to, comprising, or consisting of, SEQ ID NO: 91; and (ii) the Vβ domain may comprise an amino acid sequence having at least 80% sequence identity to, comprising, or consisting of, SEQ ID NO: 93; or
(iii) the Vα domain may comprise an amino acid sequence having at least 80% sequence identity to, comprising, or consisting of, SEQ ID NO: 7; and (ii) the Vβ domain may comprise an amino acid sequence having at least 80% sequence identity to, comprising, or consisting of, SEQ ID NO: 9.

Suitably, the composition may comprise a nucleic acid sequence that encodes a TCR Vα domain comprising a CDR3 amino acid sequence having at least 80% sequence identity to SEQ ID NO:31, or a functional fragment thereof; and a nucleic acid sequence that encodes a TCR Vβ domain comprising a CDR3 amino acid sequence having at least 80% sequence identity to SEQ ID NO:34, or a functional fragment thereof.

Suitably, the CDR3 of the Vα domain may comprise or consist of the amino acid sequence of SEQ ID NO: 31, and the CDR3 of the Vβ domain may comprise or consist of the amino acid sequence of SEQ ID NO:34.

Suitably, the Vα domain may comprise an amino acid sequence having at least 80% sequence identity to, comprising, or consisting of, SEQ ID NO: 35; and (ii) the Vβ domain may comprise an amino acid sequence having at least 80% sequence identity to, comprising, or consisting of, SEQ ID NO: 37.

Suitably, the composition may comprise:
(i) a nucleic acid sequence that encodes a TCR Vα domain comprising a CDR3 amino acid sequence having at least 80% sequence identity to SEQ ID NO:45, or a functional fragment thereof; and a nucleic acid sequence that encodes a TCR Vβ domain comprising a CDR3 amino acid sequence having at least 80% sequence identity to SEQ ID NO:48, or a functional fragment thereof; or
(ii) a nucleic acid sequence that encodes a TCR Vα domain comprising a CDR3 amino acid sequence having at least 80% sequence identity to SEQ ID NO:59, or a functional fragment thereof; and a nucleic acid sequence that encodes a TCR Vβ domain comprising a CDR3 amino acid sequence having at least 80% sequence identity to SEQ ID NO: 62, or a functional fragment thereof; or
(iii) a nucleic acid sequence that encodes a TCR Vα domain comprising a CDR3 amino acid sequence having at least 80% sequence identity to SEQ ID NO:73, or a functional fragment thereof; and a nucleic acid sequence that encodes a TCR Vβ domain comprising a CDR3 amino acid sequence having at least 80% sequence identity to SEQ ID NO: 76, or a functional fragment thereof.

Suitably:
(i) the CDR3 of the Vα domain may comprise or consist of the amino acid sequence of SEQ ID NO: 45, and the CDR3 of the Vβ domain may comprise or consist of the amino acid sequence of SEQ ID NO:48; or
(ii) the CDR3 of the Vα domain may comprise or consist of the amino acid sequence of SEQ ID NO: 59, and the CDR3 of the Vβ domain may comprise or consist of the amino acid sequence of SEQ ID NO:62; or
(iii) the CDR3 of the Vα domain may comprise or consist of the amino acid sequence of SEQ ID NO: 73, and the CDR3 of the Vβ domain may comprise or consist of the amino acid sequence of SEQ ID NO:76.

Suitably:
(i) the Vα domain may comprise an amino acid sequence having at least 80% sequence identity to, comprising, or consisting of, SEQ ID NO: 49; and (ii) the Vβ domain may comprise an amino acid sequence having at least 80% sequence identity to, comprising, or consisting of, SEQ ID NO: 51; or
(ii) the Vα domain may comprise an amino acid sequence having at least 80% sequence identity to, comprising, or consisting of, SEQ ID NO: 63; and (ii) the VB domain may comprise an amino acid sequence having at least 80% sequence identity to, comprising, or consisting of, SEQ ID NO: 65; or
(iii) the Vα domain may comprise an amino acid sequence having at least 80% sequence identity to, comprising, or consisting of, SEQ ID NO: 77; and (ii) the Vβ domain may comprise an amino acid sequence having at least 80% sequence identity to, comprising, or consisting of, SEQ ID NO: 79.

Suitably, the nucleic acid sequence may be codon optimised for expression in a host cell, optionally wherein the host cell is a human cell.

Suitably, the nucleic acid composition may further comprise a TCR a chain constant domain and/or a TCR β chain constant domain.

Suitably, the encoded binding protein may comprise a TCR, an antigen binding fragment of a TCR, a chimeric antigen receptor (CAR), or an ImmTAC.

Suitably, the antigen binding fragment of a TCR may be a single chain TCR (scTCR) or a chimeric TCR dimer in which the antigen binding fragment of the TCR is linked to an alternative transmembrane and intracellular signalling domain.

The invention also provides a vector system comprising a nucleic acid composition as described herein.

Suitably, the vector may be a plasmid, a viral vector, or a cosmid, optionally wherein the vector is selected from the group consisting of a retrovirus, lentivirus, adeno-associated virus, adenovirus, vaccinia virus, canary poxvirus, herpes virus, minicircle vector and synthetic DNA or RNA.

The invention also provides a modified cell comprising a nucleic acid composition as described herein, or a vector system as described herein.

Suitably, the modified cell may be selected from the group consisting of a CD8 T cell, a CD4 T cell, an NK cell, an NK-T cell, a gamma-delta T cell, a hematopoietic stem cell, an inducible pluripotent stem cell, a progenitor cell, a T cell line and a NK-92 cell line.

Suitably, the modified cell may be a human cell.

The invention also provides an isolated peptide comprising or consisting of an amino acid sequence selected from the group consisting of: YTAVVPLVY (SEQ ID NO:99), CYTAVVPLV (SEQ ID NO:100), ISDPTSPLRTR (SEQ ID NO:101), TAVVPLVY (SEQ ID NO:102), VLAVFIKAVHV (SEQ ID NO:103), VLAVFIKAV (SEQ ID NO:104), YTAVVPLV (SEQ ID NO:105), RIIVPLNNR (SEQ ID NO:106), and GETKMVETAL (SEQ ID NO:107).

Suitably, the peptide may be no more than 20 amino acids.

The invention also provides an isolated nucleic acid sequence encoding the peptide described herein. The invention also provides a vector system comprising the nucleic acid sequence.

The invention also provides a pharmaceutical composition comprising a nucleic acid composition as described herein, a vector system as described herein, a modified cell as described herein, an isolated peptide as described herein, or a nucleic acid sequence as described herein, and a pharmaceutically acceptable excipient, adjuvant, diluent and/or carrier.

Suitably, the composition comprises an isolated peptide as described herein, a nucleic acid sequence as described herein on, or a vector system as described herein, wherein the pharmaceutical composition is formulated as a vaccine.

The invention also provides a pharmaceutical composition as described herein for use in inducing or enhancing an immune response in human subject diagnosed with a B cell associated disease or condition.

The invention also provides a pharmaceutical composition as described herein for use in stimulating a cell mediated immune response to a target cell population or tissue in a human subject.

The invention also provides a pharmaceutical composition as described herein for use in providing anti-tumor immunity to a human subject.

The invention also provides a pharmaceutical composition as described herein for use in treating an human subject having a disease or condition associated with an elevated level of HLA-restricted Jchain antigen.

Suitably, the human subject may have at least one tumor.

Suitably, the subject may have been diagnosed with a B cell associated disease or condition. Suitably, the B cell associated disease or condition may be a hematological malignancy or an autoimmune disease or disorder.

Suitably, the hematological malignancy may be selected from the group consisting of: Multiple myeloma, plasma cell leukemia, (AL) Amyloidosis, Acute lymphoblastoid leukemia (ALL), Chronic lymphocytic leukemia (CLL), Waldenstrom macroglobulinemia and B cell lymphoma, optionally wherein the B cell lymphoma is selected from the group consisting of: Diffuse large B cell lymphoma (DLBCL), High grade B cell lymphoma, Mantel cell lymphoma (MCL), Follicular lymphoma (FL), and Burkitt Lymphoma.

Suitably, the hematological malignancy may be multiple myeloma.

Suitably, the autoimmune disease or disorder may be selected from the group consisting of: Rheumatoid arthritis, Multiple sclerosis, Vasculitis including Urticarial vasculitis, systemic vasculitis, renal vasculitis, Systemic lupus erythematosus (SLE), Autoimmune hemolytic anemia and Thrombocytopenia.

The invention also provides a method of generating a binding protein that is capable of specifically binding to a peptide containing a Jchain antigen and does not bind to a peptide that does not contain the Jchain antigen, comprising contacting a nucleic acid composition as described herein with a cell under conditions in which the nucleic acid composition is incorporated and expressed by the cell.

Suitably, the method may be ex vivo.

The invention also provides an isolated nucleic acid sequence comprising or consisting of the nucleotide sequence of any one of SEQ ID NOs: 8, 10, 12, 14, 22, 24, 26, 28, 36, 38, 40, 42, 50, 52, 54, 56, 64, 66, 68, 70, 78, 80, 82, 84, 92, 94, 96 or 98.

The invention also provides an isolated nucleic acid sequence comprising or consisting of the nucleotide sequence of any one of SEQ ID NOs: 8, 10, 12, 14, 22, 24, 26, 28, 36, 38, 40, 42, 50, 52, 54, 56, 64, 66, 68, 70, 78, 80, 82, 84, 92, 94, 96 or 98 for use in therapy.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows Jchain specific T cell clones recognizing peptide loaded as well as endogenously processed and presented peptides in HLA-A1, A24, A3 and A11. A) Recognition of endogenously processed and presented antigen by Jchain specific T cells clones after stimulation with target K562 cells separately transduced with target HLA alleles (+HLA) without or with additional transduction of the Jchain gene (+Jchain). B) IFN-y production by T cell clones overnight stimulated with antigen negative K562 cells transduced with target HLA molecules HLA-A1, A24, A3 and A11 loaded with decreasing concentration of Jchain peptides. Graphs are separated based on peptide-HLA specificities. Data obtained in the same experiment as FIG. 1A.

FIG. 2 shows an investigation of cross-reactivity by Jchain specific T cell clones. IFN-y production by T cell clones measured by ELISA after overnight co-culture, technical duplicates are depicted. A) Cross-reactivity with peptides presented in other HLA molecules than the targeted allele was investigated by stimulating T-cell clones with an EBV-LCL panel containing EBV-LCLs expressing HLA-I alleles with an allele frequency over 1% that do not express target HLA alleles. Target gene and HLA transduced K562 cells were included as positive control for T-cell function. Graphs are separated based on peptide-HLA specificities. B) To investigate cross-reactivity with peptides other than the targeted peptide presented in the target HLA alleles, T cell clones were stimulated with a panel of cell lines of non-B cell origins transduced with target HLA molecules.

FIG. 3 shows killing of MM cell lines by Jchain specific T cell clones. A) Killing of Jchain expressing U266 MM cells transduced with HLA-A1 or A24 by Jchain specific T cell clones in a 6-hour 51CR release assay. Killing assays were performed using E:T ratios 10:1 and 1:1. Average values of technical triplicates. B) IFN-y production after overnight co-culture using the same target cells as in A. Technical duplicates are shown. C,D) Experiments as in A and B performed with WT and HLA-A11 transduced U266 cells by Jchain HLA-A3 and A11 specific T cell clones. Data from A and B as well as C and D was obtained in the same experiments.

FIG. 4 shows functionality of Jchain TCRs in CD8 and CD4 T cells versus parental T cell clones. A-C) Jchain A1, A24, A3 and A11 restricted TCRs were sequenced and introduced, with murine constant beta domains (mTCR), in CD4 and CD8 T cells. After mTCR enrichment functionality was assessed alongside parental T cell clones. A) T cells were separately stained with Jchain pHLA-tetramers and analyzed by FACS. Cells were gated on living cells (parental T cell clones) and additionally on mTCR+ (TCR T cells). CMV TCR Td T cells were included as negative controls. B) Peptide titration experiments using parental T cell clones and CD8 and CD4 T cells Td with Jchain TCRs. Experiment performed as in FIG. 1B, peptides were diluted in a 10-fold instead of a 3-fold dilution series. C) Endogenous recognition of Jchain and HLA Td K562 cells using same T cells as in FIG. 4B).

FIG. 5 shows recognition of healthy hematopoietic and non-hematopoietic subsets by Jchain TCR Td CD8 T cells. A) IFN-y production after overnight co-culture of Jchain TCR Td CD8 T cells with CD40L activated B cells, immature dendritic cells, mature dendritic cells, PHA activated T cells and keratinocytes and fibroblasts pre-treated for 48h with 1001U/ml IFN-y. Symbols represent the average value (technical duplicate) of target cells isolated from different donors. Target cells not expressing the relevant HLA restriction allele are shown as depicted in the key accompanying the figure, cells expressing the HLA alleles are also shown as depicted in the key accompanying the figure. Per panel T cells Td with one of the Jchain TCRs are shown as indicated in the graph titles. K562+HLA and peptide loaded K562+HLA are included as negative and positive controls. B-C) FACS based killing experiment of CD40L activated peripheral blood B cells with Jchain A1 and Jchain A24 TCR Td T cells in an E:T ratio of 3:1, samples were measured using fixed acquisition times and fixed flow rates. B) Example of B cell survival and killing after overnight co-culture of HLA-A1^{neg}/HLA-A24^{pos} B cells with CMV TCR CD8 T cells (left), Jchain A1 TCR T cells (middle) and Jchain A24 TCR T cells (right). Gated on sytox-, single cells, CD3-, CD19+. C) Percentage surviving B cells isolated from different donors. The percentage of surviving cells was calculated relative to the average number of surviving cell in the negative control CMV TCR T cells.

FIG. 6 shows killing of patient derived bone marrow MM samples. Killing of patient derived bone marrow MM samples was assessed by FACS-based cytotoxicity experiments in which TCR Td T cells were co-cultured with MM patient BM samples in an E:T ratio 3:1. MM cell survival was analyzed after overnight co-culture. A) Example of MM phenotype and survival of an HLA-A1^{pos}/A24^{pos} patient sample after co-culture with CD8 T cells transduced with a CMV (negative control), Jchain HLA-A1 or Jchain HLA-A24 restricted TCR. In the highlighted boxes the MM cells are displayed, which were gated on: live cells 4 single cells→CD3 negative cells to exclude co-cultured T cells→CD45 negative, CD19 negative 4 CD56 positive, CD38 positive. MM cells were backgated on total CD3 negative cells which are displayed in the dot plots outside the highlighted boxes. B,C) Three letter code in MM titles represent different patients, additionally expression of target HLA molecules and Jchain expression as a fold increase relative to HKG is displayed. Jchain expression was measured by qPCR on sorted MM cells. B) Jchain HLA-A1 and HLA-A24 TCR Td T CD8 T cells co-cultured with MM patient samples from different individuals expressing HLA-A1, A24 or both. Numbers of surviving MM cells acquired per 2500 counting beads are displayed C) Jchain HLA-A3 and HLA-A11 TCR Td T CD8 T cells co-cultured with MM patient sample from different individuals expressing HLA-A3 or A11.

FIG. 7 shows in vivo antitumor efficacy of Jchain HLA-A1, A24, A3 and All restricted TCR transduced CD8 T cells. A) NSG mice engrafted with 2×10⁶ U266 multiple myeloma cells transduced with luciferase and HLA-A1, All or A24, were i.v. injected with 3-6×10⁶ TCR transduced CD8 T cells after 21 days. CD8 T cells were separately transduced with Jchain restricted TCRs 5D12.9 (A1), 5C8.16 (A3), 16C7.9 (A11), 10H11.11 (A24) or control CMV (pp 65-NLV-HLA-A2) TCR and enriched for mTCR expression by MACS. T cells were infused 10 days after re-stimulation. Tumor outgrowth was frequently tracked by bioluminescence imaging. B) Mean and standard deviations of tumor outgrowth (average radiance) over time on the ventral side of CMV versus Jchain TCR treated mice is shown. B) shows Jchain HLA-A1 TCR 5D12.9 treated mice, Jchain HLA-A3 TCR 5C8.16 treated mice, Jchain HLA-A11 TCR 16C7.9 treated mice and Jchain HLA-A24 TCR 10H11.11 treated mice.

FIG. 8 shows key experiments performed with a third YTA-A1 Jchain T cell clone (13F6.6) and TCR Td T cells. A) Safety panel performed as in FIG. 2, EBV-LCL panel (left) and non B-cell tumor panel (right). B) Peptide titration (top) and recognition of endogenously processed and presented antigen (bottom) as in FIG. 4 by 13F6.6 parental T cell clone, TCR Td CD8 T cells and TCR Td CD4 T cells. C) Lysis of Jchain expressing MM cell lines U266 (left) UM9 (middle) and Jchain negative K562 cells as in FIG. 3 and FIG. 13 by 13F6.6 parental T cell clone, 13F6.6 TCR Td CD8 T cells and as negative control CMV TCR Td CD8 T cells.

FIG. 9 shows a microarray data of Jchain expression measured in multiple cell subsets.

FIG. 10 shows qPCR of Jchain expression relative to house keeping genes (HKG) in non B cell lines, K562 cells, Jchain Td K562 cells (left), MM cell lines (UM9 and U266) and patient derived MM samples (right).

FIG. 11 shows reactivity of HLA-A1, A3 and A24 allo-HLA T cell clones using the non-B cell safety panels from FIG. 2B.

FIG. 12 shows killing of HLA Td Jchain negative K562 cells. Data obtained from same experiment as in FIG. 3. Jchain A1 and A24 TCR T cells in upper panels and Jchain A3 and A11 TCR T cells in lower panel. Allo HLA T cell clones included as positive controls.

FIG. 13 shows antigen specific killing of MM cells by Jchain TCR Td CD8 T cells. A-D) CD8 T cells were separately transduced with HLA-A1, A3, A11 or A24 restricted Jchain TCRs containing murine constant beta domains. T cells were enriched for mTCRbeta expression. T cells were used for 6-hour chromium release assays to study target cell lysis in E:T ratios 10:1 and 1:1. T cells were co-culture with U266 MM cells, UM9 MM cells and antigen negative K562 cells transduced with (+HLA) or naturally expressing (HLA^{pos}) target HLA molecules. Simultaneously, IFN-y production was measured after overnight co-culture. CMV TCR transduced CD8 T cells were used as a control for background lysis and cytokine production. A) Lysis and cytokine production of target cells co-cultured with Jchain HLA-A1 TCR transduced CD8 T cells. B) Lysis and cytokine production by Jchain HLA-A24 TCR transduced CD8 T cells. C) Lysis and cytokine production by Jchain HLA-A3 TCR transduced CD8 T cells. D) Lysis and cytokine production by Jchain HLA-A11 TCR transduced CD8 T cells.

FIG. 14 shows all target cells used in FIG. 5 co-cultured with Allo HLA-A1, A3 and A24 T cell clones. All target cells were included and are cells are split in different graphs based on cell type. Expression of HLA-A1, A3, All or A24 is indicated.

FIG. 15 shows Jchain A3 TCR T cells eradicate MM tumors in hIL-7/hIL-15 transgenic NSG mice. NSG mice expressing human IL-7 and IL-15 were engrafted with 2×10⁶ U266 multiple myeloma and i.v. injected with 5×10⁶ TCR transduced CD8 T cells after 14 days. CD8 T cells were transduced with Jchain restricted TCR 5C8.16 (A3) or control CMV (pp 65-NLV-HLA-A2) TCR and enriched for mTCR expression by MACS. T cells were infused 7 days after re-stimulation. Tumor outgrowth was frequently tracked by bioluminescence imaging. Mean and standard deviations of tumor outgrowth (average radiance) overtime on the ventral side is shown.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

Nucleic Acid Compositions that Encode Binding Protein Components

An isolated nucleic acid composition that encodes a Jchain antigen-specific binding protein having a TCR α chain variable (Vα) domain and a TCR β chain variable (Vβ) domain is provided herein, the composition comprising:
(a) a nucleic acid sequence that encodes a TCR Vα domain comprising a CDR3 amino acid sequence; and
(b) a nucleic acid sequence that encodes a TCR Vβ domain comprising a CDR3 amino acid sequence;
wherein the CDR3 amino acid sequences of (a) and (b) together specifically bind to Jchain.

As would be clear to a person of skill in the art, the CDR3 amino acid sequences described herein specifically bind to their target (in this case a Jchain peptide), when the target (i.e. the appropriate Jchain peptide) is presented in the context of HLA. The binding proteins (and CDR3 sequences specifically described herein) are therefore capable of specifically binding to a Jchain peptide: HLA complex. These complexes are described in more detail elsewhere herein.

Any of the permutations described below for (a) may be combined with the permutations described below for (b) (e.g. to form an appropriate nucleic acid composition that encodes a Jchain antigen-specific binding protein having a TCR α chain variable (Vα) domain and a TCR β chain variable (Vβ) domain).

The invention provides an isolated nucleic acid composition that encodes a binding protein comprising T cell receptor (TCR) components that specifically bind a Jchain antigen. The encoded binding protein is therefore capable of specifically binding to a peptide containing a Jchain antigen (e.g. a Jchain antigen comprising an amino acid sequence selected from the group consisting of: SEQ ID NO: 99 to 107) and does not bind to a peptide that does not contain a Jchain antigen (e.g. it does not bind to a peptide that does not contain Jchain antigen comprising an amino acid sequence selected from the group consisting of: SEQ ID NO: 99 to 107).

The nucleic acid composition comprises (a) a nucleic acid sequence that encodes a TCR Vα domain with the specified features described herein and (b) a nucleic acid sequence that encodes a TCR Vβ domain with the specified features described herein. The encoded TCR components form a Jchain antigen-specific binding protein.

The nucleic acid sequences of (a) and (b) above may be distinct nucleic acid sequences within the nucleic acid composition. The TCR components of the binding protein may therefore be encoded by two (or more) nucleic acid sequences (with distinct nucleotide sequences) which, together, encode all of the TCR components of the binding protein. In other words, some of the TCR components may be encoded by one nucleic acid sequence in the nucleic acid composition, and others may be encoded by another (distinct) nucleic acid sequence within the nucleic acid composition.

Alternatively, the nucleic acid sequences of (a) and (b) may be part of a single nucleic acid sequence. The TCR components of the binding protein may therefore all be encoded by a single nucleic acid sequence (for example with a single open reading frame, or with multiple (e.g. 2 or more, three or more etc.) open reading frames).

Nucleic acid sequences described herein may form part of a larger nucleic acid sequence that encodes a larger component part of a functioning binding protein. For example, a nucleic acid sequence that encodes a TCR Vα domain with the specified features described herein may be part of a larger nucleic acid sequence that encodes a functional TCR α chain (including the constant domain). As another example, a nucleic acid sequence that encodes a TCR Vβ domain with the specified features described herein may be part of a larger nucleic acid sequence that encodes a functional TCR B chain (including the constant domain). As a further example, both nucleic acid sequences (a) and (b) above may be part of a larger nucleic acid sequence that encodes a combination of a functional TCR α chain (including the constant domain) and a functional TCR β chain (including the constant domain), optionally wherein the sequence encoding the functional TCR α chain is separated from the sequence encoding the functional TCR β chain by a linker sequence that enables coordinate expression of two proteins or polypeptides in the same nucleic acid sequence. More details on this are provided below.

The nucleic acid sequences described herein may alternatively encode a small component of a T cell receptor e.g. a TCR Vα domain, or a TCR Vβ domain, only. The nucleic acid sequences may be considered as “building blocks” that provide essential components for peptide binding specificity. The nucleic acid sequences described herein may be incorporated into a distinct nucleic acid sequence (e.g. a vector) that encodes the other elements of a functional binding protein such as a TCR, such that when the nucleic acid sequence described herein is incorporated, a new nucleic acid sequence is generated that encodes e.g. a TCR α chain and/or a TCR β chain that specifically binds to a Jchain antigen (e.g. wherein the Jchain antigen comprises an amino acid sequence selected from the group consisting of: YTAVVPLVY (SEQ ID NO: 99), TAVVPLVY (SEQ ID NO: 102), VLAVFIKAVHV (SEQ ID NO: 103), VLAVFIKAV (SEQ ID NO: 104), YTAVVPLV (SEQ ID NO: 105), ISDPTSPLRTR (SEQ ID NO: 101), RIIVPLNNR (SEQ ID NO: 106), CYTAVVPLV (SEQ ID NO: 100) and GETKMVETAL (SEQ ID NO: 107)). The nucleic acid sequences described herein therefore have utility as essential components that confer binding specificity for a Jchain antigen, and thus can be used to generate a larger nucleic acid sequence encoding a binding protein with the required antigen binding activity and specificity.

The nucleic acid sequences described herein may be codon optimised for expression in a host cell, for example they may be codon optimised for expression in a human cell, such as a cell of the immune system, a inducible pluripotent stem cell (iPSC), a hematopoietic stem cell, a T cell, a primary T cell, a T cell line, a NK cell, or a natural killer T cell (Scholten et al, Clin. Immunol. 119: 135, 2006). The T cell can be a CD4+ or a CD8+ T cell. Codon optimisation is a well-known method in the art for maximizing expression of a nucleic acid sequence in a particular host cell. For instance, one or more cysteine residues may also be introduced into the encoded TCR alpha and beta chain components (e.g. to reduce the risk of mispairing with endogenous TCR chains).

In one example, the nucleic acid sequences described herein are codon optimised for expression in a suitable host cell, and/or are modified to introduce codons encoding one or more cysteine amino acids (e.g. into the constant domain of the encoded TCR alpha chain and/or the encoded TCR beta chain) to reduce the risk of mispairing with endogenous TCR chains. In one example, the nucleic acid sequences described herein are codon optimised for expression in a suitable host cell, optionally wherein the host cell is a human cell.

In certain examples, a TCR constant domain is modified to enhance pairing of desired TCR chains. For example, enhanced pairing between a heterologous TCR α chain and a heterologous TCR β chain due to a modification may result in the preferential assembly of a TCR comprising two heterologous chains over an undesired mispairing of a heterologous TCR chain with an endogenous TCR chain (see, e.g., Govers et al, Trends Mol. Med. 16(2):11 (2010)). Exemplary modifications to enhance pairing of heterologous TCR chains include the introduction of complementary cysteine residues in each of the heterologous TCR α chain and β chain.

A binding protein that is encoded by the nucleic acid compositions described herein is specific for a Jchain antigen and comprises Jchain antigen specific-TCR components. However, the encoded binding protein is not limited to being a TCR. Other appropriate binding proteins that comprise the specified Jchain antigen specific -TCR components are also encompassed. For example, the encoded binding protein may comprise a TCR, an antigen binding fragment of a TCR, a chimeric antigen receptor (CAR), or an immTAC. TCRs, antigen binding fragments thereof, CARs and ImmTACs are well defined in the art. A non-limiting example of an antigen binding fragment of a TCR is a single chain TCR (scTCR) or a chimeric dimer composed of the antigen binding fragments of the TCR α and TCR β chain linked to transmembrane and intracellular domains of a dimeric complex so that the complex is a chimeric dimer TCR (cdTCR). An ImmTAC comprises a TCR connected to an anti-CD3 antibody. ImmTACs are therefore bispecific, combining Jchain-recognizing TCR components with immune activating complexes.

In certain examples, an antigen-binding fragment of a TCR comprises a single chain TCR (scTCR), which comprises both the TCR Vα and TCR Vβ domains, but only a single TCR constant domain. In other examples, an antigen-binding fragment of a TCR comprises a chimeric TCR dimer in which the antigen binding fragment of the TCR is linked to an alternative transmembrane and intracellular signalling domain (where the alternative transmembrane and intracellular signalling domain are not naturally found in TCRs). In further examples, an antigen-binding fragment of a TCR or a chimeric antigen receptor is chimeric (e.g., comprises amino acid residues or motifs from more than one donor or species), humanized (e.g., comprises residues from a non-human organism that are altered or substituted so as to reduce the risk of immunogenicity in a human), or human.

“Chimeric antigen receptor” (CAR) refers to a fusion protein that is engineered to contain two or more naturally-occurring amino acid sequences linked together in a way that does not occur naturally or does not occur naturally in a host cell, which fusion protein can function as a receptor when present on a surface of a cell. CARs described herein include an extracellular portion comprising an antigen binding domain (i.e., obtained or derived from an immunoglobulin or immunoglobulin-like molecule, such as an scFv derived from an antibody or TCR specific for an antigen (e.g. a cancer antigen etc), or an antigen binding domain derived or obtained from a killer immunoreceptor from an NK cell) linked to a transmembrane domain and one or more intracellular signalling domains (optionally containing co-stimulatory domain(s)) (see, e.g., Sadelain et al, Cancer Discov., 3(4):388 (2013); see also Harris and Kranz, Trends Pharmacol. Sci., 37(3):220 (2016), and Stone et al, Cancer Immunol. Immunother., 63(11): 1163 (2014)).

Methods for producing engineered TCRs are described in, for example, Bowerman et al, Mol. Immunol, 5(15):3000 (2009). Methods for making CARs are well known in the art and are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; and Brentjens et al, 2007, Clin. Cancer Res. 73:5426.

The binding proteins described herein may also be expressed as part of a transgene construct that encodes additional accessory proteins, such as a safety switch protein, a tag, a selection marker, a CD8 co-receptor β-chain, α-chain or both, or any combination thereof.

A T cell receptor (TCR) is a molecule found on the surface of T cells (T lymphocytes) that is responsible for recognising a peptide that is bound to (presented by) a major histocompatibility complex (MHC) molecule on a target cell. The invention is directed to nucleic acid compositions that encode binding proteins comprising TCR components that interact with a particular peptide in the context of the appropriate serotype of MHC, i.e. a Jchain antigen in the context of HLA-A*01:01, HLA-A*02:01, HLA-A*03:01, HLA-A*11:01, HLA-A*24:02 and/or HLA-B*40:01 (in other words, the encoded binding protein is capable of specifically binding to a Jchain antigen: specific HLA complex). In an example, the invention is directed to nucleic acid compositions that encode binding proteins comprising TCR components that interact with a particular peptide in the context of the appropriate serotype of MHC, i.e. YTAVVPLVY (SEQ ID NO: 99) in the context of HLA-A*01:01; TAVVPLVY (SEQ ID NO: 102) in the context of HLA-A*01:01; VLAVFIKAVHV(SEQ ID NO: 103) in the context of HLA-A*02:01; VLAVFIKAV (SEQ ID NO: 104) in the context of HLA-A*02:01; YTAVVPLV (SEQ ID NO: 105) in the context HLA-A*02:01; ISDPTSPLRTR (SEQ ID NO: 101) in the context of HLA-A*03:01 or HLA-A*11:01; RIIVPLNNR (SEQ ID NO: 106) in the context of HLA-A*11:01; CYTAVVPLV (SEQ ID NO: 100) in the context of HLA-A*24:02; or GETKMVETAL (SEQ ID NO: 107) in the context of HLA-B*40:01.

HLA-A*02:01 is a globally common human leukocyte antigen serotype within the HLA-A serotype group. Peptides that are presented by HLA-A*02:01 to TCRs are described as being “HLA-A*02:01 restricted”.

HLA-A*03:01, HLA-A*11:01, HLA-A*01:01, HLA-A*24:02 and HLA-B*40:01 are also common human leukocyte antigen serotypes within the HLA-A and HLA-B serotype groups. Peptides that are presented by HLA-A*03:01 to TCRs are described as being “HLA-A*03:01 restricted”. Similarly, peptides that are presented by HLA-A*11:01 to TCRs are described as being “HLA-A*11:01 restricted”. Similarly, peptides that are presented by HLA-A*01:01 to TCRs are described as being “HLA-A*01:01 restricted”. Similarly, peptides that are presented by HLA-A*24:02 to TCRs are described as being “HLA-A*24:02 restricted”. Finally, peptides that are presented by HLA-A*40:01 to TCRs are described as being “HLA-A*40:01 restricted”.

HLA-A*01:01 is also referred to herein as HLA-A1. Similarly, HLA-A*02:01 is also referred to herein as HLA-A2; HLA-A*03:01 is also referred to herein as HLA-A3; HLA-A*11:01 is also referred to herein as HLA-A11; HLA-A*24:02 is also referred to herein as HLA-A24; and HLA-B*40:01 is also referred to herein as HLA-B40.

As described herein, the inventors have identified several Jchain derived peptides presented on MM cells in HLA-A*01:01 (HLA-A1), HLA-A*02:01 (HLA-A2), HLA-A*03:01 (HLA-A3), HLA-A*11:01 (HLA-A11), HLA-A*24:02 (HLA-A24), and HLA-B*40:01 (HLA-B40). Specifically, the inventors identified the Jchain derived peptides YTAVVPLVY (SEQ ID NO: 99), TAVVPLVY (SEQ ID NO: 102), VLAVFIKAVHV (SEQ ID NO: 103), VLAVFIKAV (SEQ ID NO: 104), YTAVVPLV (SEQ ID NO: 105), ISDPTSPLRTR (SEQ ID NO: 101), RIIVPLNNR (SEQ ID NO: 106), CYTAVVPLV (SEQ ID NO: 100) and GETKMVETAL (SEQ ID NO: 107).

Accordingly, the Jchain antigen specifically bound by a binding protein described herein may comprise an amino acid sequence selected from the group consisting of: YTAVVPLVY (SEQ ID NO: 99), TAVVPLVY (SEQ ID NO: 102), VLAVFIKAVHV (SEQ ID NO: 103), VLAVFIKAV (SEQ ID NO: 104), YTAVVPLV (SEQ ID NO: 105), ISDPTSPLRTR (SEQ ID NO: 101), RIIVPLNNR (SEQ ID NO: 106), CYTAVVPLV (SEQ ID NO: 100) and GETKMVETAL (SEQ ID NO: 107). In one example, the Jchain antigen specifically bound by a binding protein described herein comprises an amino acid sequence selected from the group consisting of: YTAVVPLVY (SEQ ID NO: 99), ISDPTSPLRTR (SEQ ID NO: 101) and CYTAVVPLV (SEQ ID NO: 100). The antigen may be an antigenic fragment (i.e. a portion) of an amino acid sequence selected from the group consisting of: YTAVVPLVY (SEQ ID NO: 99), TAVVPLVY (SEQ ID NO: 102), VLAVFIKAVHV (SEQ ID NO: 103), VLAVFIKAV (SEQ ID NO: 104), YTAVVPLV (SEQ ID NO: 105), ISDPTSPLRTR (SEQ ID NO: 101), RIIVPLNNR (SEQ ID NO: 106), CYTAVVPLV (SEQ ID NO: 100) and GETKMVETAL (SEQ ID NO: 107), it may consist of an amino acid sequence selected from the group consisting of: YTAVVPLVY (SEQ ID NO: 99), TAVVPLVY (SEQ ID NO: 102), VLAVFIKAVHV (SEQ ID NO: 103), VLAVFIKAV (SEQ ID NO: 104), YTAVVPLV (SEQ ID NO: 105), ISDPTSPLRTR (SEQ ID NO: 101), RIIVPLNNR (SEQ ID NO: 106), CYTAVVPLV (SEQ ID NO: 100) and GETKMVETAL (SEQ ID NO: 107), or it may comprise (i.e. include within a longer sequence) an amino acid sequence selected from the group consisting of: YTAVVPLVY (SEQ ID NO: 99), TAVVPLVY (SEQ ID NO: 102), VLAVFIKAVHV (SEQ ID NO: 103), VLAVFIKAV (SEQ ID NO: 104), YTAVVPLV (SEQ ID NO: 105), ISDPTSPLRTR (SEQ ID NO: 101), RIIVPLNNR (SEQ ID NO: 106), CYTAVVPLV (SEQ ID NO: 100) and GETKMVETAL (SEQ ID NO: 107).

Accordingly, in an example, the Jchain antigen comprises an amino acid sequence selected from the group consisting of: YTAVVPLVY (SEQ ID NO: 99), TAVVPLVY (SEQ ID NO: 102), VLAVFIKAVHV (SEQ ID NO: 103), VLAVFIKAV (SEQ ID NO: 104), YTAVVPLV (SEQ ID NO: 105), ISDPTSPLRTR (SEQ ID NO: 101), RIIVPLNNR (SEQ ID NO: 106), CYTAVVPLV (SEQ ID NO: 100) and GETKMVETAL (SEQ ID NO: 107).

In another example, the Jchain antigen comprises an amino acid sequence selected from the group consisting of: YTAVVPLVY (SEQ ID NO: 99), ISDPTSPLRTR (SEQ ID NO: 101) and CYTAVVPLV (SEQ ID NO: 100).

In one example, the Jchain antigen comprises the amino acid sequence YTAVVPLVY (SEQ ID NO: 99). In another example, the Jchain antigen comprises the amino acid sequence ISDPTSPLRTR (SEQ ID NO: 101). In a further example, the Jchain antigen comprises the amino acid sequence CYTAVVPLV (SEQ ID NO: 100).

The inventors identified that the Jchain derived peptides YTAVVPLVY (SEQ ID NO: 99) and TAVVPLVY (SEQ ID NO: 102) are capable of being presented by HLA-A*01:01; that the Jchain derived peptides VLAVFIKAVHV (SEQ ID NO: 103), VLAVFIKAV (SEQ ID NO: 104) and YTAVVPLV (SEQ ID NO: 105) are capable of being presented by HLA-A*02:01; that the Jchain derived peptide ISDPTSPLRTR (SEQ ID NO: 101) is capable of being presented by HLA-A*03:01 and HLA-A*11:01; that the Jchain derived peptide RIIVPLNNR (SEQ ID NO: 106) is capable of being presented by HLA-A*11:01; that the Jchain derived peptide CYTAVVPLV (SEQ ID NO: 100) is capable of being presented by HLA-A*24:02; and that the Jchain derived peptide GETKMVETAL (SEQ ID NO: 107) is capable of being presented by HLA-B*40:01.

Accordingly, in one example, the encoded binding protein is capable of specifically binding to a peptide:HLA complex selected from the group consisting of: a YTAVVPLVY:HLA-A*01:01 complex; a TAVVPLVY:HLA-A*01:01 complex; a VLAVFIKAVHV:HLA-A*02:01 complex; a VLAVFIKAV:HLA-A*02:01 complex; a YTAVVPLV:HLA-A*02:01 complex; a ISDPTSPLRTR:HLA-A*03:01 complex; a RIIVPLNNR:HLA-A*11:01 complex; a ISDPTSPLRTR:HLA-A*11:01 complex; a CYTAVVPLV:HLA-A*24:02 complex; and a GETKMVETAL:HLA-B*40:01 complex.

In another example, the encoded binding protein is capable of specifically binding to a peptide:HLA complex selected from the group consisting of: a YTAVVPLVY:HLA-A*01:01 complex; a ISDPTSPLRTR:HLA-A*03:01 complex; a ISDPTSPLRTR:HLA-A*11:01 complex; and a CYTAVVPLV:HLA-A*24:02 complex.

In a further example, the encoded binding protein is capable of specifically binding to a YTAVVPLVY:HLA-A*01:01 complex. In another example, the encoded binding protein is capable of specifically binding to a ISDPTSPLRTR:HLA-A*03:01 complex. In another example, the encoded binding protein is capable of specifically binding to a ISDPTSPLRTR:HLA-A*11:01 complex. In a further example, the encoded binding protein is capable of specifically binding to a CYTAVVPLV:HLA-A*24:02 complex.

In one example, the Jchain derived peptide of the peptide:HLA complex comprises an antigenic fragment of an amino acid sequence selected from the group consisting of: SEQ ID NO: 99 to 107. In a further example, the Jchain derived peptide of the peptide:HLA complex comprises or consists of an amino acid sequence selected from the group consisting of: SEQ ID NO:99 to 107.

The TCR is composed of two different polypeptide chains. In humans, 95% of TCRs consist of an alpha (α) chain and a beta (β) chain (encoded by TRA and TRB respectively). When the TCR engages with peptide in the context of HLA (e.g. in the context of HLA-A*01:01, HLA-A*02:01, HLA-A*03:01, HLA-A*11:01, HLA-A*24:02 and/or HLA-B*40:01, in particular in the context of HLA-A*01:01, HLA-A*03:01, HLA-A*11:01 and/or HLA-A*24:02 as appropriate), the T cell is activated through signal transduction.

The alpha and beta chains of the TCR are highly variable in sequence. Each chain is composed of two extracellular domains, a variable domain (V) and a constant domain (C). The constant domain is proximal to the T cell membrane followed by a transmembrane region and a short cytoplasmic tail while the variable domain binds to the peptide/HLA complex.

An isolated nucleic acid composition that encodes a Jchain antigen-specific binding protein is provided herein having a TCR α chain variable (Vα) domain and a TCR β chain variable (Vβ) domain. In one example the nucleic acid composition described herein may comprise a TCR α chain constant domain and/or a TCR β chain constant domain.

The variable domain of each chain has three hypervariable regions (also called complementarity determining regions (CDRs)). Accordingly, the TCR alpha variable domain (referred to herein as a TCR Vα domain, TCR V alpha domain, Vα domain or V alpha domain, alpha variable domain etc) comprises a CDR1, a CDR2 and CDR3 region. Similarly, the TCR beta variable domain (referred to herein as a TCR Vβ domain, TCR V beta domain, Vβ domain or V beta domain, beta variable domain etc) also comprises a (different) CDR1, CDR2, and CDR3 region. In each of the alpha and beta variable domains it is CDR3 that is mainly responsible for recognizing the peptide being presented by the HLA molecules.

As will be clear to a person of skill in the art, the phrase “TCR α chain variable domain” refers to the variable (V) domain (extracellular domain) of a TCR alpha chain, and thus includes three hypervariable regions (CDR1, CDR2 and the specified CDR3), as well as the intervening sequences, but does not include the constant (C) domain of the alpha chain, which does not form part of the variable domain.

As will be clear to a person of skill in the art, the phrase “TCR β chain variable domain” refers to the variable (V) domain (extracellular domain) of a TCR beta chain, and thus includes three hypervariable regions (CDR1, CDR2 and the specified CDR3), as well as the intervening sequences, but does not include the constant (C) domain of the beta chain, which does not form part of the variable domain.

### Components of the TCR α Chain Variable (Vα) Domain

The isolated nucleic acid composition described herein encodes a Jchain antigen-specific binding protein. As discussed herein, the inventors have identified several TCRs that specifically bind to a Jchain antigen selected from YTAVVPLVY (SEQ ID NO:99), ISDPTSPLRTR (SEQ ID NO:101) and CYTAVVPLV (SEQ ID NO:100).

(i) Vα Domains that Interact with YTAVVPLVY (SEQ ID NO: 99)

As provided elsewhere herein, the inventors identified TCR clone 4G8.8 which interacts with YTAVVPLVY (SEQ ID NO:99) in the context of HLA-A*01:01. The sequences provided herein that correspond to TCR clone 4G8.8 are SEQ ID NO:s 1 to 14.

As discussed above, an isolated nucleic acid composition that encodes a Jchain antigen-specific binding protein having a TCR α chain variable (Vα) domain and a TCR β chain variable (Vβ) domain is provided, the composition comprising: (a) a nucleic acid sequence that encodes a TCR Vα domain comprising a CDR3 amino acid sequence; and (b) a nucleic acid sequence that encodes a TCR Vβ domain comprising a CDR3 amino acid sequence; wherein the CDR3 amino acid sequences of (a) and (b) together specifically bind to Jchain.

An example of an appropriate TCR Vα domain CDR3 amino acid sequence that confers specific binding to a Jchain antigen (e.g. to YTAVVPLVY (SEQ ID NO: 99)) is shown in SEQ ID NO:3. As would be clear to a person of skill in the art, variants of the amino acid sequence shown in SEQ ID NO:3 may also be functional (i.e. retain their ability to confer specific binding to a Jchain antigen (e.g. to the peptide YTAVVPLVY (SEQ ID NO: 99)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 3, i.e. they may have at least 80%, at least 86%, at least 93%, or 100% sequence identity to SEQ ID NO: 3. Suitably, percent identity is calculated as the percentage of identity to the entire length of the reference sequence (e.g. SEQ ID NO:3). In other words, appropriate (functional) Vα domain CDR3 amino acid sequences may vary from the sequence shown in SEQ ID NO:3 by one or several (e.g. two etc) amino acids.

As stated above, functional variants of SEQ ID NO:3 retain their ability to confer specific binding to a Jchain antigen (e.g. the peptide shown in SEQ ID NO:99) when the CDR3 is part of TCR Vα domain.

Functional variants may be naturally occurring, synthetic, or synthetically improved functional variants of SEQ ID NO:3. The term “variant” also encompasses homologues and fragments. Functional variants will typically contain only conservative substitutions of one, two or more amino acids of SEQ ID NO:3, or substitution, deletion or insertion of non-critical amino acids in non-critical regions of the CDR3.

Non-functional variants are amino acid sequence variants of SEQ ID NO: 3 that do not specifically bind to a Jchain antigen (e.g. the peptide shown in SEQ ID NO:99). Non-functional variants will typically contain a non-conservative substitution, a deletion, or insertion or premature truncation of the amino acid sequence of SEQ ID NO:3 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 3. In examples where the TCR Vα domain CDR3 has the amino acid sequence of SEQ ID NO:3, the CDR3 may be encoded by any appropriate nucleic acid sequence.

The encoded TCR Vα domain may comprise, in addition to the specified CDR3, a CDR1 comprising an amino acid sequence of SEQ ID NO: 1, or a functional variant thereof (i.e. wherein the variant retains the ability to specifically bind to the Jchain antigen (e.g. the peptide shown in SEQ ID NO:99)). Such functional variants may be naturally occurring, synthetic, or synthetically improved functional variants of SEQ ID NO:1. The term “variant” also encompasses homologues and fragments. Functional variants will typically contain only conservative substitutions of one or more amino acids of SEQ ID NO:1, or substitution, deletion or insertion of non-critical amino acids in non-critical regions of the protein.

Non-functional variants are amino acid sequence variants of SEQ ID NO: 1 that do not specifically bind to a Jchain antigen (e.g. the peptide shown in SEQ ID NO:99). Non-functional variants will typically contain a non-conservative substitution, a deletion, or insertion or premature truncation of the amino acid sequence of SEQ ID NO:1 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 1, i.e. it may have at least 80%, at least 85%, or 100% sequence identity to SEQ ID NO: 1. Suitably, percent identity is calculated as the percentage of identity to the entire length of the reference sequence (e.g. SEQ ID NO:1). In other words, appropriate functional Vα domain CDR1 amino acid sequences may vary from the sequence shown in SEQ ID NO: 1 by one or several amino acids. As stated previously, the variant may comprise an amino acid substitution such as a conservative amino acid substitution compared to the sequence shown in SEQ ID NO:1. As stated above, functional variants of SEQ ID NO: 1 retain the ability to specifically bind to a Jchain antigen (e.g. the peptide shown in SEQ ID NO:99) when the CDR1 is part of TCR Vα domain).

In one example, the CDR1 of the Vα domain comprises or consists of the amino acid sequence of SEQ ID NO:1. In examples where the TCR Vα domain CDR1 has the amino acid sequence of SEQ ID NO:1, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO:2, or a functional variant thereof (i.e. wherein the variant retains the ability to specifically bind to HLA-A*01:01). Such functional variants may be naturally occurring, synthetic, or synthetically improved functional variants of SEQ ID NO:2. The term “variant” also encompasses homologues and fragments. Functional variants will typically contain only conservative substitutions of one or more amino acids of SEQ ID NO:2, or substitution, deletion or insertion of non-critical amino acids in non-critical regions of the protein.

Non-functional variants are amino acid sequence variants of SEQ ID NO: 2 that do not specifically bind to HLA-A*01:01. Non-functional variants will typically contain a non-conservative substitution, a deletion, or insertion or premature truncation of the amino acid sequence of SEQ ID NO: 2 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 2, i.e. it may have at least 80%, at least 87%, or 100% sequence identity to SEQ ID NO: 2. Suitably, percent identity is calculated as the percentage of identity to the entire length of the reference sequence (e.g. SEQ ID NO:2). In other words, appropriate (functional) Vα domain CDR2 amino acid sequences may vary from the sequence shown in SEQ ID NO:2 by one or several amino acids. As stated previously, the variant may comprise an amino acid substitution such as a conservative amino acid substitution compared to the sequence shown in SEQ ID NO:2). As stated above, a functional variant of SEQ ID NO: 2 retains the ability to specifically bind to HLA-A*01:01.

In one example, the CDR2 of the Vα domain comprises or consists of the amino acid sequence of SEQ ID NO: 2. In examples where the TCR Vα domain CDR2 has the amino acid sequence of SEQ ID NO:2, the CDR2 may be encoded by any appropriate nucleic acid sequence.

The encoded TCR Vα domain may therefore comprise the CDRs mentioned in detail above (by SEQ ID specifically i.e. SEQ ID NO:3, SEQ ID NO: 1 and SEQ ID NO: 2, or functional variants thereof), with appropriate intervening sequences between the CDRs.

The encoded TCR Vα domain may comprise an amino acid sequence of SEQ ID NO:7, or a functional variant thereof (i.e. wherein the variant TCR Vα domain retains the ability to specifically bind to a Jchain antigen (e.g. the peptide shown in SEQ ID NO:99) when part of a binding protein described herein). Such functional variants may be naturally occurring, synthetic, or synthetically improved functional variants of SEQ ID NO:7. The term “variant” also encompasses homologues and fragments. Functional variants will typically contain only conservative substitutions of one or more amino acids of SEQ ID NO:7, or substitution, deletion or insertion of non-critical amino acids in non-critical regions of the protein.

Non-functional variants are amino acid sequence variants of SEQ ID NO: 7 that do not specifically bind to a Jchain antigen (e.g. the peptide shown in SEQ ID NO:99). Non-functional variants will typically contain a non-conservative substitution, a deletion, or insertion or premature truncation of the amino acid sequence of SEQ ID NO:7 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 7, whilst retaining the ability to specifically bind to a Jchain antigen (e.g. the peptide shown in SEQ ID NO:99). In other words, a functional TCR Vα domain with one or several amino acid substitutions compared to the sequence of SEQ ID NO:7 is also encompassed. As stated previously, the amino acid substitution may be a conservative amino acid substitution. The variability in sequence compared to SEQ ID NO:7 may all be in regions of the TCR Vα domain that do not form CDRs (i.e. the variant may have the CDRs of SEQ ID NO: 3, SEQ ID NO: 1 and/or SEQ ID NO: 2, and still have 25% (or less) sequence variability compared to SEQ ID NO:7). In other words, the sequence of the CDRs of SEQ ID NO: 7 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 7).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 7, wherein the TCR Vα domain comprises a CDR3 having an amino acid sequence of SEQ ID NO: 3. In this example, the TCR Vα domain CDR1 may have an amino acid sequence of SEQ ID NO: 1 and the TCR Vα domain CDR2 may have an amino acid sequence of SEQ ID NO: 2.

As another example, the encoded TCR Vα domain may comprise an amino acid sequence having at the amino acid sequence of SEQ ID NO: 7, with 0 to 10 (or 0 to 5) amino acid substitutions, insertions or deletions), wherein the TCR Vα domain comprises a CDR3 having an amino acid sequence of SEQ ID NO: 3. In this example, the TCR Vα domain CDR1 may have an amino acid sequence of SEQ ID NO: 1 and the TCR Vα domain CDR2 may have an amino acid sequence of SEQ ID NO: 2.

In examples where the TCR Vα domain has the amino acid sequence of SEQ ID NO:7, the TCR Vα domain may be encoded by the nucleic acid sequence of SEQ ID NO:8, or a genetically degenerate sequence thereof (i.e. other nucleic acid sequences that encode the same protein as a result of the degeneracy of the genetic code).

The phrase “genetically degenerate sequence thereof” is used interchangeably with “derivative thereof” herein.

For the avoidance of doubt, the nucleic acid sequence encoding the TCR Vα domain may also encode a TCR α chain constant domain. An example of a suitable constant domain (for either a TCR α chain or a TCR β chain) is encoded in the MP71-TCR-flex retroviral vector. However, the invention is not limited to this specific constant domain, and encompasses any appropriate TCR α chain constant domain. The constant domain may be murine derived, human derived or humanised. Methods for identifying or generating appropriate constant domains are well known to a person of skill in the art and are well within their routine capabilities.

By way of example only, the constant domain may be encoded by or derived from a vector, such as a lentiviral, retroviral or plasmid vector but also adenovirus, adeno-associated virus, vaccinia virus, canary poxvirus or herpes virus vectors in which murine or human constant domains are pre-cloned. Recently, minicircles have also been described for TCR gene transfer (non-viral Sleeping Beauty transposition from minicircle vectors as published by R Monjezi, et al., 2017). Moreover, naked (synthetic) DNA/RNA can also be used to introduce the TCR. As an example, a pMSGV retroviral vector with pre-cloned TCR-Ca and Cb genes as described in LV Coren et al., BioTechniques 2015 may be used to provide an appropriate constant domain. Alternatively, single stranded or double stranded DNA or RNA can be inserted by homologous directed repair into the TCR locus (see Roth et a/2018 Nature vol 559; page 405). As a further option, non-homologous end joining is possible.

An example of a specific TCR α chain amino acid sequence that includes a TCR Vα domain described herein with an appropriate constant domain is shown in SEQ ID NO: 11. Appropriate functional variants of SEQ ID NO:11 are also encompassed (e.g. variants having at least 75% (e.g. at least 75%, at least 80%, at least 85%, at least 90%, at least 95% etc) sequence identity to the amino acid sequence of SEQ ID NO: 11, wherein the variant TCR α chain amino acid sequence retains its ability to specifically bind to a Jchain antigen (e.g. the peptide shown in SEQ ID NO:99) when part of a binding protein described herein). In other words, a functional TCR α chain with one or several amino acid substitutions compared to the sequence of SEQ ID NO:11 is also encompassed. As stated previously, the amino acid substitution may be a conservative amino acid substitution. The variability in sequence compared to SEQ ID NO:11 may all be in regions of the TCR α chain that do not form CDRs (i.e. the variant may have the CDRs of SEQ ID NO: 3, SEQ ID NO: 1 and/or SEQ ID NO: 2, and still have 25% (or less) sequence variability compared to SEQ ID NO:11). In other words, the sequence of the CDRs of SEQ ID NO: 11 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 11).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 11, wherein the TCR α chain comprises a CDR3 having an amino acid sequence of SEQ ID NO: 3. In this example, the TCR α chain CDR1 may have an amino acid sequence of SEQ ID NO:1 and the TCR α chain CDR2 may have an amino acid sequence of SEQ ID NO: 2.

In examples where the TCR α chain has the amino acid sequence of SEQ ID NO:11, the TCR α chain may be encoded by the nucleic acid sequence of SEQ ID NO:12, or a genetically degenerate sequence thereof (i.e. other nucleic acid sequences that encode the same protein as a result of the degeneracy of the genetic code). It is noted that SEQ ID NO:12 is the nucleic acid sequence for TCR α chain of clone 4G8.8.

In one example, the nucleic acid composition provided herein comprises a nucleic acid sequence that encodes a TCR Vα domain comprising a CDR3 amino acid sequence having at least 80% sequence identity to SEQ ID NO:3, or a functional fragment thereof.

In another example, the CDR3 of the Vα domain of a nucleic acid composition provided herein comprises or consists of the amino acid sequence of SEQ ID NO: 3.

In another example, the Vα domain of a nucleic acid composition provided herein comprises an amino acid sequence having at least 80% sequence identity to, comprising, or consisting of, SEQ ID NO: 7.

As provided elsewhere herein, the inventors have also identified TCR clone 5D12.9 which interacts with YTAVVPLVY (SEQ ID NO:99) in the context of HLA-A*01:01. The sequences provided herein that correspond to TCR clone 5D12.9 are SEQ ID NO:s 15 to 28.

Accordingly, another example of an appropriate TCR Vα domain CDR3 amino acid sequence that confers specific binding to a Jchain antigen (e.g. to YTAVVPLVY (SEQ ID NO: 99)) is shown in SEQ ID NO:17. As would be clear to a person of skill in the art, variants of the amino acid sequence shown in SEQ ID NO:17 may also be functional (i.e. retain their ability to confer specific binding to a Jchain antigen (e.g. to the peptide YTAVVPLVY (SEQ ID NO: 99)) when the CDR3 is part of TCR Vα domain). Such functional variants are therefore encompassed herein.

For example, appropriate (functional) Vα domain CDR3 amino acid sequences may have at least 80% sequence identity to SEQ ID NO:17, i.e. they may have at least 80%, at least 83%, at least 91%, or 100% sequence identity to SEQ ID NO:17. Suitably, percent identity is calculated as the percentage of identity to the entire length of the reference sequence (e.g. SEQ ID NO:17). In other words, appropriate (functional) Vα domain CDR3 amino acid sequences may vary from the sequence shown in SEQ ID NO:17 by one or several (e.g. two etc) amino acids.

As stated above, functional variants of SEQ ID NO:17 retain their ability to confer specific binding to a Jchain antigen (e.g. the peptide shown in SEQ ID NO:99) when the CDR3 is part of TCR Vα domain.

Functional variants may be naturally occurring, synthetic, or synthetically improved functional variants of SEQ ID NO:17. The term “variant” also encompasses homologues and fragments. Functional variants will typically contain only conservative substitutions of one, two or more amino acids of SEQ ID NO:17, or substitution, deletion or insertion of non-critical amino acids in non-critical regions of the CDR3.

Non-functional variants are amino acid sequence variants of SEQ ID NO:17 that do not specifically bind to a Jchain antigen (e.g. to the peptide shown in SEQ ID NO:99). Non-functional variants will typically contain a non-conservative substitution, a deletion, or insertion or premature truncation of the amino acid sequence of SEQ ID NO:17 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 17. In examples where the TCR Vα domain CDR3 has the amino acid sequence of SEQ ID NO:17, the CDR3 may be encoded by any appropriate nucleic acid sequence.

The encoded TCR Vα domain may comprise, in addition to the specified CDR3, a CDR1 comprising an amino acid sequence of SEQ ID NO: 15, or a functional variant thereof (i.e. wherein the variant retains the ability to specifically bind to a Jchain antigen (e.g. the peptide shown in SEQ ID NO:99)). Such functional variants may be naturally occurring, synthetic, or synthetically improved functional variants of SEQ ID NO:15. The term “variant” also encompasses homologues and fragments. Functional variants will typically contain only conservative substitutions of one or more amino acids of SEQ ID NO:15, or substitution, deletion or insertion of non-critical amino acids in non-critical regions of the protein.

Non-functional variants are amino acid sequence variants of SEQ ID NO: 15 that do not specifically bind to a Jchain antigen (e.g. the peptide shown in SEQ ID NO:99). Non-functional variants will typically contain a non-conservative substitution, a deletion, or insertion or premature truncation of the amino acid sequence of SEQ ID NO:15 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 15, i.e. it may have at least 80%, at least 87%, or 100% sequence identity to SEQ ID NO: 15. Suitably, percent identity is calculated as the percentage of identity to the entire length of the reference sequence (e.g. SEQ ID NO:15). In other words, appropriate functional Vα domain CDR1 amino acid sequences may vary from the sequence shown in SEQ ID NO: 15 by one or several amino acids. As stated previously, the variant may comprise an amino acid substitution such as a conservative amino acid substitution compared to the sequence shown in SEQ ID NO:15. As stated above, functional variants of SEQ ID NO: 15 retain the ability to specifically bind to a Jchain antigen (e.g. the peptide shown in SEQ ID NO:99) when the CDR1 is part of TCR Vα domain).

In one example, the CDR1 of the Vα domain comprises or consists of the amino acid sequence of SEQ ID NO:15. In examples where the TCR Vα domain CDR1 has the amino acid sequence of SEQ ID NO:15, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO:16, or a functional variant thereof (i.e. wherein the variant retains the ability to specifically bind to HLA-A*01:01). Such functional variants may be naturally occurring, synthetic, or synthetically improved functional variants of SEQ ID NO:16. The term “variant” also encompasses homologues and fragments. Functional variants will typically contain only conservative substitutions of one or more amino acids of SEQ ID NO:16, or substitution, deletion or insertion of non-critical amino acids in non-critical regions of the protein.

Non-functional variants are amino acid sequence variants of SEQ ID NO:16 that do not specifically bind to HLA-A*01:01. Non-functional variants will typically contain a non-conservative substitution, a deletion, or insertion or premature truncation of the amino acid sequence of SEQ ID NO:16 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO:16, i.e. it may have at least 80%, at least 83%, or 100% sequence identity to SEQ ID NO:16. Suitably, percent identity is calculated as the percentage of identity to the entire length of the reference sequence (e.g. SEQ ID NO:16). In other words, appropriate (functional) Vα domain CDR2 amino acid sequences may vary from the sequence shown in SEQ ID NO:16 by one or several amino acids. As stated previously, the variant may comprise an amino acid substitution such as a conservative amino acid substitution compared to the sequence shown in SEQ ID NO:16). As stated above, a functional variant of SEQ ID NO:16 retains the ability to specifically bind to HLA-A*01:01.

In one example, the CDR2 of the Vα domain comprises or consists of the amino acid sequence of SEQ ID NO: 16. In examples where the TCR Vα domain CDR2 has the amino acid sequence of SEQ ID NO:16, the CDR2 may be encoded by any appropriate nucleic acid sequence.

The encoded TCR Vα domain may therefore comprise the CDRs mentioned in detail above (by SEQ ID specifically i.e. SEQ ID NO:17, SEQ ID NO:15 and SEQ ID NO: 16, or functional variants thereof), with appropriate intervening sequences between the CDRs.

The encoded TCR Vα domain may comprise an amino acid sequence of SEQ ID NO:21, or a functional variant thereof (i.e. wherein the variant TCR Vα domain retains the ability to specifically bind to a Jchain antigen (e.g. the peptide shown in SEQ ID NO:99) when part of a binding protein described herein). Such functional variants may be naturally occurring, synthetic, or synthetically improved functional variants of SEQ ID NO:21. The term “variant” also encompasses homologues and fragments. Functional variants will typically contain only conservative substitutions of one or more amino acids of SEQ ID NO:21, or substitution, deletion or insertion of non-critical amino acids in non-critical regions of the protein.

Non-functional variants are amino acid sequence variants of SEQ ID NO:21 that do not specifically bind to a Jchain antigen (e.g. the peptide shown in SEQ ID NO:99). Non-functional variants will typically contain a non-conservative substitution, a deletion, or insertion or premature truncation of the amino acid sequence of SEQ ID NO:21 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO:21, whilst retaining the ability to specifically bind to a Jchain antigen (e.g. the peptide shown in SEQ ID NO:99). In other words, a functional TCR Vα domain with one or several amino acid substitutions compared to the sequence of SEQ ID NO:21 is also encompassed. As stated previously, the amino acid substitution may be a conservative amino acid substitution. The variability in sequence compared to SEQ ID NO:21 may all be in regions of the TCR Vα domain that do not form CDRs (i.e. the variant may have the CDRs of SEQ ID NO: 17, SEQ ID NO:15 and/or SEQ ID NO:16, and still have 25% (or less) sequence variability compared to SEQ ID NO:21). In other words, the sequence of the CDRs of SEQ ID NO: 21 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 21).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 21, wherein the TCR Vα domain comprises a CDR3 having an amino acid sequence of SEQ ID NO: 17. In this example, the TCR Vα domain CDR1 may have an amino acid sequence of SEQ ID NO: 15 and the TCR Vα domain CDR2 may have an amino acid sequence of SEQ ID NO: 16.

As another example, the encoded TCR Vα domain may comprise an amino acid sequence having at the amino acid sequence of SEQ ID NO: 21, with 0 to 10 (or 0 to 5) amino acid substitutions, insertions or deletions), wherein the TCR Vα domain comprises a CDR3 having an amino acid sequence of SEQ ID NO: 17. In this example, the TCR Vα domain CDR1 may have an amino acid sequence of SEQ ID NO: 15 and the TCR Vα domain CDR2 may have an amino acid sequence of SEQ ID NO: 16.

In examples where the TCR Vα domain has the amino acid sequence of SEQ ID NO:21, the TCR Vα domain may be encoded by the nucleic acid sequence of SEQ ID NO: 22, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

An example of a specific TCR α chain amino acid sequence that includes a TCR Vα domain described herein with an appropriate constant domain is shown in SEQ ID NO: 25. Appropriate functional variants of SEQ ID NO: 25 are also encompassed (e.g. variants having at least 75% (e.g. at least 75%, at least 80%, at least 85%, at least 90%, at least 95% etc) sequence identity to the amino acid sequence of SEQ ID NO: 25, wherein the variant TCR α chain amino acid sequence retains its ability to specifically bind to a Jchain antigen (e.g. the peptide shown in SEQ ID NO:99) when part of a binding protein described herein). In other words, a functional TCR α chain with one or several amino acid substitutions compared to the sequence of SEQ ID NO:25 is also encompassed. As stated previously, the amino acid substitution may be a conservative amino acid substitution. The variability in sequence compared to SEQ ID NO:25 may all be in regions of the TCR α chain that do not form CDRs (i.e. the variant may have the CDRs of SEQ ID NO: 17, SEQ ID NO: 15 and/or SEQ ID NO: 16, and still have 25% (or less) sequence variability compared to SEQ ID NO:25). In other words, the sequence of the CDRs of SEQ ID NO: 25 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 25).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 25, wherein the TCR α chain comprises a CDR3 having an amino acid sequence of SEQ ID NO: 17. In this example, the TCR α chain CDR1 may have an amino acid sequence of SEQ ID NO:15 and the TCR α chain CDR2 may have an amino acid sequence of SEQ ID NO: 16.

In examples where the TCR α chain has the amino acid sequence of SEQ ID NO:25, the TCR α chain may be encoded by the nucleic acid sequence of SEQ ID NO:26, or a genetically degenerate sequence thereof (i.e. other nucleic acid sequences that encode the same protein as a result of the degeneracy of the genetic code). It is noted that SEQ ID NO:26 is the nucleic acid sequence for TCR α chain of clone 5D12.9.

In one example, the nucleic acid composition provided herein comprises a nucleic acid sequence that encodes a TCR Vα domain comprising a CDR3 amino acid sequence having at least 80% sequence identity to SEQ ID NO:17, or a functional fragment thereof.

In another example, the CDR3 of the Vα domain of a nucleic acid composition provided herein comprises or consists of the amino acid sequence of SEQ ID NO: 17.

In another example, the Vα domain of a nucleic acid composition provided herein comprises an amino acid sequence having at least 80% sequence identity to, comprising, or consisting of, SEQ ID NO: 21.

As provided elsewhere herein, the inventors identified TCR clone 13F6.6 which interacts with YTAVVPLVY (SEQ ID NO:99) in the context of HLA-A*01:01. The sequences provided herein that correspond to TCR clone 13F6.6 are SEQ ID NO:s 85 to 98.

Accordingly, another example of an appropriate TCR Vα domain CDR3 amino acid sequence that confers specific binding to a Jchain antigen (e.g. to YTAVVPLVY (SEQ ID NO: 99)) is shown in SEQ ID NO:87. As would be clear to a person of skill in the art, variants of the amino acid sequence shown in SEQ ID NO:87 may also be functional (i.e. retain their ability to confer specific binding to a Jchain antigen (e.g. to the peptide YTAVVPLVY (SEQ ID NO: 99)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 87, i.e. they may have at least 80%, at least 84%, at least 92%, or 100% sequence identity to SEQ ID NO: 87. Suitably, percent identity is calculated as the percentage of identity to the entire length of the reference sequence (e.g. SEQ ID NO: 87). In other words, appropriate (functional) Vα domain CDR3 amino acid sequences may vary from the sequence shown in SEQ ID NO: 87 by one or several (e.g. two etc) amino acids.

As stated above, functional variants of SEQ ID NO: 87 retain their ability to confer specific binding to a Jchain antigen (i.e. the peptide shown in SEQ ID NO:99) when the CDR3 is part of TCR Vα domain.

Functional variants may be naturally occurring, synthetic, or synthetically improved functional variants of SEQ ID NO: 87. The term “variant” also encompasses homologues and fragments. Functional variants will typically contain only conservative substitutions of one, two or more amino acids of SEQ ID NO: 87, or substitution, deletion or insertion of non-critical amino acids in non-critical regions of the CDR3.

Non-functional variants are amino acid sequence variants of SEQ ID NO: 87 that do not specifically bind to a Jchain antigen (i.e. the peptide shown in SEQ ID NO: 99). Non-functional variants will typically contain a non-conservative substitution, a deletion, or insertion or premature truncation of the amino acid sequence of SEQ ID NO: 87 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 87. In examples where the TCR Vα domain CDR3 has the amino acid sequence of SEQ ID NO:87, the CDR3 may be encoded by any appropriate nucleic acid sequence.

The encoded TCR Vα domain may comprise, in addition to the specified CDR3, a CDR1 comprising an amino acid sequence of SEQ ID NO: 85, or a functional variant thereof (i.e. wherein the variant retains the ability to specifically bind to a Jchain antigen (e.g. the peptide shown in SEQ ID NO: 99)). Such functional variants may be naturally occurring, synthetic, or synthetically improved functional variants of SEQ ID NO: 85. The term “variant” also encompasses homologues and fragments. Functional variants will typically contain only conservative substitutions of one or more amino acids of SEQ ID NO: 85, or substitution, deletion or insertion of non-critical amino acids in non-critical regions of the protein.

Non-functional variants are amino acid sequence variants of SEQ ID NO: 85 that do not specifically bind to a Jchain antigen (e.g. the peptide shown in SEQ ID NO: 99). Non-functional variants will typically contain a non-conservative substitution, a deletion, or insertion or premature truncation of the amino acid sequence of SEQ ID NO: 85 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 85, i.e. it may have at least 80%, at least 83%, or 100% sequence identity to SEQ ID NO: 85. Suitably, percent identity is calculated as the percentage of identity to the entire length of the reference sequence (e.g. SEQ ID NO: 85). In other words, appropriate functional Vα domain CDR1 amino acid sequences may vary from the sequence shown in SEQ ID NO: 85 by one or several amino acids. As stated previously, the variant may comprise an amino acid substitution such as a conservative amino acid substitution compared to the sequence shown in SEQ ID NO: 85. As stated above, functional variants of SEQ ID NO: 85 retain the ability to specifically bind to a Jchain antigen (e.g. the peptide shown in SEQ ID NO:99) when the CDR1 is part of TCR Vα domain).

In one example, the CDR1 of the Vα domain comprises or consists of the amino acid sequence of SEQ ID NO: 85. In examples where the TCR Vα domain CDR1 has the amino acid sequence of SEQ ID NO:85, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 86, or a functional variant thereof (i.e. wherein the variant retains the ability to specifically bind to HLA-A*01:01). Such functional variants may be naturally occurring, synthetic, or synthetically improved functional variants of SEQ ID NO: 86. The term “variant” also encompasses homologues and fragments. Functional variants will typically contain only conservative substitutions of one or more amino acids of SEQ ID NO: 86, or substitution, deletion or insertion of non-critical amino acids in non-critical regions of the protein.

Non-functional variants are amino acid sequence variants of SEQ ID NO: 86 that do not specifically bind to HLA-A*01:01. Non-functional variants will typically contain a non-conservative substitution, a deletion, or insertion or premature truncation of the amino acid sequence of SEQ ID NO: 86 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 86, i.e. it may have at least 80%, at least 87, or 100% sequence identity to SEQ ID NO: 86. Suitably, percent identity is calculated as the percentage of identity to the entire length of the reference sequence (e.g. SEQ ID NO: 86). In other words, appropriate (functional) Vα domain CDR2 amino acid sequences may vary from the sequence shown in SEQ ID NO: 86 by one or several amino acids. As stated previously, the variant may comprise an amino acid substitution such as a conservative amino acid substitution compared to the sequence shown in SEQ ID NO: 86). As stated above, a functional variant of SEQ ID NO: 86 retains the ability to specifically bind to HLA-A*01:01.

In one example, the CDR2 of the Vα domain comprises or consists of the amino acid sequence of SEQ ID NO: 86. In examples where the TCR Vα domain CDR2 has the amino acid sequence of SEQ ID NO: 86, the CDR2 may be encoded by any appropriate nucleic acid sequence.

The encoded TCR Vα domain may therefore comprise the CDRs mentioned in detail above (by SEQ ID specifically i.e. SEQ ID NO:87, SEQ ID NO: 85 and SEQ ID NO: 86, or functional variants thereof), with appropriate intervening sequences between the CDRs.

The encoded TCR Vα domain may comprise an amino acid sequence of SEQ ID NO: 91, or a functional variant thereof (i.e. wherein the variant TCR Vα domain retains the ability to specifically bind to a Jchain antigen (e.g. the peptide shown in SEQ ID NO: 99) when part of a binding protein described herein). Such functional variants may be naturally occurring, synthetic, or synthetically improved functional variants of SEQ ID NO: 91. The term “variant” also encompasses homologues and fragments. Functional variants will typically contain only conservative substitutions of one or more amino acids of SEQ ID NO: 91, or substitution, deletion or insertion of non-critical amino acids in non-critical regions of the protein.

Non-functional variants are amino acid sequence variants of SEQ ID NO: 91 that do not specifically bind to a Jchain antigen (e.g. the peptide shown in SEQ ID NO: 99). Non-functional variants will typically contain a non-conservative substitution, a deletion, or insertion or premature truncation of the amino acid sequence of SEQ ID NO: 91 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 91, whilst retaining the ability to specifically bind to a Jchain antigen (e.g. the peptide shown in SEQ ID NO:99). In other words, a functional TCR Vα domain with one or several amino acid substitutions compared to the sequence of SEQ ID NO: 91 is also encompassed. As stated previously, the amino acid substitution may be a conservative amino acid substitution. The variability in sequence compared to SEQ ID NO: 91 may all be in regions of the TCR Vα domain that do not form CDRs (i.e. the variant may have the CDRs of SEQ ID NO: 87, SEQ ID NO: 85 and/or SEQ ID NO: 86, and still have 25% (or less) sequence variability compared to SEQ ID NO:91). In other words, the sequence of the CDRs of SEQ ID NO: 91 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 91).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 91, wherein the TCR Vα domain comprises a CDR3 having an amino acid sequence of SEQ ID NO: 87. In this example, the TCR Vα domain CDR1 may have an amino acid sequence of SEQ ID NO: 85 and the TCR Vα domain CDR2 may have an amino acid sequence of SEQ ID NO: 86.

As another example, the encoded TCR Vα domain may comprise an amino acid sequence having at the amino acid sequence of SEQ ID NO: 91, with 0 to 10 (or 0 to 5) amino acid substitutions, insertions or deletions), wherein the TCR Vα domain comprises a CDR3 having an amino acid sequence of SEQ ID NO: 87. In this example, the TCR Vα domain CDR1 may have an amino acid sequence of SEQ ID NO: 85 and the TCR Vα domain CDR2 may have an amino acid sequence of SEQ ID NO: 86.

In examples where the TCR Vα domain has the amino acid sequence of SEQ ID NO: 91, the TCR Vα domain may be encoded by the nucleic acid sequence of SEQ ID NO: 92, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

An example of a specific TCR α chain amino acid sequence that includes a TCR Vα domain described herein with an appropriate constant domain is shown in SEQ ID NO: 95. Appropriate functional variants of SEQ ID NO: 95 are also encompassed (e.g. variants having at least 75% (e.g. at least 75%, at least 80%, at least 85%, at least 90%, at least 95% etc) sequence identity to the amino acid sequence of SEQ ID NO: 95, wherein the variant TCR α chain amino acid sequence retains its ability to specifically bind to a Jchain antigen (e.g. the peptide shown in SEQ ID NO:99) when part of a binding protein described herein). In other words, a functional TCR α chain with one or several amino acid substitutions compared to the sequence of SEQ ID NO: 95 is also encompassed. As stated previously, the amino acid substitution may be a conservative amino acid substitution. The variability in sequence compared to SEQ ID NO: 95 may all be in regions of the TCR α chain that do not form CDRs (i.e. the variant may have the CDRs of SEQ ID NO: 87, SEQ ID NO: 85 and/or SEQ ID NO: 86, and still have 25% (or less) sequence variability compared to SEQ ID NO: 95). In other words, the sequence of the CDRs of SEQ ID NO: 95 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 95).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 95, wherein the TCR α chain comprises a CDR3 having an amino acid sequence of SEQ ID NO: 87. In this example, the TCR α chain CDR1 may have an amino acid sequence of SEQ ID NO:85 and the TCR α chain CDR2 may have an amino acid sequence of SEQ ID NO: 86.

In examples where the TCR α chain has the amino acid sequence of SEQ ID NO:95, the TCR α chain may be encoded by the nucleic acid sequence of SEQ ID NO:96, or a genetically degenerate sequence thereof (i.e. other nucleic acid sequences that encode the same protein as a result of the degeneracy of the genetic code). It is noted that SEQ ID NO:96 is the nucleic acid sequence for TCR α chain of clone 13F6.6.

In one example, the nucleic acid composition provided herein comprises a nucleic acid sequence that encodes a TCR Vα domain comprising a CDR3 amino acid sequence having at least 80% sequence identity to SEQ ID NO:87, or a functional fragment thereof.

In another example, the CDR3 of the Vα domain of a nucleic acid composition provided herein comprises or consists of the amino acid sequence of SEQ ID NO: 87.

In another example, the Vα domain of a nucleic acid composition provided herein comprises an amino acid sequence having at least 80% sequence identity to, comprising, or consisting of, SEQ ID NO: 91.

(ii) Vα Domains that Interact with ISDPTSPLRTR (SEQ ID NO: 101)

As provided elsewhere herein, the inventors identified TCR clone 5C8.16 which interacts with ISDPTSPLRTR (SEQ ID NO:101) in the context of HLA-A*03:01. The sequences provided herein that correspond to TCR clone 5C8.16 are SEQ ID NO:s 43 to 56.

An example of an appropriate TCR Vα domain CDR3 amino acid sequence that confers specific binding to a Jchain antigen (e.g. to ISDPTSPLRTR (SEQ ID NO: 101)) is shown in SEQ ID NO: 45. As would be clear to a person of skill in the art, variants of the amino acid sequence shown in SEQ ID NO: 45 may also be functional (i.e. retain their ability to confer specific binding to a Jchain antigen (e.g. to the peptide ISDPTSPLRTR (SEQ ID NO: 101)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 45, i.e. they may have at least 80%, at least 85%, at least 92%, or 100% sequence identity to SEQ ID NO: 45. Suitably, percent identity is calculated as the percentage of identity to the entire length of the reference sequence (e.g. SEQ ID NO: 45). In other words, appropriate (functional) Vα domain CDR3 amino acid sequences may vary from the sequence shown in SEQ ID NO: 45 by one or several (e.g. two etc) amino acids.

As stated above, functional variants of SEQ ID NO: 45 retain their ability to confer specific binding to a Jchain antigen (i.e. the peptide shown in SEQ ID NO: 101) when the CDR3 is part of TCR Vα domain.

Functional variants may be naturally occurring, synthetic, or synthetically improved functional variants of SEQ ID NO: 45. The term “variant” also encompasses homologues and fragments. Functional variants will typically contain only conservative substitutions of one, two or more amino acids of SEQ ID NO: 45, or substitution, deletion or insertion of non-critical amino acids in non-critical regions of the CDR3.

Non-functional variants are amino acid sequence variants of SEQ ID NO: 45 that do not specifically bind to a Jchain antigen (i.e. the peptide shown in SEQ ID NO: 101). Non-functional variants will typically contain a non-conservative substitution, a deletion, or insertion or premature truncation of the amino acid sequence of SEQ ID NO: 45 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 45. In examples where the TCR Vα domain CDR3 has the amino acid sequence of SEQ ID NO: 45, the CDR3 may be encoded by any appropriate nucleic acid sequence.

The encoded TCR Vα domain may comprise, in addition to the specified CDR3, a CDR1 comprising an amino acid sequence of SEQ ID NO: 43, or a functional variant thereof (i.e. wherein the variant retains the ability to specifically bind to a Jchain antigen (e.g. the peptide shown in SEQ ID NO: 101)). Such functional variants may be naturally occurring, synthetic, or synthetically improved functional variants of SEQ ID NO: 43. The term “variant” also encompasses homologues and fragments. Functional variants will typically contain only conservative substitutions of one or more amino acids of SEQ ID NO: 43, or substitution, deletion or insertion of non-critical amino acids in non-critical regions of the protein.

Non-functional variants are amino acid sequence variants of SEQ ID NO: 43 that do not specifically bind to a Jchain antigen (e.g. the peptide shown in SEQ ID NO: 101). Non-functional variants will typically contain a non-conservative substitution, a deletion, or insertion or premature truncation of the amino acid sequence of SEQ ID NO: 43 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 43, i.e. it may have at least 80%, at least 83%, or 100% sequence identity to SEQ ID NO: 43. Suitably, percent identity is calculated as the percentage of identity to the entire length of the reference sequence (e.g. SEQ ID NO: 43). In other words, appropriate functional Vα domain CDR1 amino acid sequences may vary from the sequence shown in SEQ ID NO: 43 by one or several amino acids. As stated previously, the variant may comprise an amino acid substitution such as a conservative amino acid substitution compared to the sequence shown in SEQ ID NO: 43. As stated above, functional variants of SEQ ID NO: 43 retain the ability to specifically bind to a Jchain antigen (e.g. the peptide shown in SEQ ID NO: 101) when the CDR1 is part of TCR Vα domain).

In one example, the CDR1 of the Vα domain comprises or consists of the amino acid sequence of SEQ ID NO: 43. In examples where the TCR Vα domain CDR1 has the amino acid sequence of SEQ ID NO: 43, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 44, or a functional variant thereof (i.e. wherein the variant retains the ability to specifically bind to HLA-A*03:01). Such functional variants may be naturally occurring, synthetic, or synthetically improved functional variants of SEQ ID NO: 44. The term “variant” also encompasses homologues and fragments. Functional variants will typically contain only conservative substitutions of one or more amino acids of SEQ ID NO: 44, or substitution, deletion or insertion of non-critical amino acids in non-critical regions of the protein.

Non-functional variants are amino acid sequence variants of SEQ ID NO: 44 that do not specifically bind to HLA-A*03:01. Non-functional variants will typically contain a non-conservative substitution, a deletion, or insertion or premature truncation of the amino acid sequence of SEQ ID NO: 44 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 44, i.e. it may have at least 80%, at least 87%, or 100% sequence identity to SEQ ID NO: 44. Suitably, percent identity is calculated as the percentage of identity to the entire length of the reference sequence (e.g. SEQ ID NO: 44). In other words, appropriate (functional) Vα domain CDR2 amino acid sequences may vary from the sequence shown in SEQ ID NO: 44 by one or several amino acids. As stated previously, the variant may comprise an amino acid substitution such as a conservative amino acid substitution compared to the sequence shown in SEQ ID NO: 44). As stated above, a functional variant of SEQ ID NO: 44 retains the ability to specifically bind to HLA-A*03:01.

In one example, the CDR2 of the Vα domain comprises or consists of the amino acid sequence of SEQ ID NO: 44. In examples where the TCR Vα domain CDR2 has the amino acid sequence of SEQ ID NO: 44, the CDR2 may be encoded by any appropriate nucleic acid sequence.

The encoded TCR Vα domain may therefore comprise the CDRs mentioned in detail above (by SEQ ID specifically i.e. SEQ ID NO:45, SEQ ID NO: 43 and SEQ ID NO: 44, or functional variants thereof), with appropriate intervening sequences between the CDRs.

The encoded TCR Vα domain may comprise an amino acid sequence of SEQ ID NO: 49, or a functional variant thereof (i.e. wherein the variant TCR Vα domain retains the ability to specifically bind to a Jchain antigen (e.g. the peptide shown in SEQ ID NO: 101) when part of a binding protein described herein). Such functional variants may be naturally occurring, synthetic, or synthetically improved functional variants of SEQ ID NO: 49. The term “variant” also encompasses homologues and fragments. Functional variants will typically contain only conservative substitutions of one or more amino acids of SEQ ID NO: 49, or substitution, deletion or insertion of non-critical amino acids in non-critical regions of the protein.

Non-functional variants are amino acid sequence variants of SEQ ID NO: 49 that do not specifically bind to a Jchain antigen (e.g. the peptide shown in SEQ ID NO: 101). Non-functional variants will typically contain a non-conservative substitution, a deletion, or insertion or premature truncation of the amino acid sequence of SEQ ID NO: 49 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 49, whilst retaining the ability to specifically bind to a Jchain antigen (e.g. the peptide shown in SEQ ID NO: 101). In other words, a functional TCR Vα domain with one or several amino acid substitutions compared to the sequence of SEQ ID NO: 49 is also encompassed. As stated previously, the amino acid substitution may be a conservative amino acid substitution. The variability in sequence compared to SEQ ID NO: 49 may all be in regions of the TCR Vα domain that do not form CDRs (i.e. the variant may have the CDRs of SEQ ID NO: 45, SEQ ID NO: 43 and/or SEQ ID NO: 44, and still have 25% (or less) sequence variability compared to SEQ ID NO:49). In other words, the sequence of the CDRs of SEQ ID NO: 49 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 49).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 49, wherein the TCR Vα domain comprises a CDR3 having an amino acid sequence of SEQ ID NO: 45. In this example, the TCR Vα domain CDR1 may have an amino acid sequence of SEQ ID NO: 43 and the TCR Vα domain CDR2 may have an amino acid sequence of SEQ ID NO: 44.

As another example, the encoded TCR Vα domain may comprise an amino acid sequence having at the amino acid sequence of SEQ ID NO: 49, with 0 to 10 (or 0 to 5) amino acid substitutions, insertions or deletions), wherein the TCR Vα domain comprises a CDR3 having an amino acid sequence of SEQ ID NO: 45. In this example, the TCR Vα domain CDR1 may have an amino acid sequence of SEQ ID NO: 43 and the TCR Vα domain CDR2 may have an amino acid sequence of SEQ ID NO: 44.

In examples where the TCR Vα domain has the amino acid sequence of SEQ ID NO: 49, the TCR Vα domain may be encoded by the nucleic acid sequence of SEQ ID NO: 50, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

An example of a specific TCR α chain amino acid sequence that includes a TCR Vα domain described herein with an appropriate constant domain is shown in SEQ ID NO: 53. Appropriate functional variants of SEQ ID NO:53 are also encompassed (e.g. variants having at least 75% (e.g. at least 75%, at least 80%, at least 85%, at least 90%, at least 95% etc) sequence identity to the amino acid sequence of SEQ ID NO: 53, wherein the variant TCR α chain amino acid sequence retains its ability to specifically bind to a Jchain antigen (e.g. the peptide shown in SEQ ID NO: 101) when part of a binding protein described herein). In other words, a functional TCR α chain with one or several amino acid substitutions compared to the sequence of SEQ ID NO:53 is also encompassed. As stated previously, the amino acid substitution may be a conservative amino acid substitution. The variability in sequence compared to SEQ ID NO:53 may all be in regions of the TCR α chain that do not form CDRs (i.e. the variant may have the CDRs of SEQ ID NO: 45, SEQ ID NO: 43 and/or SEQ ID NO: 44, and still have 25% (or less) sequence variability compared to SEQ ID NO: 53). In other words, the sequence of the CDRs of SEQ ID NO: 53 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 53).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 53, wherein the TCR α chain comprises a CDR3 having an amino acid sequence of SEQ ID NO: 45. In this example, the TCR α chain CDR1 may have an amino acid sequence of SEQ ID NO:43 and the TCR α chain CDR2 may have an amino acid sequence of SEQ ID NO: 44.

In examples where the TCR α chain has the amino acid sequence of SEQ ID NO:53, the TCR α chain may be encoded by the nucleic acid sequence of SEQ ID NO:54, or a genetically degenerate sequence thereof (i.e. other nucleic acid sequences that encode the same protein as a result of the degeneracy of the genetic code). It is noted that SEQ ID NO:54 is the nucleic acid sequence for TCR α chain of clone 5C8.16.

In one example, the nucleic acid composition provided herein comprises a nucleic acid sequence that encodes a TCR Vα domain comprising a CDR3 amino acid sequence having at least 80% sequence identity to SEQ ID NO:45, or a functional fragment thereof.

In another example, the CDR3 of the Vα domain of a nucleic acid composition provided herein comprises or consists of the amino acid sequence of SEQ ID NO: 45.

In another example, the Vα domain of a nucleic acid composition provided herein comprises an amino acid sequence having at least 80% sequence identity to, comprising, or consisting of, SEQ ID NO: 49.

As provided elsewhere herein, the inventors identified TCR clone 16C7.9 which interacts with ISDPTSPLRTR (SEQ ID NO:101) in the context of HLA-A*11:01. The sequences provided herein that correspond to TCR clone 16C7.9 are SEQ ID NO:s 57 to 70.

An example of an appropriate TCR Vα domain CDR3 amino acid sequence that confers specific binding to a Jchain antigen (e.g. to ISDPTSPLRTR (SEQ ID NO: 101)) is shown in SEQ ID NO: 59. As would be clear to a person of skill in the art, variants of the amino acid sequence shown in SEQ ID NO: 59 may also be functional (i.e. retain their ability to confer specific binding to a Jchain antigen (e.g. to the peptide ISDPTSPLRTR (SEQ ID NO: 101)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 59, i.e. they may have at least 80%, at least 86%, at least 93%, or 100% sequence identity to SEQ ID NO: 59. Suitably, percent identity is calculated as the percentage of identity to the entire length of the reference sequence (e.g. SEQ ID NO: 59). In other words, appropriate (functional) Vα domain CDR3 amino acid sequences may vary from the sequence shown in SEQ ID NO: 59 by one or several (e.g. two etc) amino acids.

As stated above, functional variants of SEQ ID NO: 59 retain their ability to confer specific binding to a Jchain antigen (i.e. the peptide shown in SEQ ID NO: 101) when the CDR3 is part of TCR Vα domain.

Functional variants may be naturally occurring, synthetic, or synthetically improved functional variants of SEQ ID NO: 59. The term “variant” also encompasses homologues and fragments. Functional variants will typically contain only conservative substitutions of one, two or more amino acids of SEQ ID NO: 59, or substitution, deletion or insertion of non-critical amino acids in non-critical regions of the CDR3.

Non-functional variants are amino acid sequence variants of SEQ ID NO: 59 that do not specifically bind to a Jchain antigen (i.e. the peptide shown in SEQ ID NO: 101). Non-functional variants will typically contain a non-conservative substitution, a deletion, or insertion or premature truncation of the amino acid sequence of SEQ ID NO: 59 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 59. In examples where the TCR Vα domain CDR3 has the amino acid sequence of SEQ ID NO: 59, the CDR3 may be encoded by any appropriate nucleic acid sequence.

The encoded TCR Vα domain may comprise, in addition to the specified CDR3, a CDR1 comprising an amino acid sequence of SEQ ID NO: 57, or a functional variant thereof (i.e. wherein the variant retains the ability to specifically bind to a Jchain antigen (e.g. the peptide shown in SEQ ID NO: 101)). Such functional variants may be naturally occurring, synthetic, or synthetically improved functional variants of SEQ ID NO: 57. The term “variant” also encompasses homologues and fragments. Functional variants will typically contain only conservative substitutions of one or more amino acids of SEQ ID NO: 57, or substitution, deletion or insertion of non-critical amino acids in non-critical regions of the protein.

Non-functional variants are amino acid sequence variants of SEQ ID NO: 57 that do not specifically bind to a Jchain antigen (e.g. the peptide shown in SEQ ID NO: 101). Non-functional variants will typically contain a non-conservative substitution, a deletion, or insertion or premature truncation of the amino acid sequence of SEQ ID NO: 57 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 57, i.e. it may have at least 80%, at least 85%, or 100% sequence identity to SEQ ID NO: 57. Suitably, percent identity is calculated as the percentage of identity to the entire length of the reference sequence (e.g. SEQ ID NO: 57). In other words, appropriate functional Vα domain CDR1 amino acid sequences may vary from the sequence shown in SEQ ID NO: 57 by one or several amino acids. As stated previously, the variant may comprise an amino acid substitution such as a conservative amino acid substitution compared to the sequence shown in SEQ ID NO: 57. As stated above, functional variants of SEQ ID NO: 57 retain the ability to specifically bind to a Jchain antigen (e.g. the peptide shown in SEQ ID NO: 101) when the CDR1 is part of TCR Vα domain).

In one example, the CDR1 of the Vα domain comprises or consists of the amino acid sequence of SEQ ID NO: 57. In examples where the TCR Vα domain CDR1 has the amino acid sequence of SEQ ID NO: 57, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 58, or a functional variant thereof (i.e. wherein the variant retains the ability to specifically bind to HLA-A*11:01). Such functional variants may be naturally occurring, synthetic, or synthetically improved functional variants of SEQ ID NO: 58. The term “variant” also encompasses homologues and fragments. Functional variants will typically contain only conservative substitutions of one or more amino acids of SEQ ID NO: 58, or substitution, deletion or insertion of non-critical amino acids in non-critical regions of the protein.

Non-functional variants are amino acid sequence variants of SEQ ID NO: 58 that do not specifically bind to HLA-A*11:01. Non-functional variants will typically contain a non-conservative substitution, a deletion, or insertion or premature truncation of the amino acid sequence of SEQ ID NO: 58 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 58, i.e. it may have at least 80%, at least 87%, or 100% sequence identity to SEQ ID NO: 58. Suitably, percent identity is calculated as the percentage of identity to the entire length of the reference sequence (e.g. SEQ ID NO: 58). In other words, appropriate (functional) Vα domain CDR2 amino acid sequences may vary from the sequence shown in SEQ ID NO: 58 by one or several amino acids. As stated previously, the variant may comprise an amino acid substitution such as a conservative amino acid substitution compared to the sequence shown in SEQ ID NO: 58). As stated above, a functional variant of SEQ ID NO: 58 retains the ability to specifically bind to HLA-A*11:01.

In one example, the CDR2 of the Vα domain comprises or consists of the amino acid sequence of SEQ ID NO: 58. In examples where the TCR Vα domain CDR2 has the amino acid sequence of SEQ ID NO: 58, the CDR2 may be encoded by any appropriate nucleic acid sequence.

The encoded TCR Vα domain may therefore comprise the CDRs mentioned in detail above (by SEQ ID specifically i.e. SEQ ID NO:59, SEQ ID NO: 57 and SEQ ID NO: 58, or functional variants thereof), with appropriate intervening sequences between the CDRs.

The encoded TCR Vα domain may comprise an amino acid sequence of SEQ ID NO: 63, or a functional variant thereof (i.e. wherein the variant TCR Vα domain retains the ability to specifically bind to a Jchain antigen (e.g. the peptide shown in SEQ ID NO: 101) when part of a binding protein described herein). Such functional variants may be naturally occurring, synthetic, or synthetically improved functional variants of SEQ ID NO: 63. The term “variant” also encompasses homologues and fragments. Functional variants will typically contain only conservative substitutions of one or more amino acids of SEQ ID NO: 63, or substitution, deletion or insertion of non-critical amino acids in non-critical regions of the protein.

Non-functional variants are amino acid sequence variants of SEQ ID NO: 63 that do not specifically bind to a Jchain antigen (e.g. the peptide shown in SEQ ID NO: 101). Non-functional variants will typically contain a non-conservative substitution, a deletion, or insertion or premature truncation of the amino acid sequence of SEQ ID NO: 63 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 63, whilst retaining the ability to specifically bind to a Jchain antigen (e.g. the peptide shown in SEQ ID NO: 101). In other words, a functional TCR Vα domain with one or several amino acid substitutions compared to the sequence of SEQ ID NO: 63 is also encompassed. As stated previously, the amino acid substitution may be a conservative amino acid substitution. The variability in sequence compared to SEQ ID NO: 63 may all be in regions of the TCR Vα domain that do not form CDRs (i.e. the variant may have the CDRs of SEQ ID NO: 59, SEQ ID NO: 57 and/or SEQ ID NO: 58, and still have 25% (or less) sequence variability compared to SEQ ID NO:7). In other words, the sequence of the CDRs of SEQ ID NO: 63 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 63).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 63, wherein the TCR Vα domain comprises a CDR3 having an amino acid sequence of SEQ ID NO: 59. In this example, the TCR Vα domain CDR1 may have an amino acid sequence of SEQ ID NO: 57 and the TCR Vα domain CDR2 may have an amino acid sequence of SEQ ID NO: 58.

As another example, the encoded TCR Vα domain may comprise an amino acid sequence having at the amino acid sequence of SEQ ID NO: 63, with 0 to 10 (or 0 to 5) amino acid substitutions, insertions or deletions), wherein the TCR Vα domain comprises a CDR3 having an amino acid sequence of SEQ ID NO: 59. In this example, the TCR Vα domain CDR1 may have an amino acid sequence of SEQ ID NO: 57 and the TCR Vα domain CDR2 may have an amino acid sequence of SEQ ID NO: 58.

In examples where the TCR Vα domain has the amino acid sequence of SEQ ID NO: 63, the TCR Vα domain may be encoded by the nucleic acid sequence of SEQ ID NO: 64, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

An example of a specific TCR α chain amino acid sequence that includes a TCR Vα domain described herein with an appropriate constant domain is shown in SEQ ID NO: 67. Appropriate functional variants of SEQ ID NO:67 are also encompassed (e.g. variants having at least 75% (e.g. at least 75%, at least 80%, at least 85%, at least 90%, at least 95% etc) sequence identity to the amino acid sequence of SEQ ID NO: 67, wherein the variant TCR α chain amino acid sequence retains its ability to specifically bind to a Jchain antigen (e.g. the peptide shown in SEQ ID NO: 101) when part of a binding protein described herein). In other words, a functional TCR α chain with one or several amino acid substitutions compared to the sequence of SEQ ID NO:67 is also encompassed. As stated previously, the amino acid substitution may be a conservative amino acid substitution. The variability in sequence compared to SEQ ID NO:67 may all be in regions of the TCR α chain that do not form CDRs (i.e. the variant may have the CDRs of SEQ ID NO: 59, SEQ ID NO: 57 and/or SEQ ID NO: 58, and still have 25% (or less) sequence variability compared to SEQ ID NO: 67). In otherwords, the sequence of the CDRs of SEQ ID NO: 67 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 67).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 67, wherein the TCR α chain comprises a CDR3 having an amino acid sequence of SEQ ID NO: 59. In this example, the TCR α chain CDR1 may have an amino acid sequence of SEQ ID NO:57 and the TCR α chain CDR2 may have an amino acid sequence of SEQ ID NO: 58.

In examples where the TCR α chain has the amino acid sequence of SEQ ID NO: 67, the TCR α chain may be encoded by the nucleic acid sequence of SEQ ID NO: 68, or a genetically degenerate sequence thereof (i.e. other nucleic acid sequences that encode the same protein as a result of the degeneracy of the genetic code). It is noted that SEQ ID NO:68 is the nucleic acid sequence for TCR α chain of clone 16C7.9.

In one example, the nucleic acid composition provided herein comprises a nucleic acid sequence that encodes a TCR Vα domain comprising a CDR3 amino acid sequence having at least 80% sequence identity to SEQ ID NO:59, or a functional fragment thereof.

In another example, the CDR3 of the Vα domain of a nucleic acid composition provided herein comprises or consists of the amino acid sequence of SEQ ID NO: 59.

In another example, the Vα domain of a nucleic acid composition provided herein comprises an amino acid sequence having at least 80% sequence identity to, comprising, or consisting of, SEQ ID NO: 63.

As provided elsewhere herein, the inventors identified TCR clone 13D4.9 which interacts with ISDPTSPLRTR (SEQ ID NO:101) in the context of HLA-A*11:01. The sequences provided herein that correspond to TCR clone 13D4.9 are SEQ ID NO:s 71 to 84.

An example of an appropriate TCR Vα domain CDR3 amino acid sequence that confers specific binding to a Jchain antigen (e.g. to ISDPTSPLRTR (SEQ ID NO: 101)) is shown in SEQ ID NO: 73. As would be clear to a person of skill in the art, variants of the amino acid sequence shown in SEQ ID NO: 73 may also be functional (i.e. retain their ability to confer specific binding to a Jchain antigen (e.g. to the peptide ISDPTSPLRTR (SEQ ID NO: 101)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 73, i.e. they may have at least 80%, at least 85%, at least 92%, or 100% sequence identity to SEQ ID NO: 73. Suitably, percent identity is calculated as the percentage of identity to the entire length of the reference sequence (e.g. SEQ ID NO: 73).

In other words, appropriate (functional) Vα domain CDR3 amino acid sequences may vary from the sequence shown in SEQ ID NO: 73 by one or several (e.g. two etc) amino acids.

As stated above, functional variants of SEQ ID NO: 73 retain their ability to confer specific binding to a Jchain antigen (i.e. the peptide shown in SEQ ID NO: 101) when the CDR3 is part of TCR Vα domain.

Functional variants may be naturally occurring, synthetic, or synthetically improved functional variants of SEQ ID NO: 73. The term “variant” also encompasses homologues and fragments. Functional variants will typically contain only conservative substitutions of one, two or more amino acids of SEQ ID NO: 73, or substitution, deletion or insertion of non-critical amino acids in non-critical regions of the CDR3.

Non-functional variants are amino acid sequence variants of SEQ ID NO: 73 that do not specifically bind to a Jchain antigen (i.e. the peptide shown in SEQ ID NO: 101). Non-functional variants will typically contain a non-conservative substitution, a deletion, or insertion or premature truncation of the amino acid sequence of SEQ ID NO: 73 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 73. In examples where the TCR Vα domain CDR3 has the amino acid sequence of SEQ ID NO: 73, the CDR3 may be encoded by any appropriate nucleic acid sequence.

The encoded TCR Vα domain may comprise, in addition to the specified CDR3, a CDR1 comprising an amino acid sequence of SEQ ID NO: 71, or a functional variant thereof (i.e. wherein the variant retains the ability to specifically bind to a Jchain antigen (e.g. the peptide shown in SEQ ID NO: 101)). Such functional variants may be naturally occurring, synthetic, or synthetically improved functional variants of SEQ ID NO: 71. The term “variant” also encompasses homologues and fragments. Functional variants will typically contain only conservative substitutions of one or more amino acids of SEQ ID NO: 71, or substitution, deletion or insertion of non-critical amino acids in non-critical regions of the protein.

Non-functional variants are amino acid sequence variants of SEQ ID NO: 71 that do not specifically bind to a Jchain antigen (e.g. the peptide shown in SEQ ID NO: 101). Non-functional variants will typically contain a non-conservative substitution, a deletion, or insertion or premature truncation of the amino acid sequence of SEQ ID NO: 71 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 71, i.e. it may have at least 80%, at least 83%, or 100% sequence identity to SEQ ID NO: 71. Suitably, percent identity is calculated as the percentage of identity to the entire length of the reference sequence (e.g. SEQ ID NO: 71). In other words, appropriate functional Vα domain CDR1 amino acid sequences may vary from the sequence shown in SEQ ID NO: 71 by one or several amino acids. As stated previously, the variant may comprise an amino acid substitution such as a conservative amino acid substitution compared to the sequence shown in SEQ ID NO: 71. As stated above, functional variants of SEQ ID NO: 71 retain the ability to specifically bind to a Jchain antigen (e.g. the peptide shown in SEQ ID NO: 101) when the CDR1 is part of TCR Vα domain).

In one example, the CDR1 of the Vα domain comprises or consists of the amino acid sequence of SEQ ID NO: 71. In examples where the TCR Vα domain CDR1 has the amino acid sequence of SEQ ID NO: 71, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 72, or a functional variant thereof (i.e. wherein the variant retains the ability to specifically bind to HLA-A*11:01). Such functional variants may be naturally occurring, synthetic, or synthetically improved functional variants of SEQ ID NO: 72. The term “variant” also encompasses homologues and fragments. Functional variants will typically contain only conservative substitutions of one or more amino acids of SEQ ID NO: 72, or substitution, deletion or insertion of non-critical amino acids in non-critical regions of the protein.

Non-functional variants are amino acid sequence variants of SEQ ID NO: 72 that do not specifically bind to HLA-A*11:01. Non-functional variants will typically contain a non-conservative substitution, a deletion, or insertion or premature truncation of the amino acid sequence of SEQ ID NO: 72 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 72, i.e. it may have at least 80%, at least 85%, or 100% sequence identity to SEQ ID NO: 72. Suitably, percent identity is calculated as the percentage of identity to the entire length of the reference sequence (e.g. SEQ ID NO: 72). In other words, appropriate (functional) Vα domain CDR2 amino acid sequences may vary from the sequence shown in SEQ ID NO: 72 by one or several amino acids. As stated previously, the variant may comprise an amino acid substitution such as a conservative amino acid substitution compared to the sequence shown in SEQ ID NO: 72). As stated above, a functional variant of SEQ ID NO: 72 retains the ability to specifically bind to HLA-A*11:01.

In one example, the CDR2 of the Vα domain comprises or consists of the amino acid sequence of SEQ ID NO: 72. In examples where the TCR Vα domain CDR2 has the amino acid sequence of SEQ ID NO: 72, the CDR2 may be encoded by any appropriate nucleic acid sequence.

The encoded TCR Vα domain may therefore comprise the CDRs mentioned in detail above (by SEQ ID specifically i.e. SEQ ID NO:73, SEQ ID NO: 71 and SEQ ID NO: 72, or functional variants thereof), with appropriate intervening sequences between the CDRs.

The encoded TCR Vα domain may comprise an amino acid sequence of SEQ ID NO: 77, or a functional variant thereof (i.e. wherein the variant TCR Vα domain retains the ability to specifically bind to a Jchain antigen (e.g. the peptide shown in SEQ ID NO: 101) when part of a binding protein described herein). Such functional variants may be naturally occurring, synthetic, or synthetically improved functional variants of SEQ ID NO: 77. The term “variant” also encompasses homologues and fragments. Functional variants will typically contain only conservative substitutions of one or more amino acids of SEQ ID NO: 77, or substitution, deletion or insertion of non-critical amino acids in non-critical regions of the protein.

Non-functional variants are amino acid sequence variants of SEQ ID NO: 77 that do not specifically bind to a Jchain antigen (e.g. the peptide shown in SEQ ID NO: 101). Non-functional variants will typically contain a non-conservative substitution, a deletion, or insertion or premature truncation of the amino acid sequence of SEQ ID NO: 77 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 77, whilst retaining the ability to specifically bind to a Jchain antigen (e.g. the peptide shown in SEQ ID NO: 101). In other words, a functional TCR Vα domain with one or several amino acid substitutions compared to the sequence of SEQ ID NO: 77 is also encompassed. As stated previously, the amino acid substitution may be a conservative amino acid substitution. The variability in sequence compared to SEQ ID NO: 77 may all be in regions of the TCR Vα domain that do not form CDRs (i.e. the variant may have the CDRs of SEQ ID NO: 73, SEQ ID NO: 71 and/or SEQ ID NO: 72, and still have 25% (or less) sequence variability compared to SEQ ID NO:77). In other words, the sequence of the CDRs of SEQ ID NO: 77 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 77).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 77, wherein the TCR Vα domain comprises a CDR3 having an amino acid sequence of SEQ ID NO: 73. In this example, the TCR Vα domain CDR1 may have an amino acid sequence of SEQ ID NO: 71 and the TCR Vα domain CDR2 may have an amino acid sequence of SEQ ID NO: 72.

As another example, the encoded TCR Vα domain may comprise an amino acid sequence having at the amino acid sequence of SEQ ID NO: 77, with 0 to 10 (or 0 to 5) amino acid substitutions, insertions or deletions), wherein the TCR Vα domain comprises a CDR3 having an amino acid sequence of SEQ ID NO: 73. In this example, the TCR Vα domain CDR1 may have an amino acid sequence of SEQ ID NO: 71 and the TCR Vα domain CDR2 may have an amino acid sequence of SEQ ID NO: 72.

In examples where the TCR Vα domain has the amino acid sequence of SEQ ID NO: 77, the TCR Vα domain may be encoded by the nucleic acid sequence of SEQ ID NO: 78, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

An example of a specific TCR α chain amino acid sequence that includes a TCR Vα domain described herein with an appropriate constant domain is shown in SEQ ID NO: 81. Appropriate functional variants of SEQ ID NO:81 are also encompassed (e.g. variants having at least 75% (e.g. at least 75%, at least 80%, at least 85%, at least 90%, at least 95% etc) sequence identity to the amino acid sequence of SEQ ID NO: 81, wherein the variant TCR α chain amino acid sequence retains its ability to specifically bind to a Jchain antigen (e.g. the peptide shown in SEQ ID NO: 101) when part of a binding protein described herein). In other words, a functional TCR α chain with one or several amino acid substitutions compared to the sequence of SEQ ID NO:81 is also encompassed. As stated previously, the amino acid substitution may be a conservative amino acid substitution. The variability in sequence compared to SEQ ID NO:81 may all be in regions of the TCR α chain that do not form CDRs (i.e. the variant may have the CDRs of SEQ ID NO: 73, SEQ ID NO: 71 and/or SEQ ID NO: 72, and still have 25% (or less) sequence variability compared to SEQ ID NO: 81). In other words, the sequence of the CDRs of SEQ ID NO: 81 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 81).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 81, wherein the TCR α chain comprises a CDR3 having an amino acid sequence of SEQ ID NO: 73. In this example, the TCR α chain CDR1 may have an amino acid sequence of SEQ ID NO:71 and the TCR α chain CDR2 may have an amino acid sequence of SEQ ID NO: 72.

In examples where the TCR α chain has the amino acid sequence of SEQ ID NO: 81, the TCR α chain may be encoded by the nucleic acid sequence of SEQ ID NO: 82, or a genetically degenerate sequence thereof (i.e. other nucleic acid sequences that encode the same protein as a result of the degeneracy of the genetic code). It is noted that SEQ ID NO:82 is the nucleic acid sequence for TCR α chain of clone 13D4.9.

In one example, the nucleic acid composition provided herein comprises a nucleic acid sequence that encodes a TCR Vα domain comprising a CDR3 amino acid sequence having at least 80% sequence identity to SEQ ID NO:73, or a functional fragment thereof.

In another example, the CDR3 of the Vα domain of a nucleic acid composition provided herein comprises or consists of the amino acid sequence of SEQ ID NO: 73.

In another example, the Vα domain of a nucleic acid composition provided herein comprises an amino acid sequence having at least 80% sequence identity to, comprising, or consisting of, SEQ ID NO: 77.

(iii) Vα Domains that Interact with CYTAVVPLV (SEQ ID NO: 100)

As provided elsewhere herein, the inventors identified TCR clone 10H11.1 which interacts with CYTAVVPLV (SEQ ID NO:100) in the context of HLA-A*24:02. The sequences provided herein that correspond to TCR clone 10H11.1 are SEQ ID NO:s 29 to 42.

An example of an appropriate TCR Vα domain CDR3 amino acid sequence that confers specific binding to a Jchain antigen (e.g. to CYTAVVPLV (SEQ ID NO: 100)) is shown in SEQ ID NO:31. As would be clear to a person of skill in the art, variants of the amino acid sequence shown in SEQ ID NO:31 may also be functional (i.e. retain their ability to confer specific binding to a Jchain antigen (e.g. to the peptide CYTAVVPLV (SEQ ID NO: 100)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 31, i.e. they may have at least 80%, at least 84%, at least 92%, or 100% sequence identity to SEQ ID NO: 31. Suitably, percent identity is calculated as the percentage of identity to the entire length of the reference sequence (e.g. SEQ ID NO:31). In other words, appropriate (functional) Vα domain CDR3 amino acid sequences may vary from the sequence shown in SEQ ID NO:31 by one or several (e.g. two etc) amino acids.

As stated above, functional variants of SEQ ID NO: 31 retain their ability to confer specific binding to a Jchain antigen (i.e. the peptide shown in SEQ ID NO: 100) when the CDR3 is part of TCR Vα domain.

Functional variants may be naturally occurring, synthetic, or synthetically improved functional variants of SEQ ID NO: 31. The term “variant” also encompasses homologues and fragments. Functional variants will typically contain only conservative substitutions of one, two or more amino acids of SEQ ID NO: 31, or substitution, deletion or insertion of non-critical amino acids in non-critical regions of the CDR3.

Non-functional variants are amino acid sequence variants of SEQ ID NO: 31 that do not specifically bind to a Jchain antigen (i.e. the peptide shown in SEQ ID NO: 100). Non-functional variants will typically contain a non-conservative substitution, a deletion, or insertion or premature truncation of the amino acid sequence of SEQ ID NO: 31 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 31. In examples where the TCR Vα domain CDR3 has the amino acid sequence of SEQ ID NO: 31, the CDR3 may be encoded by any appropriate nucleic acid sequence.

The encoded TCR Vα domain may comprise, in addition to the specified CDR3, a CDR1 comprising an amino acid sequence of SEQ ID NO: 29, or a functional variant thereof (i.e. wherein the variant retains the ability to specifically bind to a Jchain antigen (e.g. the peptide shown in SEQ ID NO: 100)). Such functional variants may be naturally occurring, synthetic, or synthetically improved functional variants of SEQ ID NO: 29. The term “variant” also encompasses homologues and fragments. Functional variants will typically contain only conservative substitutions of one or more amino acids of SEQ ID NO: 29, or substitution, deletion or insertion of non-critical amino acids in non-critical regions of the protein.

Non-functional variants are amino acid sequence variants of SEQ ID NO: 29 that do not specifically bind to a Jchain antigen (e.g. the peptide shown in SEQ ID NO: 100). Non-functional variants will typically contain a non-conservative substitution, a deletion, or insertion or premature truncation of the amino acid sequence of SEQ ID NO: 29 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 29, i.e. it may have at least 80%, at least 83%, or 100% sequence identity to SEQ ID NO: 29. Suitably, percent identity is calculated as the percentage of identity to the entire length of the reference sequence (e.g. SEQ ID NO: 29). In other words, appropriate functional Vα domain CDR1 amino acid sequences may vary from the sequence shown in SEQ ID NO: 29 by one or several amino acids. As stated previously, the variant may comprise an amino acid substitution such as a conservative amino acid substitution compared to the sequence shown in SEQ ID NO: 29. As stated above, functional variants of SEQ ID NO: 29 retain the ability to specifically bind to a Jchain antigen (e.g. the peptide shown in SEQ ID NO: 100) when the CDR1 is part of TCR Vα domain).

In one example, the CDR1 of the Vα domain comprises or consists of the amino acid sequence of SEQ ID NO: 29. In examples where the TCR Vα domain CDR1 has the amino acid sequence of SEQ ID NO: 29, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 30, or a functional variant thereof (i.e. wherein the variant retains the ability to specifically bind to HLA-A*24:02). Such functional variants may be naturally occurring, synthetic, or synthetically improved functional variants of SEQ ID NO: 30. The term “variant” also encompasses homologues and fragments. Functional variants will typically contain only conservative substitutions of one or more amino acids of SEQ ID NO: 30, or substitution, deletion or insertion of non-critical amino acids in non-critical regions of the protein.

Non-functional variants are amino acid sequence variants of SEQ ID NO: 30 that do not specifically bind to HLA-A*24:02. Non-functional variants will typically contain a non-conservative substitution, a deletion, or insertion or premature truncation of the amino acid sequence of SEQ ID NO: 30 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 30, i.e. it may have at least 80%, at least 85%, or 100% sequence identity to SEQ ID NO: 30. Suitably, percent identity is calculated as the percentage of identity to the entire length of the reference sequence (e.g. SEQ ID NO:30). In other words, appropriate (functional) Vα domain CDR2 amino acid sequences may vary from the sequence shown in SEQ ID NO:30 by one or several amino acids. As stated previously, the variant may comprise an amino acid substitution such as a conservative amino acid substitution compared to the sequence shown in SEQ ID NO: 30). As stated above, a functional variant of SEQ ID NO: 30 retains the ability to specifically bind to HLA-A*24:02.

In one example, the CDR2 of the Vα domain comprises or consists of the amino acid sequence of SEQ ID NO: 30. In examples where the TCR Vα domain CDR2 has the amino acid sequence of SEQ ID NO:30, the CDR2 may be encoded by any appropriate nucleic acid sequence.

The encoded TCR Vα domain may therefore comprise the CDRs mentioned in detail above (by SEQ ID specifically i.e. SEQ ID NO: 31, SEQ ID NO: 29 and SEQ ID NO: 30, or functional variants thereof), with appropriate intervening sequences between the CDRs.

The encoded TCR Vα domain may comprise an amino acid sequence of SEQ ID NO: 35, or a functional variant thereof (i.e. wherein the variant TCR Vα domain retains the ability to specifically bind to a Jchain antigen (e.g. the peptide shown in SEQ ID NO: 100) when part of a binding protein described herein). Such functional variants may be naturally occurring, synthetic, or synthetically improved functional variants of SEQ ID NO: 35. The term “variant” also encompasses homologues and fragments. Functional variants will typically contain only conservative substitutions of one or more amino acids of SEQ ID NO: 35, or substitution, deletion or insertion of non-critical amino acids in non-critical regions of the protein.

Non-functional variants are amino acid sequence variants of SEQ ID NO: 35 that do not specifically bind to a Jchain antigen (e.g. the peptide shown in SEQ ID NO: 100). Non-functional variants will typically contain a non-conservative substitution, a deletion, or insertion or premature truncation of the amino acid sequence of SEQ ID NO: 35 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 35, whilst retaining the ability to specifically bind to a Jchain antigen (e.g. the peptide shown in SEQ ID NO: 100). In other words, a functional TCR Vα domain with one or several amino acid substitutions compared to the sequence of SEQ ID NO: 35 is also encompassed. As stated previously, the amino acid substitution may be a conservative amino acid substitution. The variability in sequence compared to SEQ ID NO: 35 may all be in regions of the TCR Vα domain that do not form CDRs (i.e. the variant may have the CDRs of SEQ ID NO: 31, SEQ ID NO: 29 and/or SEQ ID NO: 30, and still have 25% (or less) sequence variability compared to SEQ ID NO: 35). In other words, the sequence of the CDRs of SEQ ID NO: 35 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 35).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 35, wherein the TCR Vα domain comprises a CDR3 having an amino acid sequence of SEQ ID NO: 31. In this example, the TCR Vα domain CDR1 may have an amino acid sequence of SEQ ID NO: 29 and the TCR Vα domain CDR2 may have an amino acid sequence of SEQ ID NO: 30.

As another example, the encoded TCR Vα domain may comprise an amino acid sequence having at the amino acid sequence of SEQ ID NO: 35, with 0 to 10 (or 0 to 5) amino acid substitutions, insertions or deletions), wherein the TCR Vα domain comprises a CDR3 having an amino acid sequence of SEQ ID NO: 31. In this example, the TCR Vα domain CDR1 may have an amino acid sequence of SEQ ID NO: 29 and the TCR Vα domain CDR2 may have an amino acid sequence of SEQ ID NO: 30.

In examples where the TCR Vα domain has the amino acid sequence of SEQ ID NO: 35, the TCR Vα domain may be encoded by the nucleic acid sequence of SEQ ID NO: 36, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

An example of a specific TCR α chain amino acid sequence that includes a TCR Vα domain described herein with an appropriate constant domain is shown in SEQ ID NO: 39. Appropriate functional variants of SEQ ID NO: 39 are also encompassed (e.g. variants having at least 75% (e.g. at least 75%, at least 80%, at least 85%, at least 90%, at least 95% etc) sequence identity to the amino acid sequence of SEQ ID NO: 39, wherein the variant TCR α chain amino acid sequence retains its ability to specifically bind to a Jchain antigen (e.g. the peptide shown in SEQ ID NO: 100) when part of a binding protein described herein). In other words, a functional TCR α chain with one or several amino acid substitutions compared to the sequence of SEQ ID NO: 39 is also encompassed. As stated previously, the amino acid substitution may be a conservative amino acid substitution. The variability in sequence compared to SEQ ID NO: 39 may all be in regions of the TCR α chain that do not form CDRs (i.e. the variant may have the CDRs of SEQ ID NO: 31, SEQ ID NO: 29 and/or SEQ ID NO: 30, and still have 25% (or less) sequence variability compared to SEQ ID NO: 39). In other words, the sequence of the CDRs of SEQ ID NO: 39 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 39).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 39, wherein the TCR α chain comprises a CDR3 having an amino acid sequence of SEQ ID NO: 31. In this example, the TCR α chain CDR1 may have an amino acid sequence of SEQ ID NO: 29 and the TCR α chain CDR2 may have an amino acid sequence of SEQ ID NO: 30.

In examples where the TCR α chain has the amino acid sequence of SEQ ID NO: 39, the TCR α chain may be encoded by the nucleic acid sequence of SEQ ID NO: 40, or a genetically degenerate sequence thereof (i.e. other nucleic acid sequences that encode the same protein as a result of the degeneracy of the genetic code). It is noted that SEQ ID NO:40 is the nucleic acid sequence for TCR α chain of clone 10H11.11

In one example, the nucleic acid composition provided herein comprises a nucleic acid sequence that encodes a TCR Vα domain comprising a CDR3 amino acid sequence having at least 80% sequence identity to SEQ ID NO:31, or a functional fragment thereof.

In another example, the CDR3 of the Vα domain of a nucleic acid composition provided herein comprises or consists of the amino acid sequence of SEQ ID NO: 31.

In another example, the Vα domain of a nucleic acid composition provided herein comprises an amino acid sequence having at least 80% sequence identity to, comprising, or consisting of, SEQ ID NO: 35.

### Components of the TCR β Chain Variable (Vβ) Domain

The isolated nucleic acid composition described herein encodes a Jchain antigen-specific binding protein. As discussed herein, the inventors identified several TCRs that specifically bind to a Jchain antigen selected from YTAVVPLVY (SEQ ID NO:99), ISDPTSPLRTR (SEQ ID NO:101) and CYTAVVPLV (SEQ ID NO:100). The sequences for the Vα domains are discussed above, with the corresponding sequences for the Vβ domains discussed below.

(i) Vβ Domains that Interact with YTAVVPLVY (SEQ ID NO: 99)

As provided elsewhere herein, the inventors identified TCR clone 4G8.8 which interacts with YTAVVPLVY (SEQ ID NO:99) in the context of HLA-A*01:01. The sequences provided herein that correspond to TCR clone 4G8.8 are SEQ ID NO:s 1 to 14.

Accordingly, an example of an appropriate TCR Vβ domain CDR3 amino acid sequence that confers specific binding to a Jchain antigen (e.g. to Jchain antigen YTAVVPLVY (SEQ ID NO: 99)) is shown in SEQ ID NO:6. As would be clear to a person of skill in the art, variants of the amino acid sequence shown in SEQ ID NO:6 may also be functional (i.e. retain their ability to confer specific binding to a Jchain antigen (e.g. the peptide shown in SEQ ID NO: 99) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 6, i.e. they may have at least 80%, at least 84%, at least 92%, or 100% sequence identity to SEQ ID NO: 6. Suitably, percent identity is calculated as the percentage of identity to the entire length of the reference sequence (e.g. SEQ ID NO: 6). In other words, appropriate (functional) Vβ domain CDR3 amino acid sequences may vary from the sequence shown in SEQ ID NO: 6 by one or several (e.g. two) amino acids. As stated above, functional variants of SEQ ID NO: 6 retain their ability to confer specific binding to a Jchain antigen (e.g. the peptide shown in SEQ ID NO: 99) when the CDR3 is part of TCR Vβ domain.

Functional variants may be naturally occurring, synthetic, or synthetically improved functional variants of SEQ ID NO: 6. The term “variant” also encompasses homologues and fragments. Functional variants will typically contain only conservative substitutions of one or more amino acids of SEQ ID NO: 6, or substitution, deletion or insertion of non-critical amino acids in non-critical regions of the CDR3.

Non-functional variants are amino acid sequence variants of SEQ ID NO: 6 that do not specifically bind to a Jchain antigen (e.g. the peptide shown in SEQ ID NO: 99). Non-functional variants will typically contain a non-conservative substitution, a deletion, or insertion or premature truncation of the amino acid sequence of SEQ ID NO: 6 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 6. In examples where the TCR Vβ domain CDR3 has the amino acid sequence of SEQ ID NO:6, the CDR3 may be encoded by Any appropriate nucleic acid sequence.

The encoded TCR Vβ domain may comprise, in addition to the specified CDR3, a CDR1 comprising an amino acid sequence of SEQ ID NO: 4, or a functional variant thereof (i.e. wherein the variant retains the ability to specifically bind to a Jchain antigen (e.g. the peptide shown in SEQ ID NO: 99)). Such functional variants may be naturally occurring, synthetic, or synthetically improved functional variants of SEQ ID NO: 4. The term “variant” also encompasses homologues and fragments. Functional variants will typically contain only conservative substitutions of one or more amino acids of SEQ ID NO: 4, or substitution, deletion or insertion of non-critical amino acids in non-critical regions of the protein.

Non-functional variants are amino acid sequence variants of SEQ ID NO: 4 that do not specifically bind to a Jchain antigen (e.g. the peptide shown in SEQ ID NO: 99). Non-functional variants will typically contain a non-conservative substitution, a deletion, or insertion or premature truncation of the amino acid sequence of SEQ ID NO: 4 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 4, i.e. it may have at least 80%, or 100% sequence identity to SEQ ID NO: 4. Suitably, percent identity is calculated as the percentage of identity to the entire length of the reference sequence (e.g. SEQ ID NO: 4). In other words, appropriate (functional) Vβ domain CDR1 amino acid sequences may vary from the sequence shown in SEQ ID NO:4 by one or several amino acids. As stated previously, the variant may comprise an amino acid substitution such as a conservative amino acid substitution compared to the sequence shown in SEQ ID NO:4). As stated above, functional variants of SEQ ID NO: 4 retain the ability to specifically bind to a Jchain antigen (e.g. the peptide shown in SEQ ID NO: 99) when the CDR1 is part of TCR Vβ domain).

In one example, the CDR1 of the Vβ domain comprises or consists of the amino acid sequence of SEQ ID NO: 4. In examples where the TCR Vα domain CDR1 has the amino acid sequence of SEQ ID NO:4, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 5, or a functional variant thereof (i.e. wherein the variant retains the ability to specifically bind to HLA-A*01:01). Such functional variants may be naturally occurring, synthetic, or synthetically improved functional variants of SEQ ID NO: 5. The term “variant” also encompasses homologues and fragments. Functional variants will typically contain only conservative substitutions of one or more amino acids of SEQ ID NO: 5, or substitution, deletion or insertion of non-critical amino acids in non-critical regions of the protein.

Non-functional variants are amino acid sequence variants of SEQ ID NO: 5 that do not specifically bind to HLA-A*01:01. Non-functional variants will typically contain a non-conservative substitution, a deletion, or insertion or premature truncation of the amino acid sequence of SEQ ID NO: 5 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 5, i.e. it may have at least 80%, at least 83%, or 100% sequence identity to SEQ ID NO: 5. Suitably, percent identity is calculated as the percentage of identity to the entire length of the reference sequence (e.g. SEQ ID NO: 5). In other words, appropriate (functional) Vβ domain CDR2 amino acid sequences may vary from the sequence shown in SEQ ID NO: 5 by one or several amino acids. As stated previously, the variant may comprise an amino acid substitution such as a conservative amino acid substitution compared to the sequence shown in SEQ ID NO: 5). As stated above, a functional variant of SEQ ID NO: 5 retains the ability to specifically bind to HLA-A*01:01.

In one example, the CDR2 of the Vβ domain comprises or consists of the amino acid sequence of SEQ ID NO: 5. In examples where the TCR Vβ domain CDR2 has the amino acid sequence of SEQ ID NO:5, the CDR2 may be encoded by any appropriate nucleic acid sequence.

The encoded TCR Vβ domain may therefore comprise the CDRs mentioned in detail above (by SEQ ID specifically i.e. SEQ ID NO:6, SEQ ID NO: 4 and SEQ ID NO: 5, or functional variants thereof), with appropriate intervening sequences between the CDRs.

The encoded TCR Vβ domain may have an amino acid sequence of SEQ ID NO: 9, or a functional variant thereof (i.e. wherein the variant TCR Vβ domain retains the ability to specifically bind to a Jchain antigen (e.g. the peptide shown in SEQ ID NO: 99) when part of a binding protein described herein). Such functional variants may be naturally occurring, synthetic, or synthetically improved functional variants of SEQ ID NO: 9. The term “variant” also encompasses homologues and fragments. Functional variants will typically contain only conservative substitutions of one or more amino acids of SEQ ID NO: 9, or substitution, deletion or insertion of non-critical amino acids in non-critical regions of the protein.

Non-functional variants are amino acid sequence variants of SEQ ID NO: 9 that do not specifically bind to a Jchain antigen (e.g. the peptide shown in SEQ ID NO: 99). Non-functional variants will typically contain a non-conservative substitution, a deletion, or insertion or premature truncation of the amino acid sequence of SEQ ID NO:9 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 9, whilst retaining the ability to specifically bind to a Jchain antigen (e.g. the peptide shown in SEQ ID NO: 99). In other words, a functional TCR Vβ domain with one or several amino acid substitutions compared to the sequence of SEQ ID NO: 9 is also encompassed. As stated previously, the amino acid substitution may be a conservative amino acid substitution. The variability in sequence compared to SEQ ID NO:9 may all be in regions of the TCR Vβ domain that do not form CDRs (i.e. the variant may have the CDRs of SEQ ID NO: 6, SEQ ID NO: 4 and/or SEQ ID NO: 5, and still have 25% (or less) sequence variability compared to SEQ ID NO: 9). In otherwords, the sequence of the CDRs of SEQ ID NO: 9 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 9).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 9, wherein the TCR Vβ domain comprises a CDR3 having an amino acid sequence of SEQ ID NO: 6. In this example, the TCR Vβ domain CDR1 may have an amino acid sequence of SEQ ID NO:4 and the TCR Vβ domain CDR2 may have an amino acid sequence of SEQ ID NO: 5.

In examples where the TCR Vβ domain has the amino acid sequence of SEQ ID NO:9, the TCR Vβ domain may be encoded by the nucleic acid sequence of SEQ ID NO:10, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 13. Appropriate functional variants of SEQ ID NO: 13 are also encompassed (e.g. variants having at least 75% (e.g. at least 75%, at least 80%, at least 85%, at least 90%, at least 95% etc) sequence identity to the amino acid sequence of SEQ ID NO: 13, wherein the variant TCR β chain amino acid sequence retains its ability to specifically bind to a Jchain antigen (e.g. the peptide shown in SEQ ID NO: 99) when part of a binding protein described herein). In other words, a functional TCR β chain with one or several amino acid substitutions compared to the sequence of SEQ ID NO: 13 is also encompassed. As stated previously, the amino acid substitution may be a conservative amino acid substitution. The variability in sequence compared to SEQ ID NO:13 may all be in regions of the TCR β chain that do not form CDRs (i.e. the variant may have the CDRs of SEQ ID NO: 6, SEQ ID NO: 4 and/or SEQ ID NO: 5, and still have 25% (or less) sequence variability compared to SEQ ID NO:13. In other words, the sequence of the CDRs of SEQ ID NO: 13 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 13).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 13, wherein the TCR β chain comprises a CDR3 having an amino acid sequence of SEQ ID NO: 6. In this example, the TCR 3 chain CDR1 may have an amino acid sequence of SEQ ID NO: 4 and the TCR β chain CDR2 may have an amino acid sequence of SEQ ID NO: 5.

In examples where the TCR β chain has the amino acid sequence of SEQ ID NO:13, the TCR β chain may be encoded by the nucleic acid sequence of SEQ ID NO:14, or a genetically degenerate sequence thereof (i.e. other nucleic acid sequences that encode the same protein as a result of the degeneracy of the genetic code). It is noted that SEQ ID NO:14 is the nucleic acid sequence for TCR β chain of clone 4G8.8.

In an example, the nucleic acid composition provided herein comprises a nucleic acid sequence that encodes a TCR Vβ domain comprising a CDR3 amino acid sequence having at least 80% sequence identity to SEQ ID NO:6, or a functional fragment thereof.

In another example, the CDR3 of the Vβ domain of a nucleic acid composition provided herein comprises or consists of the amino acid sequence of SEQ ID NO:6.

In a further example, the Vβ domain of a nucleic acid composition provided herein comprises an amino acid sequence having at least 80% sequence identity to, comprising, or consisting of, SEQ ID NO: 9.

The TCR Vβ domain sequences derived from TCR clone 4G8.8 discussed above are particularly compatible with the TCR Vα domain sequences derived from TCR clone 4G8.8 discussed elsewhere herein.

Accordingly, in one example, a nucleic acid composition described herein encodes a Jchain antigen-specific binding protein having TCR Vα domain comprising a CDR3 amino acid sequence having at least 80% sequence identity to SEQ ID NO:3, or a functional fragment thereof; and a nucleic acid sequence that encodes a TCR Vβ domain comprising a CDR3 amino acid sequence having at least 80% sequence identity to SEQ ID NO:6, or a functional fragment thereof.

In a particular example, a nucleic acid composition described herein encodes a Jchain antigen-specific binding protein having a TCR Vα domain with a CDR3 amino acid sequence comprising or consisting of the amino acid sequence of SEQ ID NO: 3; and a TCR Vβ domain with a CDR3 comprising or consisting of the amino acid sequence of SEQ ID NO:6. In addition, the Jchain antigen may comprise or consist of the sequence shown in SEQ ID NO: 99. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 7; and the Vβ domain may comprise an amino acid sequence having at least 80% sequence identity to, comprising, or consisting of, SEQ ID NO: 9. In one example, the Vα domain comprises the amino acid sequence of SEQ ID NO: 7 and the Vβ domain comprises the amino acid sequence of SEQ ID NO: 9. In such cases, the Vα domain may be encoded by a nucleic acid sequence comprising the sequence of SEQ ID NO: 8; and the Vβ domain may be encoded by a nucleic acid sequence comprising the sequence of SEQ ID NO: 10.

In this particular example, the TCR Vα domain may include a CDR1 amino acid sequence comprising or consisting of the amino acid sequence of SEQ ID NO:1 and a CDR2 amino acid sequence comprising or consisting of the amino acid sequence of SEQ ID NO:2. Furthermore, the TCR VB domain may include a CDR1 amino acid sequence comprising or consisting of the amino acid sequence of SEQ ID NO:4 and a CDR2 amino acid sequence comprising or consisting of the amino acid sequence of SEQ ID NO: 5.

For the avoidance of doubt, this particular example encompasses components of TCR clone 4G8.8 exemplified herein. The different components of TCR clone 4G8.8 and their respective SEQ ID Nos are summarised in Table 3 below.

As stated in more detail elsewhere herein, the nucleic acid composition described herein encodes both a TCR Vα domain and a TCR Vβ domain, which form the binding protein that is capable of specifically binding to a Jchain antigen. In examples where the TCR Vα domain and the TCR Vβ domain are encoded by the same nucleic acid sequence, the TCR Vα domain and TCR Vβ domain may be joined together via a linker, e.g. a linker that enables expression of two proteins or polypeptides from the same vector. By way of example, a linker comprising a porcine teschovirus-1 2A (P2A) sequence may be used, such as 2A sequences from foot-and-mouth disease virus (F2A), equine rhinitis A virus (E2A) or Thosea asigna virus (T2A) as published by A. L. Szymczak et al., Nature Biotechnology 22, 589-594 (2004) or 2A-like sequences. 2A and 2A-like sequences are linkers that are cleavable once the nucleic acid molecule has been transcribed and translated. Another example of a linker is an internal ribosomal entry sites (IRES) which enables translation of two proteins or polypeptides from the same transcript. Any other appropriate linker may also be used. As a further example, the nucleic acid sequence encoding the TCR Vα domain and nucleic acid sequence encoding the TCR Vβ domain may be cloned into a vector with dual internal promoters (see e.g. S Jones et al., Human Gene Ther 2009). The identification of appropriate linkers and vectors that enable expression of both the TCR Vα domain and the TCR Vβ domain is well within the routine capabilities of a person of skill in the art.

Additional appropriate polypeptide domains may also be encoded by the nucleic acid sequences that encode the TCR Vα domain and/or the TCR Vβ domain. By way of example only, the nucleic acid sequence may comprise a membrane targeting sequence that provides for transport of the encoded polypeptide to the cell surface membrane of the modified cell. Other appropriate additional domains are well known and are described, for example, in WO2016/071758.

In one example, the nucleic acid composition described herein may encode a soluble TCR. For example, the nucleic acid composition may encode the variable domain of the TCR alpha and beta chains respectively together with an immune-modulator molecule such as a CD3 agonist (e.g. an anti-CD3 scFv). The CD3 antigen is present on mature human T cells, thymocytes and a subset of natural killer cells. It is associated with the TCR and is involved in signal transduction of the TCR. Antibodies specific for the human CD3 antigen are well known. One such antibody is the murine monoclonal antibody OKT3, which is the first monoclonal antibody approved by the FDA. Other antibodies specific for CD3 have also been reported (see e.g. WO2004/106380; U.S. Patent Application Publication No. 2004/0202657; U.S. Pat. No. 6,750,325). Immune mobilising mTCR Against Cancer (ImmTAC; Immunocore Limited, Milton Partk, Abington, Oxon, United Kingdom) are bifunctional proteins that combine affinity monoclonal T cell receptor (mTCR) targeting with a therapeutic mechanism of action (i.e., an anti-CD3 scFv). In another example, a soluble TCR of the invention may be combined with a radioisotope or a toxic drug. Appropriate radioisotopes and/or toxic drugs are well known in the art and are readily identifiable by a person of ordinary skill in the art.

In one example, the nucleic acid composition may encode a chimeric single chain TCR wherein the TCR alpha chain variable domain is linked to the TCR beta chain variable domain and a constant domain which is e.g. fused to the CD3 zeta signalling domain. In this example, the linker is non-cleavable. In an alternative embodiment, the nucleic acid composition may encode a chimeric two chain TCR in which the TCR alpha chain variable domain and the TCR beta chain variable domain are each linked to a CD3 zeta signalling domain or other transmembrane and intracellular domains. Methods for preparing such single chain TCRs and two chain TCRs are well known in the art; see for example RA Willemsen et al, Gene Therapy 2000.

As provided elsewhere herein, the inventors have also identified TCR clone 5D12.9 which interacts with YTAVVPLVY (SEQ ID NO:99) in the context of HLA-A*01:01. The sequences provided herein that correspond to TCR clone 5D12.9 are SEQ ID NO:s 15 to 28.

An example of an appropriate TCR Vβ domain CDR3 amino acid sequence that confers specific binding to a Jchain antigen (e.g. to Jchain antigen YTAVVPLVY (SEQ ID NO: 99)) is shown in SEQ ID NO:20. As would be clear to a person of skill in the art, variants of the amino acid sequence shown in SEQ ID NO:20 may also be functional (i.e. retain their ability to confer specific binding to a Jchain antigen (i.e. the peptide shown in SEQ ID NO: 99) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 20, i.e. they may have at least 80%, at least 85%, at least 92%, or 100% sequence identity to SEQ ID NO: 20. Suitably, percent identity is calculated as the percentage of identity to the entire length of the reference sequence (e.g. SEQ ID NO: 20). In other words, appropriate (functional) VB domain CDR3 amino acid sequences may vary from the sequence shown in SEQ ID NO: 20 by one or several (e.g. two) amino acids. As stated above, functional variants of SEQ ID NO: 20 retain their ability to confer specific binding to a Jchain antigen (e.g. the peptide shown in SEQ ID NO: 99) when the CDR3 is part of TCR Vβ domain.

Functional variants may be naturally occurring, synthetic, or synthetically improved functional variants of SEQ ID NO: 20. The term “variant” also encompasses homologues and fragments. Functional variants will typically contain only conservative substitutions of one or more amino acids of SEQ ID NO: 20, or substitution, deletion or insertion of non-critical amino acids in non-critical regions of the CDR3.

Non-functional variants are amino acid sequence variants of SEQ ID NO: 20 that do not specifically bind to a Jchain antigen (e.g. the peptide shown in SEQ ID NO: 99). Non-functional variants will typically contain a non-conservative substitution, a deletion, or insertion or premature truncation of the amino acid sequence of SEQ ID NO: 20 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 20. In examples where the TCR Vβ domain CDR3 has the amino acid sequence of SEQ ID NO:20, the CDR3 may be encoded by any appropriate nucleic acid sequence.

The encoded TCR Vβ domain may comprise, in addition to the specified CDR3, a CDR1 comprising an amino acid sequence of SEQ ID NO: 18, or a functional variant thereof (i.e. wherein the variant retains the ability to specifically bind to a Jchain antigen (e.g. the peptide shown in SEQ ID NO: 99)). Such functional variants may be naturally occurring, synthetic, or synthetically improved functional variants of SEQ ID NO: 18. The term “variant” also encompasses homologues and fragments. Functional variants will typically contain only conservative substitutions of one or more amino acids of SEQ ID NO: 18, or substitution, deletion or insertion of non-critical amino acids in non-critical regions of the protein.

Non-functional variants are amino acid sequence variants of SEQ ID NO: 18 that do not specifically bind to a Jchain antigen (e.g. the peptide shown in SEQ ID NO: 99). Non-functional variants will typically contain a non-conservative substitution, a deletion, or insertion or premature truncation of the amino acid sequence of SEQ ID NO: 18 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 18, i.e. it may have at least 80%, or 100% sequence identity to SEQ ID NO: 18. Suitably, percent identity is calculated as the percentage of identity to the entire length of the reference sequence (e.g. SEQ ID NO: 18). In other words, appropriate (functional) Vβ domain CDR1 amino acid sequences may vary from the sequence shown in SEQ ID NO:18 by one or several amino acids. As stated previously, the variant may comprise an amino acid substitution such as a conservative amino acid substitution compared to the sequence shown in SEQ ID NO:18). As stated above, functional variants of SEQ ID NO: 18 retain the ability to specifically bind to a Jchain antigen (e.g. the peptide shown in SEQ ID NO: 99) when the CDR1 is part of TCR Vβ domain).

In one example, the CDR1 of the Vβ domain comprises or consists of the amino acid sequence of SEQ ID NO: 18. In examples where the TCR Vα domain CDR1 has the amino acid sequence of SEQ ID NO:18, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 19, or a functional variant thereof (i.e. wherein the variant retains the ability to specifically bind to HLA-A*01:01). Such functional variants may be naturally occurring, synthetic, or synthetically improved functional variants of SEQ ID NO: 19. The term “variant” also encompasses homologues and fragments. Functional variants will typically contain only conservative substitutions of one or more amino acids of SEQ ID NO: 19, or substitution, deletion or insertion of non-critical amino acids in non-critical regions of the protein.

Non-functional variants are amino acid sequence variants of SEQ ID NO: 19 that do not specifically bind to HLA-A*01:01. Non-functional variants will typically contain a non-conservative substitution, a deletion, or insertion or premature truncation of the amino acid sequence of SEQ ID NO: 19 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 19, i.e. it may have at least 80%, at least 83%, or 100% sequence identity to SEQ ID NO: 19. Suitably, percent identity is calculated as the percentage of identity to the entire length of the reference sequence (e.g. SEQ ID NO: 19). In other words, appropriate (functional) VB domain CDR2 amino acid sequences may vary from the sequence shown in SEQ ID NO: 19 by one or several amino acids. As stated previously, the variant may comprise an amino acid substitution such as a conservative amino acid substitution compared to the sequence shown in SEQ ID NO: 19). As stated above, a functional variant of SEQ ID NO: 19 retains the ability to specifically bind to HLA-A*01:01.

In one example, the CDR2 of the Vβ domain comprises or consists of the amino acid sequence of SEQ ID NO: 19. In examples where the TCR Vβ domain CDR2 has the amino acid sequence of SEQ ID NO:19, the CDR2 may be encoded by any appropriate nucleic acid sequence.

The encoded TCR Vβ domain may therefore comprise the CDRs mentioned in detail above (by SEQ ID specifically i.e. SEQ ID NO:20, SEQ ID NO: 18 and SEQ ID NO: 19, or functional variants thereof), with appropriate intervening sequences between the CDRs.

The encoded TCR Vβ domain may have an amino acid sequence of SEQ ID NO: 23, or a functional variant thereof (i.e. wherein the variant TCR Vβ domain retains the ability to specifically bind to a Jchain antigen (e.g. the peptide shown in SEQ ID NO: 99) when part of a binding protein described herein). Such functional variants may be naturally occurring, synthetic, or synthetically improved functional variants of SEQ ID NO: 23. The term “variant” also encompasses homologues and fragments. Functional variants will typically contain only conservative substitutions of one or more amino acids of SEQ ID NO: 23, or substitution, deletion or insertion of non-critical amino acids in non-critical regions of the protein.

Non-functional variants are amino acid sequence variants of SEQ ID NO: 23 that do not specifically bind to a Jchain antigen (e.g. the peptide shown in SEQ ID NO: 99). Non-functional variants will typically contain a non-conservative substitution, a deletion, or insertion or premature truncation of the amino acid sequence of SEQ ID NO:23 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 23, whilst retaining the ability to specifically bind to a Jchain antigen (e.g. the peptide shown in SEQ ID NO: 99). In other words, a functional TCR Vβ domain with one or several amino acid substitutions compared to the sequence of SEQ ID NO: 23 is also encompassed. As stated previously, the amino acid substitution may be a conservative amino acid substitution. The variability in sequence compared to SEQ ID NO:23 may all be in regions of the TCR Vβ domain that do not form CDRs (i.e. the variant may have the CDRs of SEQ ID NO: 20, SEQ ID NO: 18 and/or SEQ ID NO: 19, and still have 25% (or less) sequence variability compared to SEQ ID NO: 23). In other words, the sequence of the CDRs of SEQ ID NO: 23 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 23).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 23, wherein the TCR Vβ domain comprises a CDR3 having an amino acid sequence of SEQ ID NO: 20. In this example, the TCR Vβ domain CDR1 may have an amino acid sequence of SEQ ID NO:18 and the TCR Vβ domain CDR2 may have an amino acid sequence of SEQ ID NO: 19.

In examples where the TCR Vβ domain has the amino acid sequence of SEQ ID NO:23, the TCR Vβ domain may be encoded by the nucleic acid sequence of SEQ ID NO:24, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 27. Appropriate functional variants of SEQ ID NO: 27 are also encompassed (e.g. variants having at least 75% (e.g. at least 75%, at least 80%, at least 85%, at least 90%, at least 95% etc) sequence identity to the amino acid sequence of SEQ ID NO: 27, wherein the variant TCR β chain amino acid sequence retains its ability to specifically bind to a Jchain antigen (e.g. the peptide shown in SEQ ID NO: 99) when part of a binding protein described herein). In other words, a functional TCR β chain with one or several amino acid substitutions compared to the sequence of SEQ ID NO: 27 is also encompassed. As stated previously, the amino acid substitution may be a conservative amino acid substitution. The variability in sequence compared to SEQ ID NO:27 may all be in regions of the TCR B chain that do not form CDRs (i.e. the variant may have the CDRs of SEQ ID NO: 20, SEQ ID NO: 18 and/or SEQ ID NO: 19, and still have 25% (or less) sequence variability compared to SEQ ID NO:27. In other words, the sequence of the CDRs of SEQ ID NO: 27 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 27).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 27, wherein the TCR β chain comprises a CDR3 having an amino acid sequence of SEQ ID NO: 20. In this example, the TCR β chain CDR1 may have an amino acid sequence of SEQ ID NO: 18 and the TCR β chain CDR2 may have an amino acid sequence of SEQ ID NO: 19.

In examples where the TCR β chain has the amino acid sequence of SEQ ID NO:27, the TCR β chain may be encoded by the nucleic acid sequence of SEQ ID NO:28, or a genetically degenerate sequence thereof (i.e. other nucleic acid sequences that encode the same protein as a result of the degeneracy of the genetic code). It is noted that SEQ ID NO:28 is the nucleic acid sequence for TCR β chain of clone 5D12.9.

In an example, the nucleic acid composition provided herein comprises a nucleic acid sequence that encodes a TCR Vβ domain comprising a CDR3 amino acid sequence having at least 80% sequence identity to SEQ ID NO:20, or a functional fragment thereof.

In another example, the CDR3 of the Vβ domain of a nucleic acid composition provided herein comprises or consists of the amino acid sequence of SEQ ID NO:20.

In a further example, the Vβ domain of a nucleic acid composition provided herein comprises an amino acid sequence having at least 80% sequence identity to, comprising, or consisting of, SEQ ID NO: 23.

The TCR Vβ domain sequences derived from TCR clone 5D12.9 discussed above are particularly compatible with the TCR Vα domain sequences derived from TCR clone 5D12.9 discussed elsewhere herein.

Accordingly, in one example, a nucleic acid composition described herein encodes a Jchain antigen-specific binding protein having TCR Vα domain comprising a CDR3 amino acid sequence having at least 80% sequence identity to SEQ ID NO:17, or a functional fragment thereof; and a nucleic acid sequence that encodes a TCR Vβ domain comprising a CDR3 amino acid sequence having at least 80% sequence identity to SEQ ID NO:20, or a functional fragment thereof.

In a particular example, a nucleic acid composition described herein encodes a Jchain antigen-specific binding protein having a TCR Vα domain with a CDR3 amino acid sequence comprising or consisting of the amino acid sequence of SEQ ID NO: 17; and a TCR Vβ domain with a CDR3 comprising or consisting of the amino acid sequence of SEQ ID NO:20. In addition, the Jchain antigen may comprise or consist of the sequence shown in SEQ ID NO: 99. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 21; and the Vβ domain may comprise an amino acid sequence having at least 80% sequence identity to, comprising, or consisting of, SEQ ID NO: 23. In one example, the Vα domain comprises the amino acid sequence of SEQ ID NO: 21 and the Vβ domain comprises the amino acid sequence of SEQ ID NO: 23. In such cases, the Vα domain may be encoded by a nucleic acid sequence comprising the sequence of SEQ ID NO: 22; and the Vβ domain may be encoded by a nucleic acid sequence comprising the sequence of SEQ ID NO: 24.

In this particular example, the TCR Vα domain may include a CDR1 amino acid sequence comprising or consisting of the amino acid sequence of SEQ ID NO:15 and a CDR2 amino acid sequence comprising or consisting of the amino acid sequence of SEQ ID NO:16. Furthermore, the TCR Vβ domain may include a CDR1 amino acid sequence comprising or consisting of the amino acid sequence of SEQ ID NO:18 and a CDR2 amino acid sequence comprising or consisting of the amino acid sequence of SEQ ID NO: 19.

For the avoidance of doubt, this particular example encompasses components of TCR clone 5D12.9 exemplified herein. The different components of TCR clone 5D12.9 and their respective SEQ ID Nos are summarised in Table 4 below.

As stated in more detail elsewhere herein, the nucleic acid composition described herein encodes both a TCR Vα domain and a TCR Vβ domain, which form the binding protein that is capable of specifically binding to a Jchain antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

As provided elsewhere herein, the inventors identified TCR clone 13F6.6 which interacts with YTAVVPLVY (SEQ ID NO:99) in the context of HLA-A*01:01. The sequences provided herein that correspond to TCR clone 13F6.6 are SEQ ID NO:s 85 to 98.

An example of an appropriate TCR Vβ domain CDR3 amino acid sequence that confers specific binding to a Jchain antigen (e.g. to Jchain antigen YTAVVPLVY (SEQ ID NO: 99)) is shown in SEQ ID NO:90. As would be clear to a person of skill in the art, variants of the amino acid sequence shown in SEQ ID NO:90 may also be functional (i.e. retain their ability to confer specific binding to a Jchain antigen (i.e. the peptide shown in SEQ ID NO: 99) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 90, i.e. they may have at least 80%, at least 86%, at least 93%, or 100% sequence identity to SEQ ID NO: 90. Suitably, percent identity is calculated as the percentage of identity to the entire length of the reference sequence (e.g. SEQ ID NO: 90). In other words, appropriate (functional) Vβ domain CDR3 amino acid sequences may vary from the sequence shown in SEQ ID NO: 90 by one or several (e.g. two) amino acids. As stated above, functional variants of SEQ ID NO: 90 retain their ability to confer specific binding to a Jchain antigen (e.g. the peptide shown in SEQ ID NO: 99) when the CDR3 is part of TCR Vβ domain.

Functional variants may be naturally occurring, synthetic, or synthetically improved functional variants of SEQ ID NO: 90. The term “variant” also encompasses homologues and fragments. Functional variants will typically contain only conservative substitutions of one or more amino acids of SEQ ID NO: 90, or substitution, deletion or insertion of non-critical amino acids in non-critical regions of the CDR3.

Non-functional variants are amino acid sequence variants of SEQ ID NO: 90 that do not specifically bind to a Jchain antigen (e.g. the peptide shown in SEQ ID NO: 99). Non-functional variants will typically contain a non-conservative substitution, a deletion, or insertion or premature truncation of the amino acid sequence of SEQ ID NO: 90 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 90. In examples where the TCR Vβ domain CDR3 has the amino acid sequence of SEQ ID NO:90, the CDR3 may be encoded by Any appropriate nucleic acid sequence.

The encoded TCR Vβ domain may comprise, in addition to the specified CDR3, a CDR1 comprising an amino acid sequence of SEQ ID NO: 88, or a functional variant thereof (i.e. wherein the variant retains the ability to specifically bind to a Jchain antigen (e.g. the peptide shown in SEQ ID NO: 99)). Such functional variants may be naturally occurring, synthetic, or synthetically improved functional variants of SEQ ID NO: 88. The term “variant” also encompasses homologues and fragments. Functional variants will typically contain only conservative substitutions of one or more amino acids of SEQ ID NO: 88, or substitution, deletion or insertion of non-critical amino acids in non-critical regions of the protein.

Non-functional variants are amino acid sequence variants of SEQ ID NO: 88 that do not specifically bind to a Jchain antigen (e.g. the peptide shown in SEQ ID NO: 99). Non-functional variants will typically contain a non-conservative substitution, a deletion, or insertion or premature truncation of the amino acid sequence of SEQ ID NO: 88 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 88, i.e. it may have at least 80%, or 100% sequence identity to SEQ ID NO: 88. Suitably, percent identity is calculated as the percentage of identity to the entire length of the reference sequence (e.g. SEQ ID NO: 88). In other words, appropriate (functional) Vβ domain CDR1 amino acid sequences may vary from the sequence shown in SEQ ID NO:88 by one or several amino acids. As stated previously, the variant may comprise an amino acid substitution such as a conservative amino acid substitution compared to the sequence shown in SEQ ID NO:88). As stated above, functional variants of SEQ ID NO: 88 retain the ability to specifically bind to a Jchain antigen (e.g. the peptide shown in SEQ ID NO: 99) when the CDR1 is part of TCR Vβ domain).

In one example, the CDR1 of the Vβ domain comprises or consists of the amino acid sequence of SEQ ID NO: 88. In examples where the TCR Vα domain CDR1 has the amino acid sequence of SEQ ID NO:88, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 89, or a functional variant thereof (i.e. wherein the variant retains the ability to specifically bind to HLA-A*01:01). Such functional variants may be naturally occurring, synthetic, or synthetically improved functional variants of SEQ ID NO: 89. The term “variant” also encompasses homologues and fragments. Functional variants will typically contain only conservative substitutions of one or more amino acids of SEQ ID NO: 89, or substitution, deletion or insertion of non-critical amino acids in non-critical regions of the protein.

Non-functional variants are amino acid sequence variants of SEQ ID NO: 89 that do not specifically bind to HLA-A*01:01. Non-functional variants will typically contain a non-conservative substitution, a deletion, or insertion or premature truncation of the amino acid sequence of SEQ ID NO: 89 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 89, i.e. it may have at least 80%, at least 83%, or 100% sequence identity to SEQ ID NO: 89. Suitably, percent identity is calculated as the percentage of identity to the entire length of the reference sequence (e.g. SEQ ID NO: 89). In other words, appropriate (functional) Vβ domain CDR2 amino acid sequences may vary from the sequence shown in SEQ ID NO: 89 by one or several amino acids. As stated previously, the variant may comprise an amino acid substitution such as a conservative amino acid substitution compared to the sequence shown in SEQ ID NO: 89). As stated above, a functional variant of SEQ ID NO: 89 retains the ability to specifically bind to HLA-A*01:01.

In one example, the CDR2 of the Vβ domain comprises or consists of the amino acid sequence of SEQ ID NO: 89. In examples where the TCR Vβ domain CDR2 has the amino acid sequence of SEQ ID NO:89, the CDR2 may be encoded by any appropriate nucleic acid sequence.

The encoded TCR Vβ domain may therefore comprise the CDRs mentioned in detail above (by SEQ ID specifically i.e. SEQ ID NO:90, SEQ ID NO: 88 and SEQ ID NO: 89, or functional variants thereof), with appropriate intervening sequences between the CDRs.

The encoded TCR Vβ domain may have an amino acid sequence of SEQ ID NO: 93, or a functional variant thereof (i.e. wherein the variant TCR Vβ domain retains the ability to specifically bind to a Jchain antigen (e.g. the peptide shown in SEQ ID NO: 99) when part of a binding protein described herein). Such functional variants may be naturally occurring, synthetic, or synthetically improved functional variants of SEQ ID NO: 93. The term “variant” also encompasses homologues and fragments. Functional variants will typically contain only conservative substitutions of one or more amino acids of SEQ ID NO: 93, or substitution, deletion or insertion of non-critical amino acids in non-critical regions of the protein.

Non-functional variants are amino acid sequence variants of SEQ ID NO: 93 that do not specifically bind to a Jchain antigen (e.g. the peptide shown in SEQ ID NO: 99). Non-functional variants will typically contain a non-conservative substitution, a deletion, or insertion or premature truncation of the amino acid sequence of SEQ ID NO:93 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 93, whilst retaining the ability to specifically bind to a Jchain antigen (e.g. the peptide shown in SEQ ID NO: 99). In other words, a functional TCR Vβ domain with one or several amino acid substitutions compared to the sequence of SEQ ID NO: 93 is also encompassed. As stated previously, the amino acid substitution may be a conservative amino acid substitution. The variability in sequence compared to SEQ ID NO:93 may all be in regions of the TCR Vβ domain that do not form CDRs (i.e. the variant may have the CDRs of SEQ ID NO: 90, SEQ ID NO: 88 and/or SEQ ID NO: 89, and still have 25% (or less) sequence variability compared to SEQ ID NO: 93). In other words, the sequence of the CDRs of SEQ ID NO: 93 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 93).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 93, wherein the TCR Vβ domain comprises a CDR3 having an amino acid sequence of SEQ ID NO: 90. In this example, the TCR Vβ domain CDR1 may have an amino acid sequence of SEQ ID NO:88 and the TCR Vβ domain CDR2 may have an amino acid sequence of SEQ ID NO: 89.

In examples where the TCR Vβ domain has the amino acid sequence of SEQ ID NO:93, the TCR Vβ domain may be encoded by the nucleic acid sequence of SEQ ID NO:94, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 97. Appropriate functional variants of SEQ ID NO: 97 are also encompassed (e.g. variants having at least 75% (e.g. at least 75%, at least 80%, at least 85%, at least 90%, at least 95% etc) sequence identity to the amino acid sequence of SEQ ID NO: 97, wherein the variant TCR β chain amino acid sequence retains its ability to specifically bind to a Jchain antigen (e.g. the peptide shown in SEQ ID NO: 99) when part of a binding protein described herein). In other words, a functional TCR β chain with one or several amino acid substitutions compared to the sequence of SEQ ID NO: 97 is also encompassed. As stated previously, the amino acid substitution may be a conservative amino acid substitution. The variability in sequence compared to SEQ ID NO:97 may all be in regions of the TCR β chain that do not form CDRs (i.e. the variant may have the CDRs of SEQ ID NO: 90, SEQ ID NO: 88 and/or SEQ ID NO: 89, and still have 25% (or less) sequence variability compared to SEQ ID NO:97. In other words, the sequence of the CDRs of SEQ ID NO: 97 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 97).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 97, wherein the TCR B chain comprises a CDR3 having an amino acid sequence of SEQ ID NO: 90. In this example, the TCR β chain CDR1 may have an amino acid sequence of SEQ ID NO: 88 and the TCR β chain CDR2 may have an amino acid sequence of SEQ ID NO: 89.

In examples where the TCR β chain has the amino acid sequence of SEQ ID NO:97, the TCR β chain may be encoded by the nucleic acid sequence of SEQ ID NO:98, or a genetically degenerate sequence thereof (i.e. other nucleic acid sequences that encode the same protein as a result of the degeneracy of the genetic code). It is noted that SEQ ID NO:98 is the nucleic acid sequence for TCR β chain of clone 13F6.6.

In an example, the nucleic acid composition provided herein comprises a nucleic acid sequence that encodes a TCR Vβ domain comprising a CDR3 amino acid sequence having at least 80% sequence identity to SEQ ID NO:90, or a functional fragment thereof.

In another example, the CDR3 of the Vβ domain of a nucleic acid composition provided herein comprises or consists of the amino acid sequence of SEQ ID NO:90.

In a further example, the Vβ domain of a nucleic acid composition provided herein comprises an amino acid sequence having at least 80% sequence identity to, comprising, or consisting of, SEQ ID NO: 93.

The TCR Vβ domain sequences derived from TCR clone 13F6.6 discussed above are particularly compatible with the TCR Vα domain sequences derived from TCR clone 13F6.6 discussed elsewhere herein.

Accordingly, in one example, a nucleic acid composition described herein encodes a Jchain antigen-specific binding protein having TCR Vα domain comprising a CDR3 amino acid sequence having at least 80% sequence identity to SEQ ID NO:87, or a functional fragment thereof; and a nucleic acid sequence that encodes a TCR Vβ domain comprising a CDR3 amino acid sequence having at least 80% sequence identity to SEQ ID NO:90, or a functional fragment thereof.

In a particular example, a nucleic acid composition described herein encodes a Jchain antigen-specific binding protein having a TCR Vα domain with a CDR3 amino acid sequence comprising or consisting of the amino acid sequence of SEQ ID NO: 87; and a TCR Vβ domain with a CDR3 comprising or consisting of the amino acid sequence of SEQ ID NO:90. In addition, the Jchain antigen may comprise or consist of the sequence shown in SEQ ID NO: 99. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 91; and the Vβ domain may comprise an amino acid sequence having at least 80% sequence identity to, comprising, or consisting of, SEQ ID NO: 93. In one example, the Vα domain comprises the amino acid sequence of SEQ ID NO: 91 and the Vβ domain comprises the amino acid sequence of SEQ ID NO: 93. In such cases, the Vα domain may be encoded by a nucleic acid sequence comprising the sequence of SEQ ID NO: 92; and the Vβ domain may be encoded by a nucleic acid sequence comprising the sequence of SEQ ID NO: 94.

In this particular example, the TCR Vα domain may include a CDR1 amino acid sequence comprising or consisting of the amino acid sequence of SEQ ID NO:85 and a CDR2 amino acid sequence comprising or consisting of the amino acid sequence of SEQ ID NO:86. Furthermore, the TCR Vβ domain may include a CDR1 amino acid sequence comprising or consisting of the amino acid sequence of SEQ ID NO:88 and a CDR2 amino acid sequence comprising or consisting of the amino acid sequence of SEQ ID NO: 89.

For the avoidance of doubt, this particular example encompasses components of TCR clone 13F6.6 exemplified herein. The different components of TCR clone 13F6.6 and their respective SEQ ID Nos are summarised in Table 9 below.

As stated in more detail elsewhere herein, the nucleic acid composition described herein encodes both a TCR Vα domain and a TCR Vβ domain, which form the binding protein that is capable of specifically binding to a Jchain antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

(ii) Vβ Domains that Interact with ISDPTSPLRTR (SEQ ID NO: 101)

As provided elsewhere herein, the inventors identified TCR clone 5C8.16 which interacts with ISDPTSPLRTR (SEQ ID NO:101) in the context of HLA-A*03:01. The sequences provided herein that correspond to TCR clone 5C8.16 are SEQ ID NO:s 43 to 56.

An example of an appropriate TCR Vβ domain CDR3 amino acid sequence that confers specific binding to a Jchain antigen (e.g. to Jchain antigen ISDPTSPLRTR (SEQ ID NO: 101)) is shown in SEQ ID NO:48. As would be clear to a person of skill in the art, variants of the amino acid sequence shown in SEQ ID NO:48 may also be functional (i.e. retain their ability to confer specific binding to a Jchain antigen (i.e. the peptide shown in SEQ ID NO: 101) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 48, i.e. they may have at least 80%, at least 86%, at least 93%, or 100% sequence identity to SEQ ID NO: 48. Suitably, percent identity is calculated as the percentage of identity to the entire length of the reference sequence (e.g. SEQ ID NO: 48). In other words, appropriate (functional) Vβ domain CDR3 amino acid sequences may vary from the sequence shown in SEQ ID NO: 48 by one or several (e.g. two) amino acids. As stated above, functional variants of SEQ ID NO: 48 retain their ability to confer specific binding to a Jchain antigen (e.g. the peptide shown in SEQ ID NO: 101) when the CDR3 is part of TCR Vβ domain.

Functional variants may be naturally occurring, synthetic, or synthetically improved functional variants of SEQ ID NO: 48. The term “variant” also encompasses homologues and fragments. Functional variants will typically contain only conservative substitutions of one or more amino acids of SEQ ID NO: 48, or substitution, deletion or insertion of non-critical amino acids in non-critical regions of the CDR3.

Non-functional variants are amino acid sequence variants of SEQ ID NO: 48 that do not specifically bind to a Jchain antigen (e.g. the peptide shown in SEQ ID NO: 101). Non-functional variants will typically contain a non-conservative substitution, a deletion, or insertion or premature truncation of the amino acid sequence of SEQ ID NO: 48 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 48. In examples where the TCR Vβ domain CDR3 has the amino acid sequence of SEQ ID NO:48, the CDR3 may be encoded by Any appropriate nucleic acid sequence.

The encoded TCR Vβ domain may comprise, in addition to the specified CDR3, a CDR1 comprising an amino acid sequence of SEQ ID NO: 46, or a functional variant thereof (i.e. wherein the variant retains the ability to specifically bind to a Jchain antigen (e.g. the peptide shown in SEQ ID NO: 101)). Such functional variants may be naturally occurring, synthetic, or synthetically improved functional variants of SEQ ID NO: 46. The term “variant” also encompasses homologues and fragments. Functional variants will typically contain only conservative substitutions of one or more amino acids of SEQ ID NO: 46, or substitution, deletion or insertion of non-critical amino acids in non-critical regions of the protein.

Non-functional variants are amino acid sequence variants of SEQ ID NO: 46 that do not specifically bind to a Jchain antigen (e.g. the peptide shown in SEQ ID NO: 101). Non-functional variants will typically contain a non-conservative substitution, a deletion, or insertion or premature truncation of the amino acid sequence of SEQ ID NO: 46 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 46, i.e. it may have at least 80%, or 100% sequence identity to SEQ ID NO: 46. Suitably, percent identity is calculated as the percentage of identity to the entire length of the reference sequence (e.g. SEQ ID NO: 46). In other words, appropriate (functional) Vβ domain CDR1 amino acid sequences may vary from the sequence shown in SEQ ID NO:46 by one or several amino acids. As stated previously, the variant may comprise an amino acid substitution such as a conservative amino acid substitution compared to the sequence shown in SEQ ID NO:46). As stated above, functional variants of SEQ ID NO: 46 retain the ability to specifically bind to a Jchain antigen (e.g. the peptide shown in SEQ ID NO: 101) when the CDR1 is part of TCR Vβ domain).

In one example, the CDR1 of the Vβ domain comprises or consists of the amino acid sequence of SEQ ID NO: 46. In examples where the TCR Vα domain CDR1 has the amino acid sequence of SEQ ID NO:46, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 47, or a functional variant thereof (i.e. wherein the variant retains the ability to specifically bind to HLA-A*03:01). Such functional variants may be naturally occurring, synthetic, or synthetically improved functional variants of SEQ ID NO: 47. The term “variant” also encompasses homologues and fragments. Functional variants will typically contain only conservative substitutions of one or more amino acids of SEQ ID NO: 47, or substitution, deletion or insertion of non-critical amino acids in non-critical regions of the protein.

Non-functional variants are amino acid sequence variants of SEQ ID NO: 47 that do not specifically bind to HLA-A*03:01. Non-functional variants will typically contain a non-conservative substitution, a deletion, or insertion or premature truncation of the amino acid sequence of SEQ ID NO: 47 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 47, i.e. it may have at least 80%, at least 83%, or 100% sequence identity to SEQ ID NO: 47. Suitably, percent identity is calculated as the percentage of identity to the entire length of the reference sequence (e.g. SEQ ID NO: 47). In other words, appropriate (functional) Vβ domain CDR2 amino acid sequences may vary from the sequence shown in SEQ ID NO: 47 by one or several amino acids. As stated previously, the variant may comprise an amino acid substitution such as a conservative amino acid substitution compared to the sequence shown in SEQ ID NO: 47). As stated above, a functional variant of SEQ ID NO: 47 retains the ability to specifically bind to HLA-A*03:01.

In one example, the CDR2 of the Vβ domain comprises or consists of the amino acid sequence of SEQ ID NO: 47. In examples where the TCR Vβ domain CDR2 has the amino acid sequence of SEQ ID NO:47, the CDR2 may be encoded by any appropriate nucleic acid sequence.

The encoded TCR Vβ domain may therefore comprise the CDRs mentioned in detail above (by SEQ ID specifically i.e. SEQ ID NO:48, SEQ ID NO: 46 and SEQ ID NO: 47, or functional variants thereof), with appropriate intervening sequences between the CDRs.

The encoded TCR Vβ domain may have an amino acid sequence of SEQ ID NO: 51, or a functional variant thereof (i.e. wherein the variant TCR Vβ domain retains the ability to specifically bind to a Jchain antigen (e.g. the peptide shown in SEQ ID NO: 101) when part of a binding protein described herein). Such functional variants may be naturally occurring, synthetic, or synthetically improved functional variants of SEQ ID NO: 51. The term “variant” also encompasses homologues and fragments. Functional variants will typically contain only conservative substitutions of one or more amino acids of SEQ ID NO: 51, or substitution, deletion or insertion of non-critical amino acids in non-critical regions of the protein.

Non-functional variants are amino acid sequence variants of SEQ ID NO: 51 that do not specifically bind to a Jchain antigen (e.g. the peptide shown in SEQ ID NO: 101). Non-functional variants will typically contain a non-conservative substitution, a deletion, or insertion or premature truncation of the amino acid sequence of SEQ ID NO:51 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 51, whilst retaining the ability to specifically bind to a Jchain antigen (e.g. the peptide shown in SEQ ID NO: 101). In other words, a functional TCR Vβ domain with one or several amino acid substitutions compared to the sequence of SEQ ID NO: 51 is also encompassed. As stated previously, the amino acid substitution may be a conservative amino acid substitution. The variability in sequence compared to SEQ ID NO:51 may all be in regions of the TCR Vβ domain that do not form CDRs (i.e. the variant may have the CDRs of SEQ ID NO: 48, SEQ ID NO: 46 and/or SEQ ID NO: 47, and still have 25% (or less) sequence variability compared to SEQ ID NO: 51). In other words, the sequence of the CDRs of SEQ ID NO: 51 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 51).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 51, wherein the TCR Vβ domain comprises a CDR3 having an amino acid sequence of SEQ ID NO: 48. In this example, the TCR Vβ domain CDR1 may have an amino acid sequence of SEQ ID NO:46 and the TCR Vβ domain CDR2 may have an amino acid sequence of SEQ ID NO: 47.

In examples where the TCR Vβ domain has the amino acid sequence of SEQ ID NO:51, the TCR VB domain may be encoded by the nucleic acid sequence of SEQ ID NO:52, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 55. Appropriate functional variants of SEQ ID NO: 55 are also encompassed (e.g. variants having at least 75% (e.g. at least 75%, at least 80%, at least 85%, at least 90%, at least 95% etc) sequence identity to the amino acid sequence of SEQ ID NO: 55, wherein the variant TCR β chain amino acid sequence retains its ability to specifically bind to a Jchain antigen (e.g. the peptide shown in SEQ ID NO: 101) when part of a binding protein described herein). In other words, a functional TCR β chain with one or several amino acid substitutions compared to the sequence of SEQ ID NO: 55 is also encompassed. As stated previously, the amino acid substitution may be a conservative amino acid substitution. The variability in sequence compared to SEQ ID NO:55 may all be in regions of the TCR β chain that do not form CDRs (i.e. the variant may have the CDRs of SEQ ID NO: 48, SEQ ID NO: 46 and/or SEQ ID NO: 47, and still have 25% (or less) sequence variability compared to SEQ ID NO:55. In other words, the sequence of the CDRs of SEQ ID NO: 55 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 55).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 55, wherein the TCR β chain comprises a CDR3 having an amino acid sequence of SEQ ID NO: 48. In this example, the TCR 3 chain CDR1 may have an amino acid sequence of SEQ ID NO: 46 and the TCR β chain CDR2 may have an amino acid sequence of SEQ ID NO: 47.

In examples where the TCR β chain has the amino acid sequence of SEQ ID NO:55, the TCR β chain may be encoded by the nucleic acid sequence of SEQ ID NO:56, or a genetically degenerate sequence thereof (i.e. other nucleic acid sequences that encode the same protein as a result of the degeneracy of the genetic code). It is noted that SEQ ID NO:56 is the nucleic acid sequence for TCR B chain of clone 5C8.16.

In an example, the nucleic acid composition provided herein comprises a nucleic acid sequence that encodes a TCR Vβ domain comprising a CDR3 amino acid sequence having at least 80% sequence identity to SEQ ID NO:48, or a functional fragment thereof.

In another example, the CDR3 of the Vβ domain of a nucleic acid composition provided herein comprises or consists of the amino acid sequence of SEQ ID NO:48.

In a further example, the Vβ domain of a nucleic acid composition provided herein comprises an amino acid sequence having at least 80% sequence identity to, comprising, or consisting of, SEQ ID NO: 51.

The TCR Vβ domain sequences derived from TCR clone 5C8.16 discussed above are particularly compatible with the TCR Vα domain sequences derived from TCR clone 5C8.16 discussed elsewhere herein.

Accordingly, in one example, a nucleic acid composition described herein encodes a Jchain antigen-specific binding protein having TCR Vα domain comprising a CDR3 amino acid sequence having at least 80% sequence identity to SEQ ID NO:45, or a functional fragment thereof; and a nucleic acid sequence that encodes a TCR Vβ domain comprising a CDR3 amino acid sequence having at least 80% sequence identity to SEQ ID NO:48, or a functional fragment thereof.

In a particular example, a nucleic acid composition described herein encodes a Jchain antigen-specific binding protein having a TCR Vα domain with a CDR3 amino acid sequence comprising or consisting of the amino acid sequence of SEQ ID NO: 45; and a TCR Vβ domain with a CDR3 comprising or consisting of the amino acid sequence of SEQ ID NO:48. In addition, the Jchain antigen may comprise or consist of the sequence shown in SEQ ID NO: 101. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 49; and the Vβ domain may comprise an amino acid sequence having at least 80% sequence identity to, comprising, or consisting of, SEQ ID NO: 51. In one example, the Vα domain comprises the amino acid sequence of SEQ ID NO: 49 and the Vβ domain comprises the amino acid sequence of SEQ ID NO: 51. In such cases, the Vα domain may be encoded by a nucleic acid sequence comprising the sequence of SEQ ID NO: 50; and the Vβ domain may be encoded by a nucleic acid sequence comprising the sequence of SEQ ID NO: 52.

In this particular example, the TCR Vα domain may include a CDR1 amino acid sequence comprising or consisting of the amino acid sequence of SEQ ID NO: 43 and a CDR2 amino acid sequence comprising or consisting of the amino acid sequence of SEQ ID NO:44. Furthermore, the TCR Vβ domain may include a CDR1 amino acid sequence comprising or consisting of the amino acid sequence of SEQ ID NO:46 and a CDR2 amino acid sequence comprising or consisting of the amino acid sequence of SEQ ID NO: 47.

For the avoidance of doubt, this particular example encompasses components of TCR clone 5C8.16 exemplified herein. The different components of TCR clone 5C8.16 and their respective SEQ ID Nos are summarised in Table 6 below.

As stated in more detail elsewhere herein, the nucleic acid composition described herein encodes both a TCR Vα domain and a TCR Vβ domain, which form the binding protein that is capable of specifically binding to a Jchain antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

As provided elsewhere herein, the inventors identified TCR clone 16C7.9 which interacts with ISDPTSPLRTR (SEQ ID NO:101) in the context of HLA-A*11:01. The sequences provided herein that correspond to TCR clone 16C7.9 are SEQ ID NO:s 57 to 70.

An example of an appropriate TCR Vβ domain CDR3 amino acid sequence that confers specific binding to a Jchain antigen (e.g. to Jchain antigen ISDPTSPLRTR (SEQ ID NO: 101)) is shown in SEQ ID NO:62. As would be clear to a person of skill in the art, variants of the amino acid sequence shown in SEQ ID NO:62 may also be functional (i.e. retain their ability to confer specific binding to a Jchain antigen (i.e. the peptide shown in SEQ ID NO: 101) when the CDR3 is part of TCR Vβ domain). Such functional variants are therefore encompassed herein.

For example, appropriate (functional) VB domain CDR3 amino acid sequences may have at least 80% sequence identity to SEQ ID NO: 62, i.e. they may have at least 80%, at least 85%, at least 92%, or 100% sequence identity to SEQ ID NO: 62. Suitably, percent identity is calculated as the percentage of identity to the entire length of the reference sequence (e.g. SEQ ID NO: 62). In other words, appropriate (functional) Vβ domain CDR3 amino acid sequences may vary from the sequence shown in SEQ ID NO: 62 by one or several (e.g. two) amino acids. As stated above, functional variants of SEQ ID NO: 62 retain their ability to confer specific binding to a Jchain antigen (e.g. the peptide shown in SEQ ID NO: 101) when the CDR3 is part of TCR Vβ domain.

Functional variants may be naturally occurring, synthetic, or synthetically improved functional variants of SEQ ID NO: 62. The term “variant” also encompasses homologues and fragments. Functional variants will typically contain only conservative substitutions of one or more amino acids of SEQ ID NO: 62, or substitution, deletion or insertion of non-critical amino acids in non-critical regions of the CDR3.

Non-functional variants are amino acid sequence variants of SEQ ID NO: 62 that do not specifically bind to a Jchain antigen (e.g. the peptide shown in SEQ ID NO: 101). Non-functional variants will typically contain a non-conservative substitution, a deletion, or insertion or premature truncation of the amino acid sequence of SEQ ID NO: 62 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 62. In examples where the TCR Vβ domain CDR3 has the amino acid sequence of SEQ ID NO:62, the CDR3 may be encoded by any appropriate nucleic acid sequence.

The encoded TCR Vβ domain may comprise, in addition to the specified CDR3, a CDR1 comprising an amino acid sequence of SEQ ID NO: 60, or a functional variant thereof (i.e. wherein the variant retains the ability to specifically bind to a Jchain antigen (e.g. the peptide shown in SEQ ID NO: 101)). Such functional variants may be naturally occurring, synthetic, or synthetically improved functional variants of SEQ ID NO: 60. The term “variant” also encompasses homologues and fragments. Functional variants will typically contain only conservative substitutions of one or more amino acids of SEQ ID NO: 60, or substitution, deletion or insertion of non-critical amino acids in non-critical regions of the protein.

Non-functional variants are amino acid sequence variants of SEQ ID NO: 60 that do not specifically bind to a Jchain antigen (e.g. the peptide shown in SEQ ID NO: 101). Non-functional variants will typically contain a non-conservative substitution, a deletion, or insertion or premature truncation of the amino acid sequence of SEQ ID NO: 60 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 60, i.e. it may have at least 80%, or 100% sequence identity to SEQ ID NO: 60. Suitably, percent identity is calculated as the percentage of identity to the entire length of the reference sequence (e.g. SEQ ID NO: 60). In other words, appropriate (functional) Vβ domain CDR1 amino acid sequences may vary from the sequence shown in SEQ ID NO:60 by one or several amino acids. As stated previously, the variant may comprise an amino acid substitution such as a conservative amino acid substitution compared to the sequence shown in SEQ ID NO:60). As stated above, functional variants of SEQ ID NO: 60 retain the ability to specifically bind to a Jchain antigen (e.g. the peptide shown in SEQ ID NO: 101) when the CDR1 is part of TCR Vβ domain).

In one example, the CDR1 of the Vβ domain comprises or consists of the amino acid sequence of SEQ ID NO: 60. In examples where the TCR Vα domain CDR1 has the amino acid sequence of SEQ ID NO:60, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 61, or a functional variant thereof (i.e. wherein the variant retains the ability to specifically bind to HLA-A*11:01). Such functional variants may be naturally occurring, synthetic, or synthetically improved functional variants of SEQ ID NO: 61. The term “variant” also encompasses homologues and fragments. Functional variants will typically contain only conservative substitutions of one or more amino acids of SEQ ID NO: 61, or substitution, deletion or insertion of non-critical amino acids in non-critical regions of the protein.

Non-functional variants are amino acid sequence variants of SEQ ID NO: 61 that do not specifically bind to HLA-A*11:01. Non-functional variants will typically contain a non-conservative substitution, a deletion, or insertion or premature truncation of the amino acid sequence of SEQ ID NO: 61 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 61, i.e. it may have at least 80%, at least 83, or 100% sequence identity to SEQ ID NO: 61. Suitably, percent identity is calculated as the percentage of identity to the entire length of the reference sequence (e.g. SEQ ID NO: 61). In other words, appropriate (functional) Vβ domain CDR2 amino acid sequences may vary from the sequence shown in SEQ ID NO: 61 by one or several amino acids. As stated previously, the variant may comprise an amino acid substitution such as a conservative amino acid substitution compared to the sequence shown in SEQ ID NO: 61). As stated above, a functional variant of SEQ ID NO: 61 retains the ability to specifically bind to HLA-A*11:01.

In one example, the CDR2 of the Vβ domain comprises or consists of the amino acid sequence of SEQ ID NO: 61. In examples where the TCR Vβ domain CDR2 has the amino acid sequence of SEQ ID NO:61, the CDR2 may be encoded by any appropriate nucleic acid sequence.

The encoded TCR Vβ domain may therefore comprise the CDRs mentioned in detail above (by SEQ ID specifically i.e. SEQ ID NO:62, SEQ ID NO: 60 and SEQ ID NO: 61, or functional variants thereof), with appropriate intervening sequences between the CDRs.

The encoded TCR Vβ domain may have an amino acid sequence of SEQ ID NO: 65, or a functional variant thereof (i.e. wherein the variant TCR Vβ domain retains the ability to specifically bind to a Jchain antigen (e.g. the peptide shown in SEQ ID NO: 101) when part of a binding protein described herein). Such functional variants may be naturally occurring, synthetic, or synthetically improved functional variants of SEQ ID NO: 65. The term “variant” also encompasses homologues and fragments. Functional variants will typically contain only conservative substitutions of one or more amino acids of SEQ ID NO: 65, or substitution, deletion or insertion of non-critical amino acids in non-critical regions of the protein.

Non-functional variants are amino acid sequence variants of SEQ ID NO: 65 that do not specifically bind to a Jchain antigen (e.g. the peptide shown in SEQ ID NO: 101). Non-functional variants will typically contain a non-conservative substitution, a deletion, or insertion or premature truncation of the amino acid sequence of SEQ ID NO:65 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 65, whilst retaining the ability to specifically bind to a Jchain antigen (e.g. the peptide shown in SEQ ID NO: 101). In other words, a functional TCR Vβ domain with one or several amino acid substitutions compared to the sequence of SEQ ID NO: 65 is also encompassed. As stated previously, the amino acid substitution may be a conservative amino acid substitution. The variability in sequence compared to SEQ ID NO:65 may all be in regions of the TCR Vβ domain that do not form CDRs (i.e. the variant may have the CDRs of SEQ ID NO: 62, SEQ ID NO: 60 and/or SEQ ID NO: 61, and still have 25% (or less) sequence variability compared to SEQ ID NO: 65). In other words, the sequence of the CDRs of SEQ ID NO: 65 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 65).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 65, wherein the TCR Vβ domain comprises a CDR3 having an amino acid sequence of SEQ ID NO: 62. In this example, the TCR Vβ domain CDR1 may have an amino acid sequence of SEQ ID NO:60 and the TCR Vβ domain CDR2 may have an amino acid sequence of SEQ ID NO: 61.

In examples where the TCR Vβ domain has the amino acid sequence of SEQ ID NO:65, the TCR Vβ domain may be encoded by the nucleic acid sequence of SEQ ID NO: 66, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 69. Appropriate functional variants of SEQ ID NO: 69 are also encompassed (e.g. variants having at least 75% (e.g. at least 75%, at least 80%, at least 85%, at least 90%, at least 95% etc) sequence identity to the amino acid sequence of SEQ ID NO: 69, wherein the variant TCR β chain amino acid sequence retains its ability to specifically bind to a Jchain antigen (e.g. the peptide shown in SEQ ID NO: 101) when part of a binding protein described herein). In other words, a functional TCR β chain with one or several amino acid substitutions compared to the sequence of SEQ ID NO: 69 is also encompassed. As stated previously, the amino acid substitution may be a conservative amino acid substitution. The variability in sequence compared to SEQ ID NO:69 may all be in regions of the TCR β chain that do not form CDRs (i.e. the variant may have the CDRs of SEQ ID NO: 62, SEQ ID NO: 60 and/or SEQ ID NO: 61, and still have 25% (or less) sequence variability compared to SEQ ID NO:69. In other words, the sequence of the CDRs of SEQ ID NO: 69 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 69).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 69, wherein the TCR β chain comprises a CDR3 having an amino acid sequence of SEQ ID NO: 62. In this example, the TCR β chain CDR1 may have an amino acid sequence of SEQ ID NO: 60 and the TCR β chain CDR2 may have an amino acid sequence of SEQ ID NO: 61.

In examples where the TCR β chain has the amino acid sequence of SEQ ID NO:69, the TCR β chain may be encoded by the nucleic acid sequence of SEQ ID NO:70, or a genetically degenerate sequence thereof (i.e. other nucleic acid sequences that encode the same protein as a result of the degeneracy of the genetic code). It is noted that SEQ ID NO:70 is the nucleic acid sequence for TCR β chain of clone 16C7.9.

In an example, the nucleic acid composition provided herein comprises a nucleic acid sequence that encodes a TCR Vβ domain comprising a CDR3 amino acid sequence having at least 80% sequence identity to SEQ ID NO:62, or a functional fragment thereof.

In another example, the CDR3 of the Vβ domain of a nucleic acid composition provided herein comprises or consists of the amino acid sequence of SEQ ID NO:62.

In a further example, the Vβ domain of a nucleic acid composition provided herein comprises an amino acid sequence having at least 80% sequence identity to, comprising, or consisting of, SEQ ID NO: 65.

The TCR Vβ domain sequences derived from TCR clone 16C7.9 discussed above are particularly compatible with the TCR Vα domain sequences derived from TCR clone 16C7.9 discussed elsewhere herein.

Accordingly, in one example, a nucleic acid composition described herein encodes a Jchain antigen-specific binding protein having TCR Vα domain comprising a CDR3 amino acid sequence having at least 80% sequence identity to SEQ ID NO:59, or a functional fragment thereof; and a nucleic acid sequence that encodes a TCR Vβ domain comprising a CDR3 amino acid sequence having at least 80% sequence identity to SEQ ID NO:62, or a functional fragment thereof.

In a particular example, a nucleic acid composition described herein encodes a Jchain antigen-specific binding protein having a TCR Vα domain with a CDR3 amino acid sequence comprising or consisting of the amino acid sequence of SEQ ID NO: 59; and a TCR Vβ domain with a CDR3 comprising or consisting of the amino acid sequence of SEQ ID NO:62. In addition, the Jchain antigen may comprise or consist of the sequence shown in SEQ ID NO: 101. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 63; and the Vβ domain may comprise an amino acid sequence having at least 80% sequence identity to, comprising, or consisting of, SEQ ID NO: 65. In one example, the Vα domain comprises the amino acid sequence of SEQ ID NO: 63 and the Vβ domain comprises the amino acid sequence of SEQ ID NO: 65. In such cases, the Vα domain may be encoded by a nucleic acid sequence comprising the sequence of SEQ ID NO: 64; and the Vβ domain may be encoded by a nucleic acid sequence comprising the sequence of SEQ ID NO: 66.

In this particular example, the TCR Vα domain may include a CDR1 amino acid sequence comprising or consisting of the amino acid sequence of SEQ ID NO:57 and a CDR2 amino acid sequence comprising or consisting of the amino acid sequence of SEQ ID NO:58. Furthermore, the TCR Vβ domain may include a CDR1 amino acid sequence comprising or consisting of the amino acid sequence of SEQ ID NO:60 and a CDR2 amino acid sequence comprising or consisting of the amino acid sequence of SEQ ID NO: 61.

For the avoidance of doubt, this particular example encompasses components of TCR clone 16C7.9 exemplified herein. The different components of TCR clone 16C7.9 and their respective SEQ ID Nos are summarised in Table 7 below.

As stated in more detail elsewhere herein, the nucleic acid composition described herein encodes both a TCR Vα domain and a TCR Vβ domain, which form the binding protein that is capable of specifically binding to a Jchain antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

As provided elsewhere herein, the inventors identified TCR clone 13D4.9 which interacts with ISDPTSPLRTR (SEQ ID NO:101) in the context of HLA-A*11:01. The sequences provided herein that correspond to TCR clone 13D4.9 are SEQ ID NO:s 71 to 84.

An example of an appropriate TCR Vβ domain CDR3 amino acid sequence that confers specific binding to a Jchain antigen (e.g. to Jchain antigen ISDPTSPLRTR (SEQ ID NO: 101)) is shown in SEQ ID NO:76. As would be clear to a person of skill in the art, variants of the amino acid sequence shown in SEQ ID NO:76 may also be functional (i.e. retain their ability to confer specific binding to a Jchain antigen (i.e. the peptide shown in SEQ ID NO: 101) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 76, i.e. they may have at least 80%, at least 81%, at least 87%, at least 93%, or 100% sequence identity to SEQ ID NO: 76. Suitably, percent identity is calculated as the percentage of identity to the entire length of the reference sequence (e.g. SEQ ID NO: 76). In other words, appropriate (functional) Vβ domain CDR3 amino acid sequences may vary from the sequence shown in SEQ ID NO: 76 by one or several (e.g. two) amino acids.

As stated above, functional variants of SEQ ID NO: 76 retain their ability to confer specific binding to a Jchain antigen (e.g. the peptide shown in SEQ ID NO: 101) when the CDR3 is part of TCR Vβ domain.

Functional variants may be naturally occurring, synthetic, or synthetically improved functional variants of SEQ ID NO: 76. The term “variant” also encompasses homologues and fragments. Functional variants will typically contain only conservative substitutions of one or more amino acids of SEQ ID NO: 76, or substitution, deletion or insertion of non-critical amino acids in non-critical regions of the CDR3.

Non-functional variants are amino acid sequence variants of SEQ ID NO: 76 that do not specifically bind to a Jchain antigen (e.g. the peptide shown in SEQ ID NO: 101). Non-functional variants will typically contain a non-conservative substitution, a deletion, or insertion or premature truncation of the amino acid sequence of SEQ ID NO: 76 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 76. In examples where the TCR Vβ domain CDR3 has the amino acid sequence of SEQ ID NO:76, the CDR3 may be encoded by any appropriate nucleic acid sequence.

The encoded TCR Vβ domain may comprise, in addition to the specified CDR3, a CDR1 comprising an amino acid sequence of SEQ ID NO: 74, or a functional variant thereof (i.e. wherein the variant retains the ability to specifically bind to a Jchain antigen (e.g. the peptide shown in SEQ ID NO: 101)). Such functional variants may be naturally occurring, synthetic, or synthetically improved functional variants of SEQ ID NO: 74. The term “variant” also encompasses homologues and fragments. Functional variants will typically contain only conservative substitutions of one or more amino acids of SEQ ID NO: 74, or substitution, deletion or insertion of non-critical amino acids in non-critical regions of the protein.

Non-functional variants are amino acid sequence variants of SEQ ID NO: 74 that do not specifically bind to a Jchain antigen (e.g. the peptide shown in SEQ ID NO: 101). Non-functional variants will typically contain a non-conservative substitution, a deletion, or insertion or premature truncation of the amino acid sequence of SEQ ID NO: 74 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 74, i.e. it may have at least 80%, or 100% sequence identity to SEQ ID NO: 74. Suitably, percent identity is calculated as the percentage of identity to the entire length of the reference sequence (e.g. SEQ ID NO: 74). In other words, appropriate (functional) Vβ domain CDR1 amino acid sequences may vary from the sequence shown in SEQ ID NO:74 by one or several amino acids. As stated previously, the variant may comprise an amino acid substitution such as a conservative amino acid substitution compared to the sequence shown in SEQ ID NO:74). As stated above, functional variants of SEQ ID NO: 74 retain the ability to specifically bind to a Jchain antigen (e.g. the peptide shown in SEQ ID NO: 101) when the CDR1 is part of TCR Vβ domain).

In one example, the CDR1 of the Vβ domain comprises or consists of the amino acid sequence of SEQ ID NO: 74. In examples where the TCR Vα domain CDR1 has the amino acid sequence of SEQ ID NO:74, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 75, or a functional variant thereof (i.e. wherein the variant retains the ability to specifically bind to HLA-A*11:01). Such functional variants may be naturally occurring, synthetic, or synthetically improved functional variants of SEQ ID NO: 75. The term “variant” also encompasses homologues and fragments. Functional variants will typically contain only conservative substitutions of one or more amino acids of SEQ ID NO: 75, or substitution, deletion or insertion of non-critical amino acids in non-critical regions of the protein.

Non-functional variants are amino acid sequence variants of SEQ ID NO: 75 that do not specifically bind to HLA-A*11:01. Non-functional variants will typically contain a non-conservative substitution, a deletion, or insertion or premature truncation of the amino acid sequence of SEQ ID NO: 75 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 75, i.e. it may have at least 80%, at least 83%, or 100% sequence identity to SEQ ID NO: 75. Suitably, percent identity is calculated as the percentage of identity to the entire length of the reference sequence (e.g. SEQ ID NO: 75). In other words, appropriate (functional) Vβ domain CDR2 amino acid sequences may vary from the sequence shown in SEQ ID NO: 75 by one or several amino acids. As stated previously, the variant may comprise an amino acid substitution such as a conservative amino acid substitution compared to the sequence shown in SEQ ID NO: 75). As stated above, a functional variant of SEQ ID NO: 75 retains the ability to specifically bind to HLA-A*11:01.

In one example, the CDR2 of the Vβ domain comprises or consists of the amino acid sequence of SEQ ID NO: 75. In examples where the TCR Vβ domain CDR2 has the amino acid sequence of SEQ ID NO:75, the CDR2 may be encoded by any appropriate nucleic acid sequence.

The encoded TCR Vβ domain may therefore comprise the CDRs mentioned in detail above (by SEQ ID specifically i.e. SEQ ID NO:76, SEQ ID NO: 74 and SEQ ID NO: 75, or functional variants thereof), with appropriate intervening sequences between the CDRs.

The encoded TCR Vβ domain may have an amino acid sequence of SEQ ID NO: 79, or a functional variant thereof (i.e. wherein the variant TCR Vβ domain retains the ability to specifically bind to a Jchain antigen (e.g. the peptide shown in SEQ ID NO: 101) when part of a binding protein described herein). Such functional variants may be naturally occurring, synthetic, or synthetically improved functional variants of SEQ ID NO: 79. The term “variant” also encompasses homologues and fragments. Functional variants will typically contain only conservative substitutions of one or more amino acids of SEQ ID NO: 79, or substitution, deletion or insertion of non-critical amino acids in non-critical regions of the protein.

Non-functional variants are amino acid sequence variants of SEQ ID NO: 79 that do not specifically bind to a Jchain antigen (e.g. the peptide shown in SEQ ID NO: 101). Non-functional variants will typically contain a non-conservative substitution, a deletion, or insertion or premature truncation of the amino acid sequence of SEQ ID NO:79 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 79, whilst retaining the ability to specifically bind to a Jchain antigen (e.g. the peptide shown in SEQ ID NO: 101). In other words, a functional TCR Vβ domain with one or several amino acid substitutions compared to the sequence of SEQ ID NO: 79 is also encompassed. As stated previously, the amino acid substitution may be a conservative amino acid substitution. The variability in sequence compared to SEQ ID NO:79 may all be in regions of the TCR VB domain that do not form CDRs (i.e. the variant may have the CDRs of SEQ ID NO: 76, SEQ ID NO: 74 and/or SEQ ID NO: 75, and still have 25% (or less) sequence variability compared to SEQ ID NO: 79). In other words, the sequence of the CDRs of SEQ ID NO: 79 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 79).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 79, wherein the TCR Vβ domain comprises a CDR3 having an amino acid sequence of SEQ ID NO: 76. In this example, the TCR Vβ domain CDR1 may have an amino acid sequence of SEQ ID NO:74 and the TCR Vβ domain CDR2 may have an amino acid sequence of SEQ ID NO: 75.

In examples where the TCR Vβ domain has the amino acid sequence of SEQ ID NO:79, the TCR Vβ domain may be encoded by the nucleic acid sequence of SEQ ID NO: 80, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 83. Appropriate functional variants of SEQ ID NO: 83 are also encompassed (e.g. variants having at least 75% (e.g. at least 75%, at least 80%, at least 85%, at least 90%, at least 95% etc) sequence identity to the amino acid sequence of SEQ ID NO: 83, wherein the variant TCR β chain amino acid sequence retains its ability to specifically bind to a Jchain antigen (e.g. the peptide shown in SEQ ID NO: 101) when part of a binding protein described herein). In other words, a functional TCR β chain with one or several amino acid substitutions compared to the sequence of SEQ ID NO: 83 is also encompassed. As stated previously, the amino acid substitution may be a conservative amino acid substitution. The variability in sequence compared to SEQ ID NO:83 may all be in regions of the TCR β chain that do not form CDRs (i.e. the variant may have the CDRs of SEQ ID NO: 76, SEQ ID NO: 74 and/or SEQ ID NO: 75, and still have 25% (or less) sequence variability compared to SEQ ID NO:83. In other words, the sequence of the CDRs of SEQ ID NO: 83 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 83).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 83, wherein the TCR β chain comprises a CDR3 having an amino acid sequence of SEQ ID NO: 76. In this example, the TCR β chain CDR1 may have an amino acid sequence of SEQ ID NO: 74 and the TCR β chain CDR2 may have an amino acid sequence of SEQ ID NO: 75.

In examples where the TCR β chain has the amino acid sequence of SEQ ID NO:83, the TCR β chain may be encoded by the nucleic acid sequence of SEQ ID NO: 84, or a genetically degenerate sequence thereof (i.e. other nucleic acid sequences that encode the same protein as a result of the degeneracy of the genetic code). It is noted that SEQ ID NO:84 is the nucleic acid sequence for TCR β chain of clone 13D4.9.

In an example, the nucleic acid composition provided herein comprises a nucleic acid sequence that encodes a TCR Vβ domain comprising a CDR3 amino acid sequence having at least 80% sequence identity to SEQ ID NO:76, or a functional fragment thereof.

In another example, the CDR3 of the Vβ domain of a nucleic acid composition provided herein comprises or consists of the amino acid sequence of SEQ ID NO:76.

In a further example, the Vβ domain of a nucleic acid composition provided herein comprises an amino acid sequence having at least 80% sequence identity to, comprising, or consisting of, SEQ ID NO: 79.

The TCR Vβ domain sequences derived from TCR clone 13D4.9 discussed above are particularly compatible with the TCR Vα domain sequences derived from TCR clone 13D4.9 discussed elsewhere herein.

Accordingly, in one example, a nucleic acid composition described herein encodes a Jchain antigen-specific binding protein having TCR Vα domain comprising a CDR3 amino acid sequence having at least 80% sequence identity to SEQ ID NO:73, or a functional fragment thereof; and a nucleic acid sequence that encodes a TCR Vβ domain comprising a CDR3 amino acid sequence having at least 80% sequence identity to SEQ ID NO:76, or a functional fragment thereof.

In a particular example, a nucleic acid composition described herein encodes a Jchain antigen-specific binding protein having a TCR Vα domain with a CDR3 amino acid sequence comprising or consisting of the amino acid sequence of SEQ ID NO: 73; and a TCR Vβ domain with a CDR3 comprising or consisting of the amino acid sequence of SEQ ID NO:76. In addition, the Jchain antigen may comprise or consist of the sequence shown in SEQ ID NO: 101. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 77; and the Vβ domain may comprise an amino acid sequence having at least 80% sequence identity to, comprising, or consisting of, SEQ ID NO: 79. In one example, the Vα domain comprises the amino acid sequence of SEQ ID NO: 77 and the Vβ domain comprises the amino acid sequence of SEQ ID NO: 79. In such cases, the Vα domain may be encoded by a nucleic acid sequence comprising the sequence of SEQ ID NO: 78; and the Vβ domain may be encoded by a nucleic acid sequence comprising the sequence of SEQ ID NO: 80.

In this particular example, the TCR Vα domain may include a CDR1 amino acid sequence comprising or consisting of the amino acid sequence of SEQ ID NO:71 and a CDR2 amino acid sequence comprising or consisting of the amino acid sequence of SEQ ID NO:72. Furthermore, the TCR Vβ domain may include a CDR1 amino acid sequence comprising or consisting of the amino acid sequence of SEQ ID NO:74 and a CDR2 amino acid sequence comprising or consisting of the amino acid sequence of SEQ ID NO: 75.

For the avoidance of doubt, this particular example encompasses components of TCR clone 13D4.9 exemplified herein. The different components of TCR clone 13D4.9 and their respective SEQ ID Nos are summarised in Table 8 below.

As stated in more detail elsewhere herein, the nucleic acid composition described herein encodes both a TCR Vα domain and a TCR Vβ domain, which form the binding protein that is capable of specifically binding to a Jchain antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

(iii) Vβ Domains that Interact with CYTAVVPLV (SEQ ID NO: 100)

As provided elsewhere herein, the inventors identified TCR clone 10H11.11 which interacts with CYTAVVPLV (SEQ ID NO:100) in the context of HLA-A*24:02. The sequences provided herein that correspond to TCR clone 10H11.1 are SEQ ID NO:s 29 to 42.

An example of an appropriate TCR Vβ domain CDR3 amino acid sequence that confers specific binding to a Jchain antigen (e.g. to Jchain antigen CYTAVVPLV (SEQ ID NO: 100)) is shown in SEQ ID NO:34. As would be clear to a person of skill in the art, variants of the amino acid sequence shown in SEQ ID NO:34 may also be functional (i.e. retain their ability to confer specific binding to a Jchain antigen (i.e. the peptide shown in SEQ ID NO: 100) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 34, i.e. they may have at least 80%, at least 81%, at least 87%, at least 93%, or 100% sequence identity to SEQ ID NO: 34. Suitably, percent identity is calculated as the percentage of identity to the entire length of the reference sequence (e.g. SEQ ID NO: 34). In other words, appropriate (functional) Vβ domain CDR3 amino acid sequences may vary from the sequence shown in SEQ ID NO: 34 by one or several (e.g. two) amino acids. As stated above, functional variants of SEQ ID NO: 34 retain their ability to confer specific binding to a Jchain antigen (e.g. the peptide shown in SEQ ID NO: 100) when the CDR3 is part of TCR Vβ domain.

Functional variants may be naturally occurring, synthetic, or synthetically improved functional variants of SEQ ID NO: 34. The term “variant” also encompasses homologues and fragments. Functional variants will typically contain only conservative substitutions of one or more amino acids of SEQ ID NO: 34, or substitution, deletion or insertion of non-critical amino acids in non-critical regions of the CDR3.

Non-functional variants are amino acid sequence variants of SEQ ID NO: 34 that do not specifically bind to a Jchain antigen (e.g. the peptide shown in SEQ ID NO: 100). Non-functional variants will typically contain a non-conservative substitution, a deletion, or insertion or premature truncation of the amino acid sequence of SEQ ID NO: 34 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 34. In examples where the TCR Vβ domain CDR3 has the amino acid sequence of SEQ ID NO:34, the CDR3 may be encoded by any appropriate nucleic acid sequence.

The encoded TCR Vβ domain may comprise, in addition to the specified CDR3, a CDR1 comprising an amino acid sequence of SEQ ID NO: 32, or a functional variant thereof (i.e. wherein the variant retains the ability to specifically bind to a Jchain antigen (e.g. the peptide shown in SEQ ID NO: 100)). Such functional variants may be naturally occurring, synthetic, or synthetically improved functional variants of SEQ ID NO: 32. The term “variant” also encompasses homologues and fragments. Functional variants will typically contain only conservative substitutions of one or more amino acids of SEQ ID NO: 32, or substitution, deletion or insertion of non-critical amino acids in non-critical regions of the protein.

Non-functional variants are amino acid sequence variants of SEQ ID NO: 32 that do not specifically bind to a Jchain antigen (e.g. the peptide shown in SEQ ID NO: 100). Non-functional variants will typically contain a non-conservative substitution, a deletion, or insertion or premature truncation of the amino acid sequence of SEQ ID NO: 32 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 32, i.e. it may have at least 80%, or 100% sequence identity to SEQ ID NO: 32. Suitably, percent identity is calculated as the percentage of identity to the entire length of the reference sequence (e.g. SEQ ID NO: 32). In other words, appropriate (functional) Vβ domain CDR1 amino acid sequences may vary from the sequence shown in SEQ ID NO:32 by one or several amino acids. As stated previously, the variant may comprise an amino acid substitution such as a conservative amino acid substitution compared to the sequence shown in SEQ ID NO:32). As stated above, functional variants of SEQ ID NO: 32 retain the ability to specifically bind to a Jchain antigen (e.g. the peptide shown in SEQ ID NO: 100) when the CDR1 is part of TCR Vβ domain).

In one example, the CDR1 of the Vβ domain comprises or consists of the amino acid sequence of SEQ ID NO: 32. In examples where the TCR Vα domain CDR1 has the amino acid sequence of SEQ ID NO:32, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 33, or a functional variant thereof (i.e. wherein the variant retains the ability to specifically bind to HLA-A*24:02). Such functional variants may be naturally occurring, synthetic, or synthetically improved functional variants of SEQ ID NO: 33. The term “variant” also encompasses homologues and fragments. Functional variants will typically contain only conservative substitutions of one or more amino acids of SEQ ID NO: 33, or substitution, deletion or insertion of non-critical amino acids in non-critical regions of the protein.

Non-functional variants are amino acid sequence variants of SEQ ID NO: 33 that do not specifically bind to HLA-A*24:02. Non-functional variants will typically contain a non-conservative substitution, a deletion, or insertion or premature truncation of the amino acid sequence of SEQ ID NO: 33 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 33, i.e. it may have at least 80%, at least 83%, or 100% sequence identity to SEQ ID NO: 33. Suitably, percent identity is calculated as the percentage of identity to the entire length of the reference sequence (e.g. SEQ ID NO: 33). In other words, appropriate (functional) Vβ domain CDR2 amino acid sequences may vary from the sequence shown in SEQ ID NO: 33 by one or several amino acids. As stated previously, the variant may comprise an amino acid substitution such as a conservative amino acid substitution compared to the sequence shown in SEQ ID NO: 33). As stated above, a functional variant of SEQ ID NO: 33 retains the ability to specifically bind to HLA-A*24:02.

In one example, the CDR2 of the Vβ domain comprises or consists of the amino acid sequence of SEQ ID NO: 33. In examples where the TCR Vβ domain CDR2 has the amino acid sequence of SEQ ID NO:33, the CDR2 may be encoded by any appropriate nucleic acid sequence.

The encoded TCR Vβ domain may therefore comprise the CDRs mentioned in detail above (by SEQ ID specifically i.e. SEQ ID NO:34, SEQ ID NO: 32 and SEQ ID NO: 33, or functional variants thereof), with appropriate intervening sequences between the CDRs.

The encoded TCR Vβ domain may have an amino acid sequence of SEQ ID NO: 37, or a functional variant thereof (i.e. wherein the variant TCR Vβ domain retains the ability to specifically bind to a Jchain antigen (e.g. the peptide shown in SEQ ID NO: 100) when part of a binding protein described herein). Such functional variants may be naturally occurring, synthetic, or synthetically improved functional variants of SEQ ID NO: 37. The term “variant” also encompasses homologues and fragments. Functional variants will typically contain only conservative substitutions of one or more amino acids of SEQ ID NO: 37, or substitution, deletion or insertion of non-critical amino acids in non-critical regions of the protein.

Non-functional variants are amino acid sequence variants of SEQ ID NO: 37 that do not specifically bind to a Jchain antigen (e.g. the peptide shown in SEQ ID NO: 100). Non-functional variants will typically contain a non-conservative substitution, a deletion, or insertion or premature truncation of the amino acid sequence of SEQ ID NO:37 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 37, whilst retaining the ability to specifically bind to a Jchain antigen (e.g. the peptide shown in SEQ ID NO: 100). In other words, a functional TCR Vβ domain with one or several amino acid substitutions compared to the sequence of SEQ ID NO: 37 is also encompassed. As stated previously, the amino acid substitution may be a conservative amino acid substitution. The variability in sequence compared to SEQ ID NO:37 may all be in regions of the TCR Vβ domain that do not form CDRs (i.e. the variant may have the CDRs of SEQ ID NO: 34, SEQ ID NO: 32 and/or SEQ ID NO: 33, and still have 25% (or less) sequence variability compared to SEQ ID NO: 37). In other words, the sequence of the CDRs of SEQ ID NO: 37 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 37).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 37, wherein the TCR Vβ domain comprises a CDR3 having an amino acid sequence of SEQ ID NO: 34. In this example, the TCR Vβ domain CDR1 may have an amino acid sequence of SEQ ID NO:32 and the TCR Vβ domain CDR2 may have an amino acid sequence of SEQ ID NO: 33.

In examples where the TCR Vβ domain has the amino acid sequence of SEQ ID NO:37, the TCR Vβ domain may be encoded by the nucleic acid sequence of SEQ ID NO:38, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 41. Appropriate functional variants of SEQ ID NO: 41 are also encompassed (e.g. variants having at least 75% (e.g. at least 75%, at least 80%, at least 85%, at least 90%, at least 95% etc) sequence identity to the amino acid sequence of SEQ ID NO: 41, wherein the variant TCR β chain amino acid sequence retains its ability to specifically bind to a Jchain antigen (e.g. the peptide shown in SEQ ID NO: 100) when part of a binding protein described herein). In other words, a functional TCR β chain with one or several amino acid substitutions compared to the sequence of SEQ ID NO: 41 is also encompassed. As stated previously, the amino acid substitution may be a conservative amino acid substitution. The variability in sequence compared to SEQ ID NO:41 may all be in regions of the TCR β chain that do not form CDRs (i.e. the variant may have the CDRs of SEQ ID NO: 34, SEQ ID NO: 32 and/or SEQ ID NO: 33, and still have 25% (or less) sequence variability compared to SEQ ID NO:41. In other words, the sequence of the CDRs of SEQ ID NO: 41 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 41).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 41, wherein the TCR B chain comprises a CDR3 having an amino acid sequence of SEQ ID NO: 34. In this example, the TCR β chain CDR1 may have an amino acid sequence of SEQ ID NO: 32 and the TCR β chain CDR2 may have an amino acid sequence of SEQ ID NO: 33.

In examples where the TCR β chain has the amino acid sequence of SEQ ID NO:41, the TCR β chain may be encoded by the nucleic acid sequence of SEQ ID NO: 42, or a genetically degenerate sequence thereof (i.e. other nucleic acid sequences that encode the same protein as a result of the degeneracy of the genetic code). It is noted that SEQ ID NO:42 is the nucleic acid sequence for TCR β chain of clone 10H11.11.

In an example, the nucleic acid composition provided herein comprises a nucleic acid sequence that encodes a TCR Vβ domain comprising a CDR3 amino acid sequence having at least 80% sequence identity to SEQ ID NO:34, or a functional fragment thereof.

In another example, the CDR3 of the Vβ domain of a nucleic acid composition provided herein comprises or consists of the amino acid sequence of SEQ ID NO:34.

In a further example, the Vβ domain of a nucleic acid composition provided herein comprises an amino acid sequence having at least 80% sequence identity to, comprising, or consisting of, SEQ ID NO: 37.

The TCR Vβ domain sequences derived from TCR clone 10H11.11 discussed above are particularly compatible with the TCR Vα domain sequences derived from TCR clone 10H11.11 discussed elsewhere herein.

Accordingly, in one example, a nucleic acid composition described herein encodes a Jchain antigen-specific binding protein having TCR Vα domain comprising a CDR3 amino acid sequence having at least 80% sequence identity to SEQ ID NO:31, or a functional fragment thereof; and a nucleic acid sequence that encodes a TCR Vβ domain comprising a CDR3 amino acid sequence having at least 80% sequence identity to SEQ ID NO:34, or a functional fragment thereof.

In a particular example, a nucleic acid composition described herein encodes a Jchain antigen-specific binding protein having a TCR Vα domain with a CDR3 amino acid sequence comprising or consisting of the amino acid sequence of SEQ ID NO: 31; and a TCR Vβ domain with a CDR3 comprising or consisting of the amino acid sequence of SEQ ID NO:34. In addition, the Jchain antigen may comprise or consist of the sequence shown in SEQ ID NO: 100. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 35; and the Vβ domain may comprise an amino acid sequence having at least 80% sequence identity to, comprising, or consisting of, SEQ ID NO: 37. In one example, the Vα domain comprises the amino acid sequence of SEQ ID NO: 35 and the Vβ domain comprises the amino acid sequence of SEQ ID NO: 37. In such cases, the Vα domain may be encoded by a nucleic acid sequence comprising the sequence of SEQ ID NO: 36; and the Vβ domain may be encoded by a nucleic acid sequence comprising the sequence of SEQ ID NO: 38.

In this particular example, the TCR Vα domain may include a CDR1 amino acid sequence comprising or consisting of the amino acid sequence of SEQ ID NO: 29 and a CDR2 amino acid sequence comprising or consisting of the amino acid sequence of SEQ ID NO:30. Furthermore, the TCR Vβ domain may include a CDR1 amino acid sequence comprising or consisting of the amino acid sequence of SEQ ID NO:32 and a CDR2 amino acid sequence comprising or consisting of the amino acid sequence of SEQ ID NO: 33.

For the avoidance of doubt, this particular example encompasses components of TCR clone 10H11.11 exemplified herein. The different components of TCR clone 10H11.11 and their respective SEQ ID Nos are summarised in Table 5 below.

As stated in more detail elsewhere herein, the nucleic acid composition described herein encodes both a TCR Vα domain and a TCR Vβ domain, which form the binding protein that is capable of specifically binding to a Jchain antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

### Vector Systems

A vector system is also provided which includes a nucleic acid composition described herein. The vector system may have one or more vectors. As discussed previously, the binding protein components that are encoded by the nucleic acid composition may be encoded by one or more nucleic acid sequences in the nucleic acid composition. In examples where all of the binding protein components are encoded by a single nucleic acid sequence, the nucleic acid sequence may be present within a single vector (and thus the vector system described herein may comprise of one vector only). In examples where the binding protein components are encoded by two or more nucleic acid sequences (wherein the plurality of nucleic acid sequences, together, encode all of the components of the binding protein) these two or more nucleic acid sequences may be present within one vector (e.g. in different open reading frames of the vector), or may be distributed over two or more vectors. In this example, the vector system will comprise a plurality of distinct vectors (i.e. vectors with different nucleotide sequences).

Accordingly, in one example, a vector system is provided, comprising a nucleic acid composition described herein.

Any appropriate vector can be used. By way of example only, the vector may be a plasmid, a cosmid, or a viral vector, such as a retroviral vector or a lentiviral vector. Adenovirus, adeno-associated virus, vaccinia virus, canary poxvirus, herpes virus, minicircle vectors and naked (synthetic) DNA/RNA may also be used (for details on minicircle vectors, see for example non-viral Sleeping Beauty transposition from minicircle vectors as published by R Monjezi et al., Leukemia 2017). Alternatively, single stranded or double stranded DNA or RNA can be used to transfect lymphocytes with a TCR of interest (see Roth et al 2018 Nature vol 559; page 405).

In one example, the vector is a plasmid, a viral vector, or a cosmid, optionally wherein the vector is selected from the group consisting of a retrovirus, lentivirus, adeno-associated virus, adenovirus, vaccinia virus, canary poxvirus, herpes virus, minicircle vector and synthetic DNA or RNA.

As used herein, the term “vector” refers to a nucleic acid sequence capable of transporting another nucleic acid sequence to which it has been operably linked. The vector can be capable of autonomous replication or it can integrate into a host DNA. The vector may include restriction enzyme sites for insertion of recombinant DNA and may include one or more selectable markers or suicide genes. The vector can be a nucleic acid sequence in the form of a plasmid, a bacteriophage or a cosmid. Preferably the vector is suitable for expression in a cell (i.e. the vector is an “expression vector”). Preferably, the vector is suitable for expression in a human T cell such as a CD8⁺ T cell or CD4⁺ T cell, or stem cell, iPS cell, or NK cell. In certain aspects, the vector is a viral vector, such as a retroviral vector, a lentiviral vector or an adeno-associated vector. Optionally, the vector is selected from the group consisting of an adenovirus, vaccinia virus, canary poxvirus, herpes virus, minicircle vector and synthetic DNA or synthetic RNA.

Preferably the (expression) vector is capable of propagation in a host cell and is stably transmitted to future generations.

The vector may comprise regulatory sequences. “Regulatory sequences” as used herein, refers to, DNA or RNA elements that are capable of controlling gene expression. Examples of expression control sequences include promoters, enhancers, silencers, TATA-boxes, internal ribosomal entry sites (IRES), attachment sites for transcription factors, transcriptional terminators, polyadenylation sites etc. Optionally, the vector includes one or more regulatory sequences operatively linked to the nucleic acid sequence to be expressed. Regulatory sequences include those which direct constitutive expression, as well as tissue-specific regulatory and/or inducible sequences.

Optionally, the vector comprises the nucleic acid sequence of interest operably linked to a promoter. “Promoter”, as used herein, refers to the nucleotide sequences in DNA to which RNA polymerase binds to start transcription. The promoter may be inducible or constitutively expressed. Alternatively, the promoter is under the control of a repressor or stimulatory protein. The promoter may be one that is not naturally found in the host cell (e.g. it may be an exogenous promoter). The skilled person in the art is well aware of appropriate promoters for use in the expression of target proteins, wherein the selected promoter will depend on the host cell.

“Operably linked” refers to a single or a combination of the below-described control elements together with a coding sequence in a functional relationship with one another, for example, in a linked relationship so as to direct expression of the coding sequence.

The vector may comprise a transcriptional terminator. “Transcriptional terminator” as used herein, refers to a DNA element, which terminates the function of RNA polymerases responsible for transcribing DNA into RNA. Preferred transcriptional terminators are characterized by a run of T residues preceded by a GC rich dyad symmetrical region.

The vector may comprise a translational control element. “Translational control element”, as used herein, refers to DNA or RNA elements that control the translation of mRNA. Preferred translational control elements are ribosome binding sites. Preferably, the translational control element is from a homologous system as the promoter, for example a promoter and its associated ribozyme binding site. Preferred ribosome binding sites are known, and will depend on the chosen host cell.

The vector may comprise restriction enzyme recognition sites. “Restriction enzyme recognition site” as used herein, refers to a motif on the DNA recognized by a restriction enzyme.

The vector may comprise a selectable marker. “Selectable marker” as used herein, refers to proteins that, when expressed in a host cell, confer a phenotype onto the cell which allows a selection of the cell expressing said selectable marker gene. Generally this may be a protein that confers a new beneficial property onto the host cell (e.g. antibiotic resistance) or a protein that is expressed on the cell surface and thus accessible for antibody binding. Appropriate selectable markers are well known in the art.

Optionally, the vector may also comprise a suicide gene. “Suicide gene” as used herein encodes a protein that induce death of the modified cell upon treatment with specific drugs. By way of example, suicide can be induced in cells modified by the herpes simplex virus thymidine kinase gene upon treatment with specific nucleoside analogs including ganciclovir, cells modified by human CD20 upon treatment with anti-CD20 monoclonal antibody and cells modified with inducible Caspase9 (iCasp9) upon treatment with AP1903 (reviewed by B S Jones, L S Lamb, F Goldman, A Di Stasi; Improving the safety of cell therapy products by suicide gene transfer. Front Pharmacol. (2014) 5:254). Appropriate suicide genes are well known in the art.

Preferably the vector comprises those genetic elements which are necessary for expression of the binding proteins described herein by a host cell. The elements required for transcription and translation in the host cell include a promoter, a coding region for the protein(s) of interest, and a transcriptional terminator.

A person of skill in the art will be well aware of the molecular techniques available for the preparation of (expression) vectors and how the (expression) vectors may be transduced or transfected into an appropriate host cell (thereby generating a modified cell described further below). The (expression) vector system described herein can be introduced into cells by conventional techniques such as transformation, transfection or transduction. “Transformation”, “transfection” and “transduction” refer generally to techniques for introducing foreign (exogenous) nucleic acid sequences into a host cell, and therefore encompass methods such as electroporation, microinjection, gene gun delivery, transduction with retroviral, lentiviral or adeno-associated vectors, lipofection, superfection etc. The specific method used typically depends on both the type of vector and the cell. Appropriate methods for introducing nucleic acid sequences and vectors into host cells such as human cells are well known in the art; see for example Sambrook et al (1989) Molecular Cloning, A Laboratory Manual, Cold Spring Harbor Laboratory, Cold Spring Harbor, N.Y; Ausubel et al (1987) Current Protocols in Molecular Biology, John Wiley and Sons, Inc., NY; Cohen et al (1972) Proc. Natl. Acad. Sci. USA 69, 2110; Luchansky et al (1988) Mol. Microbiol. 2, 637-646. Further conventional methods that are suitable for preparing expression vectors and introducing them into appropriate host cells are described in detail in WO2016/071758 for example.

It is understood that it some examples, the host cell is contacted with the vector system (e.g. viral vector) in vitro, ex vivo, and in some examples, the host cell is contacted with the vector system (e.g. viral vector) in vivo.

The term “host cell” includes any cell into which the nucleic acid composition or vector system described herein may be introduced. Once a nucleic acid molecule or vector system has been introduced into the cell, it may be referred to as a “modified cell” herein. Once the nucleic acid molecule or vector is introduced into the host cell, the resultant modified cell should be capable of expressing the encoded binding protein (and e.g. correctly localising the encoded binding protein for its intended function e.g. transporting the encoded binding protein to the cell surface).

The nucleic acid composition or vector system may be introduced into the cell using any conventional method known in the art. For example, the nucleic acid composition or vector system may be introduced using CRISPR technology. Insertion of the nucleic acid sequences at the endogenous TCR locus by engineering with CRISPR/Cas9 and homologous directed repair (HDR) or non-homologous end joining (NHEJ) is therefore encompassed. Other conventional methods such as transfection, transduction or transformation of the cell may also be used.

The term “modified cell” refers to a genetically altered (e.g. recombinant) cell. The modified cell includes at least one exogenous nucleic acid sequence (i.e. a nucleic acid sequence that is not naturally found in the host cell). In the context of the invention, the exogenous sequence comprises at least one of the T cell receptor component parts described herein for any of clones 4G8.8, 5D12.9, 13F6.6, 10H11.11, 5C8.16, 16C7.9 or 13D4.9 (e.g. the sequences etc that encode the CDR3 sequences that are specific for a Jchain antigen (e.g. the peptide of SEQ ID NO: 99, SEQ ID NO: 100 or SEQ ID NO: 101)). The term “modified cell” refers to the particular subject cell and also to the progeny or potential progeny of such a cell. Because certain modifications may occur in succeeding generations due to either mutation or environmental influences, such progeny may not, in fact, be identical to the parent cell, but are still included within the scope of the term as used herein.

In one example, a modified cell comprises a nucleic acid composition or a vector system provided herein.

The host cell (and thus the modified cell) is typically a eukaryotic cell, and particularly a human cell (e.g. a T cell such as a CD8⁺ T cell or a CD4⁺ T cell, or a mixture thereof, or a hematopoietic stem cell, an iPSC, or gamma-delta T cell, or a pluripotent stem cell, or a NK-T cell or NK cell). The host cell (and thus the modified cell) may be an autologous or allogeneic cell (e.g. such as a CD8⁺ T cell or a CD4⁺ T cell, or a mixture thereof, or a hematopoietic stem cell, an iPSC, or gamma-delta T cell, or a pluripotent stem cell, or a NK-T cell or NK cell). “Allogeneic cell” refers to a cell derived from a different individual to the individual to which it is later administered. In other words, the host cell (and thus the modified cell) may be an isolated cell from a distinct individual compared to the subject to be treated. “Autologous cell” refers to a cell derived from the individual to which it is also later administered. In other words, the host cell (and thus the modified cell) may be an isolated cell from the subject that is to be treated.

Accordingly, in an example, the modified cell is a human cell.

The host cell (and thus the modified cell) may be any cell that is able to confer anti-tumour immunity after TCR gene transfer. Non limiting examples of appropriate cells include autologous or allogeneic CD8 T cells, CD4 T cells, Natural Killer (NK) cells, NKT cells, gamma-delta T cells, inducible pluripotent stem cells (iPSCs), hematopoietic stem cells or other progenitor cells and any other autologous or allogeneic cell or cell line (NK-92 for example or T cell lines) that is able to confer anti-tumor immunity after TCR gene transfer.

Accordingly, in one example the modified cell is selected from the group consisting of a CD8 T cell, a CD4 T cell, an NK cell, an NK-T cell, a gamma-delta T cell, a hematopoietic stem cell, an inducible pluripotent stem cell, a progenitor cell, a T cell line and a NK-92 cell line.

In the context of the methods of treatment described herein, the host cell (and thus the modified cell) is typically for administration to a HLA-A*01:01, HLA-A*02:01, HLA-A*03:01, HLA-A*11:01, HLA-A*24:02 and/or HLA-B*40:01 positive human subject (e.g. to a HLA-A*01:01, HLA-A*02:01, HLA-A*03:01, HLA-A*11:01 and/or HLA-A*24:02 positive human subject). In view of this, the host cell (and thus the modified cell) is typically HLA-A*01:01, HLA-A*02:01, HLA-A*03:01, HLA-A*11:01, HLA-A*24:02 and/or HLA-B*40:01 positive but needs to be Jchain antigen negative (i.e. modified cells can either be HLA-A*01:01, HLA-A*02:01, HLA-A*03:01, HLA-A*11:01, HLA-A*24:02 and/or HLA-B*40:01 positive or negative).

In the context of the methods of treatment described herein, the host cell (and thus the modified cell) that is to be administered to the subject can either be autologous or allogeneic.

Advantageously, the modified cell is capable of expressing the binding protein encoded by the nucleic acid composition or vector system described herein (i.e. the TCR component parts) such that the modified cell provides an immunotherapy that specifically targets cells that express a Jchain antigen, and thus can be used to treat or prevent B cell associated diseases or conditions in a corresponding HLA-A*01:01, HLA-A*02:01, HLA-A*03:01, HLA-A*11:01 and/or HLA-A*24:02 positive human subject. More details on this use are given below.

### Immunogenic Peptides

The inventors have identified several Jchain derived peptides that are presented on MM cells in HLA-A*01:01 (HLA-A1), HLA-A*02:01 (HLA-A2), HLA-A*03:01 (HLA-A3), HLA-A*11:01 (HLA-A11), HLA-A*24:02 (HLA-A24), and HLA-B*40:01 (HLA-B40). Specifically, the inventors identified the Jchain derived peptide antigens YTAVVPLVY (SEQ ID NO:99),TAVVPLVY (SEQ ID NO:102), VLAVFIKAVHV (SEQ ID NO:103), VLAVFIKAV (SEQ ID NO:104), YTAVVPLV (SEQ ID NO:105), ISDPTSPLRTR (SEQ ID NO:101), RIIVPLNNR (SEQ ID NO:106), CYTAVVPLV (SEQ ID NO:100) and GETKMVETAL (SEQ ID NO:107).

In the context of an isolated peptide, the invention specifically provides for an isolated peptide comprising or consisting of an amino acid sequence selected from the group consisting of: YTAVVPLVY (SEQ ID NO:99), TAVVPLVY (SEQ ID NO: 102), VLAVFIKAVHV (SEQ ID NO: 103), VLAVFIKAV (SEQ ID NO:104), YTAVVPLV (SEQ ID NO:105), ISDPTSPLRTR (SEQ ID NO:101), RIIVPLNNR (SEQ ID NO:106), CYTAVVPLV (SEQ ID NO:100) and GETKMVETAL (SEQ ID NO:107).

In one example, the invention specifically provides for an isolated peptide comprising or consisting of an amino acid sequence selected from the group consisting of: YTAVVPLVY (SEQ ID NO:99), ISDPTSPLRTR (SEQ ID NO:101) and CYTAVVPLV (SEQ ID NO:100).

In a particular example, the invention specifically provides for an isolated peptide comprising or consisting of an amino acid sequence of YTAVVPLVY (SEQ ID NO:99).

In a particular example, the invention specifically provides for an isolated peptide comprising or consisting of an amino acid sequence of ISDPTSPLRTR (SEQ ID NO:101).

In a particular example, the invention specifically provides for an isolated peptide comprising or consisting of an amino acid sequence of CYTAVVPLV (SEQ ID NO:100).

As used herein, an “isolated peptide” refers to a peptide that is not in its natural environment. The peptide may therefore be of synthetic origin (or alternatively, of natural original, but isolated from its natural environment).

The isolated peptide may be relatively short (i.e. no more than 20 amino acids; e.g. no more than 19, 18, 17, 16, 15, 14, 13, 12, 11, or 10 amino acids).

In one example, the peptide may be no more than 20 amino acids long.

The peptide may consist of the amino acid sequence of SEQ ID NO:99, SEQ ID NO:100, SEQ ID NO:101 SEQ ID NO:102, SEQ ID NO:103, SEQ ID NO:104, SEQ ID NO:105, SEQ ID NO:106 or SEQ ID NO:107 only.

In a particular example, the peptide may consist of the amino acid sequence of SEQ ID NO:99, SEQ ID NO:100 or SEQ ID NO:101.

In a particular example, the peptide may consist of the amino acid sequence of SEQ ID NO:99.

In a particular example, the peptide may consist of the amino acid sequence of SEQ ID NO:100.

In a particular example, the peptide may consist of the amino acid sequence of SEQ ID NO:101.

The isolated peptide may be administered to a human subject in order to treat or prevent a B cell associated disease or condition, such as MM. For example, the isolated peptide may be administered to the subject in order to induce or enhance their immune response. The peptide may therefore be administered to the subject to induce T cell activation (e.g. in vivo T cell activation) in the subject, wherein the activated T cells are specific for the peptide (and thus will specifically target Jchain positive cells).

The isolated peptide may be administered as a peptide vaccine for treating or preventing a B cell associated disease or condition, such as MM. The isolated peptide may be administered to induce or enhance activation of T cells specific for Jchain positive cells.

The inventors have shown that YTAVVPLVY (SEQ ID NO:99), TAVVPLVY (SEQ ID NO: 102), VLAVFIKAVHV (SEQ ID NO: 103), VLAVFIKAV (SEQ ID NO:104), YTAVVPLV (SEQ ID NO:105), ISDPTSPLRTR (SEQ ID NO:101), RIIVPLNNR (SEQ ID NO:106), CYTAVVPLV (SEQ ID NO:100) and GETKMVETAL (SEQ ID NO:107) are Jchain derived peptides that are presented at the cell surface in the context of HLA and are recognized by T cells. Binding of these peptides to T cells has been demonstrated herein. These peptides therefore represent bonafide immunogenic Jchain-specific antigens that may be further exploited in the development of personalized vaccines, which may be particularly useful as an adjunct to other therapies (e.g. ACT as described herein). These immunogenic peptides can therefore be used as an immunotherapy in the form of peptide, RNA, DNA, dendritic cell based therapies, and adoptive TCR transgenic T cell-based therapies.

An isolated peptide as described herein may therefore be useful as an immunotherapy. For example, such isolated peptides may be used as an immunotherapy for subjects with, at risk of developing, or suspected of having a B cell associated disease or condition. Nucleic acid sequences and vectors encoding these peptides may also be useful for this purpose. Nucleic acid sequences and vectors encoding such isolated peptides are therefore also provided herein. A general discussion of vectors and nucleic acid sequences is provided elsewhere herein and applies equally to this aspect.

The particular peptide for administration may be chosen based on the HLA-A status of the subject. As explained elsewhere herein, a peptide comprising the sequence of SEQ ID NO:99 or SEQ ID NO: 102 may be particularly suitable for administration to a subject that is HLA-A*01:01 positive, whereas a peptide comprising the sequence of SEQ ID NO:103, SEQ ID NO:104, or SEQ ID NO:105 may be particularly suitable for administration to a subject that is positive for HLA-A*02:01.

Furthermore, a peptide comprising the sequence of SEQ ID NO:100 may be particularly suitable for administration to a subject that is positive for HLA-A*24:02, whereas a peptide comprising the sequence of SEQ ID NO:101 may be particularly suitable for administration to a subject that is positive for HLA-A*03:01 or HLA-A*11:01.

Furthermore, a peptide comprising the sequence of SEQ ID NO:106 may be particularly suitable for administration to a subject that is positive for HLA-A*11:01, whereas a peptide comprising the sequence of SEQ ID NO:107 may be particularly suitable for administration to a subject that is positive for HLA-B*40:01.

Isolated peptides of the invention may also be provided in compositions that comprise more than one of the peptides discussed above. By way of example, an isolated peptide may be provided (and/or administered) as a composition that comprises a mixture of (a) an isolated peptide comprising the amino acid sequence SEQ ID NO:99; and (b) an isolated peptide comprising the amino acid sequence SEQ ID NO:102. This composition may be particularly useful for administration to a subject that is HLA-A*01:01 positive as these peptides are both presented by this HLA-A serotype.

Alternatively, an isolated peptide may be provided (and/or administered) as a composition that comprises a mixture of two or three of: (a) an isolated peptide comprising the amino acid sequence SEQ ID NO:103; (b) an isolated peptide comprising the amino acid sequence SEQ ID NO:104; and (c) an isolated peptide comprising the amino acid sequence SEQ ID NO:105. This composition may be particularly useful for administration to a subject that is HLA-A*02:01 positive as these peptides are all presented by this HLA-A serotype.

In another example, an isolated peptide may be provided (and/or administered) as a composition that comprises a mixture of (a) an isolated peptide comprising the amino acid sequence SEQ ID NO:106; and (b) an isolated peptide comprising the amino acid sequence SEQ ID NO:101. This composition may be particularly useful for administration to a subject that is HLA-A*11:01 positive as these peptides are both presented by this HLA-A serotype.

### Pharmaceutical Compositions

A nucleic acid composition, vector system, modified cell, isolated peptide or isolated nucleic acid sequence described herein may be provided as part of a pharmaceutical composition. Advantageously, such compositions may be administered to a human subject in need thereof (as described elsewhere herein). A particularly suitable composition may be selected based on the HLA serotype of the human subject, as described in detail elsewhere herein.

A pharmaceutical composition may comprise a nucleic acid composition, vector system, modified cell, isolated peptide or isolated nucleic acid sequence described herein along with a pharmaceutically acceptable excipient, adjuvant, diluent and/or carrier.

Compositions may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected nucleic acid composition, vector system, modified cell, isolated peptide or isolated nucleic acid sequence without causing any undesirable biological effects or interacting in a deleterious manner with any of the other components of the pharmaceutical composition in which it is contained.

Excipients are natural or synthetic substances formulated alongside an active ingredient (e.g. a nucleic acid sequence, a nucleic acid composition, vector or vector system, modified cell, isolated peptide or isolated nucleic acid as provided herein), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art.

Diluents are diluting agents. Pharmaceutically acceptable diluents are well known in the art. A suitable diluent is therefore easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

In one example, the pharmaceutical composition comprises an isolated peptide described herein, an isolated nucleic acid sequence encoding the peptide or a vector system comprising said nucleic acid sequence, wherein the pharmaceutical composition is formulated as a vaccine (e.g. a composition that it used to stimulate the immune system and provide immunity against one or several diseases, where the composition acts as an antigen without inducing the disease).

### Treatment of a Subject

Pharmaceutical compositions described herein may advantageously be administered to a HLA-A*01:01, HLA-A*02:01, HLA-A*03:01, HLA-A* 11:01, HLA-A*24:02 and/or HLA-B*40:01 positive human subject in need thereof (where certain compositions are more suitable for certain human subjects, based on their HLA status, as described in more detail elsewhere herein). In some examples, the pharmaceutical compositions described herein may advantageously be administered to a HLA-A*01:01, HLA-A*03:01, HLA-A*11:01 or HLA-A*24:02 positive human subject in need thereof.

Typically, the subject in need of treatment has a disease or condition that is associated with an elevated level of HLA-restricted Jchain antigens (i.e. Jchain antigens that are presented at the cell surface in the context of an HLA).

The disease or condition is typically a B cell associated disease or condition. As used herein, a B cell associated disease or condition is a disease or condition that is of plasma B cell, B cell or B cell precursor origin.

In one example, the B cell associated disease or condition may be a hematological malignancy. In other words, it may be a hematological malignancy of plasma cell, B-cell and B-cell precursor origin. Examples of appropriate hematological malignancies are well known in the art, and include, for example Multiple myeloma, plasma cell leukemia, (AL) Amyloidosis, Acute lymphoblastoid leukemia (ALL), Chronic lymphocytic leukemia (CLL), Waldenstrom macroglobulinemia or B cell lymphomas. Examples of B cell lymphomas include but are not limited to: Diffuse large B cell lymphoma (DLBCL), High grade B cell lymphoma, Mantel cell lymphoma (MCL), Follicular lymphoma (FL), and Burkitt Lymphoma.

In one example, the B cell associated disease or condition is multiple myeloma.

In an alternative example, the B cell associated disease or condition may be an autoimmune disease or disorder. In other words, it may be an autoimmune disease or disorder of plasma cell, B-cell and B-cell precursor origin. Examples of appropriate autoimmune diseases or disorders are well known in the art, and include, for example Rheumatoid arthritis, Multiple sclerosis, Vasculitis including Urticarial vasculitis, systemic vasculitis, renal vasculitis, Systemic lupus erythematosus (SLE), Autoimmune hemolytic anemia and Thrombocytopenia.

The B cell associated disease or condition may be a hyperproliferative disease or condition. For example, the B cell associated disease or condition may be a HLA-restricted Jchain antigen expressing tumor or cancer.

In an example, the pharmaceutical composition provided herein is for use in inducing or enhancing an immune response in human subject diagnosed with a B cell associated disease or condition.

As would be clear to a person skilled in the art, an appropriate therapy for subject in need thereof (e.g. an appropriate pharmaceutical composition described herein) may be selected based on the HLA serotype of the subject.

In one example, if the subject in need thereof is HLA-A*01:01 positive, an appropriate therapy (e.g. an appropriate pharmaceutical composition described herein) may comprise (i) components of TCR clone 4G8.8 exemplified herein; (ii) components of TCR clone 5D12.9 exemplified herein; or (iii) components of TCR clone 13F6.6 exemplified herein. Accordingly, TCRs comprising components of (i) TCR clone 4G8.8 exemplified herein; (ii) TCR clone 5D12.9 exemplified herein; or (iii) TCR clone 13F6.6 exemplified herein are particularly suitable for administration, or treating, stimulating, providing appropriate immunity (e.g. anti-tumor immunity etc) in HLA-A*01:01 positive human subjects. In a further example, an appropriate therapy (e.g. an appropriate pharmaceutical composition described herein) for a HLA-A*01:01 positive subject in need thereof may comprise an isolated peptide comprising or consisting of the amino acid sequence YTAVVPLVY (SEQ ID NO:99), an isolated nucleic acid encoding said peptide or a vector system encoding said nucleic acid.

In one example, if the subject in need thereof is HLA-A*24:02 positive, an appropriate therapy (e.g. an appropriate pharmaceutical composition described herein) may comprise components of TCR clone 10H11.11 exemplified herein. Accordingly, TCRs comprising components of TCR clone 10H11.11 exemplified herein are particularly suitable for administration, or treating, stimulating, providing appropriate immunity (e.g. anti-tumor immunity etc) in HLA-A*24:02 positive human subjects. In a further example, an appropriate therapy (e.g. an appropriate pharmaceutical composition described herein) for a HLA-A*24:02 positive subject in need thereof may comprise an isolated peptide comprising or consisting of the amino acid sequence CYTAVVPLV (SEQ ID NO:100), an isolated nucleic acid encoding said peptide or a vector system encoding said nucleic acid.

In one example, if the subject in need thereof is HLA-A*03:01 positive, an appropriate therapy (e.g. an appropriate pharmaceutical composition described herein) may comprise components of TCR clone 5C8.16 exemplified herein. Accordingly, TCRs comprising components of TCR clone 5C8.16 exemplified herein are particularly suitable for administration, or treating, stimulating, providing appropriate immunity (e.g. anti-tumor immunity etc) in HLA-A*03:01 positive human subjects. In a further example, an appropriate therapy (e.g. an appropriate pharmaceutical composition described herein) for a HLA-A*03:01 positive subject in need thereof may comprise an isolated peptide comprising or consisting of the amino acid sequence ISDPTSPLRTR (SEQ ID NO:101), an isolated nucleic acid encoding said peptide or a vector system encoding said nucleic acid.

In one example, if the subject in need thereof is HLA-A*11:01 positive, an appropriate therapy (e.g. an appropriate pharmaceutical composition described herein) may comprise (i) components of TCR clone 16C7.9 exemplified herein; or (ii) components of TCR clone 13D4.9 exemplified herein. Accordingly, TCRs comprising components of (i) TCR clone 16C7.9 exemplified herein; or (ii) TCR clone 13D4.9 exemplified herein are particularly suitable for administration, or treating, stimulating, providing appropriate immunity (e.g. anti-tumor immunity etc) in HLA-A*11:01 positive human subjects. In a further example, an appropriate therapy (e.g. an appropriate pharmaceutical composition described herein) for a HLA-A*11:01 positive subject in need thereof may comprise an isolated peptide comprising or consisting of the amino acid sequence ISDPTSPLRTR (SEQ ID NO:101), an isolated nucleic acid encoding said peptide or a vector system encoding said nucleic acid.

In a further example, an appropriate therapy (e.g. an appropriate pharmaceutical composition described herein) for a HLA-A*01:01 positive subject in need thereof may comprise an isolated peptide comprising or consisting of the amino acid sequence TAVVPLVY (SEQ ID NO:102), an isolated nucleic acid encoding said peptide or a vector system encoding said nucleic acid.

In a further example, an appropriate therapy (e.g. an appropriate pharmaceutical composition described herein) for a HLA-A*02:01 positive subject in need thereof may comprise an isolated peptide comprising or consisting of the amino acid sequence VLAVFIKAVHV (SEQ ID NO:103), an isolated nucleic acid encoding said peptide or a vector system encoding said nucleic acid.

In a further example, an appropriate therapy (e.g. an appropriate pharmaceutical composition described herein) for a HLA-A*02:01 positive subject in need thereof may comprise an isolated peptide comprising or consisting of the amino acid sequence VLAVFIKAV (SEQ ID NO:104), an isolated nucleic acid encoding said peptide or a vector system encoding said nucleic acid.

In a further example, an appropriate therapy (e.g. an appropriate pharmaceutical composition described herein) for a HLA-A*02:01 positive subject in need thereof may comprise an isolated peptide comprising or consisting of the amino acid sequence YTAVVPLV (SEQ ID NO:105), an isolated nucleic acid encoding said peptide or a vector system encoding said nucleic acid.

In a further example, an appropriate therapy (e.g. an appropriate pharmaceutical composition described herein) for a HLA-A*11:01 positive subject in need thereof may comprise an isolated peptide comprising or consisting of the amino acid sequence RIIVPLNNR (SEQ ID NO:106), an isolated nucleic acid encoding said peptide or a vector system encoding said nucleic acid.

In a further example, an appropriate therapy (e.g. an appropriate pharmaceutical composition described herein) for a HLA-B*40:01 positive subject in need thereof may comprise an isolated peptide comprising or consisting of the amino acid sequence GETKMVETAL (SEQ ID NO:107), an isolated nucleic acid encoding said peptide or a vector system encoding said nucleic acid.

The phrase “induced or enhanced immune response” refers to an increase in the immune response (e.g. a cell mediated immune response such as a T cell mediated immune response) of the subject during or after treatment compared to their immune response prior to treatment. An “induced or enhanced” immune response therefore encompasses any measurable increase in the immune response that is directly or indirectly targeted to the disease or condition being treated (or prevented).

In another example, the pharmaceutical composition may be for use in stimulating a cell mediated immune response to a target cell population or tissue in a human subject.

In another example, the pharmaceutical composition may be for use in stimulating a cell mediated immune response to a target cell population or tissue in a human subject. In such an example, the target cell population or tissue may be a HLA-restricted Jchain antigen expressing target cell population or tissue. Typically, it is a HLA-restricted Jchain antigen expressing target cell population or tissue. For example, it may be a target cell population or tissue comprising a HLA-restricted Jchain antigen expressing tumor or cancer.

The pharmaceutical composition may also be for use in providing anti-tumor immunity to a human subject.

In another example, the pharmaceutical composition may be for use in treating a human subject having a disease or condition associated with an elevated level of HLA-restricted Jchain antigen. Typically, the disease or condition associated with an elevated level of HLA-restricted Jchain antigen may be a B cell associated disease or condition.

A person of skill in the art will be fully aware of B cell associated diseases or conditions that may be treated in accordance with the invention. Appropriate examples of such diseases or conditions are discussed elsewhere herein.

As would be clear to a person skilled in the art, the B cell associated diseases or conditions may comprise at least one tumor (particularly, at least one HLA-restricted Jchain antigen expressing tumor).

As used herein, the terms “treat”, “treating” and “treatment” are taken to include an intervention performed with the intention of preventing the development or altering the pathology of a condition, disorder or symptom (e.g. a B cell associated disease or condition). Accordingly, “treatment” refers to both therapeutic treatment and prophylactic or preventative measures, wherein the object is to prevent or slow down (lessen) the targeted condition, disorder or symptom. “Treatment” therefore encompasses a reduction, slowing or inhibition of the amount or concentration of target cells, for example as measured in a sample obtained from the subject, of at least 5%, 10%, 20%, 30%, 40%, 50%, 60%, 70%, 80%, 90% or 100% when compared to the amount or concentration of target cells before treatment. Methods of measuring the amount or concentration of target cells include, for example, qRT-PCR, and quantification of disease specific biomarkers in a sample obtained from the subject.

As used herein the term “subject” refers to an individual, e.g., a human, having or at risk of having a specified condition, disorder or symptom. The subject may be a patient i.e. a subject in need of treatment in accordance with the invention. The subject may have received treatment for the condition, disorder or symptom. Alternatively, the subject has not been treated prior to treatment in accordance with the present invention.

The compositions described herein can be administered to the subject by any conventional route, including injection or by gradual infusion over time. The administration may, for example, be by infusion or by intramuscular, intravascular, intracavity, intracerebral, intralesional, rectal, subcutaneous, intradermal, epidural, intrathecal, percutaneous administration.

The compositions described herein may be in any form suitable for the above modes of administration. For example, compositions comprising modified cells may in any form suitable for infusion. As further examples, suitable forms for parenteral injection (including, subcutaneous, intramuscular, intravascular or infusion) include a sterile solution, suspension or emulsion.

Alternatively, the route of administration may be by direct injection into the target area, or by regional delivery or by local delivery. The identification of suitable dosages of the compositions of the invention is well within the routine capabilities of a person of skill in the art.

Advantageously, the compositions described herein may be formulated for use in T cell receptor (TCR) gene transfer, an approach that is rapid, reliable and capable of generating large quantities of T cells with specificity for the Jchain antigenic peptides (e.g. the peptides shown in any one of SEQ ID NO:99 to 107), regardless of the patient's pre-existing immune repertoire. Using TCR gene transfer, modified cells suitable for infusion may be generated within a few days.

The compositions described herein are for administration in an effective amount. An “effective amount” is an amount that alone, or together with further doses, produces the desired (therapeutic or non-therapeutic) response. The effective amount to be used will depend, for example, upon the therapeutic (or non-therapeutic) objectives, the route of administration, and the condition of the patient/subject. For example, the suitable dosage of the composition of the invention for a given patient/subject will be determined by the attending physician (or person administering the composition), taking into consideration various factors known to modify the action of the composition of the invention for example severity and type of haematological malignancy, body weight, sex, diet, time and route of administration, other medications and other relevant clinical factors. The dosages and schedules may be varied according to the particular condition, disorder or symptom the overall condition of the patient/subject. Effective dosages may be determined by either in vitro or in vivo methods.

The pharmaceutical compositions described herein are advantageously presented in unit dosage form.

Methods of Generating Binding Proteins (e.g. TCRs)

A method of generating a binding protein that is capable of specifically binding to a peptide containing a Jchain antigen and does not bind to a peptide that does not contain the Jchain antigen is also provided, comprising contacting a nucleic acid composition (or vector system) described herein with a cell under conditions in which the nucleic acid composition is incorporated and expressed by the cell.

In the context of the binding proteins described herein, the Jchain antigen comprises or consists of a sequence comprising an amino acid sequence selected from the group consisting of: SEQ ID NO:99 to 107, or a functional fragment or variant thereof.

The method may be carried out on the (host) cell ex vivo or in vitro. Alternatively, the method may be performed in vivo, wherein the nucleic acid composition (or vector system) is administered to the subject and is contacted with the cell in vivo, under conditions in which the nucleic acid sequence is incorporated and expressed by the cell to generate the binding protein. In one example, the method is not a method of treatment of the human or animal body.

Appropriate in vivo, in vitro and ex vivo methods for contacting a nucleic acid sequence (or vector systems) with a cell under conditions in which the nucleic acid sequence (or vector) is incorporated and expressed by the cell are well known, as described elsewhere herein.

As stated elsewhere herein, the binding protein comprise a TCR, an antigen binding fragment of a TCR, a ImmTAC or a chimeric antigen receptor (CAR). Further details are provided elsewhere herein.

The binding proteins described herein may be used therapeutically, as described elsewhere herein. Furthermore, the binding proteins may be used in a diagnostic setting, e.g. to detect the presence of Jchain presented in the context of an appropriate HLA at the cell surface of diseased/malignant tissues.

### General Definitions

As used herein “nucleic acid sequence”, “polynucleotide”, “nucleic acid” and “nucleic acid molecule” are used interchangeably to refer to an oligonucleotide sequence or polynucleotide sequence. The nucleotide sequence may be of genomic, synthetic or recombinant origin, and may be double-stranded or single-stranded (representing the sense or antisense strand). The term “nucleotide sequence” includes genomic DNA, cDNA, synthetic DNA, and RNA (e.g. mRNA) and analogs of the DNA or RNA generated, e.g., by the use of nucleotide analogs. In one example, the nucleotide sequence lacks introns. In other words, it is an intronless nucleic acid sequence. For example, the nucleotide sequence may be a DNA sequence that does not comprise intron sequences.

As used herein, “isolated nucleic acid sequence” or “isolated nucleic acid composition” refers to a nucleic acid sequence that is not in its natural environment when it is linked to its naturally associated sequence(s) that is/are also in its/their natural environment. In otherwords, an isolated nucleic acid sequence/composition is not a native nucleotide sequence/composition, wherein “native nucleotide sequence/composition” means an entire nucleotide sequence that is in its native environment and when operatively linked to an entire promoter with which it is naturally associated, which promoter is also in its native environment. Such a nucleic acid could be part of a vector and/or such nucleic acid or polypeptide could be part of a composition {e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

The nucleic acid sequences of the invention may be a non-naturally occurring nucleic acid sequence (e.g. it may be that the entire sequence does not occur in its entirety in nature). For example, the nucleic acid sequence of the invention may be operably linked to a promoter, wherein the promoter is not naturally associated with equivalent human nucleic acid sequences in nature (e.g. human TCR sequences or fragments thereof); i.e. it is not the entire promoter that is naturally associated with the nucleic acid in its natural environment. In this context, such promoters may be considered exogenous promoters. Examples of appropriate promoters are described elsewhere.

As used herein “specifically binds” or “specific for” refers to an association or union of a binding protein (e.g., TCR receptor) or a binding domain (or fusion protein thereof) to a target molecule with an affinity or Kₐ (i.e., an equilibrium association constant of a particular binding interaction with units of 1/M) equal to or greater than 10⁵M⁻¹ (which equals the ratio of the on-rate [kₒₙ] to the off-rate [k_{off}] for this association reaction), while not significantly associating or uniting with any other molecules or components in a sample. Binding proteins or binding domains (or fusion proteins thereof) may be classified as “high affinity” binding proteins or binding domains (or fusion proteins thereof) or as “low affinity” binding proteins or binding domains (or fusion proteins thereof). “High affinity” binding proteins or binding domains refer to those binding proteins or binding domains having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. Low affinity” binding proteins or binding domains refer to those binding proteins or binding domains having a Kₐ of up to 107 M⁻¹ up to 10⁶ M⁻¹, up to 10⁵ M⁻¹. Alternatively, affinity can be defined as an equilibrium dissociation constant (K_{d}) of a particular binding interaction with units of M (e.g., 10⁻⁵ M to 10⁻¹³ M).

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof. In certain embodiments, enhanced affinity TCRs can be codon optimized to enhance expression in a particular host cell, such as a cell of the immune system, a inducible pluripotent stem cell (iPSC), a hematopoietic stem cell, a T cell, a primary T cell, a T cell line, a NK cell, or a natural killer T cell (Scholten et al, Clin. Immunol. 119: 135, 2006). The T cell can be a CD4+ or a CD8+ T cell, or gamma-delta T cell.

As used herein, the term “Jchain antigen” or “Jchain peptide antigen” or “Jchain-containing peptide antigen” refers to a naturally or synthetically produced peptide portion of a Jchain protein ranging in length from about 7 amino acids, about 8 amino acids, about 9 amino acids, about 10 amino acids, up to about 20 amino acids, which can form a complex with a MHC (e.g., HLA) molecule, and a binding protein of this disclosure specific for a Jchain peptide:MHC (e.g., HLA) complex can specifically bind to such as complex. Typically, for the purposes of this disclosure, the Jchain peptide antigen comprises or consists of an amino acid sequence selected from the group consisting of: YTAVVPLVY (SEQ ID NO:99), TAVVPLVY (SEQ ID NO:102), VLAVFIKAVHV (SEQ ID NO:103), VLAVFIKAV (SEQ ID NO:104), YTAVVPLV (SEQ ID NO:105), ISDPTSPLRTR (SEQ ID NO:101), RIIVPLNNR (SEQ ID NO:106), CYTAVVPLV (SEQ ID NO:100) and GETKMVETAL (SEQ ID NO:107). In some examples, the Jchain peptide antigen comprises or consists of an amino acid sequence selected from the group consisting of: YTAVVPLVY (SEQ ID NO:99), ISDPTSPLRTR (SEQ ID NO:101) and CYTAVVPLV (SEQ ID NO:100). Additionally, for the purposes of this disclosure, the Jchain peptide antigen:HLA complex typically comprises a peptide:HLA complex selected from the group consisting of: a YTAVVPLVY:HLA-A*01:01 complex; a TAVVPLVY:HLA-A*01:01 complex; a VLAVFIKAVHV:HLA-A*02:01 complex; a VLAVFIKAV:HLA-A*02:01 complex; a YTAVVPLV:HLA-A*02:01 complex; a ISDPTSPLRTR:HLA-A*03:01 complex; a RIIVPLNNR:HLA-A*11:01 complex; a ISDPTSPLRTR:HLA-A*11:01 complex; a CYTAVVPLV:HLA-A*24:02 complex; and a GETKMVETAL:HLA-B*40:01 complex. In some examples, the Jchain peptide antigen:HLA complex comprises a peptide:HLA complex selected from the group consisting of: a YTAVVPLVY:HLA-A*01:01 complex; a ISDPTSPLRTR:HLA-A*03:01 complex; a ISDPTSPLRTR:HLA-A*11:01 complex; and a CYTAVVPLV:HLA-A*24:02 complex.

The term “Jchain-specific binding protein,” as used herein, refers to a protein or polypeptide, such as a TCR or CAR, that specifically binds to a Jchain peptide antigen (or to a Jchain peptide antigen:HLA complex, e.g., on a cell surface), and does not bind a peptide sequence that does not include the Jchain peptide antigen. Typically, for the purposes of this disclosure, the Jchain peptide antigen comprises or consists of an amino acid sequence selected from the group consisting of: YTAVVPLVY (SEQ ID NO:99), TAVVPLVY (SEQ ID NO:102), VLAVFIKAVHV (SEQ ID NO:103), VLAVFIKAV (SEQ ID NO:104), YTAVVPLV (SEQ ID NO:105), ISDPTSPLRTR (SEQ ID NO:101), RIIVPLNNR (SEQ ID NO:106), CYTAVVPLV (SEQ ID NO:100) and GETKMVETAL (SEQ ID NO:107), and the Jchain peptide antigen:HLA complex comprises a peptide:HLA complex selected from the group consisting of: a YTAVVPLVY:HLA-A*01:01 complex; a TAVVPLVY:HLA-A*01:01 complex; a VLAVFIKAVHV:HLA-A*02:01 complex; a VLAVFIKAV:HLA-A*02:01 complex; a YTAVVPLV:HLA-A*02:01 complex; a ISDPTSPLRTR:HLA-A*03:01 complex; a RIIVPLNNR:HLA-A*11:01 complex; a ISDPTSPLRTR:HLA-A*11:01 complex; a CYTAVVPLV:HLA-A*24:02 complex; and a GETKMVETAL:HLA-B*40:01 complex, as appropriate.

In certain embodiments, a Jchain-specific binding protein specifically binds to a Jchain peptide antigen (or a Jchain peptide antigen:HLA complex) with a Kd of less than about 10⁻⁸ M, less than about 10⁻⁹ M, less than about 10⁻¹⁰ M, less than about 10⁻¹¹ M, less than about 10⁻¹² M, or less than about 10⁻¹³ M, or with an affinity that is about the same as, at least about the same as, or is greater than at or about the affinity exhibited by an exemplary Jchain-specific binding protein provided herein, such as any of the Jchain-specific TCRs provided herein, for example, as measured by the same assay. In certain embodiments, a Jchain-specific binding protein comprises a Jchain-specific immunoglobulin superfamily binding protein or binding portion thereof. Typically, for the purposes of this disclosure, the Jchain peptide antigen comprises or consists of an amino acid sequence selected from the group consisting of: YTAVVPLVY (SEQ ID NO:99), TAVVPLVY (SEQ ID NO:102), VLAVFIKAVHV (SEQ ID NO:103), VLAVFIKAV (SEQ ID NO:104), YTAVVPLV (SEQ ID NO:105), ISDPTSPLRTR (SEQ ID NO:101), RIIVPLNNR (SEQ ID NO:106), CYTAVVPLV (SEQ ID NO:100) and GETKMVETAL (SEQ ID NO:107), and the Jchain peptide antigen:HLA complex comprises a peptide:HLA complex selected from the group consisting of: a YTAVVPLVY:HLA-A*01:01 complex; a TAVVPLVY:HLA-A*01:01 complex; a VLAVFIKAVHV:HLA-A*02:01 complex; a VLAVFIKAV:HLA-A*02:01 complex; a YTAVVPLV:HLA-A*02:01 complex; a ISDPTSPLRTR:HLA-A*03:01 complex; a RIIVPLNNR:HLA-A*11:01 complex; a ISDPTSPLRTR:HLA-A*11:01 complex; a CYTAVVPLV:HLA-A*24:02 complex; and a GETKMVETAL:HLA-B*40:01 complex, as appropriate.

The selective binding may be in the context of Jchain antigen presentation by HLA-A*01:01, HLA-A*02:01, HLA-A*03:01, HLA-A*11:01, HLA-A*24:02 and/or HLA-B*40:01, in particular in the context of HLA-A*01:01, HLA-A*03:01, HLA-A*11:01 and/or HLA-A*24:02. In other words, in certain embodiments, a binding protein that “specifically binds to a Jchain antigen” may only do so when it is being presented (i.e. it is bound by) by a specific HLA or is in an equivalent structural formation as when it is being presented by the specific HLA. As discussed elsewhere herein, the inventors identified that Jchain derived peptides YTAVVPLVY (SEQ ID NO:99) and TAVVPLVY (SEQ ID NO:102) are capable of being presented by HLA-A*01:01; that Jchain derived peptides VLAVFIKAVHV (SEQ ID NO:103), VLAVFIKAV (SEQ ID NO:104) and YTAVVPLV (SEQ ID NO:105) are capable of being presented by HLA-A*02:01; that Jchain derived peptide ISDPTSPLRTR (SEQ ID NO:101) is capable of being presented by HLA-A*03:01 and HLA-A*11:01; that Jchain derived peptide RIIVPLNNR (SEQ ID NO:106) is capable of being presented by HLA-A*11:01; that Jchain derived peptide CYTAVVPLV (SEQ ID NO:100) is capable of being presented by HLA-A*24:02; and that Jchain derived peptide GETKMVETAL (SEQ ID NO:107) is capable of being presented by HLA-B*40:01.

Accordingly, for example, in certain examples, a binding protein that “specifically binds to a Jchain antigen”, in particular YTAVVPLVY (SEQ ID NO:99), may only do so when it is being presented (i.e. it is bound by) HLA-A*01:01 or is in an equivalent structural formation as when it is being presented by HLA-A*01:01. In another example, a binding protein that “specifically binds to a Jchain antigen”, in particular ISDPTSPLRTR (SEQ ID NO:101) may only do so when it is being presented (i.e. it is bound by) HLA-A*11:01 and/or HLA-A*03:01, or is in an equivalent structural formation as when it is being presented by HLA-A*11:01 and/or HLA-A*03:01. In another example, a binding protein that “specifically binds to a Jchain antigen”, in particular CYTAVVPLV (SEQ ID NO:100) may only do so when it is being presented (i.e. it is bound by) HLA-A*24:02 or is in an equivalent structural formation as when it is being presented by HLA-A*24:02.

By “specifically bind(s) to” as it relates to a T cell receptor, or as it refers to a recombinant T cell receptor, nucleic acid fragment, variant, or analog, or a modified cell, such as, for example, the Jchain T cell receptors, and Jchain-expressing modified cells herein, is meant that the T cell receptor, or fragment thereof, recognizes, or binds selectively to a Jchain antigen (e.g. wherein the Jchain antigen comprises an amino acid sequence selected from the group consisting of: YTAVVPLVY (SEQ ID NO:99), TAVVPLVY (SEQ ID NO:102), VLAVFIKAVHV (SEQ ID NO:103), VLAVFIKAV (SEQ ID NO:104), YTAVVPLV (SEQ ID NO:105), ISDPTSPLRTR (SEQ ID NO:101), RIIVPLNNR (SEQ ID NO:106), CYTAVVPLV (SEQ ID NO:100) and GETKMVETAL (SEQ ID NO:107)). Under certain conditions, for example, in an immunoassay, for example an immunoassay discussed herein, the T cell receptor binds to JChain (e.g. an amino acid sequence selected from the group consisting of: YTAVVPLVY (SEQ ID NO:99), TAVVPLVY (SEQ ID NO:102), VLAVFIKAVHV (SEQ ID NO:103), VLAVFIKAV (SEQ ID NO:104), YTAVVPLV (SEQ ID NO:105), ISDPTSPLRTR (SEQ ID NO:101), RIIVPLNNR (SEQ ID NO:106), CYTAVVPLV (SEQ ID NO:100) and GETKMVETAL (SEQ ID NO:107)) and does not bind in a significant amount to other polypeptides. Thus the T cell receptor may bind to Jchain (e.g. an amino acid sequence selected from the group consisting of: YTAVVPLVY (SEQ ID NO:99), TAVVPLVY (SEQ ID NO:102), VLAVFIKAVHV (SEQ ID NO:103), VLAVFIKAV (SEQ ID NO:104), YTAVVPLV (SEQ ID NO:105), ISDPTSPLRTR (SEQ ID NO:101), RIIVPLNNR (SEQ ID NO:106), CYTAVVPLV (SEQ ID NO:100) and GETKMVETAL (SEQ ID NO:107)) with at least 10, 100, or 1000, fold more affinity than to a control antigenic polypeptide. This binding may also be determined indirectly in the context of a modified T cell that expresses a Jchain TCR. In assays such as, for example, an assay discussed herein, the modified T cell is specifically reactive against a multiple myeloma cell line and at least one malignant B cell lines such as, for example, ALL, CLL and mantle cell lymphoma cell lines. Thus, the modified Jchain-expressing T cell binds to a multiple myeloma cell line or a malignant B cell line with at least 10, 100, or 1000, fold more reactivity when compared to its reactivity against a control cell line that is not a multiple myeloma cell line or a malignant B cell line.

A “non-essential” (or “non-critical”) amino acid residue is a residue that can be altered from the wild-type sequence of (e.g., the sequence identified by SEQ ID NO herein) without abolishing or, more preferably, without substantially altering a biological activity, whereas an “essential” (or “critical”) amino acid residue results in such a change. For example, amino acid residues that are conserved are predicted to be particularly non-amenable to alteration, except that amino acid residues within the hydrophobic core of domains can generally be replaced by other residues having approximately equivalent hydrophobicity without significantly altering activity.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential (or non-critical) amino acid residue in a protein is preferably replaced with another amino acid residue from the same side chain family. Alternatively, in another embodiment, mutations can be introduced randomly, and the resultant mutants can be screened for activity to identify mutants that retain activity.

Calculations of sequence homology or identity (the terms are used interchangeably herein) between sequences are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970) J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

Alternatively, the percent identity between two amino acid or nucleotide sequences can be determined using the algorithm of Meyers et al. (1989) CABIOS 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-410). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, gapped BLAST can be utilized as described in Altschul et al. (1997, Nucl. Acids Res. 25:3389-3402). When using BLAST and gapped BLAST programs, the default parameters of the respective programs (e.g., XBLAST and NBLAST) can be used. See <http://www.ncbi.nlm.nih.gov>.

The polypeptides and nucleic acid molecules described herein can have amino acid sequences or nucleic acid sequences sufficiently or substantially identical to the sequences identified by SEQ ID NO. The terms “sufficiently identical” or “substantially identical” are used herein to refer to a first amino acid or nucleotide sequence that contains a sufficient or minimum number of identical or equivalent (e.g. with a similar side chain) amino acid residues or nucleotides to a second amino acid or nucleotide sequence such that the first and second amino acid or nucleotide sequences have a common structural domain or common functional activity. In other words, amino acid sequences or nucleic acid sequences having one or several (e.g. two, three, four etc) amino acid or nucleic acid substitutions compared to the corresponding sequences identified by SEQ ID NO may be sufficiently or substantially identical to the sequences identified by SEQ ID NO (provided that they retain the requisite functionality). In such examples, the one or several (e.g. two, three, four etc) amino acid or nucleic acid substitutions may be conservative substitutions. For example, amino acid or nucleotide sequences that contain a common structural domain having at least about 60%, or 65% identity, likely 75% identity, more likely 85%, 90%, 91%, 92%, 93%, 94%, 95%, 96%, 97%, 98% or 99% identity are defined herein as sufficiently or substantially identical.

TCR sequences are defined according to IMGT. See the LeFranc references herein for further details i.e. [1]Lefranc M.-P. “Unique database numbering system for immunogenetic analysis” Immunology Today, 18: 509 (1997). [2]Lefranc M.-P. “The IMGT unique numbering for immunoglobulins, T cell Receptors and Ig-like domains” The immunologist, 7, 132-136 (1999). [3]Lefranc M.-P. et al. “IMGT unique numbering for immunoglobulin and Tcell receptor variable domains and Ig superfamily V-like domains” Dev. Comp. Immunol., 27, 55-77 (2003). [4]Lefranc M.-P. et al. “IMGT unique numbering for immunoglobulin and T cell receptor constant domains and Ig superfamily C-like domains” Dev. Comp. Immunol., 2005, 29, 185-203 PMID: 15572068.

As used herein, the term “ex vivo” refers to “outside” the body. The term “in vitro” can be used to encompass “ex vivo” components, compositions and methods.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### Example 1—TCR Gene Therapy of Multiple Myeloma Through Newly Identified TCRs with High Allele Coverage Targeting the Jchain

### Introduction

The inventors recently identified the joining chain (Jchain) as a potential target antigen for MM, as it was highly expressed in patient MM samples but not in healthy tissues of non-B cell origins(11). As Jchain is located intracellularly, TCR mediated targeting of the Jchain derived peptides presented in HLA on the surface of target cells is required for them to be bonafide antigens for T cell based targeting of MM.

As described herein, the inventors identified Jchain derived peptides presented on MM cells in HLA-A*01:01 (HLA-A1), HLA-A*02:01 (HLA-A2), HLA-A*03:01 (HLA-A3), HLA-A*11:01 (HLA-A11), HLA-A*24:02 (HLA-A24), and HLA-B*40:01 (HLA-B40) and subsequently successfully isolated T cells with Jchain specific recognition of peptides presented in HLA-A1, HLA-A3, HLA-A11 and HLA-A24. To allow identification of potent T cells recognizing non-mutated self-peptides the inventors performed a method previously described by their group(10, 11). In this method, the inventors made use of the immunogenicity of peptides in the HLA mismatched setting, for which the inventors previously demonstrated that healthy donor PBMCs from individuals not expressing a certain HLA allele can be used to isolate potent T cell clones recognizing non-mutated peptide sequences presented in this HLA allele(10, 11). Upon sequencing and transfer of TCRs from selected T cell clones, in vitro as well as in vivo killing of MM was demonstrated without off-target effects, demonstrating that the TCRs identified herein are useful in the treatment of B cell associated diseases or conditions such as MM.

### Results

### Jchain as Target Gene for MM and Epitope Discovery by Mass Spectrometry

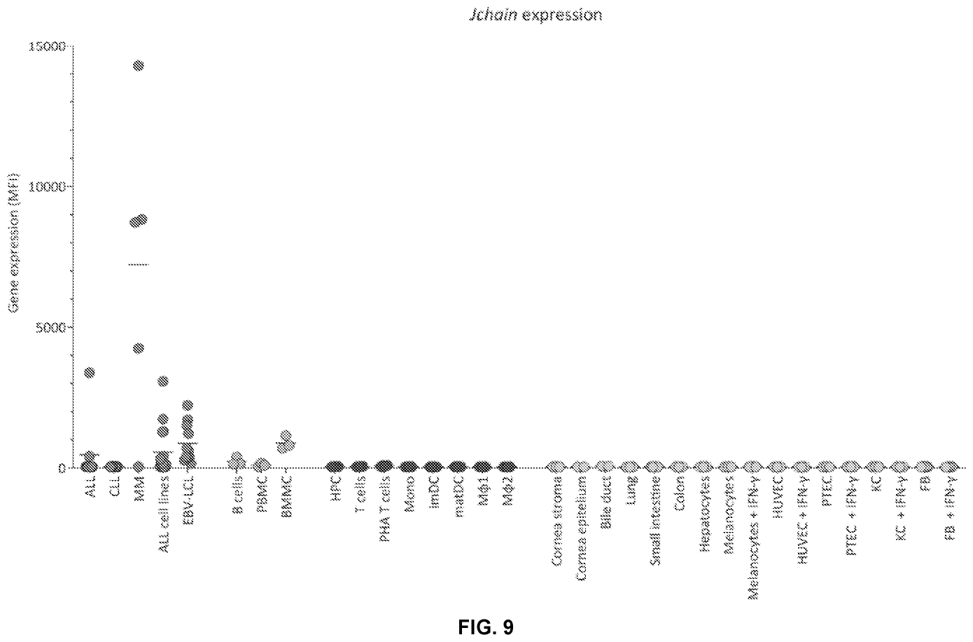

In a previous study, the inventors identified target genes for cellular therapy of B cell malignancies using Illumina microarray data(11, 19). The Jchain, was identified as a promising target since it was highly expressed in 4 out of 5 patient derived MM samples and expression in healthy tissues of non-B cell origin was absent (FIG. 9). As provided herein, the inventors performed epitope discovery for TCR based targeting of the Jchain. To this end, peptides derived from the Jchain protein presented in HLA on the surface of MM cell lines U266 and UM9 were identified. U266 naturally expresses HLA-A2, -A3, -B7, -B40, -C3, and -C7. U266 cells were additionally transduced (Td) with HLA-A24 to allow identification of peptides presented in other common HLA-I alleles and potentially broaden the HLA-allele coverage of Jchain TCRs. UM9 cells express HLA-A1, -A11, -B7, -B55, -C3, and -C7 and were additionally Td with HLA-A2. First, peptide-HLA class I (pHLA-1) complexes were isolated from U266 and UM9 cells and peptides eluted from HLA-I were analyzed by mass spectrometry. Subsequently, mass spectrometry data was inspected for peptides originating from the Jchain protein according to uniprot. To validate that Jchain peptides were correctly identified by mass spectrometry, mass spectra of the eluted peptides were compared with mass spectra of synthetically generated peptides of the same sequence. To determine HLA-I origin of identified Jchain peptides, peptides were assessed for presence of HLA-I anker residues, according to netMHC4.0, for binding to HLA-I alleles expressed by U266 and UM9. This resulted in identification of 10 peptides for which the most likely HLA-I origins were HLA-A1, -A2, -A3, -All, -A24 and -B40 (Table 1). Most peptides were predicted strong or weak binders to the respective HLA molecule according to netMHC4.0, while for some peptides binding was not predicted. However, for all peptides, binding to the respective HLA allele was bio-chemically confirmed upon stable peptide-HLA (pHLA) monomer refolding (data not shown). Subsequently, pHLA monomers were used to generate phycoerythrin (PE)-labelled pHLA-tetramers.

TABLE 1
Jchain derived peptides identified by mass
spectrometry from MM cell lines UM9 or U266
presented in HLA-A1, A2, A3, A11, A24 or B40.

| | | Jchain | | | | Eluted |
|---|---|---|---|---|---|---|
| | | aa | | | | from |
| | Se- | posi- | | Affinity | | MM cell |
| | quence | tion^{a} | HLA-I^{b} | (nM)^{c} | WB/SB^{c} | line^{d} |
| YTA- | YTAVVP | 132-140 | A1 | 6 | SB | UM9 |
| A1 | LVY | | | | | |
| TAV- | TAVVPL | 133-140 | A1 | 457 | SB | UM9 |
| A1 | VY | | | | | |
| VLA- | VLAVFI | 10-20 | A2 | 767 | <WB | UM9 |
| A2¹ | KAVHV | | | | | |
| VLA- | VLAVFI | 10-18 | A2 | 80 | WB | UM9 |
| A2² | KAV | | | | | |
| YTA- | YTAVVP | 132-139 | A2 | 503 | <WB | UM9 |
| A2 | LV | | | | | |
| ISD- | ISDPTS | 72-82 | A3 | 21436 | <WB | U266 |
| A3 | PLRTR | | | | | |
| RII- | RIIVPL | 61-69 | A11 | 299 | WB | UM9 |
| A11 | NNR | | | | | |
| ISD- | ISDPTS | 72-82 | A11 | 12330 | <WB | UM9 |
| A11 | PLRTR | | | | | |
| CYT- | CYTAVV | 131-139 | A24 | 655 | WB | U266 |
| A24 | PLV | | | | | |
| GET- | GETKMV | 142-151 | B40 | 25 | SB | U266 |
| B40 | ETAL | | | | | |

ªAmino acid (aa) position of identified peptides within the jchain protein according to uniprot.
^{b}HLA-I origin of peptides according to binding motifs of netMHC4.0 and HLA typing of MM cells from which peptides were eluted.
^{c}Affinity (nM) for the respective HLA-I allele according to NetMHC 4.0. SB: strong binder, WB: weak binder, <WB: less than weak binder.
^{d}UM9 cells (HLA-A1, -A11, -B7, -B35, -C3 and -C7 positive) were HLA-A2 transduced and U266 cells (HLA-A2, -A3, -B7, -B40, -C3 and -C7 positive) were HLA-A24 transduced.

Jchain Specific T Cell Clones Identified from HLA Mismatched Healthy Donors

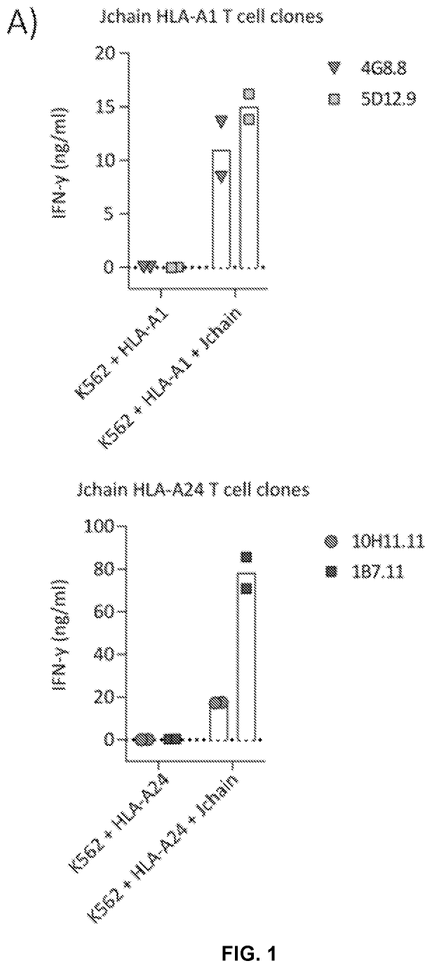

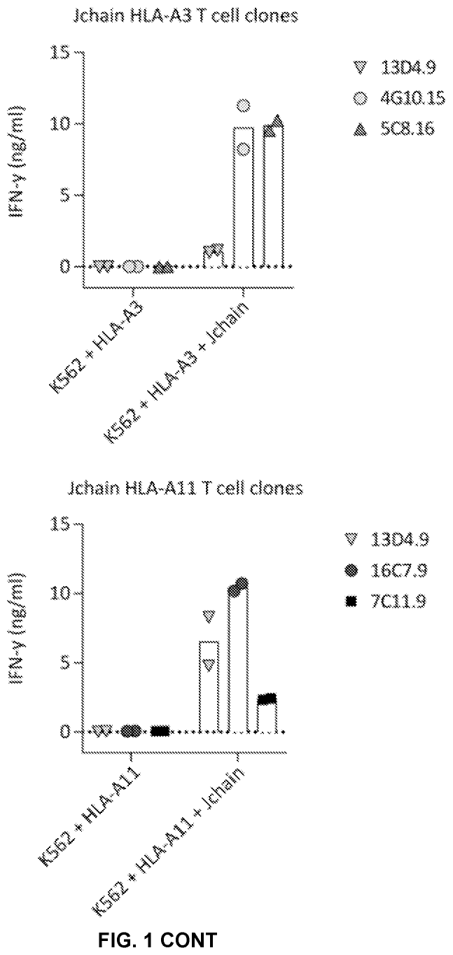

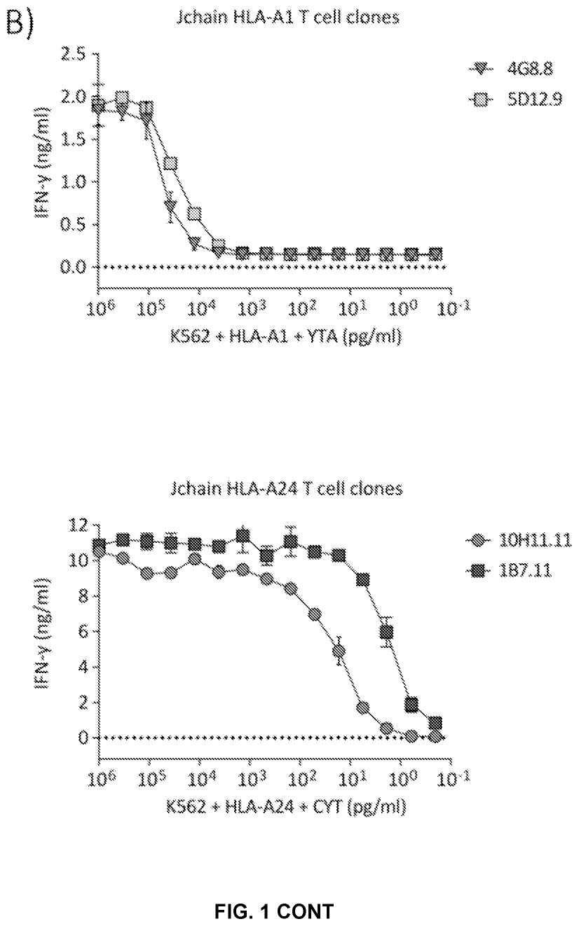

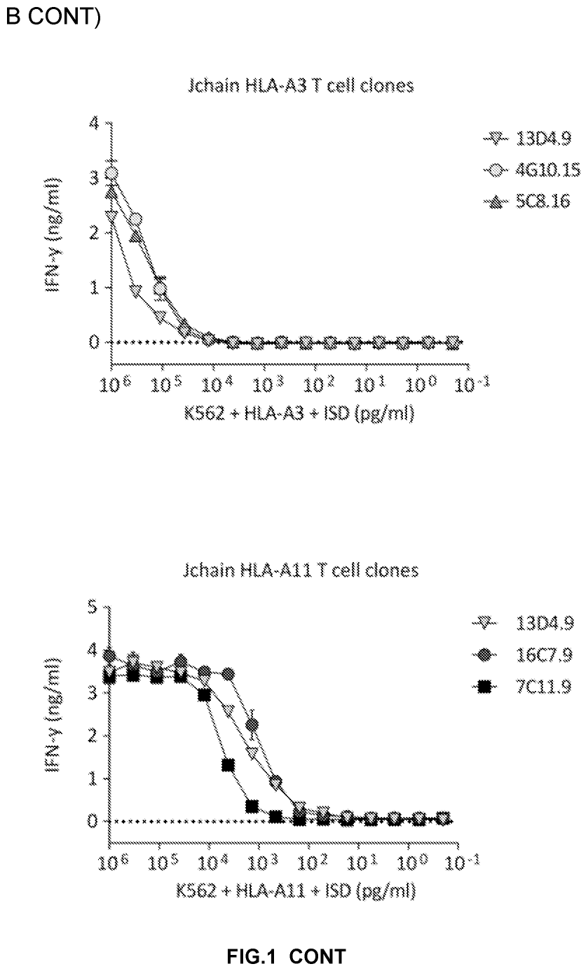

As previously described, pHLA-tetramers were used to isolate T cells from healthy donors which were selected to not express target HLA molecules(11). PBMCs, isolated from complete buffy coats, were incubated with PE-labelled pHLA-tetramers (Table 1) and PE magnetic activated cell sorting (MACS) was performed, after which pHLA-tetramer^{pos}, CD8^{pos} T cells were single-cell sorted and clonally expanded. In total, 26 buffy coats were used, each pHLA-tetramer was included between 4 and 19 times for T cell isolations (data not shown). Overall, 29000 pHLA-tetramer^{pos}, CD8^{pos} T cells were sorted of which 17000 T cell clones expanded. For expanded clones, peptide specificity followed by recognition of endogenous antigen was tested in high throughput screenings (data not shown). Previous studies demonstrated that recognition of target gene transduced cells is a good predictor for recognition of malignant cells naturally expressing the target antigen. Therefore, the 10 most potent T cell clones were selected based on recognition of Jchain Td K562 cells, these T cell clones were specific for Jchain peptides YTAVVPLVY (SEQ ID NO:99) in HLA-A1 (YTA-A1), CYTAVVPLV (SEQ ID NO:100) (CYT-A24), and ISDPTSPLRTR (SEQ ID NO:101) in HLA-A3 (ISD-A3) as well as in HLA-A11 (ISD-A11) (FIG. 1A).

Target-peptide specific recognition was demonstrated in peptide titration experiments. Jchain negative K562 cells were separately transduced with HLA alleles and loaded with decreasing concentration of Jchain peptides. All selected T cell clones exhibited peptide specific recognition, defined by IFN-γ production after overnight co-culture, which decreased with lower peptide concentrations while unloaded K562 cells were not recognized (FIG. 1B). Of note, T cell clone 13D4.9 recognized ISD-A3 as well ISD-A11. Within specificities, recognition of endogenously processed antigen correlated with avidity as observed in peptide titration experiments. For each specificity, the two most potent T cell clones were selected for further screenings of safety and potency. T cell clone 13D4.9 was the least promising ISD-A3 T cell clone but one of the best ISD-A11 T cell clones. Clone 13D4.9 was therefore excluded from the selection of ISD-A3 T cell clones while it was further screened for recognition of ISD-A11. T cell clone 7C11.9 was the weakest ISD-A11 T cell clones and was not investigated in follow-up experiments.

Safety Screenings Revealed T Cell Clones with Restricted On-Target Recognition

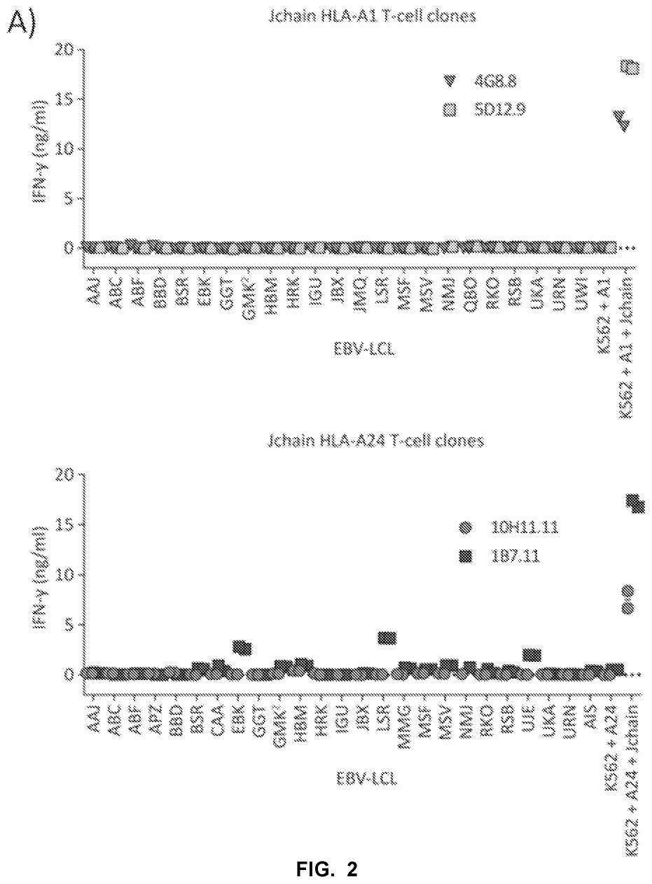

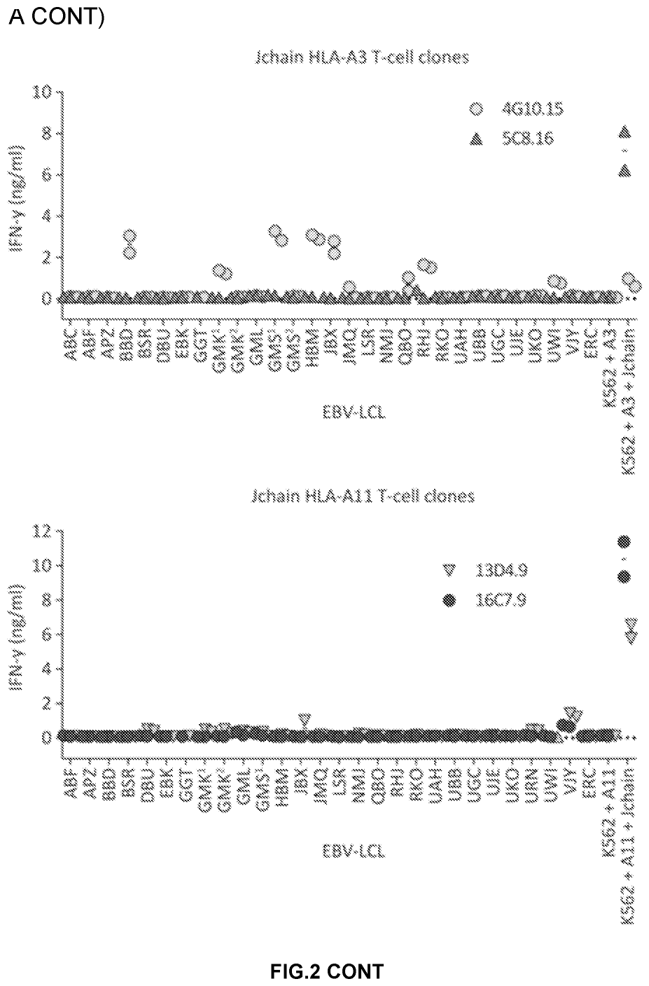

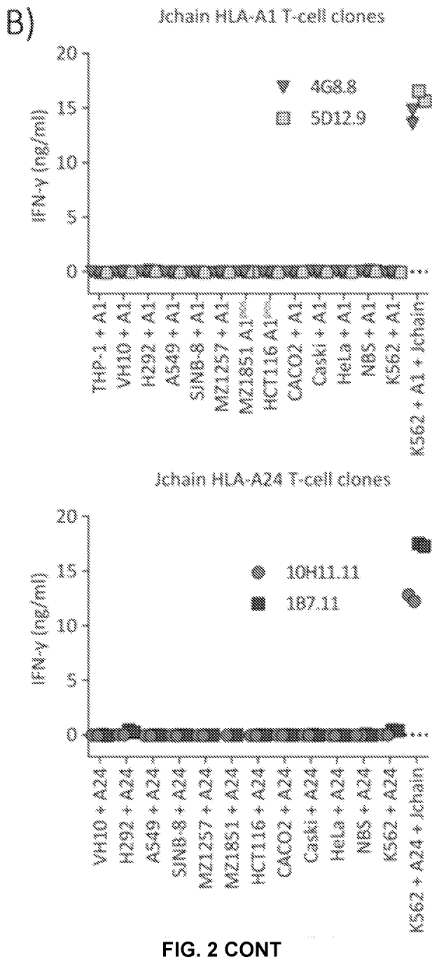

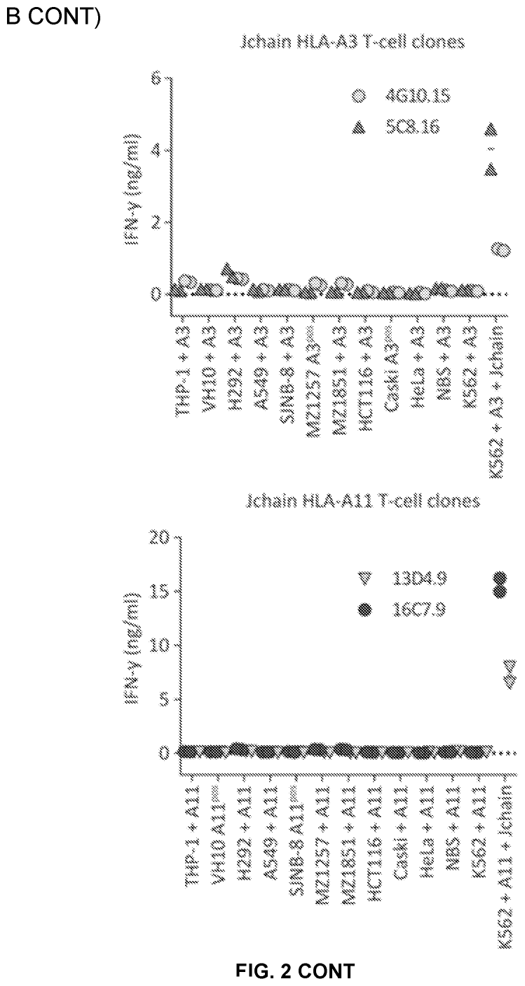

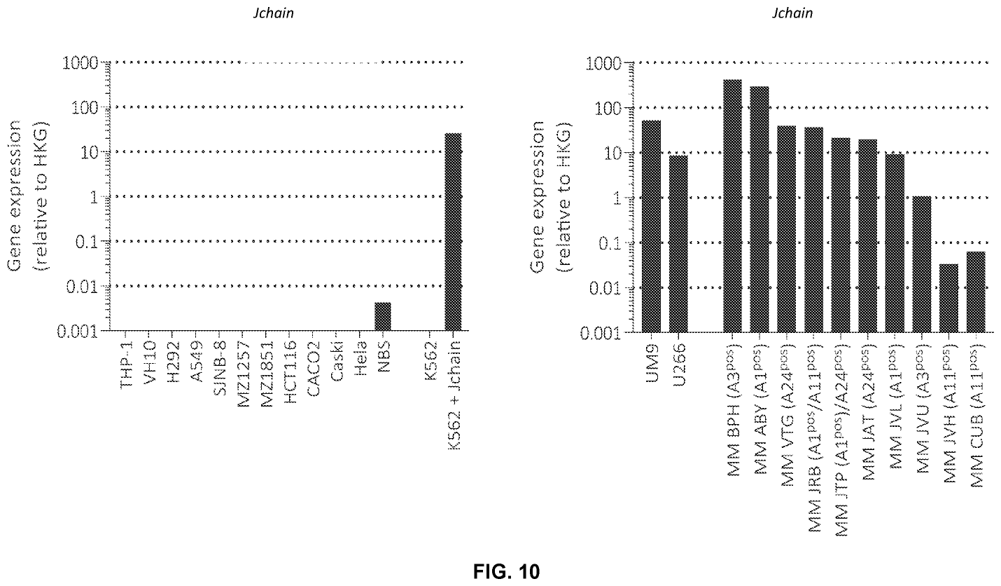

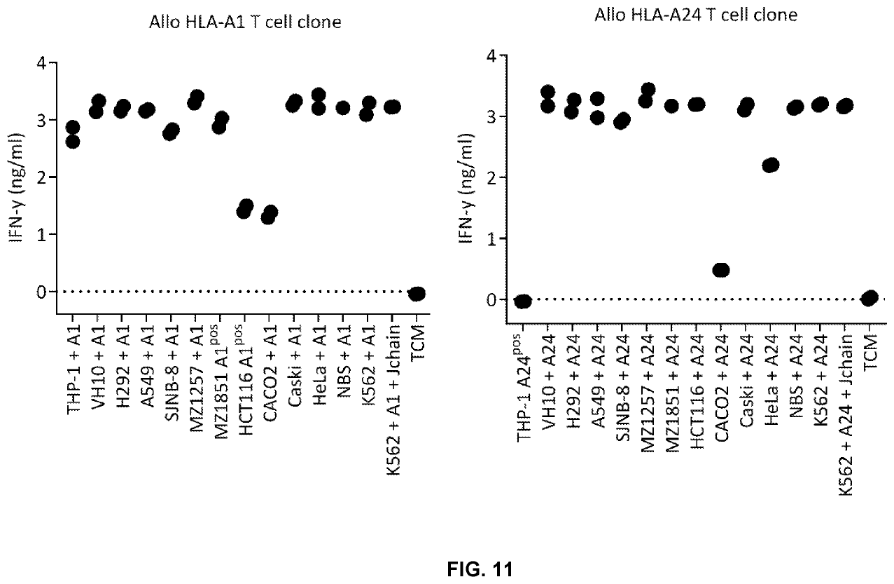

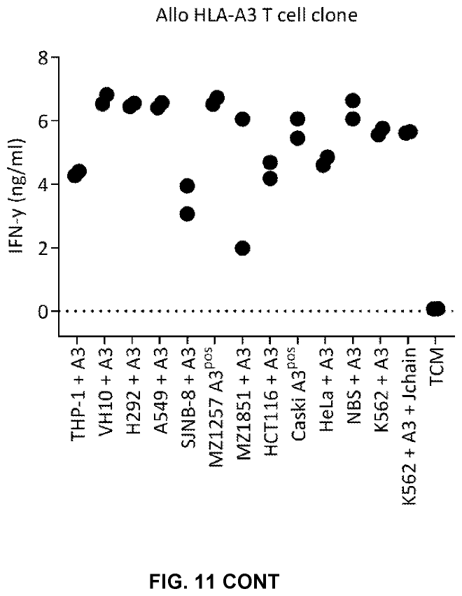

As TCRs have the intrinsic characteristics that pHLA recognition is often promiscuous, Jchain pHLA specificity of selected T cell clones was investigated to identify potentially harmful cross-reactivities. To uncover cross-reactivity with peptides presented in other HLA alleles, T cell clones were stimulated with a panel of EBV transformed lymphoblastoid cell lines (EBV-LCLs) designed, per HLA restriction, to express as many common HLA-I alleles as possible without expressing the target restriction allele (FIG. 2A, Table 2). Additionally, recognition of other peptides presented in the target HLA alleles was investigated by stimulation with a panel of non B-cell cell lines derived from multiple different tissue origins, which were transduced with target HLA alleles when target alleles were not expressed by wild type (WT) cells (FIG. 2B). Lack of Jchain expression in cell lines used was confirmed by qPCR (FIG. 10). Allo-HLA T cell clones, recognizing peptides derived from unknown housekeeping genes (HKGs) in specific HLA alleles, were used to demonstrate that target cell lines generally expressed sufficient HLA to allow T cell recognition (FIG. 11). In both experiments, Jchain and target HLA transduced K562 cells were used as positive controls for T cell function.

TABLE 2
HLA typing of EBV-LCLs used in EBV-LCL panels.

| | Included in Jchain EBV- | | | | | | |
|---|---|---|---|---|---|---|---|
| | LCL panel | | | | | | |
| EBV- | HLA- | HLA- | HLA- | HLA- | HLA typing | | |
| LCL | A1 | A3 | A11 | A24 | HLA-A | HLA-B | HLA-C |
| ABF | x | x | x | x | 30:04-68:02 | 38:01-55:01 | 03:03-12:03 |
| APZ | | x | x | x | 01:01-68:01 | 44:02-44:02 | 05:01-07:04 |
| BBD | x | x | x | x | 02:01-02:05 | 15:01-45:01 | 01:02-06:02 |
| BSR | x | x | x | x | 02:01-68:01 | 35:03-37:01 | 04:01-06:02 |
| DBU | | x | x | | 02:01-02:01 | 57:01-57:01 | 06:02-06:02 |
| EBK | x | x | x | x | 02:05-02:05 | 58:01-58:01 | undetermined |
| GGT | x | x | x | x | 26:01/26:08/26:02- | 14:01-49:01 | 07:01/07:05/07:06- |
| | | | | | 31:01/31:02/31:06 | | 08:02/08:07 |
| GMK¹ | | x | x | | 02:01-01:01 | 50:01-07:02 | 07:02-06:02:01:01 |
| GMK² | x | x | x | x | 23:01-02:01 | 41:01-40:01 | 17:01:01:01- |
| | | | | | | | 03:04:01:01 |
| GML | | x | x | | 02:01-01:01 | 35:01-08:01 | 07:01-04:01 |
| GMS¹ | | x | x | | 25:01-02:01 | 44:02:01:01- | 07:02-05:01 |
| | | | | | | 07:02 | |
| HBM | x | x | x | x | 02:01-02:01 | 15:01:01:01- | 03:03:01- |
| | | | | | | 51:01:01 | 15:02:01 |
| JBX | x | x | x | x | 02:01-30:02 | 15:01-39:01 | 03:03-12:03 |
| JMQ | x | x | x | | 02:01-24:02 | 35:02-44:02 | 04:01-05:01 |
| LSR | x | x | x | x | 32:01-68:01 | 35:03-52:01 | 12:02-12:03 |
| NMJ | x | x | x | x | 02:01- | 40:01/40:11/40:14- | 03:04/03:08/03:09-17 |
| | | | | | 66:01/66:04 | 41:02 | |
| QBO | x | x | x | | 24:02-31:01 | 07:02/07:61-35:08 | 04:01-07:02 |
| RHJ | | x | x | | 02:01/02:07/02:09- | 15:01/15:33/15:34- | 03:04/03:08/03:09- |
| | | | | | 31:01/31:02/31:06 | 15:17 | 07:01/07:05/07:06 |
| RKO | x | x | x | x | 02:05-29:02 | 27:05-44:03 | 01:02-16:01:01 |
| UAH | | x | x | | 24:02-68:02 | 14:02-38:01 | 08:02-12:03:01 |
| UBB | | x | x | | 26:01-01:01 | 38:01-18:01 | 12:03-07:01/07:06 |
| UGC | | x | x | | 01:01-23:01/23:17 | 08:01-41:02 | 07:01-17:03 |
| UJE | | x | x | x | 01:01-33:03:01 | 44:03-51:01 | 07:06/07:18-14:02 |
| UKO | | x | x | | 29:02-30:01 | 13:02-44:03 | 06:02-16:01 |
| URN | x | | x | x | 02:01-03:01 | 08:01-50:01 | 06:02-07:01 |
| UWI | x | x | x | | 02:01-24:02 | 07:02-40:02 | 02:02-07:02 |
| VJY | | x | x | | 23:01-36:01 | 15:03-53:01 | 02:10-04:01 |
| ERC | | x | x | | 02:01-02:01 | 13:02-44:02 | 05:01-06:02 |

In short, Jchain HLA-A1 specific T cell clones did not recognize any EBV-LCLs or cell lines of the non-B cell panel, demonstrating absence of general cross-reactivity for these T cell clones (FIG. 2). Jchain HLA-A24 specific T cell clone 10H11.11 demonstrated no cross-reactivity, whereas T cell clone 1B7.11 recognized 3 EBV-LCLs, these EBV-LCLs did not share one HLA allele indicating that recognition resulted from cross-reactivity with multiple alleles, T cell clone 1B7.11 was therefore excluded (FIG. 2A). Furthermore, Jchain HLA-A3 specific T cell clone 4G10.15 recognized all EBV-LCLs expressing HLA-A*02:01, indicating cross-reactivity with peptide presented in the prevalent HLA-A*02:01 allele (FIG. 2A). Jchain HLA-A3 specific T cell clone 5C8.16 did not recognize any EBV-LCL or cell lines of the non-B cell panel, demonstrating absence of general cross-reactivity for this T cell clone. Jchain HLA-A11 specific T cell clones 13D4.9 and 16C7.9 demonstrated only very low recognition of EBV-LCL VJY (FIG. 2A). Since both T cell clones recognized this EBV-LCL and EBV-LCLs express Jchain (FIG. 9), this low recognition could most likely be explained by recognition of Jchain peptide presented in an HLA-A11 look-a-like HLA allele. Alternatively, recognition could result from cross-reactivity with another peptide presented in one of the non-prevalent HLA alleles expressed by EBV-LCL VJY. No recognition of the HLA-A11^{pos} non-B cell panel was observed (FIG. 2B). To conclude, 6 out of 8 tested T cell clones demonstrated promising safety profiles. T cell clones 4G10.15 (Jchain HLA-A3) and 1B7.11 (Jchain HLA-A24) demonstrated off-target recognition, and were therefore excluded from further analysis. For Jchain HLA-A1 and Jchain HLA-A11 both tested T cell clones were safe, but to reduce the number of clones for further analysis, the inventors selected one T cell clone per HLA restriction. For Jchain HLA-A1, T cell clone 5D12.9 and for Jchain HLA-A11, T cell clone 16C7.9 were selected for further studies given their favourable recognition profiles.

### Potent Recognition and Cytotoxicity of MM Cell Lines by Jchain T Cell Clones

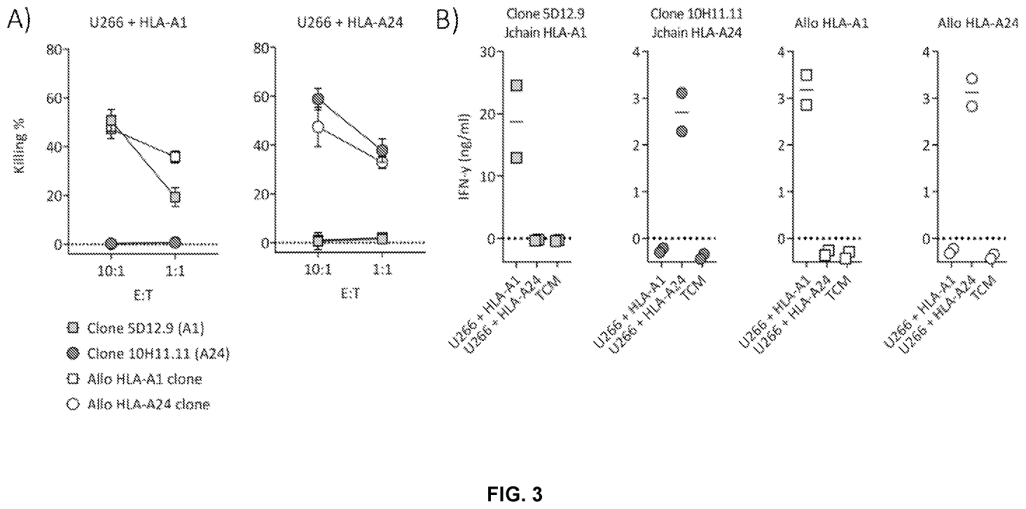

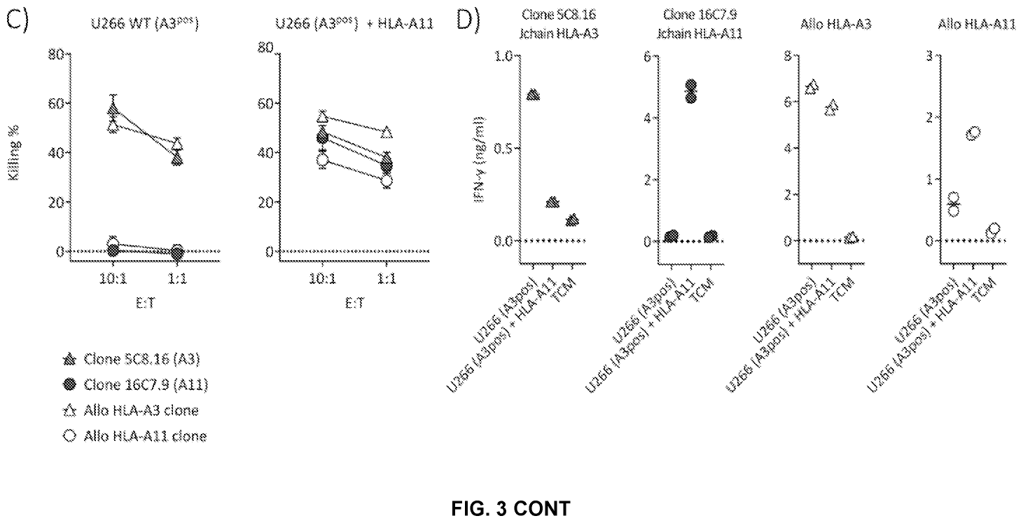

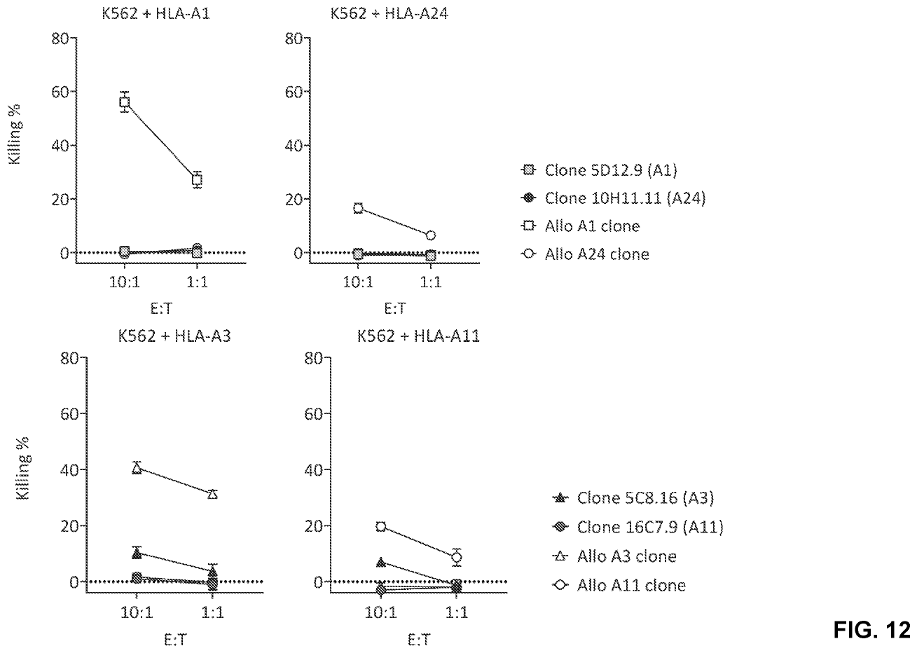

To determine the relevance of the identified T cell clones, recognition and killing of the Jchain expressing MM cell line U266 was investigated. Jchain expression in U266 cells was approximately 10-fold higher than expression of HKGs as measured by qPCR (FIG. 10). U266 WT cells are HLA-A3^{pos} but negative for the other target alleles, HLA-A1, All and A24 were therefore separately Td to allow recognition by all Jchain specific T cell clones. Target HLA expression and sensitivity to T cell mediated lysis was confirmed, in a 6-hour chromium release assay, by specific lysis upon co-culture with allo-HLA T cell clones (FIG. 3A). HLA-A1 and HLA-A24 Jchain specific T cell clones potently lysed HLA-A1^{pos} or HLA-A24^{pos} U266 cells (FIG. 3A). U266 lysis required target HLA expression as Jchain HLA-A1 specific clone 5D12.9 did not lyse HLA-A24^{pos} U266 and Jchain HLA-A24 specific clone 10H11.11 did not lyse HLA-A1^{pos} U266 cells. Additionally, Jchain negative HLA-A1^{pos} or HLA-A24^{pos} K562 cells were not lysed by Jchain specific T cell clones (FIG. 12). Target cell lysis corresponded with IFN-γ production upon overnight co-culture (FIG. 3B). Furthermore, Jchain HLA-A3 T cell clone 5C8.16 lysed WT HLA-A3^{pos} U266 cells, as well as U266 cells additionally Td with HLA-A11. Jchain HLA-A11 T cell clone 16C7.9 lysed HLA-A11^{pos} but not HLA-A11^{neg} WT U266 cells (FIG. 3C). A similar pattern was observed when IFN-γ production was measured (FIG. 3D). For T cell clone 5C8.16 cytokine production was reduced when U266 were Td with irrelevant HLA-11 allele likely resulting from competition for surface expression with endogenous HLA-A3. Combined, these data demonstrate that all identified Jchain specific T cell clones recognized and lysed Jchain expressing MM cells and that recognition depended on target HLA as well as Jchain expression. Accordingly, TCR sequencing of these T cell clones was performed to further investigate the potential relevance for TCR gene therapy of MM.

TCR Sequencing and Transfer Installs Jchain Specific Effector Functions onto CD8 T Cells

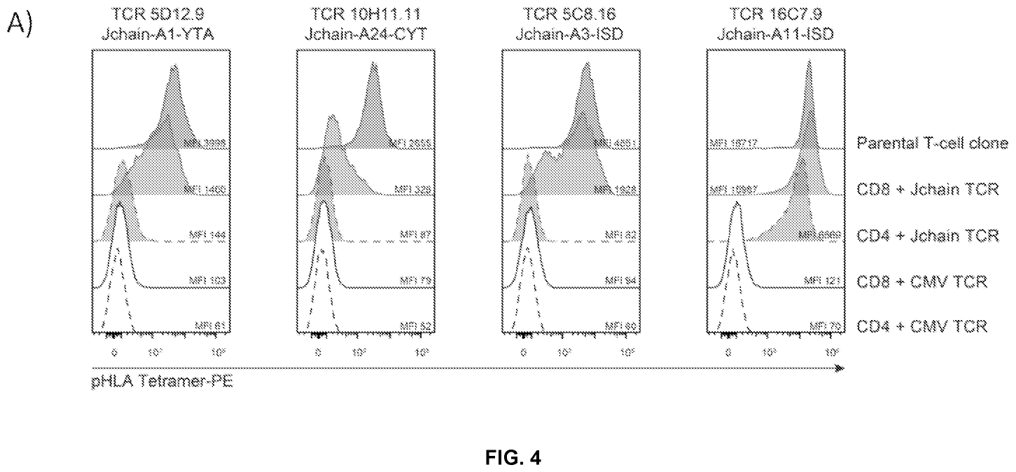

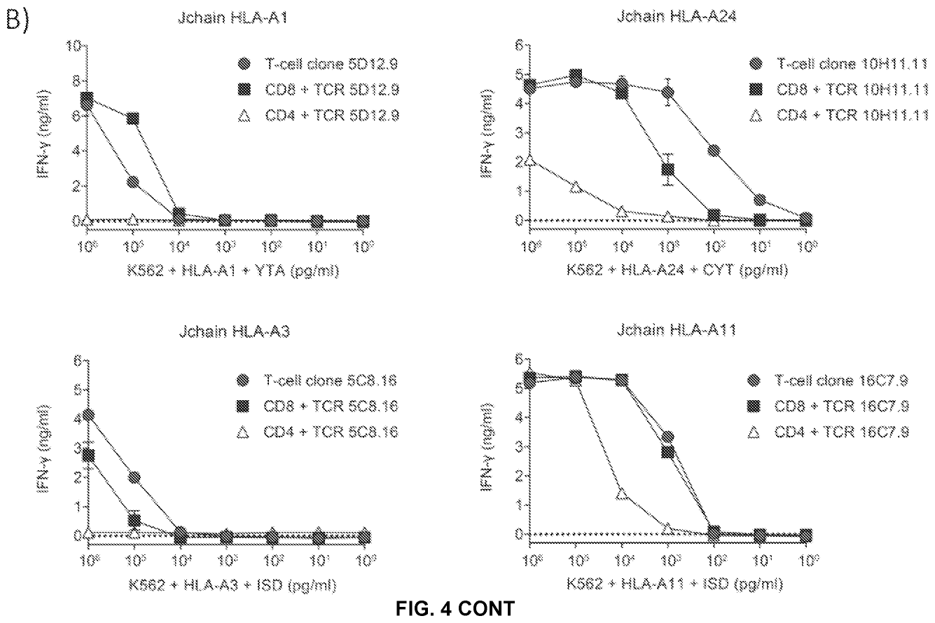

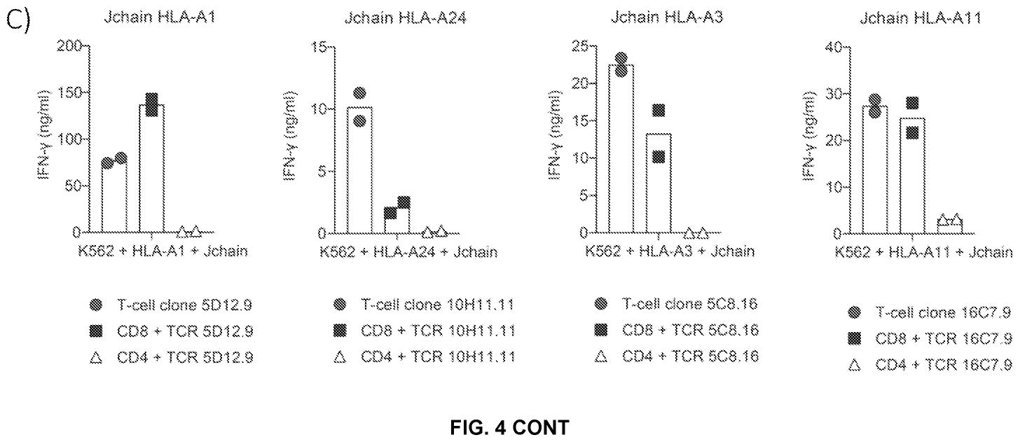

To investigate the potential for TCR gene transfer, TCRs of selected T cell clones were sequenced and retrovirally introduced in CD4 and CD8 T cells. For all four TCRs, introduction into healthy donor CD4^{pos} and CD8^{pos} T cells followed by TCR enrichment resulted in TCR cell surface expression as demonstrated by pH LA-tetramer binding (FIG. 4A). Staining intensity was lower in CD8 T cells compared to parental T cell clones. TCR from T cell clone 16C7.9 (ISD-A11) also bound pHLA-Tetramer in CD4 T cells, albeit to a lower level indicating partial dependence on the CD8 co-receptor for pHLA-Tetramer binding.

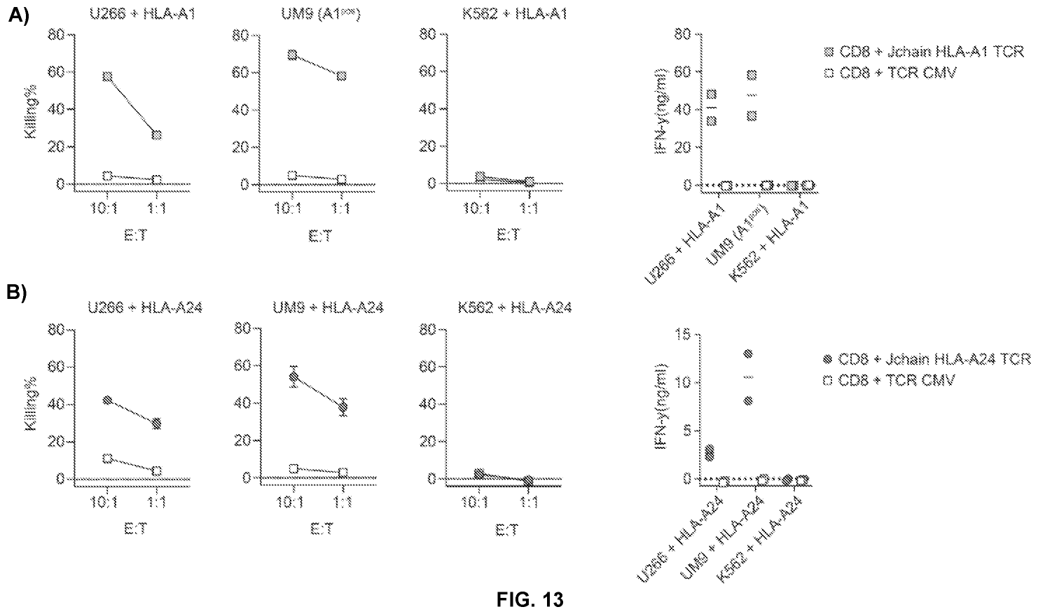

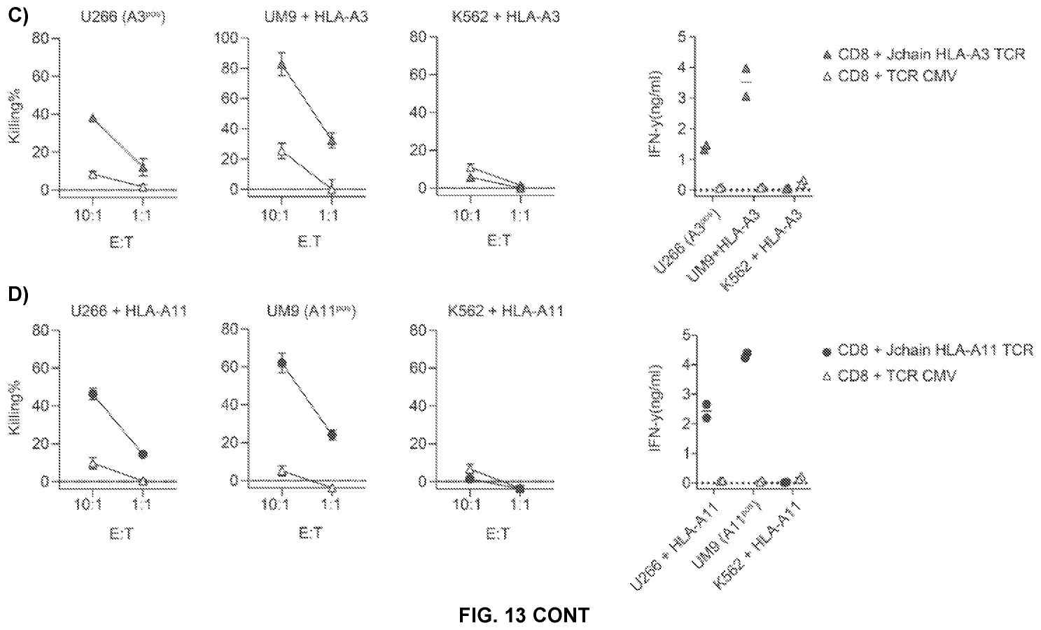

Peptide titration experiments and recognition of endogenously processed and presented antigen using Jchain Td K562 cells demonstrated functionality of all Jchain TCR Td CD8 T cells (FIG. 4B-C). Despite tetramer binding by TCR 16C7.19 (ISD-A11) Td CD4 T cells, cytokines were only produced when stimulated with target cells loaded with high concentrations of Jchain peptide. This corresponded to low cytokine production upon co-culture with Jchain Td K562 cells, therefore Jchain TCRs in CD4 T cells were not investigated further. Lytic potential of Jchain TCR Td CD8 T cells was confirmed using Jchain expressing MM cell lines U266 and UM9, and Jchain negative K562 cells. Jchain HLA-A1, A24, A3, and All TCR Td CD8 T cells induced potent lysis of Jchain^{pos} U266 as well as UM9 cells (FIG. 13), whereas antigen negative K562 cells were not lysed. Lytic capacity of the TCR Td CD8 T cells correlated with IFN-γ production upon stimulation with these cell lines. In conclusion, Jchain TCR Td CD8 T cells demonstrate Jchain specific functionality.

### Jchain TCR T Cells Target Healthy B Cells but not Jchain Negative Healthy Tissue

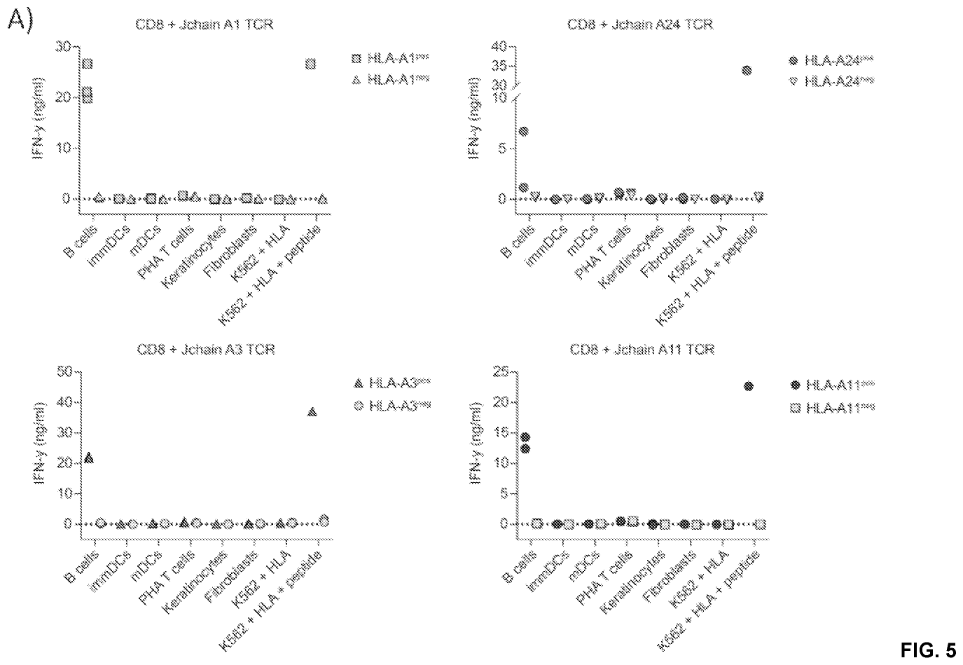

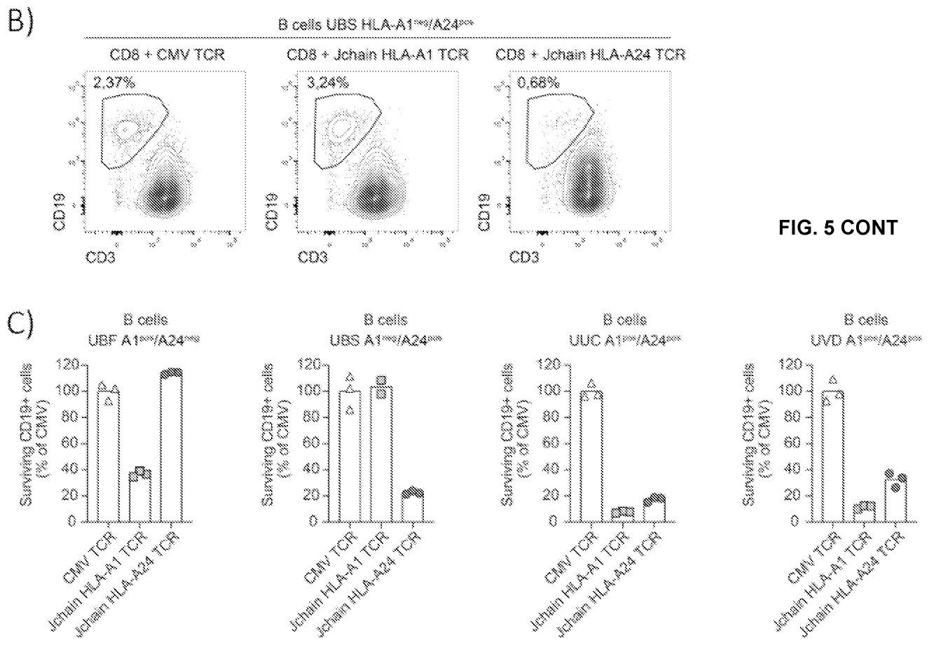

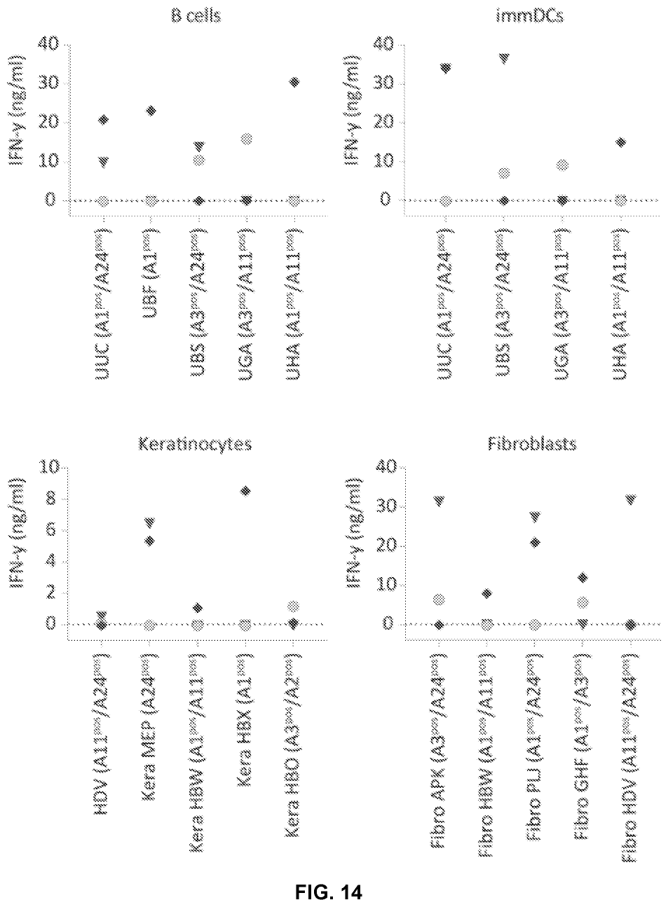

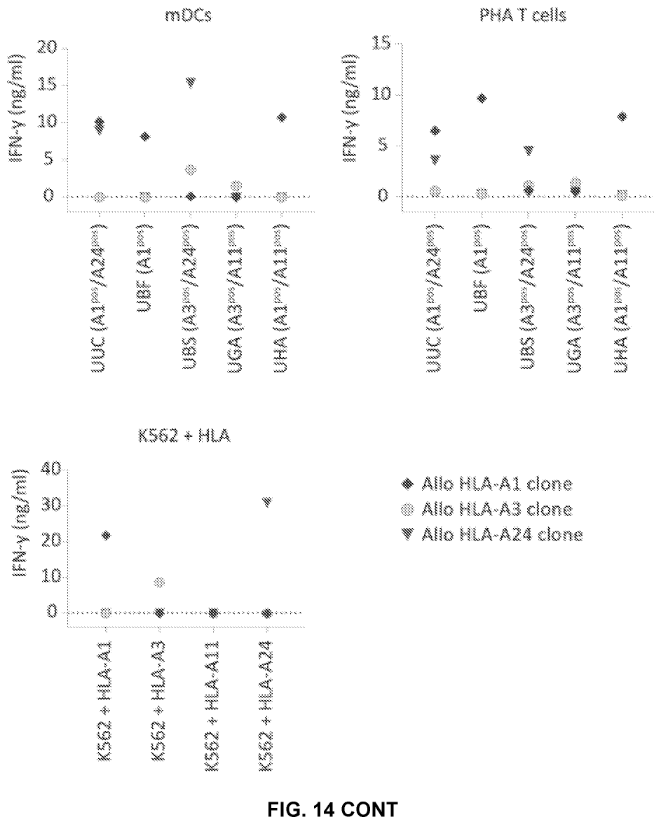

To assess off-tumor targeting, Jchain TCR Td T cells were co-cultured with healthy subsets of hematopoietic and non-hematopoietic origin. For each subset, cells from multiple donors positive and negative for HLA-A1, -A3, -A11 and -A24 were included. Jchain TCR Td CD8 T cells did not recognize primary immature dendritic cells (DCs), mature DCs, PHA activated T cells, fibroblasts, and keratinocytes (FIG. 5A), that were negative for Jchain (FIG. 9). Stimulatory capacity of used cell subsets varied but was confirmed by allo-HLA T cell clones (FIG. 14). Jchain positive peripheral blood B cells isolated from healthy donors were recognized by Jchain TCR T cells, but only when target HLA alleles were expressed (FIG. 5A). To investigate if this recognition resulted in depletion of B cells in vitro, Jchain A1 and Jchain A24 TCR T cells were co-cultured with B cells from multiple donors. FACS analysis after overnight co-culture revealed specific depletion of B cells from donors positive for target HLA alleles (FIG. 5B, C). Overall, Jchain TCR T cells maintained Jchain restricted recognition as previously seen for parental T cell clones.

### Jchain TCR T Cells Lyse Patient Derived MM Materials as Well as MM Cells In Vivo

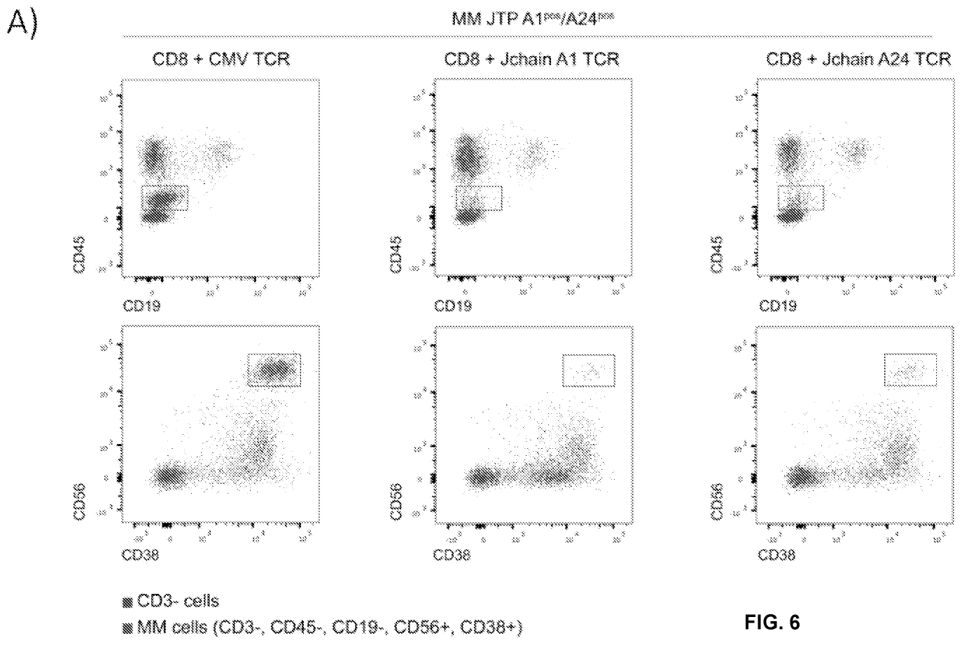

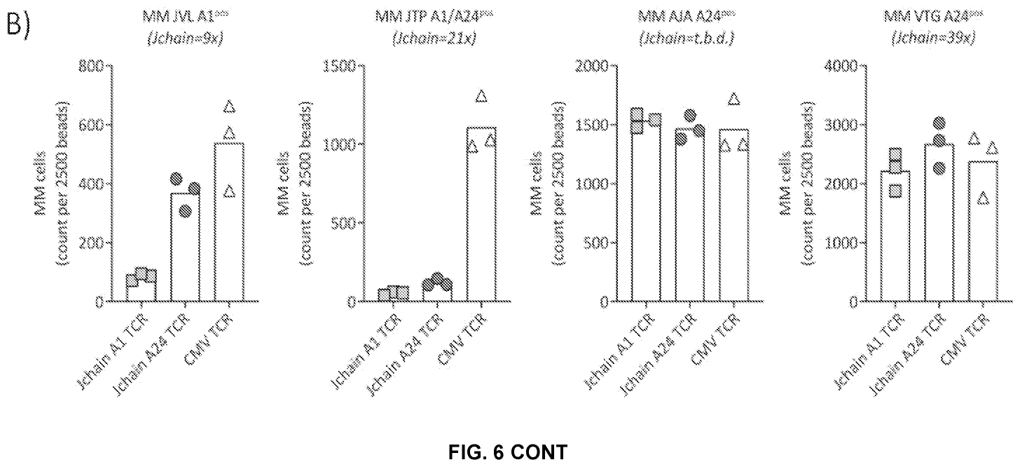

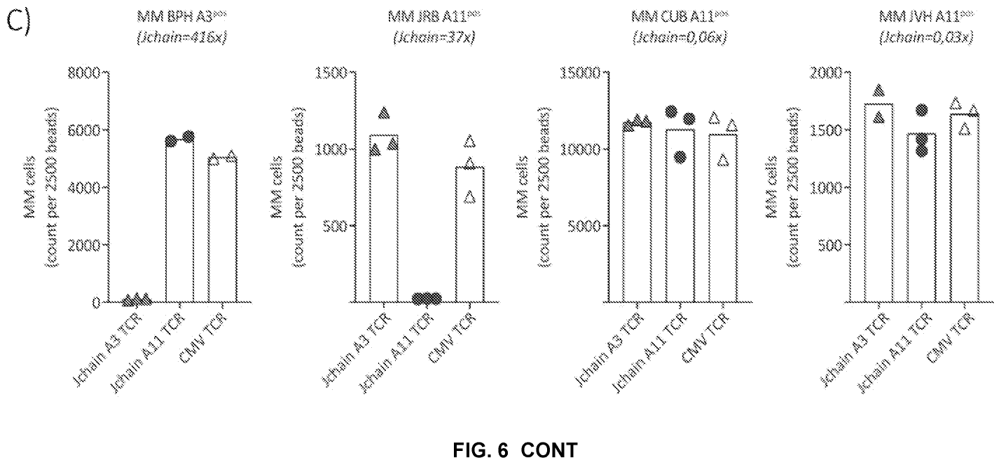

To gain further insight in the relevance of Jchain targeting TCR gene therapy for patients with MM, in vitro lysis of target HLA positive patient derived MM bone marrow (BM) samples was investigated. Due to the low percentage of malignant cells in the BM samples, FACS based killing assays were performed. MM cells were visualized using a marker panel staining for CD3, CD19, CD56, CD38 and CD45. MM cells were defined as CD3^{neg}, CD19^{neg}, CD45^{low}, CD56^{pos} and CD38^{pos}. Bone marrow samples were incubated overnight with CD8 T cells Td with HLA-A1, -24, -A3 or -All Jchain targeting TCRs or an irrelevant CMV TCR as negative control. Jchain expression in sorted MM cells ranged from 0.03 to 416 times relative to housekeeping genes, 2 out of 8 MM samples were negative for Jchain (<0.1 relative to HKG) (FIG. 10). Jchain TCR T cells efficiently lysed patient derived MM cells in BM samples, as exemplified by co-culture of Jchain A1 and A24 TCR T cells with material from HLA-A1^{pos}, A24^{pos} patient (FIG. 6A). Overall, 2 out of 2 HLA-A1^{pos} Jchain^{pos} MM samples, 1 out of 2 HLA-A24^{pos} Jchain^{pos} MM samples, 1 out of 1 HLA-A3^{pos} Jchain^{pos} sample, and 1 out of 1 HLA-A11^{pos} Jchain^{pos} sample were lysed after overnight co-culture with Jchain TCR T cells (FIG. 6B, 6C). Jchain^{neg} samples or samples not expressing the HLA restriction allele were not lysed by Jchain TCR T cells.

Finally, the inventors investigated in vivo targeting of MM cells using a previously established U266 xenograft model. NSG mice were separately inoculated with HLA-A1 Td, HLA-A24 Td, HLA-All Td or WT HLA-A3^{pos} U266 cells 3 weeks prior to T cell infusion to generate a model for established MM.

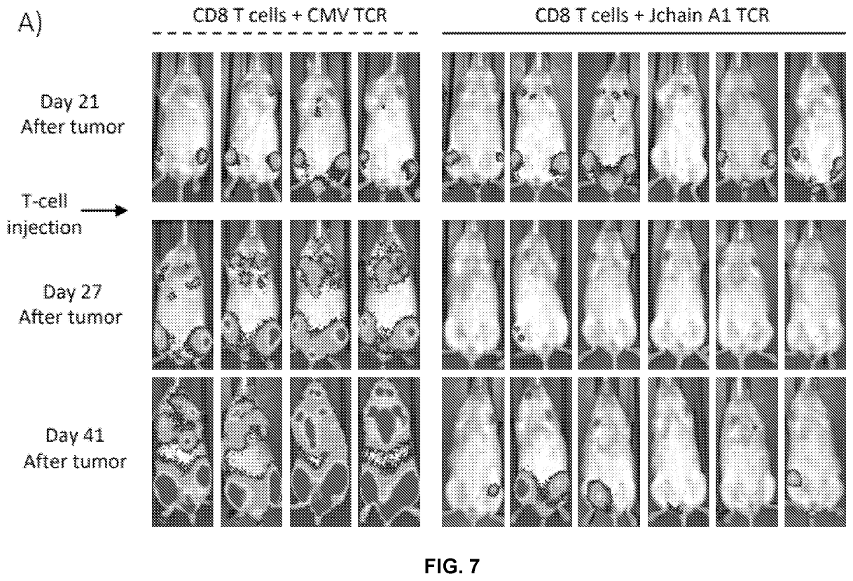

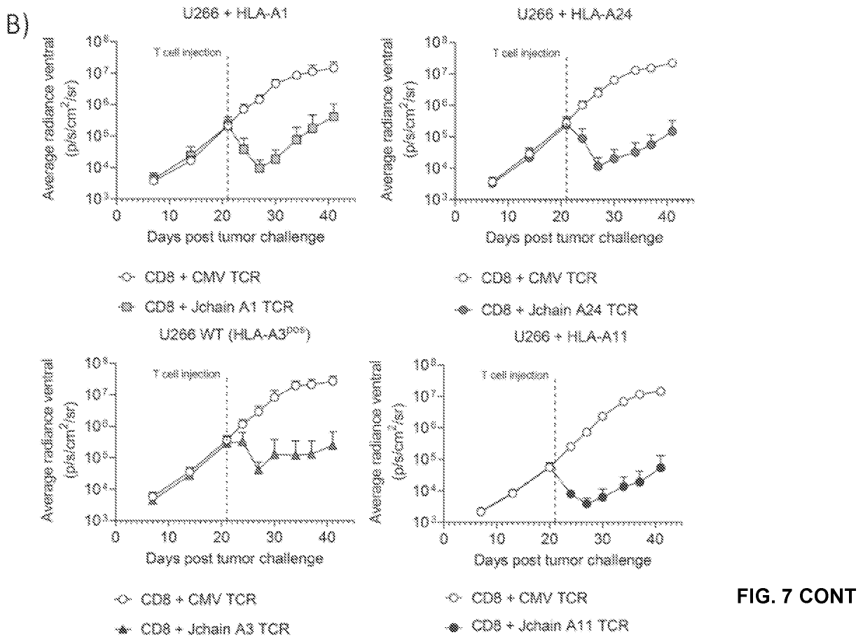

Subsequently, mice were infused with 3-6×10\hat{ }6 Jchain HLA-A1, -A24, -A3 or -A11 TCR or irrelevant CMV TCR Td CD8 T cells after which tumor outgrowth was followed measuring bioluminescence at regular intervals (FIG. 7A,B). All four Jchain TCRs readily reduced MM tumor burden upon infusion, lowest tumor burdens were reached 6 days after treatment. Compared to control mice, tumor load in treated mice was 154, 70, 190 and 209-fold lower for Jchain specific HLA-A1, A3, A11 and A24 TCRs respectively demonstrating strong in vivo anti-tumor responses (FIG. 7B). In this murine model, human T cells do not persist probably due to absence of a supportive human cytokine environment, which ultimately leads to tumor outgrowth at localized sites (FIG. 7A). To conclude, our data demonstrated that identified Jchain A1, A3 A11 and A24 TCR demonstrate potent anti-MM responses when transferred to CD8 T cells, both in vitro against patient derived MM samples as well as in vivo against Jchain^{pos} MM cell line U266.

### A Third YTA-A1 Jchain Specific TCR was Included in Key Screenings and Demonstrated Comparable Functionality to the More Extensively Studies TCRs

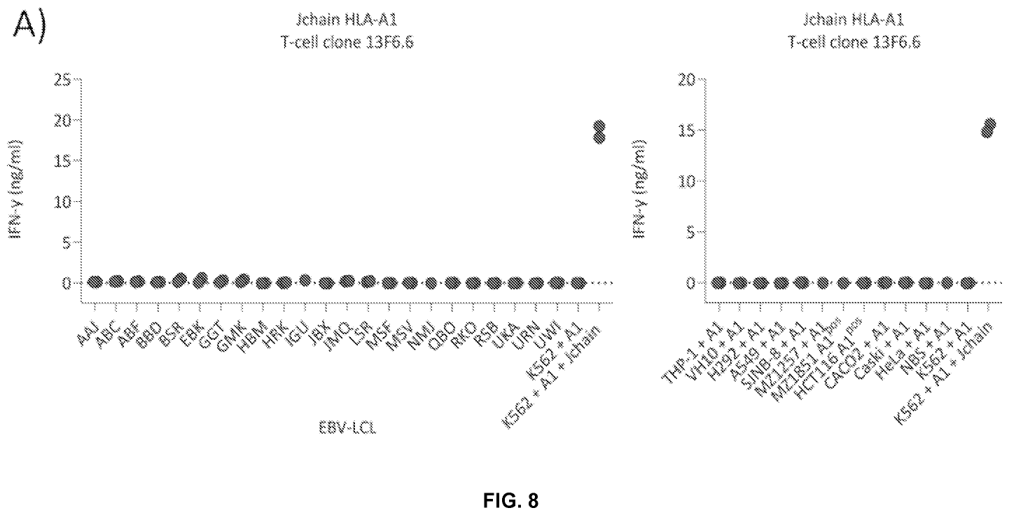

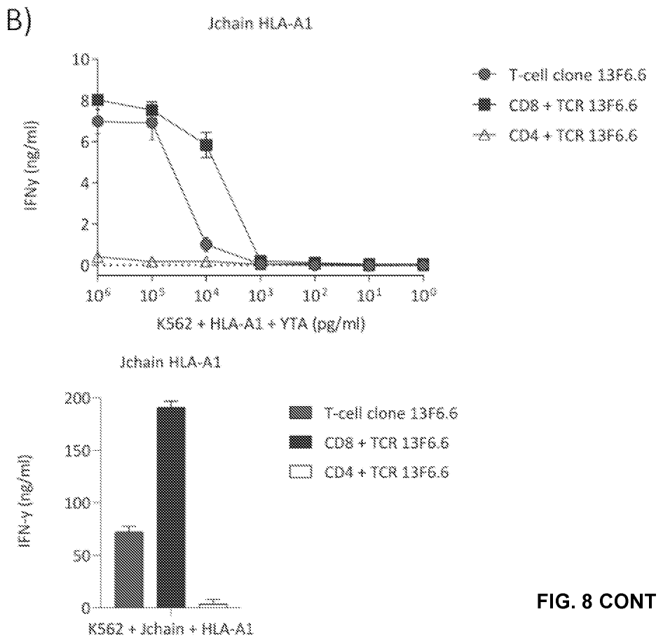

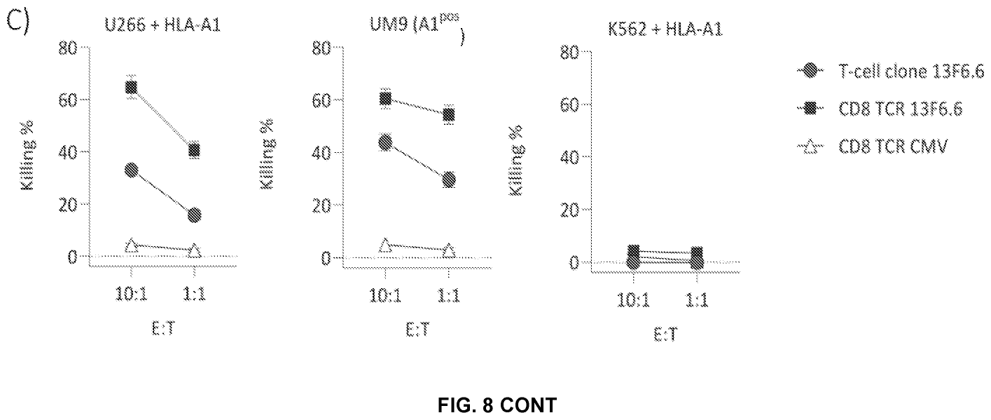

In addition to T cell clones 5D12.9 and 4G8.8, a third promising YTA-A1 Jchain specific T cell clone was identified. For practical reasons, the recognition profile of this T cell clone was less extensively studied. Nevertheless, safety as well as potency screenings demonstrated comparable and promising functionality. T cell clone 13F6.6 did not demonstrate any off-target recognition while Jchain Td K562 cells were highly recognized (FIG. 8A). Upon TCR sequencing and transfer to CD8 and CD4 T cells, Jchain specificity was maintained as observed in a peptide titration experiment (FIG. 8B). Similar to the parental T cell clones, TCR Td CD8 cells recognized endogenously processed and presented antigen on Jchain Td K562 cells (FIG. 8B). Additionally, TCR 13F6.6 Td CD8 T cells potently lysed Jchain expressing MM cell lines U266 and UM9 but not Jchain negative K562 cells (FIG. 8C). Together these data indicate that TCR 13F6.6, in addition to the previously described YTA-A1 Jchain specific TCRs, provides great promise for further pre-clinical development towards TCR gene therapy of MM.

### Additional Data

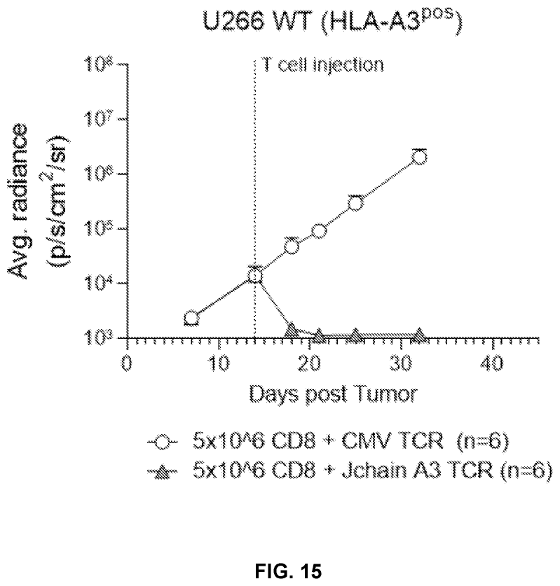

FIG. 15 demonstrates that J Chain specific TCR T cells can completely eradicate multiple myeloma in an in vivo model, in which NSG mice produce human IL-7 and IL-15.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### Sequences

TABLE 3
HLA-A*01:01, Jchain, YTA peptide: TCR 4G8.8
Sequences for HLA-A*01:01, Jchain, YTA peptide: TCR 4G8.8

| SEQ | TCR | AA | |
|---|---|---|---|
| ID | POLY- | or | |
| NO | PEPTIDE | NT | SEQUENCE |
| 1 | α CDR1 | AA | TSENNYY |
| 2 | α CDR2 | AA | QEAYKQQN |
| 3 | α CDR3 | AA | CAFMKHVEYGNKLVF |
| 4 | β CDR1 | AA | ENHRY |
| 5 | β CDR2 | AA | SYGVKD |
| 6 | β CDR3 | AA | CAARDSANEKLFF |
| 7 | α VJ | AA | MTRVSLLWAVVVSTCLESGMAQTVTQSQPEMSVQEAETVTLS |
| | | | CTYDTSENNYYLFWYKQPPSRQMILVIRQEAYKQQNATENRFS |
| | | | VNFQKAAKSFSLKISDSQLGDTAMYFCAFMKHVEYGNKLVFGA |
| | | | GTILRVKS |
| 8 | α VJ | NT | ATGACACGAGTTAGCTTGCTGTGGGCAGTCGTGGTCTCCAC |
| | | | CTGTCTTGAATCCGGCATGGCCCAGACAGTCACTCAGTCTC |
| | | | AACCAGAGATGTCTGTGCAGGAGGCAGAGACTGTGACCCTG |
| | | | AGTTGCACATATGACACCAGTGAGAATAATTATTATTTGTT |
| | | | CTGGTACAAGCAGCCTCCCAGCAGGCAGATGATTCTCGTTA |
| | | | TTCGCCAAGAAGCTTATAAGCAACAGAATGCAACGGAGAAT |
| | | | CGTTTCTCTGTGAACTTCCAGAAAGCAGCCAAATCCTTCAG |
| | | | TCTCAAGATCTCAGACTCACAGCTGGGGGACACTGCGATGT |
| | | | ATTTCTGTGCTTTCATGAAGCATGTGGAATATGGAAACAAA |
| | | | CTGGTCTTTGGCGCAGGAACCATTCTGAGAGTCAAGTCC |
| 9 | β VDJ | AA | MGTRLFFYVALCLLWTGHMDAGITQSPRHKVTETGTPVTLRCH |
| | | | QTENHRYMYWYRQDPGHGLRLIHYSYGVKDTDKGEVSDGYSVS |
| | | | RSKTEDFLLTLESATSSQTSVYFCAARDSANEKLFFGSGTQL |
| | | | SVL |
| 10 | β VDJ | NT | ATGGGCACAAGGTTGTTCTTCTATGTGGCCCTTTGTCTCCTG |
| | | | TGGACAGGACACATGGATGCTGGAATCACCCAGAGCCCAAG |
| | | | ACACAAGGTCACAGAGACAGGAACACCAGTGACTCTGAGAT |
| | | | GTCATCAGACTGAGAACCACCGCTATATGTACTGGTATCGAC |
| | | | AAGACCCGGGGCATGGGCTGAGGCTGATCCATTACTCATAT |
| | | | GGTGTTAAAGATACTGACAAAGGAGAAGTCTCAGATGGCTAT |
| | | | AGTGTCTCTAGATCAAAGACAGAGGATTTCCTCCTCACTCTG |
| | | | GAGTCCGCTACCAGCTCCCAGACATCTGTGTACTTCTGTGC |
| | | | CGCACGGGACAGCGCTAATGAAAAACTGTTTTTTGGCAGTG |
| | | | GAACCCAGCTCTCTGTCTTG |
| 11 | α VJ and | AA | MTRVSLLWAVVVSTCLESGMAQTVTQSQPEMSVQEAETVTLS |
| | constant | | CTYDTSENNYYLFWYKQPPSRQMILVIRQEAYKQQNATENRF |
| | | | SVNFQKAAKSFSLKISDSQLGDTAMYFCAFMKHVEYGNKLVF |
| | | | GAGTILRVKSYIQNPDPAVYQLRDSKSSDKSVCLFTDFDSQT |
| | | | NVSQSKDSDVYITDKTVLDMRSMDFKSNSAVAWSNKSDFACA |
| | | | NAFNNSIIPEDTFFPSPESSCDVKLVEKSFETDTNLNFQNLS |
| | | | VIGFRILLLKVAGFNLLMTLRLWSS |
| 12 | α VJ and | NT | ATGACACGAGTTAGCTTGCTGTGGGCAGTCGTGGTCTCCAC |
| | constant | | CTGTCTTGAATCCGGCATGGCCCAGACAGTCACTCAGTCTC |
| | | | AACCAGAGATGTCTGTGCAGGAGGCAGAGACTGTGACCCTG |
| | | | AGTTGCACATATGACACCAGTGAGAATAATTATTATTTGTTC |
| | | | TGGTACAAGCAGCCTCCCAGCAGGCAGATGATTCTCGTTATT |
| | | | CGCCAAGAAGCTTATAAGCAACAGAATGCAACGGAGAATCG |
| | | | TTTCTCTGTGAACTTCCAGAAAGCAGCCAAATCCTTCAGTCT |
| | | | CAAGATCTCAGACTCACAGCTGGGGGACACTGCGATGTATT |
| | | | TCTGTGCTTTCATGAAGCATGTGGAATATGGAAACAAACTGG |
| | | | TCTTTGGCGCAGGAACCATTCTGAGAGTCAAGTCCTATATCC |
| | | | AGAACCCTGACCCTGCCGTGTACCAGCTGAGAGACTCTAAA |
| | | | TCCAGTGACAAGTCTGTCTGCCTATTCACCGATTTTGATTCT |
| | | | CAAACAAATGTGTCACAAAGTAAGGATTCTGATGTGTATATC |
| | | | ACAGACAAAACTGTGCTAGACATGAGGTCTATGGACTTCAAG |
| | | | AGCAACAGTGCTGTGGCCTGGAGCAACAAATCTGACTTTGC |
| | | | ATGTGCAAACGCCTTCAACAACAGCATTATTCCAGAAGACAC |
| | | | CTTCTTCCCCAGCCCAGAAAGTTCCTGTGATGTCAAGCTGGT |
| | | | CGAGAAAAGCTTTGAAACAGATACGAACCTAAACTTTCAAAA |
| | | | CCTGTCAGTGATTGGGTTCCGAATCCTCCTCCTGAAAGTGG |
| | | | CCGGGTTTAATCTGCTCATGACGCTGCGGTTGTGGTCCAGC |
| | | | TGA |
| 13 | β VDJ | AA | MGTRLFFYVALCLLWTGHMDAGITQSPRHKVTETGTPVTLRC |
| | and | | HQTENHRYMYWYRQDPGHGLRLIHYSYGVKDTDKGEVSDGYS |
| | constant | | VSRSKTEDFLLTLESATSSQTSVYFCAARDSANEKLFFGSGT |
| | | | QLSVLEDLNKVFPPEVAVFEPSEAEISHTQKATLVCLATGFF |
| | | | PDHVELSWWWVNGKEVHSGVSTDPQPLKEQPALNDSRYCLSS |
| | | | RLRVSATFWQNPRNHFRCQVQFYGLSENDEWTQDRAKPVTQI |
| | | | VSAEAWGRADCGFTSVSYQQGVLSATILYEILLGKATLYAVL |
| | | | VSALVLMAMVKRKDF |
| 14 | VDJ | NT | ATGGGCACAAGGTTGTTCTTCTATGTGGCCCTTTGTCTCCTG |
| | and | | TGGACAGGACACATGGATGCTGGAATCACCCAGAGCCCAAG |
| | constant | | ACACAAGGTCACAGAGACAGGAACACCAGTGACTCTGAGAT |
| | | | GTCATCAGACTGAGAACCACCGCTATATGTACTGGTATCGAC |
| | | | AAGACCCGGGGCATGGGCTGAGGCTGATCCATTACTCATAT |
| | | | GGTGTTAAAGATACTGACAAAGGAGAAGTCTCAGATGGCTAT |
| | | | AGTGTCTCTAGATCAAAGACAGAGGATTTCCTCCTCACTCTG |
| | | | GAGTCCGCTACCAGCTCCCAGACATCTGTGTACTTCTGTGC |
| | | | CGCACGGGACAGCGCTAATGAAAAACTGTTTTTTGGCAGTG |
| | | | GAACCCAGCTCTCTGTCTTGGAGGACCTGAACAAGGTGTTC |
| | | | CCACCCGAGGTCGCTGTGTTTGAGCCATCAGAAGCAGAGAT |
| | | | CTCCCACACCCAAAAGGCCACACTGGTGTGCCTGGCCACAG |
| | | | GCTTCTTCCCCGACCACGTGGAGCTGAGCTGGTGGGTGAAT |
| | | | GGGAAGGAGGTGCACAGTGGGGTCAGCACAGACCCGCAGC |
| | | | CCCTCAAGGAGCAGCCCGCCCTCAATGACTCCAGATACTGC |
| | | | CTGAGCAGCCGCCTGAGGGTCTCGGCCACCTTCTGGCAGA |
| | | | ACCCCCGCAACCACTTCCGCTGTCAAGTCCAGTTCTACGGG |
| | | | CTCTCGGAGAATGACGAGTGGACCCAGGATAGGGCCAAAC |
| | | | CCGTCACCCAGATCGTCAGCGCCGAGGCCTGGGGTAGAGC |
| | | | AGACTGTGGCTTTACCTCGGTGTCCTACCAGCAAGGGGTCC |
| | | | TGTCTGCCACCATCCTCTATGAGATCCTGCTAGGGAAGGCC |
| | | | ACCCTGTATGCTGTGCTGGTCAGCGCCCTTGTGTTGATGGC |
| | | | CATGGTCAAGAGAAAGGATTTCTGA |

TABLE 4
HLA-A*01:01, Jchain, YTA peptide: TCR 5D12.9
Sequences for HLA-A*01:01, Jchain, YTA peptide: TCR 5D12.9

| SEQ | TCR | AA | |
|---|---|---|---|
| ID | POLY- | or | |
| NO | PEPTIDE | NT | SEQUENCE |
| 15 | α CDR1 | AA | DRGSQSFF |
| 16 | α CDR2 | AA | IYSNGD |
| 17 | α CDR3 | AA | CAVMDSNYQLIW |
| 18 | β CDR1 | AA | SGHVS |
| 19 | β CDR2 | AA | FQNEAQ |
| 20 | β CDR3 | AA | CASSPGEFGETQYF |
| 21 | α VJ | AA | MKSLRVLLVILWLQLSWVWSQQKEVEQNSGPLSVPEGAIASLNC |
| | | | TYSDRGSQSFFWYRQYSGKSPELIMFIYSNGDKEDGRFTAQLNK |
| | | | ASQYVSLLIRDSQPSDSATYLCAVMDSNYQLIWGAGTKLIIKP |
| 22 | α VJ | NT | ATGAAATCCTTGAGAGTTTTACTAGTGATCCTGTGGCTTCAGTT |
| | | | GAGCTGGGTTTGGAGCCAACAGAAGGAGGTGGAGCAGAATTC |
| | | | TGGACCCCTCAGTGTTCCAGAGGGAGCCATTGCCTCTCTCAAC |
| | | | TGCACTTACAGTGACCGAGGTTCCCAGTCCTTCTTCTGGTACA |
| | | | GACAATATTCTGGGAAAAGCCCTGAGTTGATAATGTTCATATAC |
| | | | TCCAATGGTGACAAAGAAGATGGAAGGTTTACAGCACAGCTCA |
| | | | ATAAAGCCAGCCAGTATGTTTCTCTGCTCATCAGAGACTCCCA |
| | | | GCCCAGTGATTCAGCCACCTACCTCTGTGCCGTAATGGATAGC |
| | | | AACTATCAGTTAATCTGGGGCGCTGGGACCAAGCTAATTATAA |
| | | | AGCCA |
| 23 | β VDJ | AA | MGTRLLCWVVLGFLGTDHTGAGVSQSPRYKVAKRGQDVALRCD |
| | | | PISGHVSLFWYQQALGQGPEFLTYFQNEAQLDKSGLPSDRFFAE |
| | | | RPEGSVSTLKIQRTQQEDSAVYLCASSPGEFGETQYFGPGTRLLV |
| | | | L |
| 24 | β VDJ | NT | ATGGGCACCAGGCTCCTCTGCTGGGTGGTCCTGGGTTTCCTA |
| | | | GGGACAGATCACACAGGTGCTGGAGTCTCCCAGTCCCCTAGG |
| | | | TACAAAGTCGCAAAGAGAGGACAGGATGTAGCTCTCAGGTGT |
| | | | GATCCAATTTCGGGTCATGTATCCCTTTTTTGGTACCAACAGGC |
| | | | CCTCGGGCAGGGGCCAGAGTTTCTGACTTATTTCCAGAATGAA |
| | | | GCTCAACTAGACAAATCGGGGCTGCCCAGTGATCGCTTCTTTG |
| | | | CAGAAAGGCCTGAGGGATCCGTCTCCACTCTGAAGATCCAGC |
| | | | GCACACAGCAGGAGGACTCCGCCGTGTATCTCTGTGCCAGCA |
| | | | GCCCGGGGGAATTTGGGGAGACCCAGTACTTCGGGCCAGGC |
| | | | ACGCGGCTCCTGGTGCTC |
| 25 | α VJ and | AA | MKSLRVLLVILWLQLSWVWSQQKEVEQNSGPLSVPEGAIASLNC |
| | constant | | TYSDRGSQSFFWYRQYSGKSPELIMFIYSNGDKEDGRFTAQLNK |
| | | | ASQYVSLLIRDSQPSDSATYLCAVMDSNYQLIWGAGTKLIIKPD |
| | | | IQNPDPAVYQLRDSKSSDKSVCLFTDFDSQTNVSQSKDSDVYIT |
| | | | DKTVLDMRSMDFKSNSAVAWSNKSDFACANAFNNSIIPEDTFFP |
| | | | SPESSCDVKLVEKSFETDTNLNFQNLSVIGFRILLLKVAGFNLL |
| | | | MTLRLWSS |
| 26 | α VJ and | NT | ATGAAATCCTTGAGAGTTTTACTAGTGATCCTGTGGCTTCAGT |
| | constant | | TGAGCTGGGTTTGGAGCCAACAGAAGGAGGTGGAGCAGAATTC |
| | | | TGGACCCCTCAGTGTTCCAGAGGGAGCCATTGCCTCTCTCAAC |
| | | | TGCACTTACAGTGACCGAGGTTCCCAGTCCTTCTTCTGGTACA |
| | | | GACAATATTCTGGGAAAAGCCCTGAGTTGATAATGTTCATATAC |
| | | | TCCAATGGTGACAAAGAAGATGGAAGGTTTACAGCACAGCTCA |
| | | | ATAAAGCCAGCCAGTATGTTTCTCTGCTCATCAGAGACTCCCA |
| | | | GCCCAGTGATTCAGCCACCTACCTCTGTGCCGTAATGGATAGC |
| | | | AACTATCAGTTAATCTGGGGCGCTGGGACCAAGCTAATTATAA |
| | | | AGCCAGATATCCAGAACCCTGACCCTGCCGTGTACCAGCTGA |
| | | | GAGACTCTAAATCCAGTGACAAGTCTGTCTGCCTATTCACCGA |
| | | | TTTTGATTCTCAAACAAATGTGTCACAAAGTAAGGATTCTGATG |
| | | | TGTATATCACAGACAAAACTGTGCTAGACATGAGGTCTATGGA |
| | | | CTTCAAGAGCAACAGTGCTGTGGCCTGGAGCAACAAATCTGAC |
| | | | TTTGCATGTGCAAACGCCTTCAACAACAGCATTATTCCAGAAGA |
| | | | CACCTTCTTCCCCAGCCCAGAAAGTTCCTGTGATGTCAAGCTG |
| | | | GTCGAGAAAAGCTTTGAAACAGATACGAACCTAAACTTTCAAAA |
| | | | CCTGTCAGTGATTGGGTTCCGAATCCTCCTCCTGAAAGTGGCC |
| | | | GGGTTTAATCTGCTCATGACGCTGCGGTTGTGGTCCAGCTGA |
| 27 | VDJ | AA | MGTRLLCWVVLGFLGTDHTGAGVSQSPRYKVAKRGQDVALRCDP |
| | and | | ISGHVSLFWYQQALGQGPEFLTYFQNEAQLDKSGLPSDRFFAER |
| | constant | | PEGSVSTLKIQRTQQEDSAVYLCASSPGEFGETQYFGPGTRLLV |
| | | | LEDLNKVFPPEVAVFEPSEAEISHTQKATLVCLATGFFPDHVEL |
| | | | SWWVNGKEVHSGVSTDPQPLKEQPALNDSRYCLSSRLRVSATFW |
| | | | QNPRNHFRCQVQFYGLSENDEWTQDRAKPVTQIVSAEAWGRAD |
| | | | CGFTSVSYQQGVLSATILYEILLGKATLYAVLVSALVLMAMVK |
| | | | RKDF |
| 28 | β VDJ | NT | ATGGGCACCAGGCTCCTCTGCTGGGTGGTCCTGGGTTTCCTA |
| | and | | GGGACAGATCACACAGGTGCTGGAGTCTCCCAGTCCCCTAGG |
| | constant | | TACAAAGTCGCAAAGAGAGGACAGGATGTAGCTCTCAGGTGT |
| | | | GATCCAATTTCGGGTCATGTATCCCTTTTTTGGTACCAACAGGC |
| | | | CCTCGGGCAGGGGCCAGAGTTTCTGACTTATTTCCAGAATGAA |
| | | | GCTCAACTAGACAAATCGGGGCTGCCCAGTGATCGCTTCTTTG |
| | | | CAGAAAGGCCTGAGGGATCCGTCTCCACTCTGAAGATCCAGC |
| | | | GCACACAGCAGGAGGACTCCGCCGTGTATCTCTGTGCCAGCA |
| | | | GCCCGGGGGAATTTGGGGAGACCCAGTACTTCGGGCCAGGC |
| | | | ACGCGGCTCCTGGTGCTCGAGGACCTGAACAAGGTGTTCCCA |
| | | | CCCGAGGTCGCTGTGTTTGAGCCATCAGAAGCAGAGATCTCC |
| | | | CACACCCAAAAGGCCACACTGGTGTGCCTGGCCACAGGCTTC |
| | | | TTCCCCGACCACGTGGAGCTGAGCTGGTGGGTGAATGGGAAG |
| | | | GAGGTGCACAGTGGGGTCAGCACAGACCCGCAGCCCCTCAAG |
| | | | GAGCAGCCCGCCCTCAATGACTCCAGATACTGCCTGAGCAGC |
| | | | CGCCTGAGGGTCTCGGCCACCTTCTGGCAGAACCCCCGCAAC |
| | | | CACTTCCGCTGTCAAGTCCAGTTCTACGGGCTCTCGGAGAATG |
| | | | ACGAGTGGACCCAGGATAGGGCCAAACCCGTCACCCAGATCG |
| | | | TCAGCGCCGAGGCCTGGGGTAGAGCAGACTGTGGCTTTACCT |
| | | | CGGTGTCCTACCAGCAAGGGGTCCTGTCTGCCACCATCCTCTA |
| | | | TGAGATCCTGCTAGGGAAGGCCACCCTGTATGCTGTGCTGGT |
| | | | CAGCGCCCTTGTGTTGATGGCCATGGTCAAGAGAAAGGATTTC |
| | | | TGA |

TABLE 5
HLA-A*24:02, Jchain, CYT peptide: TCR 10H11.11
Sequences for HLA-A*24:02, Jchain, CYT peptide: TCR 10H11.11

| SEQ | TCR | AA | |
|---|---|---|---|
| ID | POLY- | or | |
| NO | PEPTIDE | NT | SEQUENCE |
| 29 | α CDR1 | AA | VSGLRG |
| 30 | α CDR2 | AA | LYSAGEE |
| 31 | α CDR3 | AA | CAVLSGGSYIPTF |
| 32 | β CDR1 | AA | SEHNR |
| 33 | β CDR2 | AA | FQNEAQ |
| 34 | β CDR3 | AA | CASSPLLRDSEEKLFF |
| 35 | α VJ | AA | MEKMLECAFIVLWLQLGWLSGEDQVTQSPEALRLQEGESSSLNC |
| | | | SYTVSGLRGLFWYRQDPGKGPEFLFTLYSAGEEKEKERLKATLT |
| | | | KKESFLHITAPKPEDSATYLCAVLSGGSYIPTFGRGTSLIVHP |
| 36 | α VJ | NT | ATGGAGAAAATGTTGGAGTGTGCATTCATAGTCTTGTGGCTTC |
| | | | AGCTTGGCTGGTTGAGTGGAGAAGACCAGGTGACGCAGAGTC |
| | | | CCGAGGCCCTGAGACTCCAGGAGGGAGAGAGTAGCAGTCTTA |
| | | | ACTGCAGTTACACAGTCAGCGGTTTAAGAGGGCTGTTCTGGTA |
| | | | TAGGCAAGATCCTGGGAAAGGCCCTGAATTCCTCTTCACCCTG |
| | | | TATTCAGCTGGGGAAGAAAAGGAGAAAGAAAGGCTAAAAGCCA |
| | | | CATTAACAAAGAAGGAAAGCTTTCTGCACATCACAGCCCCTAA |
| | | | ACCTGAAGACTCAGCCACTTATCTCTGTGCTGTGTTATCAGGA |
| | | | GGAAGCTACATACCTACATTTGGAAGAGGAACCAGCCTTATTG |
| | | | TTCATCCG |
| 37 | β VDJ | AA | MGTSLLCWMALCLLGADHADTGVSQDPRHKITKRGQNVTFRCDP |
| | | | ISEHNRLYWYRQTLGQGPEFLTYFQNEAQLEKSRLLSDRFSAERP |
| | | | KGSFSTLEIQRTEQGDSAMYLCASSPLLRDSEEKLFFGSGTQLSV |
| | | | L |
| 38 | β VDJ | NT | ATGGGCACCAGCCTCCTCTGCTGGATGGCCCTGTGTCTCCTG |
| | | | GGGGCAGATCACGCAGATACTGGAGTCTCCCAGGACCCCAGA |
| | | | CACAAGATCACAAAGAGGGGACAGAATGTAACTTTCAGGTGTG |
| | | | ATCCAATTTCTGAACACAACCGCCTTTATTGGTACCGACAGACC |
| | | | CTGGGGCAGGGCCCAGAGTTTCTGACTTACTTCCAGAATGAAG |
| | | | CTCAACTAGAAAAATCAAGGCTGCTCAGTGATCGGTTCTCTGC |
| | | | AGAGAGGCCTAAGGGATCTTTCTCCACCTTGGAGATCCAGCGC |
| | | | ACAGAGCAGGGGGACTCGGCCATGTATCTCTGTGCCAGCAGC |
| | | | CCCCTCCTCCGGGACAGCGAAGAAAAACTGTTTTTTGGCAGTG |
| | | | GAACCCAGCTCTCTGTCTTG |
| 39 | α VJ and | AA | MEKMLECAFIVLWLQLGWLSGEDQVTQSPEALRLQEGESSSLNC |
| | constant | | SYTVSGLRGLFWYRQDPGKGPEFLFTLYSAGEEKEKERLKATLTK |
| | | | KESFLHITAPKPEDSATYLCAVLSGGSYIPTFGRGTSLIVHPYIQ |
| | | | NPDPAVYQLRDSKSSDKSVCLFTDFDSQTNVSQSKDSDVYITDKT |
| | | | VLDMRSMDFKSNSAVAWSNKSDFACANAFNNSIIPEDTFFPSPES |
| | | | SCDVKLVEKSFETDTNLNFQNLSVIGFRILLLKVAGFNLLMTLRL |
| | | | WSS |
| 40 | α VJ and | NT | ATGGAGAAAATGTTGGAGTGTGCATTCATAGTCTTGTGGCTTC |
| | constant | | AGCTTGGCTGGTTGAGTGGAGAAGACCAGGTGACGCAGAGTC |
| | | | CCGAGGCCCTGAGACTCCAGGAGGGAGAGAGTAGCAGTCTTA |
| | | | ACTGCAGTTACACAGTCAGCGGTTTAAGAGGGCTGTTCTGGTA |
| | | | TAGGCAAGATCCTGGGAAAGGCCCTGAATTCCTCTTCACCCTG |
| | | | TATTCAGCTGGGGAAGAAAAGGAGAAAGAAAGGCTAAAAGCCA |
| | | | CATTAACAAAGAAGGAAAGCTTTCTGCACATCACAGCCCCTAA |
| | | | ACCTGAAGACTCAGCCACTTATCTCTGTGCTGTGTTATCAGGA |
| | | | GGAAGCTACATACCTACATTTGGAAGAGGAACCAGCCTTATTG |
| | | | TTCATCCGTATATCCAGAACCCTGACCCTGCCGTGTACCAGCT |
| | | | GAGAGACTCTAAATCCAGTGACAAGTCTGTCTGCCTATTCACC |
| | | | GATTTTGATTCTCAAACAAATGTGTCACAAAGTAAGGATTCTGA |
| | | | TGTGTATATCACAGACAAAACTGTGCTAGACATGAGGTCTATG |
| | | | GACTTCAAGAGCAACAGTGCTGTGGCCTGGAGCAACAAATCTG |
| | | | ACTTTGCATGTGCAAACGCCTTCAACAACAGCATTATTCCAGAA |
| | | | GACACCTTCTTCCCCAGCCCAGAAAGTTCCTGTGATGTCAAGC |
| | | | TGGTCGAGAAAAGCTTTGAAACAGATACGAACCTAAACTTTCAA |
| | | | AACCTGTCAGTGATTGGGTTCCGAATCCTCCTCCTGAAAGTGG |
| | | | CCGGGTTTAATCTGCTCATGACGCTGCGGTTGTGGTCCAGCTG |
| | | | A |
| 41 | β VDJ | AA | MGTSLLCWMALCLLGADHADTGVSQDPRHKITKRGQNVTFRCDP |
| | and | | ISEHNRLYWYRQTLGQGPEFLTYFQNEAQLEKSRLLSDRFSAERP |
| | constant | | KGSFSTLEIQRTEQGDSAMYLCASSPLLRDSEEKLFFGSGTQLSV |
| | | | LEDLNKVFPPEVAVFEPSEAEISHTQKATLVCLATGFFPDHVELS |
| | | | WWVNGKEVHSGVSTDPQPLKEQPALNDSRYCLSSRLRVSATFWQN |
| | | | PRNHFRCQVQFYGLSENDEWTQDRAKPVTQIVSAEAWGRADCGFT |
| | | | SVSYQQGVLSATILYEILLGKATLYAVLVSALVLMAMVKRKDF |
| 42 | β VDJ | NT | ATGGGCACCAGCCTCCTCTGCTGGATGGCCCTGTGTCTCCTG |
| | and | | GGGGCAGATCACGCAGATACTGGAGTCTCCCAGGACCCCAGA |
| | constant | | CACAAGATCACAAAGAGGGGACAGAATGTAACTTTCAGGTGTG |
| | | | ATCCAATTTCTGAACACAACCGCCTTTATTGGTACCGACAGACC |
| | | | CTGGGGCAGGGCCCAGAGTTTCTGACTTACTTCCAGAATGAAG |
| | | | CTCAACTAGAAAAATCAAGGCTGCTCAGTGATCGGTTCTCTGC |
| | | | AGAGAGGCCTAAGGGATCTTTCTCCACCTTGGAGATCCAGCGC |
| | | | ACAGAGCAGGGGGACTCGGCCATGTATCTCTGTGCCAGCAGC |
| | | | CCCCTCCTCCGGGACAGCGAAGAAAAACTGTTTTTTGGCAGTG |
| | | | GAACCCAGCTCTCTGTCTTGGAGGACCTGAACAAGGTGTTCCC |
| | | | ACCCGAGGTCGCTGTGTTTGAGCCATCAGAAGCAGAGATCTCC |
| | | | CACACCCAAAAGGCCACACTGGTGTGCCTGGCCACAGGCTTC |
| | | | TTCCCCGACCACGTGGAGCTGAGCTGGTGGGTGAATGGGAAG |
| | | | GAGGTGCACAGTGGGGTCAGCACAGACCCGCAGCCCCTCAAG |
| | | | GAGCAGCCCGCCCTCAATGACTCCAGATACTGCCTGAGCAGC |
| | | | CGCCTGAGGGTCTCGGCCACCTTCTGGCAGAACCCCCGCAAC |
| | | | CACTTCCGCTGTCAAGTCCAGTTCTACGGGCTCTCGGAGAATG |
| | | | ACGAGTGGACCCAGGATAGGGCCAAACCCGTCACCCAGATCG |
| | | | TCAGCGCCGAGGCCTGGGGTAGAGCAGACTGTGGCTTTACCT |
| | | | CGGTGTCCTACCAGCAAGGGGTCCTGTCTGCCACCATCCTCTA |
| | | | TGAGATCCTGCTAGGGAAGGCCACCCTGTATGCTGTGCTGGT |
| | | | CAGCGCCCTTGTGTTGATGGCCATGGTCAAGAGAAAGGATTTC |
| | | | TGA |

TABLE 6
HLA-A*03:01, Jchain, ISD peptide: TCR 5C8.16
Sequences for HLA-A*03:01, Jchain, ISD peptide: TCR 5C8.16

| SEQ | TCR | AA | |
|---|---|---|---|
| ID | POLYPE | or | |
| NO | PTIDE | NT | SEQUENCE |
| 43 | α CDR1 | AA | SSYSPS |
| 44 | α CDR2 | AA | YTSAATLV |
| 45 | α CDR3 | AA | CVVKWADSWGKLQF |
| 46 | β CDR1 | AA | PRHDT |
| 47 | β CDR2 | AA | FYEKMQ |
| 48 | β CDR3 | AA | CASSLGTGNFYEQYF |
| 49 | α VJ | AA | MLLLLVPVLEVIFTLGGTRAQSVTQLDSHVSVSEGTPVLLRCNYSS |
| | | | SYSPSLFWYVQHPNKGLQLLLKYTSAATLVKGINGFEAEFKKSET |
| | | | SFHLTKPSAHMSDAAEYFCVVKWADSWGKLQFGAGTQVVVTP |
| 50 | α VJ | NT | ATGCTCCTGCTGCTCGTCCCAGTGCTCGAGGTGATTTTTACTC |
| | | | TGGGAGGAACCAGAGCCCAGTCGGTGACCCAGCTTGACAGCC |
| | | | ACGTCTCTGTCTCTGAAGGAACCCCGGTGCTGCTGAGGTGCA |
| | | | ACTACTCATCTTCTTATTCACCATCTCTCTTCTGGTATGTGCAA |
| | | | CACCCCAACAAAGGACTCCAGCTTCTCCTGAAGTACACATCAG |
| | | | CGGCCACCCTGGTTAAAGGCATCAACGGTTTTGAGGCTGAATT |
| | | | TAAGAAGAGTGAAACCTCCTTCCACCTGACGAAACCCTCAGCC |
| | | | CATATGAGCGACGCGGCTGAGTACTTCTGTGTTGTGAAATGGG |
| | | | CTGACAGCTGGGGGAAATTGCAGTTTGGAGCAGGGACCCAGG |
| | | | TTGTGGTCACCCCA |
| 51 | β VDJ | AA | MLSPDLPDSAWNTRLLCHVMLCLLGAVSVAAGVIQSPRHLIKEKR |
| | | | ETATLKCYPIPRHDTVYWYQQGPGQDPQFLISFYEKMQSDKGSIP |
| | | | DRFSAQQFSDYHSELNMSSLELGDSALYFCASSLGTGNFYEQYF |
| | | | GPGTRLTVT |
| 52 | β VDJ | NT | ATGCTTAGTCCTGACCTGCCTGACTCTGCCTGGAACACCAGGC |
| | | | TCCTCTGCCATGTCATGCTTTGTCTCCTGGGAGCAGTTTCAGT |
| | | | GGCTGCTGGAGTCATCCAGTCCCCAAGACATCTGATCAAAGAA |
| | | | AAGAGGGAAACAGCCACTCTGAAATGCTATCCTATCCCTAGAC |
| | | | ACGACACTGTCTACTGGTACCAGCAGGGTCCAGGTCAGGACC |
| | | | CCCAGTTCCTCATTTCGTTTTATGAAAAGATGCAGAGCGATAAA |
| | | | GGAAGCATCCCTGATCGATTCTCAGCTCAACAGTTCAGTGACT |
| | | | ATCATTCTGAACTGAACATGAGCTCCTTGGAGCTGGGGGACTC |
| | | | AGCCCTGTACTTCTGTGCCAGCAGCTTAGGAACAGGGAATTTT |
| | | | TACGAGCAGTACTTCGGGCCGGGCACCAGGCTCACGGTCACA |
| 53 | α VJ and | AA | MLLLLVPVLEVIFTLGGTRAQSVTQLDSHVSVSEGTPVLLRCNYSS |
| | constant | | SYSPSLFWYVQHPNKGLQLLLKYTSAATLVKGINGFEAEFKKSET |
| | | | SFHLTKPSAHMSDAAEYFCVVKWADSWGKLQFGAGTQVVVTPDI |
| | | | QNPDPAVYQLRDSKSSDKSVCLFTDFDSQTNVSQSKDSDVYITD |
| | | | KTVLDMRSMDFKSNSAVAWSNKSDFACANAFNNSIIPEDTFFPSP |
| | | | ESSCDVKLVEKSFETDTNLNFQNLSVIGFRILLLKVAGFNLLMTL |
| | | | RLWSS |
| 54 | α VJ and | NT | ATGCTCCTGCTGCTCGTCCCAGTGCTCGAGGTGATTTTTACTC |
| | constant | | TGGGAGGAACCAGAGCCCAGTCGGTGACCCAGCTTGACAGCC |
| | | | ACGTCTCTGTCTCTGAAGGAACCCCGGTGCTGCTGAGGTGCA |
| | | | ACTACTCATCTTCTTATTCACCATCTCTCTTCTGGTATGTGCAA |
| | | | CACCCCAACAAAGGACTCCAGCTTCTCCTGAAGTACACATCAG |
| | | | CGGCCACCCTGGTTAAAGGCATCAACGGTTTTGAGGCTGAATT |
| | | | TAAGAAGAGTGAAACCTCCTTCCACCTGACGAAACCCTCAGCC |
| | | | CATATGAGCGACGCGGCTGAGTACTTCTGTGTTGTGAAATGGG |
| | | | CTGACAGCTGGGGGAAATTGCAGTTTGGAGCAGGGACCCAGG |
| | | | TTGTGGTCACCCCAGATATCCAGAACCCTGACCCTGCCGTGTA |
| | | | CCAGCTGAGAGACTCTAAATCCAGTGACAAGTCTGTCTGCCTA |
| | | | TTCACCGATTTTGATTCTCAAACAAATGTGTCACAAAGTAAGGA |
| | | | TTCTGATGTGTATATCACAGACAAAACTGTGCTAGACATGAGGT |
| | | | CTATGGACTTCAAGAGCAACAGTGCTGTGGCCTGGAGCAACAA |
| | | | ATCTGACTTTGCATGTGCAAACGCCTTCAACAACAGCATTATTC |
| | | | CAGAAGACACCTTCTTCCCCAGCCCAGAAAGTTCCTGTGATGT |
| | | | CAAGCTGGTCGAGAAAAGCTTTGAAACAGATACGAACCTAAAC |
| | | | TTTCAAAACCTGTCAGTGATTGGGTTCCGAATCCTCCTCCTGAA |
| | | | AGTGGCCGGGTTTAATCTGCTCATGACGCTGCGGTTGTGGTCC |
| | | | AGCTGA |
| 55 | β VDJ | AA | MLSPDLPDSAWNTRLLCHVMLCLLGAVSVAAGVIQSPRHLIKEKR |
| | and | | ETATLKCYPIPRHDTVYWYQQGPGQDPQFLISFYEKMQSDKGSIP |
| | constant | | DRFSAQQFSDYHSELNMSSLELGDSALYFCASSLGTGNFYEQYF |
| | | | GPGTRLTVTEDLNKVFPPEVAVFEPSEAEISHTQKATLVCLATGFF |
| | | | PDHVELSWWVNGKEVHSGVSTDPQPLKEQPALNDSRYCLSSRL |
| | | | RVSATFWQNPRNHFRCQVQFYGLSENDEWTQDRAKPVTQIVSAE |
| | | | AWGRADCGFTSVSYQQGVLSATILYEILLGKATLYAVLVSALVLM |
| | | | AMVKRKDF |
| 56 | B VDJ | NT | ATGCTTAGTCCTGACCTGCCTGACTCTGCCTGGAACACCAGGC |
| | and | | TCCTCTGCCATGTCATGCTTTGTCTCCTGGGAGCAGTTTCAGT |
| | constant | | GGCTGCTGGAGTCATCCAGTCCCCAAGACATCTGATCAAAGAA |
| | | | AAGAGGGAAACAGCCACTCTGAAATGCTATCCTATCCCTAGAC |
| | | | ACGACACTGTCTACTGGTACCAGCAGGGTCCAGGTCAGGACC |
| | | | CCCAGTTCCTCATTTCGTTTTATGAAAAGATGCAGAGCGATAAA |
| | | | GGAAGCATCCCTGATCGATTCTCAGCTCAACAGTTCAGTGACT |
| | | | ATCATTCTGAACTGAACATGAGCTCCTTGGAGCTGGGGGACTC |
| | | | AGCCCTGTACTTCTGTGCCAGCAGCTTAGGAACAGGGAATTTT |
| | | | TACGAGCAGTACTTCGGGCCGGGCACCAGGCTCACGGTCACA |
| | | | GAGGACCTGAACAAGGTGTTCCCACCCGAGGTCGCTGTGTTT |
| | | | GAGCCATCAGAAGCAGAGATCTCCCACACCCAAAAGGCCACA |
| | | | CTGGTGTGCCTGGCCACAGGCTTCTTCCCCGACCACGTGGAG |
| | | | CTGAGCTGGTGGGTGAATGGGAAGGAGGTGCACAGTGGGGTC |
| | | | AGCACAGACCCGCAGCCCCTCAAGGAGCAGCCCGCCCTCAAT |
| | | | GACTCCAGATACTGCCTGAGCAGCCGCCTGAGGGTCTCGGCC |
| | | | ACCTTCTGGCAGAACCCCCGCAACCACTTCCGCTGTCAAGTCC |
| | | | AGTTCTACGGGCTCTCGGAGAATGACGAGTGGACCCAGGATA |
| | | | GGGCCAAACCCGTCACCCAGATCGTCAGCGCCGAGGCCTGG |
| | | | GGTAGAGCAGACTGTGGCTTTACCTCGGTGTCCTACCAGCAAG |
| | | | GGGTCCTGTCTGCCACCATCCTCTATGAGATCCTGCTAGGGAA |
| | | | GGCCACCCTGTATGCTGTGCTGGTCAGCGCCCTTGTGTTGATG |
| | | | GCCATGGTCAAGAGAAAGGATTTCTGA |

TABLE 7
HLA-A*11:01, Jchain, ISD peptide: TCR 16C7.9
Sequences for HLA-A*11:01, Jchain, ISD peptide: TCR 16C7.9

| SEQ | TCR | AA | |
|---|---|---|---|
| ID | POLY- | or | |
| NO | PEPTIDE | NT | SEQUENCE |
| 57 | α CDR1 | AA | TSDPSYG |
| 58 | α CDR2 | AA | QGSYDQQN |
| 59 | α CDR3 | AA | CAMREDYTGGFKTIF |
| 60 | β CDR1 | AA | SGHTA |
| 61 | β CDR2 | AA | FQGTGA |
| 62 | β CDR3 | AA | CASSLISGGNEQFF |
| 63 | α VJ | AA | MSLSSLLKVVTASLWLGPGIAQKITQTQPGMFVQEKEAVTLDCTY |
| | | | DTSDPSYGLFWYKQPSSGEMIFLIYQGSYDQQNATEGRYSLNFQ |
| | | | KARKSANLVISASQLGDSAMYFCAMREDYTGGFKTIFGAGTRLFV |
| | | | KA |
| 64 | α VJ | NT | ATGTCACTTTCTAGCCTGCTGAAGGTGGTCACAGCTTCACTGT |
| | | | GGCTAGGACCTGGCATTGCCCAGAAGATAACTCAAACCCAACC |
| | | | AGGAATGTTCGTGCAGGAAAAGGAGGCTGTGACTCTGGACTG |
| | | | CACATATGACACCAGTGATCCAAGTTATGGTCTATTCTGGTACA |
| | | | AGCAGCCCAGCAGTGGGGAAATGATTTTTCTTATTTATCAGGG |
| | | | GTCTTATGACCAGCAAAATGCAACAGAAGGTCGCTACTCATTG |
| | | | AATTTCCAGAAGGCAAGAAAATCCGCCAACCTTGTCATCTCCG |
| | | | CTTCACAACTGGGGGACTCAGCAATGTACTTCTGTGCAATGAG |
| | | | AGAGGACTATACTGGAGGCTTCAAAACTATCTTTGGAGCAGGA |
| | | | ACAAGACTATTTGTTAAAGCA |
| 65 | β VDJ | AA | MGTRLLCWAALCLLGADHTGAGVSQTPSNKVTEKGKYVELRCDP |
| | | | ISGHTALYWYRQSLGQGPEFLIYFQGTGAADDSGLPNDRFFAVR |
| | | | PEGSVSTLKIQRTERGDSAVYLCASSLISGGNEQFFGPGTRLTVL |
| 66 | β VDJ | NT | ATGGGCACCAGGCTCCTCTGCTGGGCAGCCCTGTGCCTCCTG |
| | | | GGGGCAGATCACACAGGTGCTGGAGTCTCCCAGACCCCCAGT |
| | | | AACAAGGTCACAGAGAAGGGAAAATATGTAGAGCTCAGGTGTG |
| | | | ATCCAATTTCAGGTCATACTGCCCTTTACTGGTACCGACAAAGC |
| | | | CTGGGGCAGGGCCCAGAGTTTCTAATTTACTTCCAAGGCACGG |
| | | | GTGCGGCAGATGACTCAGGGCTGCCCAACGATCGGTTCTTTG |
| | | | CAGTCAGGCCTGAGGGATCCGTCTCTACTCTGAAGATCCAGC |
| | | | GCACAGAGCGGGGGGACTCAGCCGTGTATCTCTGTGCCAGCA |
| | | | GCTTAATATCCGGGGGCAATGAGCAGTTCTTCGGGCCAGGGA |
| | | | CACGGCTCACCGTGCTA |
| 67 | α VJ and | AA | MSLSSLLKVVTASLWLGPGIAQKITQTQPGMFVQEKEAVTLDCTY |
| | constant | | DTSDPSYGLFWYKQPSSGEMIFLIYQGSYDQQNATEGRYSLNFQ |
| | | | KARKSANLVISASQLGDSAMYFCAMREDYTGGFKTIFGAGTRLFV |
| | | | KANIQNPDPAVYQLRDSKSSDKSVCLFTDFDSQTNVSQSKDSDV |
| | | | YITDKTVLDMRSMDFKSNSAVAWSNKSDFACANAFNNSIIPEDTF |
| | | | FPSPESSCDVKLVEKSFETDTNLNFQNLSVIGFRILLLKVAGFNLL |
| | | | MTLRLWSS |
| 68 | α VJ and | NT | ATGTCACTTTCTAGCCTGCTGAAGGTGGTCACAGCTTCACTGT |
| | constant | | GGCTAGGACCTGGCATTGCCCAGAAGATAACTCAAACCCAACC |
| | | | AGGAATGTTCGTGCAGGAAAAGGAGGCTGTGACTCTGGACTG |
| | | | CACATATGACACCAGTGATCCAAGTTATGGTCTATTCTGGTACA |
| | | | AGCAGCCCAGCAGTGGGGAAATGATTTTTCTTATTTATCAGGG |
| | | | GTCTTATGACCAGCAAAATGCAACAGAAGGTCGCTACTCATTG |
| | | | AATTTCCAGAAGGCAAGAAAATCCGCCAACCTTGTCATCTCCG |
| | | | CTTCACAACTGGGGGACTCAGCAATGTACTTCTGTGCAATGAG |
| | | | AGAGGACTATACTGGAGGCTTCAAAACTATCTTTGGAGCAGGA |
| | | | ACAAGACTATTTGTTAAAGCAAATATCCAGAACCCTGACCCTGC |
| | | | CGTGTACCAGCTGAGAGACTCTAAATCCAGTGACAAGTCTGTC |
| | | | TGCCTATTCACCGATTTTGATTCTCAAACAAATGTGTCACAAAG |
| | | | TAAGGATTCTGATGTGTATATCACAGACAAAACTGTGCTAGACA |
| | | | TGAGGTCTATGGACTTCAAGAGCAACAGTGCTGTGGCCTGGA |
| | | | GCAACAAATCTGACTTTGCATGTGCAAACGCCTTCAACAACAG |
| | | | CATTATTCCAGAAGACACCTTCTTCCCCAGCCCAGAAAGTTCCT |
| | | | GTGATGTCAAGCTGGTCGAGAAAAGCTTTGAAACAGATACGAA |
| | | | CCTAAACTTTCAAAACCTGTCAGTGATTGGGTTCCGAATCCTCC |
| | | | TCCTGAAAGTGGCCGGGTTTAATCTGCTCATGACGCTGCGGTT |
| | | | GTGGTCCAGCTGA |
| 69 | β VDJ | AA | MGTRLLCWAALCLLGADHTGAGVSQTPSNKVTEKGKYVELRCDP |
| | and | | ISGHTALYWYRQSLGQGPEFLIYFQGTGAADDSGLPNDRFFAVR |
| | constant | | PEGSVSTLKIQRTERGDSAVYLCASSLISGGNEQFFGPGTRLTVL |
| | | | EDLNKVFPPEVAVFEPSEAEISHTQKATLVCLATGFFPDHVELSW |
| | | | WVNGKEVHSGVSTDPQPLKEQPALNDSRYCLSSRLRVSATFWQNP |
| | | | RNHFRCQVQFYGLSENDEWTQDRAKPVTQIVSAEAWGRADCGFTS |
| | | | VSYQQGVLSATILYEILLGKATLYAVLVSALVLMAMVKRKDF |
| 70 | β VDJ | NT | ATGGGCACCAGGCTCCTCTGCTGGGCAGCCCTGTGCCTCCTG |
| | and | | GGGGCAGATCACACAGGTGCTGGAGTCTCCCAGACCCCCAGT |
| | constant | | AACAAGGTCACAGAGAAGGGAAAATATGTAGAGCTCAGGTGTG |
| | | | ATCCAATTTCAGGTCATACTGCCCTTTACTGGTACCGACAAAGC |
| | | | CTGGGGCAGGGCCCAGAGTTTCTAATTTACTTCCAAGGCACGG |
| | | | GTGCGGCAGATGACTCAGGGCTGCCCAACGATCGGTTCTTTG |
| | | | CAGTCAGGCCTGAGGGATCCGTCTCTACTCTGAAGATCCAGC |
| | | | GCACAGAGCGGGGGGACTCAGCCGTGTATCTCTGTGCCAGCA |
| | | | GCTTAATATCCGGGGGCAATGAGCAGTTCTTCGGGCCAGGGA |
| | | | CACGGCTCACCGTGCTAGAGGACCTGAACAAGGTGTTCCCAC |
| | | | CCGAGGTCGCTGTGTTTGAGCCATCAGAAGCAGAGATCTCCCA |
| | | | CACCCAAAAGGCCACACTGGTGTGCCTGGCCACAGGCTTCTT |
| | | | CCCCGACCACGTGGAGCTGAGCTGGTGGGTGAATGGGAAGG |
| | | | AGGTGCACAGTGGGGTCAGCACAGACCCGCAGCCCCTCAAGG |
| | | | AGCAGCCCGCCCTCAATGACTCCAGATACTGCCTGAGCAGCC |
| | | | GCCTGAGGGTCTCGGCCACCTTCTGGCAGAACCCCCGCAACC |
| | | | ACTTCCGCTGTCAAGTCCAGTTCTACGGGCTCTCGGAGAATGA |
| | | | CGAGTGGACCCAGGATAGGGCCAAACCCGTCACCCAGATCGT |
| | | | CAGCGCCGAGGCCTGGGGTAGAGCAGACTGTGGCTTTACCTC |
| | | | GGTGTCCTACCAGCAAGGGGTCCTGTCTGCCACCATCCTCTAT |
| | | | GAGATCCTGCTAGGGAAGGCCACCCTGTATGCTGTGCTGGTC |
| | | | AGCGCCCTTGTGTTGATGGCCATGGTCAAGAGAAAGGATTTCT |
| | | | GA |

TABLE 8
HLA-A*11:01, Jchain, ISD peptide: TCR 13D4.9
Sequences for HLA-A*11:01, Jchain, ISD peptide: TCR 13D4.9

| SEQ | TCR | AA | |
|---|---|---|---|
| ID | POLY- | or | |
| NO | PEPTIDE | NT | SEQUENCE |
| 71 | α CDR1 | AA | DSASNY |
| 72 | α CDR2 | AA | IRSNVGE |
| 73 | α CDR3 | AA | CAASNSGGYNKLIF |
| 74 | β CDR1 | AA | KGHSY |
| 75 | β CDR2 | AA | FQNENV |
| 76 | β CDR3 | AA | CASSQGRGPNGEKLFF |
| 77 | α VJ | AA | MTSIRAVFIFLWLQLDLVNGENVEQHPSTLSVQEGDSAVIKCTYSD |
| | | | SASNYFPWYKQELGKRPQLIIDIRSNVGEKKDQRIAVTLNKTAKHF |
| | | | SLHITETQPEDSAVYFCAASNSGGYNKLIFGAGTRLAVHP |
| 78 | α VJ | NT | ATGACATCCATTCGAGCTGTATTTATATTCCTGTGGCTGCAGCT |
| | | | GGACTTGGTGAATGGAGAGAATGTGGAGCAGCATCCTTCAACC |
| | | | CTGAGTGTCCAGGAGGGAGACAGCGCTGTTATCAAGTGTACTT |
| | | | ATTCAGACAGTGCCTCAAACTACTTCCCTTGGTATAAGCAAGAA |
| | | | CTTGGAAAAAGACCTCAGCTTATTATAGACATTCGTTCAAATGT |
| | | | GGGCGAAAAGAAAGACCAACGAATTGCTGTTACATTGAACAAG |
| | | | ACAGCCAAACATTTCTCCCTGCACATCACAGAGACCCAACCTG |
| | | | AAGACTCGGCTGTCTACTTCTGTGCAGCAAGTAACTCTGGTGG |
| | | | CTACAATAAGCTGATTTTTGGAGCAGGGACCAGGCTGGCTGTA |
| | | | CACCCA |
| 79 | β VDJ | AA | MSPIFTCITILCLLAAGSPGEEVAQTPKHLVRGEGQKAKLYCAP |
| | | | IKGHSYVFWYQQVLKNEFKFLISFQNENVFDETGMPKERFSAKC |
| | | | LPNSPCSLEIQATKLEDSAVYFCASSQGRGPNGEKLFFGSGTQL |
| | | | SVL |
| 80 | β VDJ | NT | ATGAGCCCAATATTCACCTGCATCACAATCCTTTGTCTGCTGGC |
| | | | TGCAGGTTCTCCTGGTGAAGAAGTCGCCCAGACTCCAAAACAT |
| | | | CTTGTCAGAGGGGAAGGACAGAAAGCAAAATTATATTGTGCCC |
| | | | CAATAAAAGGACACAGTTATGTTTTTTGGTACCAACAGGTCCTG |
| | | | AAAAACGAGTTCAAGTTCTTGATTTCCTTCCAGAATGAAAATGT |
| | | | CTTTGATGAAACAGGTATGCCCAAGGAAAGATTTTCAGCTAAGT |
| | | | GCCTCCCAAATTCACCCTGTAGCCTTGAGATCCAGGCTACGAA |
| | | | GCTTGAGGATTCAGCAGTGTATTTTTGTGCCAGCAGCCAAGGG |
| | | | AGAGGCCCAAACGGAGAAAAACTGTTTTTTGGCAGTGGAACCC |
| | | | AGCTCTCTGTCTTG |
| 81 | α VJ and | AA | MTSIRAVFIFLWLQLDLVNGENVEQHPSTLSVQEGDSAVIKCTYSD |
| | constant | | SASNYFPWYKQELGKRPQLIIDIRSNVGEKKDQRIAVTLNKTAKHF |
| | | | SLHITETQPEDSAVYFCAASNSGGYNKLIFGAGTRLAVHPYIQNPD |
| | | | PAVYQLRDSKSSDKSVCLFTDFDSQTNVSQSKDSDVYITDKTVLD |
| | | | MRSMDFKSNSAVAWSNKSDFACANAFNNSIIPEDTFFPSPESSCD |
| | | | VKLVEKSFETDTNLNFQNLSVIGFRILLLKVAGFNLLMTLRLWSS |
| 82 | α VJ and | NT | ATGACATCCATTCGAGCTGTATTTATATTCCTGTGGCTGCAGCT |
| | constant | | GGACTTGGTGAATGGAGAGAATGTGGAGCAGCATCCTTCAACC |
| | | | CTGAGTGTCCAGGAGGGAGACAGCGCTGTTATCAAGTGTACTT |
| | | | ATTCAGACAGTGCCTCAAACTACTTCCCTTGGTATAAGCAAGAA |
| | | | CTTGGAAAAAGACCTCAGCTTATTATAGACATTCGTTCAAATGT |
| | | | GGGCGAAAAGAAAGACCAACGAATTGCTGTTACATTGAACAAG |
| | | | ACAGCCAAACATTTCTCCCTGCACATCACAGAGACCCAACCTG |
| | | | AAGACTCGGCTGTCTACTTCTGTGCAGCAAGTAACTCTGGTGG |
| | | | CTACAATAAGCTGATTTTTGGAGCAGGGACCAGGCTGGCTGTA |
| | | | CACCCATATATCCAGAACCCTGACCCTGCCGTGTACCAGCTGA |
| | | | GAGACTCTAAATCCAGTGACAAGTCTGTCTGCCTATTCACCGA |
| | | | TTTTGATTCTCAAACAAATGTGTCACAAAGTAAGGATTCTGATG |
| | | | TGTATATCACAGACAAAACTGTGCTAGACATGAGGTCTATGGA |
| | | | CTTCAAGAGCAACAGTGCTGTGGCCTGGAGCAACAAATCTGAC |
| | | | TTTGCATGTGCAAACGCCTTCAACAACAGCATTATTCCAGAAGA |
| | | | CACCTTCTTCCCCAGCCCAGAAAGTTCCTGTGATGTCAAGCTG |
| | | | GTCGAGAAAAGCTTTGAAACAGATACGAACCTAAACTTTCAAAA |
| | | | CCTGTCAGTGATTGGGTTCCGAATCCTCCTCCTGAAAGTGGCC |
| | | | GGGTTTAATCTGCTCATGACGCTGCGGTTGTGGTCCAGCTGA |
| 83 | β VDJ | AA | MSPIFTCITILCLLAAGSPGEEVAQTPKHLVRGEGQKAKLYCAPI |
| | and | | KGHSYVFWYQQVLKNEFKFLISFQNENVFDETGMPKERFSAKCLP |
| | constant | | NSPCSLEIQATKLEDSAVYFCASSQGRGPNGEKLFFGSGTQLSVL |
| | | | EDLNKVFPPEVAVFEPSEAEISHTQKATLVCLATGFFPDHVELSW |
| | | | WVNGKEVHSGVSTDPQPLKEQPALNDSRYCLSSRLRVSATFWQNP |
| | | | RNHFRCQVQFYGLSENDEWTQDRAKPVTQIVSAEAWGRADCGFT |
| | | | SVSYQQGVLSATILYEILLGKATLYAVLVSALVLMAMVKRKDF |
| 84 | β VDJ | NT | ATGAGCCCAATATTCACCTGCATCACAATCCTTTGTCTGCTGGC |
| | and | | TGCAGGTTCTCCTGGTGAAGAAGTCGCCCAGACTCCAAAACAT |
| | constant | | CTTGTCAGAGGGGAAGGACAGAAAGCAAAATTATATTGTGCCC |
| | | | CAATAAAAGGACACAGTTATGTTTTTTGGTACCAACAGGTCCTG |
| | | | AAAAACGAGTTCAAGTTCTTGATTTCCTTCCAGAATGAAAATGT |
| | | | CTTTGATGAAACAGGTATGCCCAAGGAAAGATTTTCAGCTAAGT |
| | | | GCCTCCCAAATTCACCCTGTAGCCTTGAGATCCAGGCTACGAA |
| | | | GCTTGAGGATTCAGCAGTGTATTTTTGTGCCAGCAGCCAAGGG |
| | | | AGAGGCCCAAACGGAGAAAAACTGTTTTTTGGCAGTGGAACCC |
| | | | AGCTCTCTGTCTTGGAGGACCTGAACAAGGTGTTCCCACCCGA |
| | | | GGTCGCTGTGTTTGAGCCATCAGAAGCAGAGATCTCCCACACC |
| | | | CAAAAGGCCACACTGGTGTGCCTGGCCACAGGCTTCTTCCCC |
| | | | GACCACGTGGAGCTGAGCTGGTGGGTGAATGGGAAGGAGGT |
| | | | GCACAGTGGGGTCAGCACAGACCCGCAGCCCCTCAAGGAGCA |
| | | | GCCCGCCCTCAATGACTCCAGATACTGCCTGAGCAGCCGCCT |
| | | | GAGGGTCTCGGCCACCTTCTGGCAGAACCCCCGCAACCACTT |
| | | | CCGCTGTCAAGTCCAGTTCTACGGGCTCTCGGAGAATGACGA |
| | | | GTGGACCCAGGATAGGGCCAAACCCGTCACCCAGATCGTCAG |
| | | | CGCCGAGGCCTGGGGTAGAGCAGACTGTGGCTTTACCTCGGT |
| | | | GTCCTACCAGCAAGGGGTCCTGTCTGCCACCATCCTCTATGAG |
| | | | ATCCTGCTAGGGAAGGCCACCCTGTATGCTGTGCTGGTCAGC |
| | | | GCCCTTGTGTTGATGGCCATGGTCAAGAGAAAGGATTTCTGA |

TABLE 9
HLA-A*01:01, Jchain, YTA: TCR 13F6.6
Sequences for HLA-A*01:01, Jchain, YTA: TCR 13F6.6

| SEQ | TCR | AA | |
|---|---|---|---|
| ID | POLYPE | or | |
| NO | PTIDE | NT | SEQUENCE |
| 85 | α CDR1 | AA | YGGTVN |
| 86 | α CDR2 | AA | YFSGDPLV |
| 87 | α CDR3 | AA | CAASSGGYQKVTF |
| 88 | β CDR1 | AA | MDHEN |
| 89 | β CDR2 | AA | SYDVKM |
| 90 | β CDR3 | AA | CASSLRREVGDTQYF |
| 91 | α VJ | AA | MLLLLIPVLGMIFALRDARAQSVSQHNHHVILSEAASLELGCNYS |
| | | | YGGTVNLFWYVQYPGQHLQLLLKYFSGDPLVKGIKGFEAEFIKS |
| | | | KFSFNLRKPSVQWSDTAEYFCAASSGGYQKVTFGIGTKLQVIP |
| 92 | α VJ | NT | ATGCTCCTGTTGCTCATACCAGTGCTGGGGATGATTTTTGCC |
| | | | CTGAGAGATGCCAGAGCCCAGTCTGTGAGCCAGCATAACCA |
| | | | CCACGTAATTCTCTCTGAAGCAGCCTCACTGGAGTTGGGAT |
| | | | GCAACTATTCCTATGGTGGAACTGTTAATCTCTTCTGGTATG |
| | | | TCCAGTACCCTGGTCAACACCTTCAGCTTCTCCTCAAGTACT |
| | | | TTTCAGGGGATCCACTGGTTAAAGGCATCAAGGGCTTTGAG |
| | | | GCTGAATTTATAAAGAGTAAATTCTCCTTTAATCTGAGGAAAC |
| | | | CCTCTGTGCAGTGGAGTGACACAGCTGAGTACTTCTGTGCC |
| | | | GCCTCTTCTGGGGGTTACCAGAAAGTTACCTTTGGAATTGGA |
| | | | ACAAAGCTCCAAGTCATCCCA |
| 93 | β VDJ | AA | MGIRLLCRVAFCFLAVGLVDVKVTQSSRYLVKRTGEKVFLECV |
| | | | QDMDHENMFWYRQDPGLGLRLIYFSYDVKMKEKGDIPEGYSV |
| | | | SREKKERFSLILESASTNQTSMYLCASSLRREVGDTQYFGPGT |
| | | | RLTVL |
| 94 | β VDJ | NT | ATGGGAATCAGGCTCCTGTGTCGTGTGGCCTTTTGTTTCCTG |
| | | | GCTGTAGGCCTCGTAGATGTGAAAGTAACCCAGAGCTCGAG |
| | | | ATATCTAGTCAAAAGGACGGGAGAGAAAGTTTTTCTGGAATG |
| | | | TGTCCAGGATATGGACCATGAAAATATGTTCTGGTATCGACA |
| | | | AGACCCAGGTCTGGGGCTACGGCTGATCTATTTCTCATATG |
| | | | ATGTTAAAATGAAAGAAAAAGGAGATATTCCTGAGGGGTACA |
| | | | GTGTCTCTAGAGAGAAGAAGGAGCGCTTCTCCCTGATTCTG |
| | | | GAGTCCGCCAGCACCAACCAGACATCTATGTACCTCTGTGC |
| | | | CAGCAGTTTAAGGCGGGAGGTGGGAGATACGCAGTATTTTG |
| | | | GCCCAGGCACCCGGCTGACAGTGCTC |
| 95 | α VJ and | AA | MLLLLIPVLGMIFALRDARAQSVSQHNHHVILSEAASLELGCN |
| | constant | | YSYGGTVNLFWYVQYPGQHLQLLLKYFSGDPLVKGIKGFEAEF |
| | | | IKSKFSFNLRKPSVQWSDTAEYFCAASSGGYQKVTFGIGTKLQ |
| | | | VIPNIQNPDPAVYQLRDSKSSDKSVCLFTDFDSQTNVSQSKDS |
| | | | DVYITDKTVLDMRSMDFKSNSAVAWSNKSDFACANAFNNSIIP |
| | | | EDTFFPSPESSCDVKLVEKSFETDTNLNFQNLSVIGFRILLLK |
| | | | VAGFNLLMTLRLWSS |
| 96 | α VJ and | NT | ATGCTCCTGTTGCTCATACCAGTGCTGGGGATGATTTTTGCC |
| | constant | | CTGAGAGATGCCAGAGCCCAGTCTGTGAGCCAGCATAACCA |
| | | | CCACGTAATTCTCTCTGAAGCAGCCTCACTGGAGTTGGGAT |
| | | | GCAACTATTCCTATGGTGGAACTGTTAATCTCTTCTGGTATG |
| | | | TCCAGTACCCTGGTCAACACCTTCAGCTTCTCCTCAAGTACT |
| | | | TTTCAGGGGATCCACTGGTTAAAGGCATCAAGGGCTTTGAGG |
| | | | CTGAATTTATAAAGAGTAAATTCTCCTTTAATCTGAGGAAAC |
| | | | CCTCTGTGCAGTGGAGTGACACAGCTGAGTACTTCTGTGCC |
| | | | GCCTCTTCTGGGGGTTACCAGAAAGTTACCTTTGGAATTGGA |
| | | | ACAAAGCTCCAAGTCATCCCAAATATCCAGAACCCTGACCCT |
| | | | GCCGTGTACCAGCTGAGAGACTCTAAATCCAGTGACAAGTCT |
| | | | GTCTGCCTATTCACCGATTTTGATTCTCAAACAAATGTGTCA |
| | | | CAAAGTAAGGATTCTGATGTGTATATCACAGACAAAACTGTG |
| | | | CTAGACATGAGGTCTATGGACTTCAAGAGCAACAGTGCTGT |
| | | | GGCCTGGAGCAACAAATCTGACTTTGCATGTGCAAACGCCT |
| | | | TCAACAACAGCATTATTCCAGAAGACACCTTCTTCCCCAGCC |
| | | | CAGAAAGTTCCTGTGATGTCAAGCTGGTCGAGAAAAGCTTT |
| | | | GAAACAGATACGAACCTAAACTTTCAAAACCTGTCAGTGATT |
| | | | GGGTTCCGAATCCTCCTCCTGAAAGTGGCCGGGTTTAATCT |
| | | | GCTCATGACGCTGCGGTTGTGGTCCAGCTGA |
| 97 | β VDJ | AA | MGIRLLCRVAFCFLAVGLVDVKVTQSSRYLVKRTGEKVFLECV |
| | and | | QDMDHENMFWYRQDPGLGLRLIYFSYDVKMKEKGDIPEGYSV |
| | constant | | SREKKERFSLILESASTNQTSMYLCASSLRREVGDTQYFGPGT |
| | | | RLTVLEDLNKVFPPEVAVFEPSEAEISHTQKATLVCLATGFFPD |
| | | | HVELSWWVNGKEVHSGVSTDPQPLKEQPALNDSRYCLSSRLR |
| | | | VSATFWQNPRNHFRCQVQFYGLSENDEWTQDRAKPVTQIVSA |
| | | | EAWGRADCGFTSVSYQQGVLSATILYEILLGKATLYAVLVSALV |
| | | | LMAMVKRKDF |
| 98 | β VDJ | NT | ATGGGAATCAGGCTCCTGTGTCGTGTGGCCTTTTGTTTCCTG |
| | and | | GCTGTAGGCCTCGTAGATGTGAAAGTAACCCAGAGCTCGAG |
| | constant | | ATATCTAGTCAAAAGGACGGGAGAGAAAGTTTTTCTGGAATG |
| | | | TGTCCAGGATATGGACCATGAAAATATGTTCTGGTATCGACA |
| | | | AGACCCAGGTCTGGGGCTACGGCTGATCTATTTCTCATATG |
| | | | ATGTTAAAATGAAAGAAAAAGGAGATATTCCTGAGGGGTACA |
| | | | GTGTCTCTAGAGAGAAGAAGGAGCGCTTCTCCCTGATTCTG |
| | | | GAGTCCGCCAGCACCAACCAGACATCTATGTACCTCTGTGC |
| | | | CAGCAGTTTAAGGCGGGAGGTGGGAGATACGCAGTATTTTG |
| | | | GCCCAGGCACCCGGCTGACAGTGCTCGAGGACCTGAACAA |
| | | | GGTGTTCCCACCCGAGGTCGCTGTGTTTGAGCCATCAGAAG |
| | | | CAGAGATCTCCCACACCCAAAAGGCCACACTGGTGTGCCTG |
| | | | GCCACAGGCTTCTTCCCCGACCACGTGGAGCTGAGCTGGT |
| | | | GGGTGAATGGGAAGGAGGTGCACAGTGGGGTCAGCACAGA |
| | | | CCCGCAGCCCCTCAAGGAGCAGCCCGCCCTCAATGACTCC |
| | | | AGATACTGCCTGAGCAGCCGCCTGAGGGTCTCGGCCACCTT |
| | | | CTGGCAGAACCCCCGCAACCACTTCCGCTGTCAAGTCCAGT |
| | | | TCTACGGGCTCTCGGAGAATGACGAGTGGACCCAGGATAG |
| | | | GGCCAAACCCGTCACCCAGATCGTCAGCGCCGAGGCCTGG |
| | | | GGTAGAGCAGACTGTGGCTTTACCTCGGTGTCCTACCAGCA |
| | | | AGGGGTCCTGTCTGCCACCATCCTCTATGAGATCCTGCTAG |
| | | | GGAAGGCCACCCTGTATGCTGTGCTGGTCAGCGCCCTTGTG |
| | | | TTGATGGCCATGGTCAAGAGAAAGGATTTCTGA |

SEQ ID NO: 99 (YTA peptide):
YTAVVPLVY
SEQ ID NO: 100 (CYT peptide):
CYTAVVPLV
SEQ ID NO: 101 (ISD peptide):
ISDPTSPLRTR
SEQ ID NO: 102 (TAV peptide):
TAVVPLVY
SEQ ID NO: 103 (VLA1 peptide):
VLAVFIKAVHV
SEQ ID NO: 104 (VLA2 peptide):
VLAVFIKAV
SEQ ID NO: 105:
YTAVVPLV
SEQ ID NO: 106 (RII peptide):
RIIVPLNNR
SEQ ID NO: 107 (GET peptide):
GETKMVETAL

### REFERENCES

1. Nandakumar B, Binder M, Dispenzieri A, Kapoor P, Buadi F, Gertz M A, et al. Continued improvement in survival in multiple myeloma (MM) including high-risk patients. Journal of Clinical Oncology. 2019; 37(15_suppl):8039-.
2. Raje N, Berdeja J, Lin Y, Siegel D, Jagannath S, Madduri D, et al. Anti-BCMA CAR T-Cell Therapy bb2121 in Relapsed or Refractory Multiple Myeloma. The New England journal of medicine. 2019; 380(18):1726-37.
3. Zhao W H, Liu J, Wang B Y, Chen Y X, Cao X M, Yang Y, et al. A phase 1, open-label study of LCAR-B38M, a chimeric antigen receptor T cell therapy directed against B cell maturation antigen, in patients with relapsed or refractory multiple myeloma. Journal of hematology & oncology. 2018; 11(1):141.
4. Berdeja J G, Madduri D, Usmani S Z, Jakubowiak A, Agha M, Cohen A D, et al. Ciltacabtagene autoleucel, a B-cell maturation antigen-directed chimeric antigen receptor T-cell therapy in patients with relapsed or refractory multiple myeloma (CARTITUDE-1): a phase 1b/2 open-label study. Lancet (London, England). 2021; 398(10297):314-24.
5. Cohen A D, Garfall A L, Stadtmauer E A, Melenhorst J J, Lacey S F, Lancaster E, et al. B cell maturation antigen-specific CAR T cells are clinically active in multiple myeloma. The Journal of clinical investigation. 2019; 129(6):2210-21.
6. Samur M K, Fulciniti M, Aktas Samur A, Bazarbachi A H, Tai Y T, Prabhala R, et al. Biallelic loss of BCMA as a resistance mechanism to CAR T cell therapy in a patient with multiple myeloma. Nat Commun. 2021; 12(1):868.
7. Mikkilineni L, Kochenderfer J N. CAR T cell therapies for patients with multiple myeloma. Nat Rev Clin Oncol. 2021; 18(2):71-84.
8. Bouchon A, Cella M, Grierson H L, Cohen J I, Colonna M. Activation of NK cell-mediated cytotoxicity by a SAP-independent receptor of the CD2 family. Journal of immunology (Baltimore, Md: 1950). 2001; 167(10):5517-21.
9. Smith E L, Harrington K, Staehr M, Masakayan R, Jones J, Long T J, et al. GPRC5D is a target for the immunotherapy of multiple myeloma with rationally designed CAR T cells. Science translational medicine. 2019; 11(485):eaau7746.
10. Jahn L, Hombrink P, Hagedoorn R S, Kester M G, van der Steen D M, Rodriguez T, et al. TCR-based therapy for multiple myeloma and other B-cell malignancies targeting intracellular transcription factor BOB1. Blood. 2017; 129(10):1284-95.
11. Meeuwsen M H, Wouters A K, Jahn L, Hagedoorn R S, Kester M G D, Remst D F G, et al. A broad and systematic approach to identify B-cell malignancy targeting TCRs for multi-antigen based T-cell therapy. Molecular therapy: the journal of the American Society of Gene Therapy. 2021.
12. Rapoport A P, Stadtmauer E A, Binder-Scholl G K, Goloubeva O, Vogl D T, Lacey S F, et al. NY-ESO-1-specific TCR-engineered T cells mediate sustained antigen-specific antitumor effects in myeloma. Nature medicine. 2015; 21(8):914-21.
13. Stadtmauer E A, Faitg T H, Lowther D E, Badros A Z, Chagin K, Dengel K, et al. Long-term safety and activity of NY-ESO-1 SPEAR T cells after autologous stem cell transplant for myeloma. Blood Adv. 2019; 3(13):2022-34.
14. Xu A, Barbosa R, Calado D. <em>Jchain</em>-driven cre enables specific genetic manipulation and timestamping of plasma cell in their niche. bioRxiv. 2020:2020.04.12.038380.
15. Nilssen D E, Brandtzaeg P, Frøland S S, Fausa O. Subclass composition and J-chain expression of the ‘compensatory’ gastrointestinal IgG cell population in selective IgA deficiency. Clin Exp Immunol. 1992; 87(2):237-45.
16. Brandtzaeg P. Mucosal immunity: induction, dissemination, and effector functions. Scand J Immunol. 2009; 70(6):505-15.
17. Lokhorst H M, Dekker A W, Baarlen J V, Bast E J. J chain disease: an aggressive evolution of multiple myeloma. Am J Med. 1990; 88(4):417-20.
18. Kelenyi G. Intracellular J chains in lymphoproliferative diseases. Virchows Archiv A. 1985; 405(3):365-78.
19. Pont M J, Honders M W, Kremer A N, van Kooten C, Out C, Hiemstra P S, et al. Microarray Gene Expression Analysis to Evaluate Cell Type Specific Expression of Targets Relevant for Immunotherapy of Hematological Malignancies. PloS one. 2016; 11(5):e0155165.

1. An isolated nucleic acid composition that encodes a Jchain antigen-specific binding protein having a TCR α chain variable (Vα) domain and a TCR β chain variable (Vβ) domain, the composition comprising:
(a) a nucleic acid sequence that encodes a TCR Vα domain comprising a CDR3 amino acid sequence; and
(b) a nucleic acid sequence that encodes a TCR Vβ domain comprising a CDR3 amino acid sequence;
wherein the CDR3 amino acid sequences of (a) and (b) together specifically bind to Jchain.

2. The nucleic acid composition of claim 1, wherein the Jchain antigen comprises an amino acid sequence selected from the group consisting of: YTAVVPLVY (SEQ ID NO:99), CYTAVVPLV (SEQ ID NO:100), ISDPTSPLRTR (SEQ ID NO:101), TAVVPLVY (SEQ ID NO:102), VLAVFIKAVHV (SEQ ID NO:103), VLAVFIKAV (SEQ ID NO:104), YTAVVPLV (SEQ ID NO:105), RIIVPLNNR (SEQ ID NO:106), and GETKMVETAL (SEQ ID NO:107).

3. The nucleic acid composition of claim 2, wherein the Jchain antigen comprises an amino acid sequence selected from the group consisting of: YTAVVPLVY (SEQ ID NO:99), CYTAVVPLV (SEQ ID NO:100), and ISDPTSPLRTR (SEQ ID NO:101).

4. The nucleic acid composition of any one of the preceding claims, wherein the encoded binding protein is capable of specifically binding to a peptide:HLA complex selected from the group consisting of: a YTAVVPLVY:HLA-A*01:01 complex; a CYTAVVPLV:HLA-A*24:02 complex; a ISDPTSPLRTR:HLA-A*03:01 complex; a ISDPTSPLRTR:HLA-A*11:01 complex; a TAVVPLVY:HLA-A*01:01 complex; a VLAVFIKAVHV:HLA-A*02:01 complex; a VLAVFIKAV:HLA-A*02:01 complex; a YTAVVPLV:HLA-A*02:01 complex; a RIIVPLNNR:HLA-A*11:01 complex; and a GETKMVETAL:HLA-B*40:01 complex.

5. The nucleic acid composition of claim 4, wherein the peptide:HLA complex is selected from the group consisting of: a YTAVVPLVY:HLA-A*01:01 complex; a CYTAVVPLV:HLA-A*24:02 complex; a ISDPTSPLRTR:HLA-A*03:01 complex; and a ISDPTSPLRTR:HLA-A*11:01 complex.

6. The nucleic acid composition of any one of the preceding claims wherein the composition comprises:
(i) a nucleic acid sequence that encodes a TCR Vα domain comprising a CDR3 amino acid sequence having at least 80% sequence identity to SEQ ID NO:17, or a functional fragment thereof; and a nucleic acid sequence that encodes a TCR Vβ domain comprising a CDR3 amino acid sequence having at least 80% sequence identity to SEQ ID NO: 20, or a functional fragment thereof; or
(ii) a nucleic acid sequence that encodes a TCR Vα domain comprising a CDR3 amino acid sequence having at least 80% sequence identity to SEQ ID NO:87, or a functional fragment thereof; and a nucleic acid sequence that encodes a TCR Vβ domain comprising a CDR3 amino acid sequence having at least 80% sequence identity to SEQ ID NO: 90, or a functional fragment thereof; or
(iii) a nucleic acid sequence that encodes a TCR Vα domain comprising a CDR3 amino acid sequence having at least 80% sequence identity to SEQ ID NO:3, or a functional fragment thereof; and a nucleic acid sequence that encodes a TCR Vβ domain comprising a CDR3 amino acid sequence having at least 80% sequence identity to SEQ ID NO:6, or a functional fragment thereof.

7. The nucleic acid composition of any preceding claim, wherein:
(i) the CDR3 of the Vα domain comprises or consists of the amino acid sequence of SEQ ID NO: 17, and the CDR3 of the Vβ domain comprises or consists of the amino acid sequence of SEQ ID NO:20; or
(ii) the CDR3 of the Vα domain comprises or consists of the amino acid sequence of SEQ ID NO: 87, and the CDR3 of the Vβ domain comprises or consists of the amino acid sequence of SEQ ID NO:90; or
(iii) the CDR3 of the Vα domain comprises or consists of the amino acid sequence of SEQ ID NO: 3, and the CDR3 of the Vβ domain comprises or consists of the amino acid sequence of SEQ ID NO:6.

8. The nucleic acid composition of any preceding claim, wherein:
(i) the Vα domain comprises an amino acid sequence having at least 80% sequence identity to, comprising, or consisting of, SEQ ID NO: 21; and (ii) the Vβ domain comprises an amino acid sequence having at least 80% sequence identity to, comprising, or consisting of, SEQ ID NO: 23; or
(ii) the Vα domain comprises an amino acid sequence having at least 80% sequence identity to, comprising, or consisting of, SEQ ID NO: 91; and (ii) the Vβ domain comprises an amino acid sequence having at least 80% sequence identity to, comprising, or consisting of, SEQ ID NO: 93; or
(iii) the Vα domain comprises an amino acid sequence having at least 80% sequence identity to, comprising, or consisting of, SEQ ID NO: 7; and (ii) the VB domain comprises an amino acid sequence having at least 80% sequence identity to, comprising, or consisting of, SEQ ID NO: 9.

9. The nucleic acid composition of any one of the preceding claims wherein the composition comprises a nucleic acid sequence that encodes a TCR Vα domain comprising a CDR3 amino acid sequence having at least 80% sequence identity to SEQ ID NO:31, or a functional fragment thereof; and a nucleic acid sequence that encodes a TCR Vβ domain comprising a CDR3 amino acid sequence having at least 80% sequence identity to SEQ ID NO:34, or a functional fragment thereof.

10. The nucleic acid composition of any preceding claim, wherein the CDR3 of the Vα domain comprises or consists of the amino acid sequence of SEQ ID NO: 31, and the CDR3 of the Vβ domain comprises or consists of the amino acid sequence of SEQ ID NO:34.

11. The nucleic acid composition of any preceding claim, wherein the Vα domain comprises an amino acid sequence having at least 80% sequence identity to, comprising, or consisting of, SEQ ID NO: 35; and (ii) the Vβ domain comprises an amino acid sequence having at least 80% sequence identity to, comprising, or consisting of, SEQ ID NO: 37.

12. The nucleic acid composition of any one of the preceding claims wherein the composition comprises:
(i) a nucleic acid sequence that encodes a TCR Vα domain comprising a CDR3 amino acid sequence having at least 80% sequence identity to SEQ ID NO:45, or a functional fragment thereof; and a nucleic acid sequence that encodes a TCR Vβ domain comprising a CDR3 amino acid sequence having at least 80% sequence identity to SEQ ID NO:48, or a functional fragment thereof; or
(ii) a nucleic acid sequence that encodes a TCR Vα domain comprising a CDR3 amino acid sequence having at least 80% sequence identity to SEQ ID NO:59, or a functional fragment thereof; and a nucleic acid sequence that encodes a TCR Vβ domain comprising a CDR3 amino acid sequence having at least 80% sequence identity to SEQ ID NO: 62, or a functional fragment thereof; or
(iii) a nucleic acid sequence that encodes a TCR Vα domain comprising a CDR3 amino acid sequence having at least 80% sequence identity to SEQ ID NO:73, or a functional fragment thereof; and a nucleic acid sequence that encodes a TCR Vβ domain comprising a CDR3 amino acid sequence having at least 80% sequence identity to SEQ ID NO: 76, or a functional fragment thereof.

13. The nucleic acid composition of any preceding claim, wherein:
(i) the CDR3 of the Vα domain comprises or consists of the amino acid sequence of SEQ ID NO: 45, and the CDR3 of the Vβ domain comprises or consists of the amino acid sequence of SEQ ID NO:48; or
(ii) the CDR3 of the Vα domain comprises or consists of the amino acid sequence of SEQ ID NO: 59, and the CDR3 of the Vβ domain comprises or consists of the amino acid sequence of SEQ ID NO:62; or
(iii) the CDR3 of the Vα domain comprises or consists of the amino acid sequence of SEQ ID NO: 73, and the CDR3 of the Vβ domain comprises or consists of the amino acid sequence of SEQ ID NO:76.

14. The nucleic acid composition of any preceding claim, wherein:
(i) the Vα domain comprises an amino acid sequence having at least 80% sequence identity to, comprising, or consisting of, SEQ ID NO: 49; and (ii) the Vβ domain comprises an amino acid sequence having at least 80% sequence identity to, comprising, or consisting of, SEQ ID NO: 51; or
(ii) the Vα domain comprises an amino acid sequence having at least 80% sequence identity to, comprising, or consisting of, SEQ ID NO: 63; and (ii) the Vβ domain comprises an amino acid sequence having at least 80% sequence identity to, comprising, or consisting of, SEQ ID NO: 65; or
(iii) the Vα domain comprises an amino acid sequence having at least 80% sequence identity to, comprising, or consisting of, SEQ ID NO: 77; and (ii) the Vβ domain comprises an amino acid sequence having at least 80% sequence identity to, comprising, or consisting of, SEQ ID NO: 79.

15. The nucleic acid composition of any preceding claim, wherein the nucleic acid sequence is codon optimised for expression in a host cell, optionally wherein the host cell is a human cell.

16. The nucleic acid composition of any preceding claim, further comprising a TCR α chain constant domain and/or a TCR β chain constant domain.

17. The nucleic acid composition of any preceding claim, wherein the encoded binding protein comprises a TCR, an antigen binding fragment of a TCR, a chimeric antigen receptor (CAR), or an ImmTAC.

18. The nucleic acid composition of claim 17, wherein the antigen binding fragment of a TCR is a single chain TCR (scTCR) or a chimeric TCR dimer in which the antigen binding fragment of the TCR is linked to an alternative transmembrane and intracellular signalling domain.

19. A vector system comprising a nucleic acid composition according to any one of claims 1 to 18.

20. The vector system of claim 19, wherein the vector is a plasmid, a viral vector, or a cosmid, optionally wherein the vector is selected from the group consisting of a retrovirus, lentivirus, adeno-associated virus, adenovirus, vaccinia virus, canary poxvirus, herpes virus, minicircle vector and synthetic DNA or RNA.

21. A modified cell comprising a nucleic acid composition according to any of claims 1 to 18, or a vector system according to claim 19 or 20.

22. The modified cell of claim 21, wherein the modified cell is selected from the group consisting of a CD8 T cell, a CD4 T cell, an NK cell, an NK-T cell, a gamma-delta T cell, a hematopoietic stem cell, an inducible pluripotent stem cell, a progenitor cell, a T cell line and a NK-92 cell line.

23. The modified cell of claim 21 or 22, wherein the modified cell is a human cell.

24. An isolated peptide comprising or consisting of an amino acid sequence selected from the group consisting of: YTAVVPLVY (SEQ ID NO:99), CYTAVVPLV (SEQ ID NO:100), ISDPTSPLRTR (SEQ ID NO:101), TAVVPLVY (SEQ ID NO:102), VLAVFIKAVHV (SEQ ID NO:103), VLAVFIKAV (SEQ ID NO:104), YTAVVPLV (SEQ ID NO:105), RIIVPLNNR (SEQ ID NO:106), and GETKMVETAL (SEQ ID NO:107).

25. The peptide of claim 24, wherein the peptide has no more than 20 amino acids.

26. An isolated nucleic acid sequence encoding the peptide of any one of claims 24 to 25.

27. A vector system comprising the nucleic acid sequence of claim 26.

28. A pharmaceutical composition comprising a nucleic acid composition according to any of claims 1 to 18, a vector system according to claim 19, 20 or 27, a modified cell according to any of claims 21 to 23, an isolated peptide of any of claim 24 or 25, or a nucleic acid sequence according to claim 26, and a pharmaceutically acceptable excipient, adjuvant, diluent and/or carrier.

29. The pharmaceutical composition of claim 28, wherein the composition comprises an isolated peptide according to claim 24 or 25, a nucleic acid sequence according to claim 26, or a vector system according to claim 27, wherein the pharmaceutical composition is formulated as a vaccine.

30. A pharmaceutical composition according to claim 28 or 29 for use in inducing or enhancing an immune response in human subject diagnosed with a B cell associated disease or condition.

31. A pharmaceutical composition according to claim 28 or 29 for use in stimulating a cell mediated immune response to a target cell population or tissue in a human subject.

32. A pharmaceutical composition according to claim 28 or 29 for use in providing anti-tumor immunity to a human subject.

33. A pharmaceutical composition according to claim 28 or 29 for use in treating an human subject having a disease or condition associated with an elevated level of HLA-restricted Jchain antigen.

34. The pharmaceutical composition for use according to any of claims 30 to 33 wherein the human subject has at least one tumor.

35. The pharmaceutical composition for use according to any of claims 31 to 34, wherein the subject has been diagnosed with a B cell associated disease or condition.

36. The pharmaceutical composition for use according to claim 30 or 35, wherein the B cell associated disease or condition is a hematological malignancy or an autoimmune disease or disorder.

37. The pharmaceutical composition for use according to claim 36, wherein the hematological malignancy is selected from the group consisting of: Multiple myeloma, plasma cell leukemia, (AL) Amyloidosis, Acute lymphoblastoid leukemia (ALL), Chronic lymphocytic leukemia (CLL), Waldenstrom macroglobulinemia and B cell lymphoma, optionally wherein the B cell lymphoma is selected from the group consisting of: Diffuse large B cell lymphoma (DLBCL), High grade B cell lymphoma, Mantel cell lymphoma (MCL), Follicular lymphoma (FL), and Burkitt Lymphoma.

38. The pharmaceutical composition for use according to claim 36, wherein the hematological malignancy is multiple myeloma.

39. The pharmaceutical composition for use according to claim 36, wherein the autoimmune disease or disorder is selected from the group consisting of: Rheumatoid arthritis, Multiple sclerosis, Vasculitis including Urticarial vasculitis, systemic vasculitis, renal vasculitis, Systemic lupus erythematosus (SLE), Autoimmune hemolytic anemia and Thrombocytopenia.

40. A method of generating a binding protein that is capable of specifically binding to a peptide containing a Jchain antigen and does not bind to a peptide that does not contain the Jchain antigen, comprising contacting a nucleic acid composition according to any of claims 1 to 18 with a cell under conditions in which the nucleic acid composition is incorporated and expressed by the cell.

41. The method of claim 40, wherein the method is ex vivo.

42. An isolated nucleic acid sequence comprising or consisting of the nucleotide sequence of any one of SEQ ID NOs: 8, 10, 12, 14, 22, 24, 26, 28, 36, 38, 40, 42, 50, 52, 54, 56, 64, 66, 68, 70, 78, 80, 82, 84, 92, 94, 96 or 98.

43. An isolated nucleic acid sequence comprising or consisting of the nucleotide sequence of any one of SEQ ID NOs: 8, 10, 12, 14, 22, 24, 26, 28, 36, 38, 40, 42, 50, 52, 54, 56, 64, 66, 68, 70, 78, 80, 82, 84, 92, 94, 96 or 98 for use in therapy.


SEQUENCE LISTING (107 sequences)

SEQ ID NO: 1 - protein, 7 aa, Homo sapiens
TSENNYY

SEQ ID NO: 2 - protein, 8 aa, Homo sapiens
QEAYKQQN

SEQ ID NO: 3 - protein, 15 aa, Homo sapiens
CAFMKHVEYGNKLVF

SEQ ID NO: 4 - protein, 5 aa, Homo sapiens
ENHRY

SEQ ID NO: 5 - protein, 6 aa, Homo sapiens
SYGVKD

SEQ ID NO: 6 - protein, 13 aa, Homo sapiens
CAARDSANEKLFF

SEQ ID NO: 7 - protein, 136 aa, Homo sapiens
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMKHVEYGNKLVFGAGTILRVKS

SEQ ID NO: 8 - DNA, 408 bp, Homo sapiens
  source 1..408: mol_type = genomic DNA
atgacacgagttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcatgaagcatgtggaatatggaaacaaactggtctttggcgcaggaaccattctgagagtcaagtcc

SEQ ID NO: 9 - protein, 131 aa, Homo sapiens
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAARDSANEKLFFGSGTQLSVL

SEQ ID NO: 10 - DNA, 393 bp, Homo sapiens
  source 1..393: mol_type = genomic DNA
atgggcacaaggttgttcttctatgtggccctttgtctcctgtggacaggacacatggatgctggaatcacccagagcccaagacacaaggtcacagagacaggaacaccagtgactctgagatgtcatcagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccgcacgggacagcgctaatgaaaaactgttttttggcagtggaacccagctctctgtcttg

SEQ ID NO: 11 - protein, 277 aa, synthetic construct
  REGION 1..277: note = TCR V alpha domain with an constant domain
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMKHVEYGNKLVFGAGTILRVKSYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 12 - DNA, 834 bp, synthetic construct
  misc_feature 1..834: note = TCR alpha chain of clone 4G8.8
  source 1..834: mol_type = other DNA
atgacacgagttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcatgaagcatgtggaatatggaaacaaactggtctttggcgcaggaaccattctgagagtcaagtcctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 13 - protein, 308 aa, synthetic construct
  REGION 1..308: note = TCR V beta domain and a constant domain
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAARDSANEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 14 - DNA, 927 bp, synthetic construct
  misc_feature 1..927: note = TCR beta chain of clone 4G8.8
  source 1..927: mol_type = other DNA
atgggcacaaggttgttcttctatgtggccctttgtctcctgtggacaggacacatggatgctggaatcacccagagcccaagacacaaggtcacagagacaggaacaccagtgactctgagatgtcatcagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccgcacgggacagcgctaatgaaaaactgttttttggcagtggaacccagctctctgtcttggaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 15 - protein, 8 aa, Homo sapiens
DRGSQSFF

SEQ ID NO: 16 - protein, 6 aa, Homo sapiens
IYSNGD

SEQ ID NO: 17 - protein, 12 aa, Homo sapiens
CAVMDSNYQLIW

SEQ ID NO: 18 - protein, 5 aa, Homo sapiens
SGHVS

SEQ ID NO: 19 - protein, 6 aa, Homo sapiens
FQNEAQ

SEQ ID NO: 20 - protein, 14 aa, Homo sapiens
CASSPGEFGETQYF

SEQ ID NO: 21 - protein, 131 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVMDSNYQLIWGAGTKLIIKP

SEQ ID NO: 22 - DNA, 393 bp, Homo sapiens
  source 1..393: mol_type = genomic DNA
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtaatggatagcaactatcagttaatctggggcgctgggaccaagctaattataaagcca

SEQ ID NO: 23 - protein, 133 aa, Homo sapiens
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSPGEFGETQYFGPGTRLLVL

SEQ ID NO: 24 - DNA, 399 bp, Homo sapiens
  source 1..399: mol_type = genomic DNA
atgggcaccaggctcctctgctgggtggtcctgggtttcctagggacagatcacacaggtgctggagtctcccagtcccctaggtacaaagtcgcaaagagaggacaggatgtagctctcaggtgtgatccaatttcgggtcatgtatcccttttttggtaccaacaggccctcgggcaggggccagagtttctgacttatttccagaatgaagctcaactagacaaatcggggctgcccagtgatcgcttctttgcagaaaggcctgagggatccgtctccactctgaagatccagcgcacacagcaggaggactccgccgtgtatctctgtgccagcagcccgggggaatttggggagacccagtacttcgggccaggcacgcggctcctggtgctc

SEQ ID NO: 25 - protein, 272 aa, synthetic construct
  REGION 1..272: note = TCR V alpha domain with a constant domain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVMDSNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 26 - DNA, 819 bp, synthetic construct
  misc_feature 1..819: note = TCR alpha chain of clone 5D12.9
  source 1..819: mol_type = other DNA
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtaatggatagcaactatcagttaatctggggcgctgggaccaagctaattataaagccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 27 - protein, 310 aa, synthetic construct
  REGION 1..310: note = TCR V beta domain and a constant domain
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSPGEFGETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 28 - DNA, 933 bp, synthetic construct
  misc_feature 1..933: note = TCR beta chain of clone 5D12.9
  source 1..933: mol_type = other DNA
atgggcaccaggctcctctgctgggtggtcctgggtttcctagggacagatcacacaggtgctggagtctcccagtcccctaggtacaaagtcgcaaagagaggacaggatgtagctctcaggtgtgatccaatttcgggtcatgtatcccttttttggtaccaacaggccctcgggcaggggccagagtttctgacttatttccagaatgaagctcaactagacaaatcggggctgcccagtgatcgcttctttgcagaaaggcctgagggatccgtctccactctgaagatccagcgcacacagcaggaggactccgccgtgtatctctgtgccagcagcccgggggaatttggggagacccagtacttcgggccaggcacgcggctcctggtgctcgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 29 - protein, 6 aa, Homo sapiens
VSGLRG

SEQ ID NO: 30 - protein, 7 aa, Homo sapiens
LYSAGEE

SEQ ID NO: 31 - protein, 13 aa, Homo sapiens
CAVLSGGSYIPTF

SEQ ID NO: 32 - protein, 5 aa, Homo sapiens
SEHNR

SEQ ID NO: 33 - protein, 6 aa, Homo sapiens
FQNEAQ

SEQ ID NO: 34 - protein, 16 aa, Homo sapiens
CASSPLLRDSEEKLFF

SEQ ID NO: 35 - protein, 131 aa, Homo sapiens
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVLSGGSYIPTFGRGTSLIVHP

SEQ ID NO: 36 - DNA, 393 bp, Homo sapiens
  source 1..393: mol_type = genomic DNA
atggagaaaatgttggagtgtgcattcatagtcttgtggcttcagcttggctggttgagtggagaagaccaggtgacgcagagtcccgaggccctgagactccaggagggagagagtagcagtcttaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgttatcaggaggaagctacatacctacatttggaagaggaaccagccttattgttcatccg

SEQ ID NO: 37 - protein, 135 aa, Homo sapiens
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSPLLRDSEEKLFFGSGTQLSVL

SEQ ID NO: 38 - DNA, 405 bp, Homo sapiens
  source 1..405: mol_type = genomic DNA
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagccccctcctccgggacagcgaagaaaaactgttttttggcagtggaacccagctctctgtcttg

SEQ ID NO: 39 - protein, 272 aa, synthetic construct
  REGION 1..272: note = TCR V alpha domain with a constant domain
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVLSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 40 - DNA, 819 bp, synthetic construct
  misc_feature 1..819: note = TCR alpha chain of clone 10H11.11
  source 1..819: mol_type = other DNA
atggagaaaatgttggagtgtgcattcatagtcttgtggcttcagcttggctggttgagtggagaagaccaggtgacgcagagtcccgaggccctgagactccaggagggagagagtagcagtcttaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgttatcaggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 41 - protein, 312 aa, synthetic construct
  REGION 1..312: note = TCR V beta domain and a constant domain
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSPLLRDSEEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 42 - DNA, 939 bp, synthetic construct
  misc_feature 1..939: note = TCR beta chain of clone 10H11.11
  source 1..939: mol_type = other DNA
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagccccctcctccgggacagcgaagaaaaactgttttttggcagtggaacccagctctctgtcttggaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 43 - protein, 6 aa, Homo sapiens
SSYSPS

SEQ ID NO: 44 - protein, 8 aa, Homo sapiens
YTSAATLV

SEQ ID NO: 45 - protein, 14 aa, Homo sapiens
CVVKWADSWGKLQF

SEQ ID NO: 46 - protein, 5 aa, Homo sapiens
PRHDT

SEQ ID NO: 47 - protein, 6 aa, Homo sapiens
FYEKMQ

SEQ ID NO: 48 - protein, 15 aa, Homo sapiens
CASSLGTGNFYEQYF

SEQ ID NO: 49 - protein, 133 aa, Homo sapiens
MLLLLVPVLEVIFTLGGTRAQSVTQLDSHVSVSEGTPVLLRCNYSSSYSPSLFWYVQHPNKGLQLLLKYTSAATLVKGINGFEAEFKKSETSFHLTKPSAHMSDAAEYFCVVKWADSWGKLQFGAGTQVVVTP

SEQ ID NO: 50 - DNA, 399 bp, Homo sapiens
  source 1..399: mol_type = genomic DNA
atgctcctgctgctcgtcccagtgctcgaggtgatttttactctgggaggaaccagagcccagtcggtgacccagcttgacagccacgtctctgtctctgaaggaaccccggtgctgctgaggtgcaactactcatcttcttattcaccatctctcttctggtatgtgcaacaccccaacaaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgttgtgaaatgggctgacagctgggggaaattgcagtttggagcagggacccaggttgtggtcacccca

SEQ ID NO: 51 - protein, 143 aa, Homo sapiens
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGTGNFYEQYFGPGTRLTVT

SEQ ID NO: 52 - DNA, 429 bp, Homo sapiens
  source 1..429: mol_type = genomic DNA
atgcttagtcctgacctgcctgactctgcctggaacaccaggctcctctgccatgtcatgctttgtctcctgggagcagtttcagtggctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagcttaggaacagggaatttttacgagcagtacttcgggccgggcaccaggctcacggtcaca

SEQ ID NO: 53 - protein, 274 aa, synthetic construct
  REGION 1..274: note = TCR V alpha domain with a constant domain
MLLLLVPVLEVIFTLGGTRAQSVTQLDSHVSVSEGTPVLLRCNYSSSYSPSLFWYVQHPNKGLQLLLKYTSAATLVKGINGFEAEFKKSETSFHLTKPSAHMSDAAEYFCVVKWADSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 54 - DNA, 825 bp, synthetic construct
  misc_feature 1..825: note = TCR alpha chain of clone 5C8.16
  source 1..825: mol_type = other DNA
atgctcctgctgctcgtcccagtgctcgaggtgatttttactctgggaggaaccagagcccagtcggtgacccagcttgacagccacgtctctgtctctgaaggaaccccggtgctgctgaggtgcaactactcatcttcttattcaccatctctcttctggtatgtgcaacaccccaacaaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgttgtgaaatgggctgacagctgggggaaattgcagtttggagcagggacccaggttgtggtcaccccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 55 - protein, 320 aa, synthetic construct
  REGION 1..320: note = TCR V beta domain and a constant domain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGTGNFYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 56 - DNA, 963 bp, synthetic construct
  misc_feature 1..963: note = TCR beta chain of clone 5C8.16
  source 1..963: mol_type = other DNA
atgcttagtcctgacctgcctgactctgcctggaacaccaggctcctctgccatgtcatgctttgtctcctgggagcagtttcagtggctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagcttaggaacagggaatttttacgagcagtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 57 - protein, 7 aa, Homo sapiens
TSDPSYG

SEQ ID NO: 58 - protein, 8 aa, Homo sapiens
QGSYDQQN

SEQ ID NO: 59 - protein, 15 aa, Homo sapiens
CAMREDYTGGFKTIF

SEQ ID NO: 60 - protein, 5 aa, Homo sapiens
SGHTA

SEQ ID NO: 61 - protein, 6 aa, Homo sapiens
FQGTGA

SEQ ID NO: 62 - protein, 14 aa, Homo sapiens
CASSLISGGNEQFF

SEQ ID NO: 63 - protein, 136 aa, Homo sapiens
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMREDYTGGFKTIFGAGTRLFVKA

SEQ ID NO: 64 - DNA, 408 bp, Homo sapiens
  source 1..408: mol_type = genomic DNA
atgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatccaagttatggtctattctggtacaagcagcccagcagtggggaaatgatttttcttatttatcaggggtcttatgaccagcaaaatgcaacagaaggtcgctactcattgaatttccagaaggcaagaaaatccgccaaccttgtcatctccgcttcacaactgggggactcagcaatgtacttctgtgcaatgagagaggactatactggaggcttcaaaactatctttggagcaggaacaagactatttgttaaagca

SEQ ID NO: 65 - protein, 133 aa, Homo sapiens
MGTRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSLISGGNEQFFGPGTRLTVL

SEQ ID NO: 66 - DNA, 399 bp, Homo sapiens
  source 1..399: mol_type = genomic DNA
atgggcaccaggctcctctgctgggcagccctgtgcctcctgggggcagatcacacaggtgctggagtctcccagacccccagtaacaaggtcacagagaagggaaaatatgtagagctcaggtgtgatccaatttcaggtcatactgccctttactggtaccgacaaagcctggggcagggcccagagtttctaatttacttccaaggcacgggtgcggcagatgactcagggctgcccaacgatcggttctttgcagtcaggcctgagggatccgtctctactctgaagatccagcgcacagagcggggggactcagccgtgtatctctgtgccagcagcttaatatccgggggcaatgagcagttcttcgggccagggacacggctcaccgtgcta

SEQ ID NO: 67 - protein, 277 aa, synthetic construct
  REGION 1..277: note = TCR V alpha domain with a constant domain
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMREDYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 68 - DNA, 834 bp, synthetic construct
  misc_feature 1..834: note = TCR alpha chain of clone 16C7.9
  source 1..834: mol_type = other DNA
atgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatccaagttatggtctattctggtacaagcagcccagcagtggggaaatgatttttcttatttatcaggggtcttatgaccagcaaaatgcaacagaaggtcgctactcattgaatttccagaaggcaagaaaatccgccaaccttgtcatctccgcttcacaactgggggactcagcaatgtacttctgtgcaatgagagaggactatactggaggcttcaaaactatctttggagcaggaacaagactatttgttaaagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 69 - protein, 310 aa, synthetic construct
  REGION 1..310: note = TCR V beta domain and a constant domain
MGTRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSLISGGNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 70 - DNA, 933 bp, synthetic construct
  misc_feature 1..933: note = TCR beta chain of clone 16C7.9
  source 1..933: mol_type = other DNA
atgggcaccaggctcctctgctgggcagccctgtgcctcctgggggcagatcacacaggtgctggagtctcccagacccccagtaacaaggtcacagagaagggaaaatatgtagagctcaggtgtgatccaatttcaggtcatactgccctttactggtaccgacaaagcctggggcagggcccagagtttctaatttacttccaaggcacgggtgcggcagatgactcagggctgcccaacgatcggttctttgcagtcaggcctgagggatccgtctctactctgaagatccagcgcacagagcggggggactcagccgtgtatctctgtgccagcagcttaatatccgggggcaatgagcagttcttcgggccagggacacggctcaccgtgctagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 71 - protein, 6 aa, Homo sapiens
DSASNY

SEQ ID NO: 72 - protein, 7 aa, Homo sapiens
IRSNVGE

SEQ ID NO: 73 - protein, 14 aa, Homo sapiens
CAASNSGGYNKLIF

SEQ ID NO: 74 - protein, 5 aa, Homo sapiens
KGHSY

SEQ ID NO: 75 - protein, 6 aa, Homo sapiens
FQNENV

SEQ ID NO: 76 - protein, 16 aa, Homo sapiens
CASSQGRGPNGEKLFF

SEQ ID NO: 77 - protein, 132 aa, Homo sapiens
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASNSGGYNKLIFGAGTRLAVHP

SEQ ID NO: 78 - DNA, 396 bp, Homo sapiens
  source 1..396: mol_type = genomic DNA
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtaactctggtggctacaataagctgatttttggagcagggaccaggctggctgtacaccca

SEQ ID NO: 79 - protein, 135 aa, Homo sapiens
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSQGRGPNGEKLFFGSGTQLSVL

SEQ ID NO: 80 - DNA, 405 bp, Homo sapiens
  source 1..405: mol_type = genomic DNA
atgagcccaatattcacctgcatcacaatcctttgtctgctggctgcaggttctcctggtgaagaagtcgcccagactccaaaacatcttgtcagaggggaaggacagaaagcaaaattatattgtgccccaataaaaggacacagttatgttttttggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctacgaagcttgaggattcagcagtgtatttttgtgccagcagccaagggagaggcccaaacggagaaaaactgttttttggcagtggaacccagctctctgtcttg

SEQ ID NO: 81 - protein, 273 aa, synthetic construct
  REGION 1..273: note = TCR V alpha domain with a constant domain
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASNSGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 82 - DNA, 822 bp, synthetic construct
  misc_feature 1..822: note = TCR alpha chain of clone 13D4.9
  source 1..822: mol_type = other DNA
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtaactctggtggctacaataagctgatttttggagcagggaccaggctggctgtacacccatatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 83 - protein, 312 aa, synthetic construct
  REGION 1..312: note = TCR V beta domain and a constant domain
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSQGRGPNGEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 84 - DNA, 939 bp, synthetic construct
  misc_feature 1..939: note = TCR beta chain of clone 13D4.9
  source 1..939: mol_type = other DNA
atgagcccaatattcacctgcatcacaatcctttgtctgctggctgcaggttctcctggtgaagaagtcgcccagactccaaaacatcttgtcagaggggaaggacagaaagcaaaattatattgtgccccaataaaaggacacagttatgttttttggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctacgaagcttgaggattcagcagtgtatttttgtgccagcagccaagggagaggcccaaacggagaaaaactgttttttggcagtggaacccagctctctgtcttggaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 85 - protein, 6 aa, Homo sapiens
YGGTVN

SEQ ID NO: 86 - protein, 8 aa, Homo sapiens
YFSGDPLV

SEQ ID NO: 87 - protein, 13 aa, Homo sapiens
CAASSGGYQKVTF

SEQ ID NO: 88 - protein, 5 aa, Homo sapiens
MDHEN

SEQ ID NO: 89 - protein, 6 aa, Homo sapiens
SYDVKM

SEQ ID NO: 90 - protein, 15 aa, Homo sapiens
CASSLRREVGDTQYF

SEQ ID NO: 91 - protein, 132 aa, Homo sapiens
MLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAASSGGYQKVTFGIGTKLQVIP

SEQ ID NO: 92 - DNA, 396 bp, Homo sapiens
  source 1..396: mol_type = genomic DNA
atgctcctgttgctcataccagtgctggggatgatttttgccctgagagatgccagagcccagtctgtgagccagcataaccaccacgtaattctctctgaagcagcctcactggagttgggatgcaactattcctatggtggaactgttaatctcttctggtatgtccagtaccctggtcaacaccttcagcttctcctcaagtacttttcaggggatccactggttaaaggcatcaagggctttgaggctgaatttataaagagtaaattctcctttaatctgaggaaaccctctgtgcagtggagtgacacagctgagtacttctgtgccgcctcttctgggggttaccagaaagttacctttggaattggaacaaagctccaagtcatccca

SEQ ID NO: 93 - protein, 133 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLRREVGDTQYFGPGTRLTVL

SEQ ID NO: 94 - DNA, 399 bp, Homo sapiens
  source 1..399: mol_type = genomic DNA
atgggaatcaggctcctgtgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtttaaggcgggaggtgggagatacgcagtattttggcccaggcacccggctgacagtgctc

SEQ ID NO: 95 - protein, 273 aa, synthetic construct
  REGION 1..273: note = TCR V alpha domain with a constant domain
MLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAASSGGYQKVTFGIGTKLQVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 96 - DNA, 822 bp, synthetic construct
  misc_feature 1..822: note = TCR alpha chain of clone 13F6.6
  source 1..822: mol_type = other DNA
atgctcctgttgctcataccagtgctggggatgatttttgccctgagagatgccagagcccagtctgtgagccagcataaccaccacgtaattctctctgaagcagcctcactggagttgggatgcaactattcctatggtggaactgttaatctcttctggtatgtccagtaccctggtcaacaccttcagcttctcctcaagtacttttcaggggatccactggttaaaggcatcaagggctttgaggctgaatttataaagagtaaattctcctttaatctgaggaaaccctctgtgcagtggagtgacacagctgagtacttctgtgccgcctcttctgggggttaccagaaagttacctttggaattggaacaaagctccaagtcatcccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 97 - protein, 310 aa, synthetic construct
  REGION 1..310: note = TCR V beta domain and a constant domain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLRREVGDTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 98 - DNA, 933 bp, synthetic construct
  misc_feature 1..933: note = TCR beta chain of clone 13F6.6
  source 1..933: mol_type = other DNA
atgggaatcaggctcctgtgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtttaaggcgggaggtgggagatacgcagtattttggcccaggcacccggctgacagtgctcgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 99 - protein, 9 aa, Homo sapiens
YTAVVPLVY

SEQ ID NO: 100 - protein, 9 aa, Homo sapiens
CYTAVVPLV

SEQ ID NO: 101 - protein, 11 aa, Homo sapiens
ISDPTSPLRTR

SEQ ID NO: 102 - protein, 8 aa, Homo sapiens
TAVVPLVY

SEQ ID NO: 103 - protein, 11 aa, Homo sapiens
VLAVFIKAVHV

SEQ ID NO: 104 - protein, 9 aa, Homo sapiens
VLAVFIKAV

SEQ ID NO: 105 - protein, 8 aa, Homo sapiens
YTAVVPLV

SEQ ID NO: 106 - protein, 9 aa, Homo sapiens
RIIVPLNNR

SEQ ID NO: 107 - protein, 10 aa, Homo sapiens
GETKMVETAL